(19) United States
(12) Patent Application Publication
(10) Pub. No.: US 2024/0302741 A1
(43) Pub. Date: Sep. 12, 2024

(54) RESIST COMPOSITION AND METHOD FOR FORMING RESIST PATTERN

(71) Applicant: TOKYO OHKA KOGYO CO., LTD., Kawasaki-shi (JP)

(72) Inventors: Shuichi Ishii, Kawasaki-shi (JP); Hiroki Kato, Kawasaki-shi (JP); KhanhTin Nguyen, Kawasaki-shi (JP); Takuya Ikeda, Kawasaki-shi (JP); Koshi Onishi, Kawasaki-shi (JP); Rin Odashima, Kawasaki-shi (JP); Tetsuo Fujinami, Kawasaki-shi (JP); Seiji Todoroki, Kawasaki-shi (JP); Ryo Kawatani, Kawasaki-shi (JP)

(21) Appl. No.: 18/568,071

(22) PCT Filed: Jun. 10, 2022
(86) PCT No.: PCT/JP2022/023477

§ 371 Date: Dec. 7, 2023

(30) Foreign Application Priority Data
Jun. 15, 2021 (JP) 2021-099658
Jun. 15, 2021 (JP) 2021-099663
Jun. 15, 2021 (JP) 2021-099669
Jun. 9, 2022 (JP) 2022-093920

(51) Int. Cl. G03F 7/004

(52) U.S. Cl. CPC G03F 7/0045

(57) ABSTRACT

A resist composition that contains a resin component having a constitutional unit containing an acid-dissociable group represented by General Formula (a01-r) below and contains an acid generator component containing a compound represented by General Formula (b0) below. In General Formula (a01-r), Ra⁰¹ and Ra⁰² represent a saturated aliphatic hydrocarbon group, Ra⁰¹ and Ra⁰² may be bonded to each other to form an alicyclic group. Ra⁰³ to Ra⁰⁵ represent an aliphatic hydrocarbon group and two or more of Ra⁰³ to Ra⁰⁵ may be bonded to each other to form an alicyclic group. In General Formula (b-0), X⁰ represents an iodine atom, R^{m} represents a hydroxy group, nb1 represents an integer in a range of 1 to 5, nb2 represents an integer in a range of 0 to 4, 1≤nb1+nb2≤5, Yb⁰ represents a divalent linking group, Vb⁰ represents an alkylene group, R⁰ represents a fluorinated alkyl group having 1 to 5 carbon atoms, and M^{m+} represents an m-valent organic cation

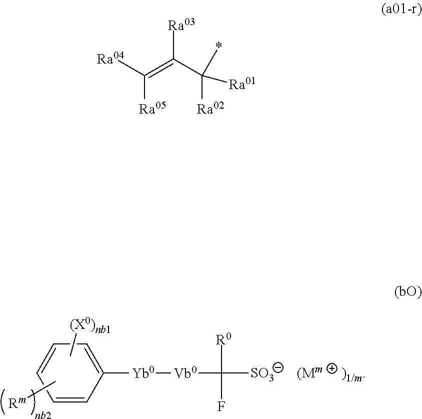

### TECHNICAL FIELD

The present invention relates to a resist composition and a method for forming a resist pattern.

Priority is claimed on Japanese Patent Application Nos. 2021-099658, 2021-099663, and 2021-099669, filed Jun. 15, 2021, and Japanese Patent Application No. 2022-093920 filed Jun. 9, 2022, the contents of which are incorporated herein by reference.

### BACKGROUND ART

In recent years, in the production of semiconductor elements and liquid crystal display elements, with advances in lithography techniques, rapid progress in the field of pattern fining has been achieved. Typically, these pattern fining techniques involve shortening the wavelength (increasing the energy) of the light source for exposure.

Resist materials have been required to have lithography characteristics such as sensitivity to these light sources for exposure and resolution capable of reproducing a fine-sized pattern.

As a resist material that satisfies these requirements, a chemical amplification-type resist composition that contains a resin component having solubility in a developing solution, which is changed under action of acid, and an acid generator component that generates acid upon exposure has been used in the related art.

In the chemical amplification-type resist composition, a resin having a plurality of constitutional units is generally used in order to improve lithography characteristics.

In addition, in the formation of the resist pattern, the behavior of an acid generated from an acid generator component upon exposure is also considered as one factor that has a great influence on lithography characteristics.

As the acid generator that is used in the chemical amplification-type resist composition, a wide variety of acid generators have been proposed so far. For example, onium salt-based acid generators such as an iodonium salt and a sulfonium salt, oxime sulfonate-based acid generators, diazomethane-based acid generators, nitrobenzyl sulfonate-based acid generators, iminosulfonate-based acid generators, and disulfonate-based acid generators are known.

For example, Patent Document 1 discloses a resist composition that contains a resin component having a tertiary alkyl ester-type acid-dissociable group containing an alicyclic group and a vinyl group and contains an onium salt-based acid generator.

For example, Patent Document 2 discloses a resist composition that contains a resin component having a repeating unit containing a structural moiety that is decomposed under action of acid to generate an alkali-soluble group and a structural moiety that is decomposed under action of an alkali developing solution to increase a dissolution rate in the alkali developing solution, and contains an onium salt-based acid generator.

For example, Patent Document 3 discloses a resist composition that contains a resin component having a tertiary alkyl ester-type acid-dissociable group containing an alicyclic group and an aromatic hydrocarbon group and contains an onium salt-based acid generator.

### CITATION LIST

### Patent Documents

### [Patent Document 1]

Japanese Unexamined Patent Application, First Publication No. 2017-3920

### [Patent Document 2]

Japanese Unexamined Patent Application, First Publication No. 2012-13835

### [Patent Document 3]

Japanese Unexamined Patent Application, First Publication No. 2016-133547

### SUMMARY OF INVENTION

### Technical Problem

With the further progress of lithography technology and resist pattern fining, for example, it is aimed to form a fine pattern of several tens of nanometers in lithography by EUV and EB. As the resist pattern dimensions become smaller, there is a demand for a resist composition that has high sensitivity and reduced roughness.

However, in the resist compositions in the related art, there is a trade-off relationship between sensitivity and roughness, and thus there is a problem in that the roughness is increased in a case where the sensitivity is improved, and the sensitivity is reduced in a case where the roughness is reduced. In addition, due to the fact that a fine pattern can be stably formed, the resist composition is also required to have a wide exposure margin. In addition, the resist composition is also required to be improved in resolution, pattern shape, and the like.

The present invention has been made in consideration of the above circumstances, and an object of the present invention is to provide a resist composition having good sensitivity and good roughness, and a method for forming a resist pattern using the resist composition.

In addition, another object of the present invention is to provide a resist composition having good sensitivity, good roughness, and a good exposure margin, and a method for forming a resist pattern using the resist composition.

In addition, another object of the present invention is to provide a resist composition having good sensitivity, good roughness, and good resolution, and a method for forming a resist pattern using the resist composition.

In addition, the present invention has been made in consideration of the above circumstances, and one of the objects of the present invention is to provide a resist composition having good sensitivity, good roughness, and good pattern shape, and a method for forming a resist pattern using the resist composition.

### Solution to Problem

In order to achieve the above-described objects, the present invention employs the following configurations.

That is, a first aspect according to the present invention is a resist composition that generates acid upon exposure and exhibits changed solubility in a developing solution under action of acid, which contains a resin component (A1) that exhibits changed solubility in a developing solution under action of acid and an acid generator component (B) that generates acid upon exposure, in which the resin component (A1) has a constitutional unit (a01) containing an acid-dissociable group represented by General Formula (a01-r) and the acid generator component (B) contains a compound (B0) represented by General Formula (b0).

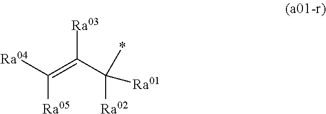

[In the formula, Ra⁰¹ and Ra⁰² each independently represents a saturated aliphatic hydrocarbon group which may have a substituent, and Ra⁰¹ and Ra⁰² may be bonded to each other to form an alicyclic group, where the alicyclic group may contain an oxygen atom or a sulfur atom in a ring skeleton, and part or all of hydrogen atoms contained in the alicyclic group may be substituted with a substituent. Ra⁰³ to Ra⁰⁵ each independently represents a hydrogen atom or an aliphatic hydrocarbon group which may have a substituent, two or more of Ra⁰³ to Ra⁰⁵ may be bonded to each other to form an alicyclic group, where a part or all of hydrogen atoms contained in the alicyclic group may be substituted with a substituent. * represents a bonding site.]

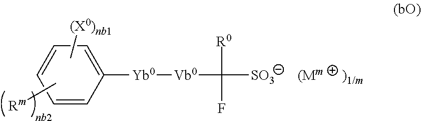

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. Yb⁰ represents a divalent linking group or a single bond. Vb⁰ represents a single bond, an alkylene group, or a fluorinated alkylene group. R⁰ represents a hydrogen atom, a fluorinated alkyl group having 1 to 5 carbon atoms, or a fluorine atom. M^{m+} represents an m-valent organic cation, where m represents an integer of 1 or more.]

A second aspect of the present invention is a resist composition that generates acid upon exposure and exhibits changed solubility in a developing solution under action of acid, the resist composition including a resin component (A1) that exhibits changed solubility in a developing solution under action of acid, and an acid generator component (B) that generates acid upon exposure, in which the resin component (A1) has a constitutional unit (a02) that contains a lactone-containing cyclic group containing an acid-dissociable group, an —SO₂—-containing cyclic group containing an acid-dissociable group, or a carbonate-containing cyclic group containing an acid-dissociable group, and the acid generator component (B) contains a compound (B0) represented by General Formula (b0).

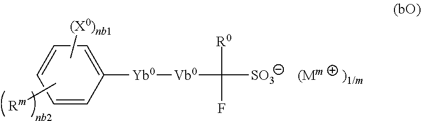

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. Yb⁰ represents a divalent linking group or a single bond. Vb⁰ represents a single bond, an alkylene group, or a fluorinated alkylene group. R⁰ represents a hydrogen atom, a fluorinated alkyl group having 1 to 5 carbon atoms, or a fluorine atom. M^{m+} represents an m-valent organic cation, where m represents an integer of 1 or more.]

A third aspect of the present invention is a resist composition that generates acid upon exposure and exhibits changed solubility in a developing solution under action of acid, the resist composition including a resin component (A1) that exhibits changed solubility in a developing solution under action of acid, and an acid generator component (B) that generates acid upon exposure, in which the resin component (A1) has a constitutional unit (a031) containing an acid-dissociable group represented by General Formula (a03-r1), and a constitutional unit (a032) containing an acid-dissociable group represented by General Formula (a03-r2), and the acid generator component (B) contains a compound (B0) represented by General Formula (b0).

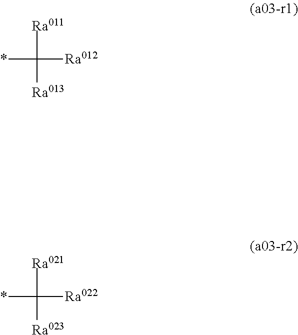

[In General Formula (a03-r1), Ra⁰¹¹ to Ra⁰¹³ each independently represents a saturated aliphatic hydrocarbon group which may have a substituent, where Ra⁰¹² and Ra⁰¹³ may be bonded to each other to form a ring.
in General Formula (a03-r2), Ra⁰²¹ represents a hydrocarbon group containing an aromatic ring, which may have a substituent, and Ra⁰²² and Ra⁰²³ each independently represents a hydrocarbon group which may have a substituent, where Ra⁰²² and Ra⁰²³ may be bonded to each other to form a ring.
* represents a bonding site.]

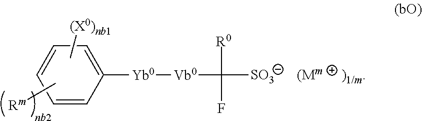

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. Yb⁰ represents a divalent linking group or a single bond. Vb⁰ represents a single bond, an alkylene group, or a fluorinated alkylene group. R⁰ represents a hydrogen atom, a fluorinated alkyl group having 1 to 5 carbon atoms, or a fluorine atom. M^{m+} represents an m-valent organic cation, where m represents an integer of 1 or more.]

A fourth aspect according to the present invention is a method for forming a resist pattern which includes a step of forming a resist film on a support using the resist composition according to any one of the first to third aspects, a step of exposing the resist film, and a step of developing the exposed resist film to form a resist pattern.

### Advantageous Effects of Invention

According to the present invention, it is possible to provide a resist composition having good sensitivity and good roughness, and a method for forming a resist pattern using the resist composition.

In addition, according to the first aspect of the present invention, it is possible to provide a resist composition having good sensitivity, good roughness, and a good exposure margin.

In addition, according to the second aspect of the present invention, it is possible to provide a resist composition having good sensitivity, good roughness, and good resolution.

In addition, according to the third aspect of the present invention, it is possible to provide a resist composition having good sensitivity, good roughness, and good pattern shape.

In addition, according to the fourth aspect of the present invention, it is possible to provide a method for forming a resist pattern using the resist composition according to any one of the first to third aspects.

### DESCRIPTION OF EMBODIMENTS

In the present specification and the scope of the present patent claims, the term “aliphatic” is a relative concept used with respect to the term “aromatic” and defines a group or compound that has no aromaticity.

The term “alkyl group” includes a monovalent saturated hydrocarbon group that is linear, branched, or cyclic unless otherwise specified. The same applies to the alkyl group of an alkoxy group.

The term “alkylene group” includes a divalent saturated hydrocarbon group that is linear, branched, or cyclic unless otherwise specified.

Examples of the “halogen atom” include a fluorine atom, a chlorine atom, a bromine atom, and an iodine atom.

The term “constitutional unit” means a monomer unit (a monomeric unit) that contributes to the formation of a polymeric compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “exposure” is used as a general concept that includes irradiation with any form of radiation.

The term “acid-decomposable group” is a group having acid decomposability, in which at least a part of bonds in the structure of the acid-decomposable group can be cleaved under action of acid.

Examples of the acid-decomposable group having a polarity that is increased under the action of an acid include groups that are decomposed under the action of an acid to generate a polar group.

Examples of the polar group include a carboxy group, a hydroxyl group, an amino group, and a sulfo group (—SO₃H).

More specific examples of the acid-decomposable group include a group (for example, a group obtained by protecting a hydrogen atom of the OH-containing polar group with an acid-dissociable group) obtained by protecting the above-described polar group with an acid-dissociable group.

The term “acid-dissociable group” refers to any one of (i) a group having acid decomposability, in which a bond between the acid-dissociable group and an atom adjacent to the acid-dissociable group can be cleaved under action of acid; and (ii) a group in which a part of bonds are cleaved under action of acid, and then a decarboxylation reaction occurs, thereby cleaving the bond between the acid-dissociable group and the atom adjacent to the acid-dissociable group.

It is necessary that the acid-dissociable group that constitutes the acid-decomposable group be a group that exhibits a lower polarity than the polar group generated by the dissociation of the acid-dissociable group. Thus, in a case where the acid-dissociable group is dissociated under action of acid, a polar group that exhibits a higher polarity than the acid-dissociable group is generated, thereby increasing the polarity. As a result of the above, the polarity of the total component (A1) is increased. With the increase in the polarity, the solubility in a developing solution relatively changes. The solubility in a developing solution is increased in a case where the developing solution is an alkali developing solution, whereas the solubility in a developing solution is decreased in a case where the developing solution is an organic developing solution.

The term “base material component” is an organic compound having a film-forming ability. The organic compounds used as the base material component are roughly classified into a non-polymer and a polymer. As the non-polymer, a compound (hereinafter referred to as a “low molecular weight compound”) having a molecular weight of 500 or more and less than 4,000 is usually used. As the polymer, those having a molecular weight of 1,000 or more are generally used. Hereinafter, a “resin”, a “polymeric compound”, or a “polymer” refers to a polymer having a molecular weight of 1,000 or more. As the molecular weight of the polymer, a weight-average molecular weight in terms of the polystyrene equivalent value determined by gel permeation chromatography (GPC) is used.

The term “constitutional unit derived from” means a constitutional unit that is formed by the cleavage of a multiple bond between carbon atoms, for example, an ethylenic double bond.

In the “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. In addition, itaconic acid diester in which the substituent (R^{αx}) has been substituted with a substituent having an ester bond or α-hydroxyacryl ester in which the substituent (R^{αx}) has been substituted with a hydroxyalkyl group or a group obtained by modifying a hydroxyl group thereof can be exemplified as an acrylic acid ester. A carbon atom at the α-position of acrylic acid ester indicates the carbon atom bonded to the carbonyl group of acrylic acid unless otherwise specified.

Hereinafter, acrylic acid ester in which the hydrogen atom bonded to the carbon atom at the α-position is substituted with a substituent is also referred to as an α-substituted acrylic acid ester.

The term “derivative” is used as a concept that includes a compound obtained by substituting a hydrogen atom at the α-position of an object compound with another substituent such as an alkyl group or a halogenated alkyl group; and a derivative thereof. Examples of the derivatives thereof include a derivative in which the hydrogen atom of the hydroxyl group of the object compound in which the hydrogen atom at the α-position may be substituted with a substituent is substituted with an organic group; and a derivative in which a substituent other than a hydroxyl group is bonded to the object compound in which the hydrogen atom at the α-position may be substituted with a substituent. The α-position refers to the first carbon atom adjacent to the functional group unless otherwise specified.

Examples of the substituent that substitutes the hydrogen atom at the α-position of hydroxystyrene include those for R^{αx}.

In the present specification and the scope of the present patent claims, asymmetric carbon atoms may be present, and thus enantiomers or diastereomers may be present depending on the structures represented by the chemical formula. In that case, these isomers are represented by one chemical formula. These isomers may be used alone or in the form of a mixture.

### (Resist Composition)

The resist composition according to the present embodiment is a resist composition that generates acid upon exposure and exhibits changed solubility in a developing solution under action of acid.

Such a resist composition contains a base material component (A) (hereinafter, also referred to as a “component (A)”) that exhibits changed solubility in a developing solution under action of acid, and an acid generator component (B) that generates acid upon exposure (hereinafter, also referred to as a “component (B)”).

In a case where a resist film is formed using the resist composition according to the present embodiment and the formed resist film is subjected to selective exposure, acid is generated from the component (B) at exposed portions of the resist film, and the generated acid acts on the component (A) to change the solubility of the component (A) in a developing solution, whereas the solubility of the component (A) in a developing solution is not changed at unexposed portions, thereby that generates the difference in solubility in the developing solution between exposed portions and unexposed portions of the resist film. Therefore, by subjecting the resist film to development, exposed portions of the resist film are dissolved and removed to form a positive-tone resist pattern in a case where the resist composition is a positive-tone type, whereas unexposed portions of the resist film are dissolved and removed to form a negative-tone resist pattern in a case where the resist composition is a negative-tone type.

In the present specification, a resist composition which forms a positive-tone resist pattern by dissolving and removing exposed portions of the resist film is called a positive-tone resist composition, and a resist composition which forms a negative-tone resist pattern by dissolving and removing unexposed portions of the resist film is called a negative-tone resist composition. The resist composition according to the present embodiment may be a positive-tone resist composition or a negative-tone resist composition. In addition, in the formation of a resist pattern, the resist composition according to the present embodiment can be applied to an alkali developing process using an alkali developing solution in the developing treatment, or a solvent developing process using a developing solution containing an organic solvent (organic developing solution) in the developing treatment.

In the resist composition according to the present embodiment, the component (A) contains a resin component (A1) (hereinafter, also referred to as a “component (A1)”) that exhibits changed solubility in a developing solution under action of acid.

Not only in the alkali developing process, but also in the solvent developing process, since the polarity of the base material component before and after the exposure is changed by using the component (A1), an excellent development contrast can be obtained.

As the component (A), another polymeric compound and/or a low molecular weight compound may be used in combination with the component (A1).

In a case of applying an alkali developing process, a base material component containing the component (A1) is insoluble in an alkali developing solution prior to exposure, and, for example, in a case where acid is generated from the component (B) upon exposure, the action of this acid causes an increase in the polarity of the base material component, thereby increasing the solubility of the base material component in an alkali developing solution. Therefore, in the resist pattern formation, in a case of carrying out selective exposure of a resist film formed by applying the resist composition onto a support, exposed portions of the resist film change from an insoluble state to a soluble state in an alkali developing solution, whereas unexposed portions of the resist film remain insoluble in an alkali developing solution, and thus, a positive-tone resist pattern is formed by alkali developing.

On the other hand, in a case of a solvent developing process, the base material component containing the component (A1) exhibits high solubility in an organic developing solution prior to exposure, and in a case where an acid is generated from the component (B) upon exposure, the polarity of the component (A1) is increased by the action of the generated acid, thereby decreasing the solubility of the component (A1) in an organic developing solution. Therefore, in the resist pattern formation, by carrying out selective exposure of a resist film formed by applying the resist composition onto a support, exposed portions of the resist film change from a soluble state to an insoluble state in an organic developing solution, whereas unexposed portions of the resist film remain soluble and do not change, thereby a contrast between exposed portions and unexposed portions can be obtained, and thus a negative-tone resist pattern is formed by developing in the organic developing solution.

In the resist composition according to the present embodiment, the component (A) may be used alone or in a combination of two or more kinds thereof.

### [Resist Composition According to First Aspect]

### <Component (A)>

### In Regard to Component (A1)

In the resist composition according to the first aspect, the component (A1) has a constitutional unit (a01) containing the acid-dissociable group represented by General Formula (a01-r).

<Constitutional Unit (a01)>

The constitutional unit (a01) is a constitutional unit containing the acid-dissociable group which is represented by General Formula (a01-r).

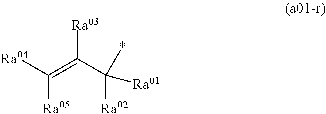

[In the formula, Ra⁰¹ and Ra⁰² each independently represents a saturated aliphatic hydrocarbon group which may have a substituent, and Ra⁰¹ and Ra⁰² may be bonded to each other to form an alicyclic group, where the alicyclic group may contain an oxygen atom or a sulfur atom in a ring skeleton, and part or all of hydrogen atoms contained in the alicyclic group may be substituted with a substituent. Ra⁰³ to Ra⁰⁵ each independently represents a hydrogen atom or an aliphatic hydrocarbon group which may have a substituent, two or more of Ra⁰³ to Ra⁰⁵ may be bonded to each other to form an alicyclic group, where part or all of hydrogen atoms contained in the alicyclic group may be substituted with a substituent. * represents a bonding site.]

In General Formula (a01-r), Ra⁰¹ and Ra⁰² each independently represents a saturated aliphatic hydrocarbon group which may have a substituent. Ra⁰¹ and Ra⁰² may be bonded to each other to form an alicyclic group. The alicyclic group may contain an oxygen atom or a sulfur atom in a ring skeleton. Part or all of hydrogen atoms contained in the alicyclic group may be substituted with a substituent.

Examples of the saturated aliphatic hydrocarbon group as Ra⁰¹ and Ra⁰² include a linear, branched, or cyclic alkyl group.

Examples of the linear alkyl group include a linear alkyl group having 1 to 15 carbon atoms. The linear alkyl group preferably has 1 to 12 carbon atoms, more preferably has 1 to 10 carbon atoms, still more preferably has 1 to 6 carbon atoms, and particularly preferably has 1 to 3 carbon atoms. Examples of the linear alkyl group include a methyl group, an ethyl group, an n-propyl group, an n-butyl group, and an n-pentyl group.

Examples of the branched alkyl group include a branched alkyl group having 3 to 15 carbon atoms. The branched alkyl group preferably has 3 to 12 carbon atoms, more preferably has 3 to 10 carbon atoms, still more preferably has 3 to 6 carbon atoms, and particularly preferably has 3 to 5 carbon atoms. Examples of the branched alkyl group include an isopropyl group, an isobutyl group, a tert-butyl group, an isopentyl group, a neopentyl group a 1,1-diethylpropyl group, and a 2,2-dimethylbutyl group.

The linear or branched alkyl group as Ra⁰¹ and Ra⁰² may have a substituent. Examples of the substituent include a halogen atom, a hydroxy group, a carboxy group, and an alkoxy group. In the linear or branched alkyl group as Ra⁰¹ and Ra⁰², part of methylene groups constituting the alkyl chain may be substituted with a hetero atom-containing group. Examples of the hetero atom include an oxygen atom, a nitrogen atom, and a sulfur atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

The cyclic alkyl group (the cycloalkyl group) may be a monocyclic group or may be a polycyclic group. Examples of the cyclic alkyl group include a cyclic alkyl group having 3 to 15 carbon atoms. The cyclic alkyl group preferably has 3 to 12 carbon atoms, more preferably has 3 to 10 carbon atoms, still more preferably has 3 to 6 carbon atoms, and particularly preferably has 5 or 6 carbon atoms.

Specific examples of the monocyclic cycloalkyl group include a cyclopropyl group, a cyclobutyl group, a cyclopentyl group, a cyclohexyl group, a cycloheptyl group, a cyclooctyl group, a cyclodecyl group, and a cyclododecyl group. Examples of the polycyclic cycloalkyl group include a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, an adamantyl group, a norbornyl group, and an isobornyl group.

The cyclic alkyl group as Ra⁰¹ and Ra⁰² may have a substituent. Examples of the substituent include a linear or branched alkyl group, a halogen atom, a hydroxy group, a carboxy group, and a linear or branched alkoxy group. The linear alkyl group or the linear alkoxy group, as the substituent, preferably has 1 to 5 carbon atoms, more preferably has 1 to 3 carbon atoms, and still more preferably has 1 or 2 carbon atoms. The branched alkyl group or the branched alkoxy group, as the substituent, preferably has 3 to 6 carbon atoms, more preferably has 3 to 5 carbon atoms, and still more preferably has 3 or 4 carbon atoms.

Ra⁰¹ and Ra⁰² may be bonded to each other to form an alicyclic group. Examples of the alicyclic group that is formed by bonding Ra⁰¹ and Ra⁰² to each other include an alicyclic group having 3 to 20 carbon atoms. The alicyclic group may be a monocyclic group or may be a polycyclic group.

Examples of the monocyclic alicyclic group include a group in which one hydrogen atom has been removed from a monocycloalkane. The monocycloalkane preferably has 3 to 10 carbon atoms, more preferably has 4 to 8 carbon atoms, and still more preferably has 5 or 6 carbon atoms. Specific examples of the monocycloalkane include cyclopropane, cyclobutane, cyclopentane, cyclohexane, cycloheptane, and cyclooctane. Among them, cyclopentane or cyclohexane are preferable, and cyclopentane is more preferable.

Examples of the polycyclic alicyclic group include a group obtained by removing one hydrogen atom from a polycycloalkane. The polycycloalkane preferably has 7 to 15 carbon atoms, more preferably has 7 to 12 carbon atoms, and still more preferably has 7 to 10 carbon atoms. Specific examples of the polycycloalkane include adamantane, norbornane, isobornane, tricyclodecane, and tetracyclododecane. Among them, norbornane is preferable.

The alicyclic group that is formed by bonding Ra⁰¹ and Ra⁰² to each other may have an oxygen atom or a sulfur atom in the ring skeleton. That is, part of carbon atoms in the ring skeleton of the alicyclic group may be substituted with a substituent containing an oxygen atom or a sulfur atom. Examples of the substituent containing an oxygen atom or a sulfur atom include —O—, —C(═O)—O—, —S—, —S(═O)₂—, and —S(═O)₂—O—. Among them, the substituent containing an oxygen atom or a sulfur atom is preferably —O— or —S—, and more preferably —O—. Examples of the alicyclic group containing an oxygen atom in the ring skeleton include a tetrahydrofuranyl group and a tetrahydropyranyl group. Examples of the alicyclic group having a sulfur atom in the ring skeleton include a tetrahydrothiophenyl group and a tetrahydrothiopyranyl group.

In the alicyclic group that is formed by bonding Ra⁰¹ and Ra⁰² to each other, part or all of hydrogen atoms may be substituted with a substituent. Examples of the substituent include —R^{P01}, —R^{P02}—O—R^{P01}, —R^{P02}—CO—R^{P01}, —R^{P02}—CO—OR^{P01}, —R^{P02}—O—CO—R^{P01}, —R^{P02}—OH, —R^{P02}—CN, and —R^{P02}—COOH (hereinafter, these substituents will be also collectively referred to as “Ra^{x05}”). R^{P01} represents a monovalent linear or branched saturated aliphatic hydrocarbon group having 1 to 10 carbon atoms. R^{P02} represents a single bond or a divalent linear or branched saturated aliphatic hydrocarbon group having 1 to 10 carbon atoms. However, part or all of hydrogen atoms contained in the chain-like saturated aliphatic hydrocarbon group as R^{P01} and R^{P02}, the aliphatic cyclic saturated hydrocarbon group, and the aromatic hydrocarbon group may be substituted with a fluorine atom. The alicyclic group that is formed by bonding Ra⁰¹ and Ra⁰² to each other may contain, as a single kind, one or more substituents of the above-described substituents, or may contain one or more substituents of each of two or more kinds of the above-described substituents.

The monovalent linear saturated aliphatic hydrocarbon group having 1 to 10 carbon atoms R^{P01} preferably has 1 to 6 carbon atoms, more preferably has 1 to 3 carbon atoms, and still more preferably has 1 or 2 carbon atoms. The monovalent branched saturated aliphatic hydrocarbon group having 1 to 10 carbon atoms R^{P01} preferably has 3 to 6 carbon atoms, more preferably has 3 or 4 carbon atoms, and still more preferably has 3 carbon atoms. Specific examples of R^{P01} include a methyl group, an ethyl group, a propyl group, a butyl group, a pentyl group, a hexyl group, a heptyl group, an octyl group, and a decyl group.

The divalent linear saturated aliphatic hydrocarbon group having 1 to 10 carbon atoms R^{P02} preferably has 1 to 6 carbon atoms, more preferably has 1 to 3 carbon atoms, and still more preferably has 1 or 2 carbon atoms. The monovalent branched saturated aliphatic hydrocarbon group having 1 to 10 carbon atoms R^{P02} preferably has 2 to 6 carbon atoms, more preferably has 2 to 4 carbon atoms, and still more preferably has 2 or 3 carbon atoms. Specific examples of R^{P02} include a single bond, a methylene group, an ethylene group, a propylene group, a butylene group, a pentylene group, a hexylene group, a heptylene group, an octylene group, and a decylene group.

It is preferable that Ra⁰¹ and Ra⁰² represent a linear or branched alkyl group or be bonded to each other to form an alicyclic group, and it is more preferable that Ra⁰¹ and Ra⁰² be bonded to each other to form an alicyclic group.

In Formula (a01-r), Ra⁰³ to Ra⁰⁵ each independently represents a hydrogen atom or an aliphatic hydrocarbon group which may have a substituent. Two or more of Ra⁰³ to Ra⁰⁵ may be bonded to each other to form an alicyclic group. Part or all of hydrogen atoms contained in the alicyclic group may be substituted with a substituent.

The aliphatic hydrocarbon group as Ra⁰³ to Ra⁰⁵ may be saturated or unsaturated; however, it is preferably saturated.

Examples of the saturated aliphatic hydrocarbon group as Ra⁰³ to Ra⁰⁵ include a linear, branched, or cyclic alkyl group. Examples of the saturated aliphatic hydrocarbon group as Ra⁰³ to Ra⁰⁵ include the same as those described as the saturated aliphatic hydrocarbon group as Ra⁰¹ and Ra⁰².

In Ra⁰³ to Ra⁰⁵, two or more thereof may be bonded to each other to form an alicyclic group.

In a case where Ra⁰³, Ra⁰⁴, and Ra⁰⁵, or Ra⁰³ and either Ra⁰⁴ or Ra⁰⁵ are bonded to each other to form an alicyclic group, the alicyclic group contains a carbon-carbon double bond represented by General Formula (a01-r) in the ring structure. Examples of the alicyclic group having a carbon-carbon double bond in the ring structure include an alicyclic group having 3 to 20 carbon atoms. The alicyclic group containing a carbon-carbon double bond in the ring structure preferably has 4 to 12 carbon atoms, more preferably has 5 to 10 carbon atoms, still more preferably has 5 to 8 carbon atoms, and particularly preferably has 5 or 6 carbon atoms. The alicyclic group may be a monocyclic group or may be a polycyclic group; however, it is preferably a monocyclic group. Examples of the alicyclic group include a cyclopentenyl group, a cyclohexenyl group, a methylcyclopentenyl group, a methylcyclohexenyl group, a cyclopentylideneethenyl group, and a cyclohexylideneethenyl group. Among them, a cyclopentenyl group, a cyclohexenyl group, or a cyclopentylideneethenyl group is preferable from the viewpoint of easy synthesis.

In a case where Ra⁰⁴ and Ra⁰⁵ are bonded to each other to form an alicyclic group, examples of the alicyclic group include an alicyclic group having 3 to 20 carbon atoms. The alicyclic group may be a monocyclic group or may be a polycyclic group. Examples of the alicyclic group that is formed by bonding Ra⁰⁴ and Ra⁰⁵ to each other include the same one as the alicyclic group that is formed by bonding Ra⁰¹ and Ra⁰² to each other.

In the alicyclic group that is formed by bonding two or more of Ra⁰³ to Ra⁰⁵ to each other, part or all of hydrogen atoms contained in the alicyclic group may be substituted with a substituent. Examples of the substituent include Ra^{x05}.

It is preferable that Ra⁰³ be a hydrogen atom or be bonded to Ra⁰⁴ and/or Ra⁰⁵ to form an alicyclic group.

It is preferable that Ra⁰⁴ and Ra⁰⁵ represent a hydrogen atom or a linear or branched alkyl group or be bonded to Ra⁰³ to form an alicyclic group. The linear or branched alkyl group is preferably a linear alkyl group, more preferably a linear alkyl group having 1 to 3 carbon atoms, still more preferably a methyl group or an ethyl group, and even still more preferably a methyl group.

The alicyclic group that is formed by bonding two or more of Ra⁰³ to Ra⁰⁵ to each other is preferably a cyclopentenyl group or a cyclohexenyl group, and it is more preferably a cyclopentenyl group.

Specific examples of the acid-dissociable group represented by General Formula (a01-r) are shown below.

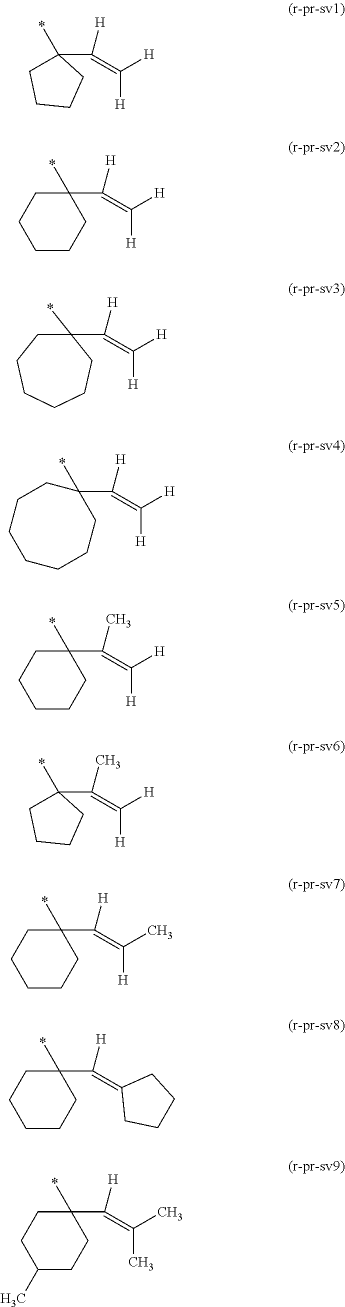

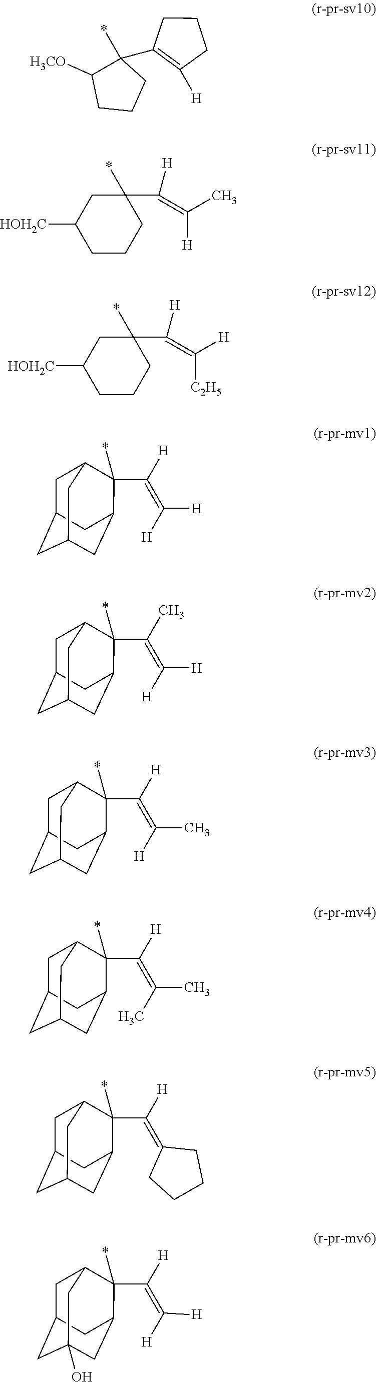

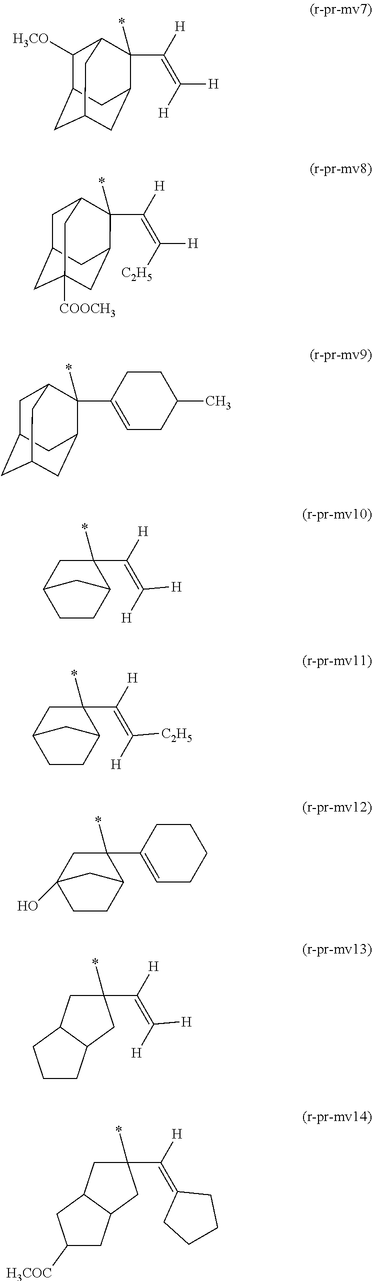

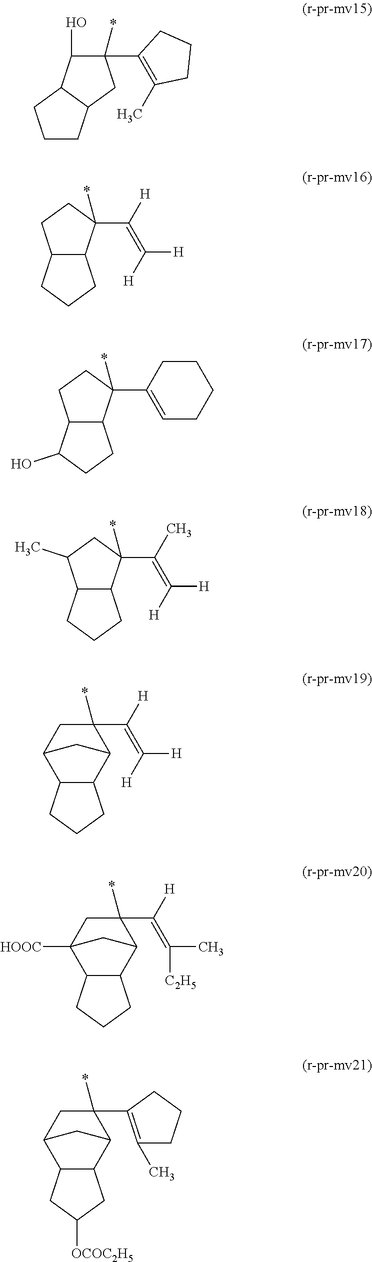

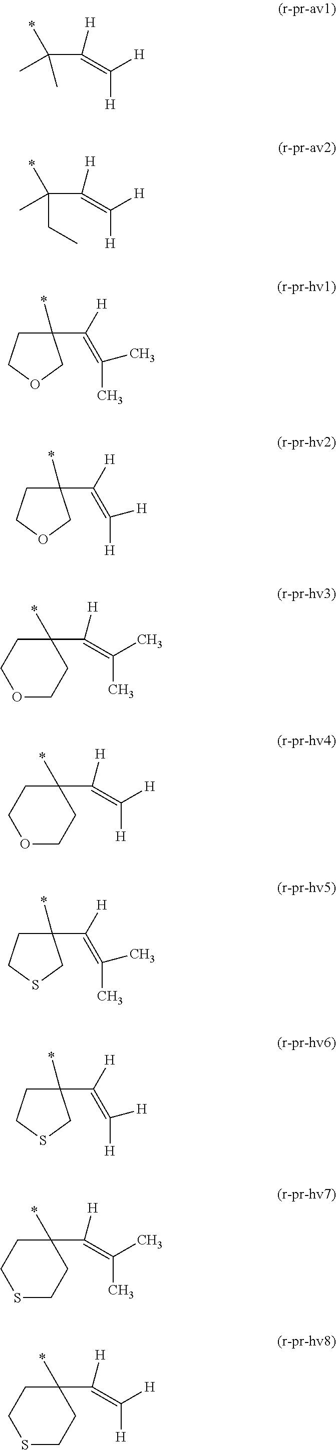

Examples of the constitutional unit (a01) include a constitutional unit represented by General Formula (a01-1).

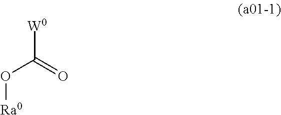

[In the formula, W⁰ represents a polymerizable group-containing group. Ra⁰ represents the acid-dissociable group represented by General Formula (a01-r).]

In General Formula (a01-1), W⁰ represents a polymerizable group-containing group.

The “polymerizable group” is a group that enables a compound having the polymerizable group to be polymerized by radical polymerization or the like, and it includes a group containing a multiple bond between carbon atoms, such as an ethylenic double bond. In the constitutional unit (a01), the multiple bonds in the polymerizable group are cleaved to form a main chain.

Examples of the polymerizable group include a vinyl group, an allyl group, acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, and a vinylnaphthyl group, a fluorine-containing styryl group, a fluorine-containing vinylnaphthyl group, a norbornyl group, a fluorine-containing norbornyl group, and a silyl group.

The term “polymerizable group-containing group” is a group containing a polymerizable group. The “polymerizable group-containing group” may be a group composed of only a polymerizable group, or a group composed of a polymerizable group and a group other than the polymerizable group. Examples of the group other than the polymerizable group include a divalent hydrocarbon group which may have a substituent and a divalent linking group containing a hetero atom.

Divalent Hydrocarbon Group which May have Substituent:

In a case where the group other than the polymerizable group represents a divalent hydrocarbon group which may have a substituent, the hydrocarbon group may be an aliphatic hydrocarbon group or an aromatic hydrocarbon group.

Aliphatic Hydrocarbon Group as a Group Other than the Polymerizable Group

The aliphatic hydrocarbon group indicates a hydrocarbon group that has no aromaticity. The aliphatic hydrocarbon group may be saturated or unsaturated. In general, the aliphatic hydrocarbon group is preferably saturated.

Examples of the aliphatic hydrocarbon group include a linear or branched aliphatic hydrocarbon group, and an aliphatic hydrocarbon group containing a ring in the structure thereof.

### Linear or Branched Aliphatic Hydrocarbon Group

The linear aliphatic hydrocarbon group described above preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group described above preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

The above linear or branched aliphatic hydrocarbon group may have or may not have a substituent. Examples of the substituent include a fluorine atom, a fluorinated alkyl group having 1 to 5 carbon atoms, which has been substituted with a fluorine atom, and a carbonyl group.

### Aliphatic Hydrocarbon Group Containing Ring in Structure Thereof

Examples of the aliphatic hydrocarbon group containing a ring in the structure thereof include a cyclic aliphatic hydrocarbon group which may have a substituent containing a hetero atom in the ring structure thereof (a group obtained by removing two hydrogen atoms from an aliphatic hydrocarbon ring), a group obtained by bonding the cyclic aliphatic hydrocarbon group to the terminal of a linear or branched aliphatic hydrocarbon group, and a group obtained by interposing the cyclic aliphatic hydrocarbon group in a linear or branched aliphatic hydrocarbon group. Examples of the above linear or branched aliphatic hydrocarbon group include the same as those described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane is preferably a monocycloalkane having 3 to 6 carbon atoms, and specific examples thereof include cyclopentane and cyclohexane. The polycyclic alicyclic hydrocarbon group is preferably a group obtained by removing two hydrogen atoms from a polycycloalkane, and the polycycloalkane preferably has 7 to 12 carbon atoms. Specific examples of the polycycloalkane include adamantane, norbornane, isobornane, tricyclodecane, and tetracyclododecane.

The cyclic aliphatic hydrocarbon group may have or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include a group obtained by substituting part or all of hydrogen atoms in the above-described alkyl groups with the above-described halogen atoms.

In the cyclic aliphatic hydrocarbon group, a part of carbon atoms constituting the ring structure thereof may be substituted with a substituent containing a hetero atom.

The substituent containing a hetero atom is preferably —O—, —C(═O)—O—, —S—, —S(═O)₂—, or —S(═O)₂—O—.

Aromatic Hydrocarbon Group as Group Other than the Polymerizable Group

The aromatic hydrocarbon group is a hydrocarbon group having at least one aromatic ring.

The aromatic ring is not particularly limited as long as it is a cyclic conjugated system having (4n+2) π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms in a substituent is not included in the number of carbon atoms in the aromatic ring. Specific examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The above-described alkylene group bonded to the aryl group or heteroaryl group preferably has 1 to 4 carbon atoms, more preferably has 1 or 2 carbon atoms, and particularly preferably has 1 carbon atom.

In the aromatic hydrocarbon group, the hydrogen atom contained in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and most preferably a methyl group, an ethyl group, a propyl group, an n-butyl group, or a tert-butyl group.

Examples of the alkoxy group, the halogen atom, and the halogenated alkyl group, as the substituent, include those exemplified as the substituent that is substituted for a hydrogen atom contained in the cyclic aliphatic hydrocarbon group.

### Divalent Linking Group Containing Hetero Atom

In a case where the group other than the polymerizable group represents a divalent linking group containing a hetero atom, preferred examples of the linking group include —O—, —C(═O)—O—, —C(═O)—, —O—C(═O)—O—, —C(═O)—NH—, —NH—, —NH—C(═NH)—(H may be substituted with a substituent such as an alkyl group, an acyl group, or the like), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²— or —Y²¹—S(═O)₂—O—Y²²— [in the formulae, Y²¹ and Y²² each independently represents a divalent hydrocarbon group which may have a substituent, O represents an oxygen atom, and m″ represents an integer in a range of 0 to 3].

In a case where the above divalent linking group containing a hetero atom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) preferably has 1 to 10 carbon atoms, more preferably has 1 to 8 carbon atoms, and particularly preferably has 1 to 5 carbon atoms.

In Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²², Y²¹ and Y²² each independently represents a divalent hydrocarbon group which may have a substituent. Examples of the divalent hydrocarbon group include the same one as “the divalent hydrocarbon group which may have a substituent” described in the explanation of the above-described divalent linking group.

Y²¹ is preferably a linear aliphatic hydrocarbon group, more preferably a linear alkylene group, still more preferably a linear alkylene group having 1 to 5 carbon atoms, and particularly preferably a methylene group or an ethylene group.

Y²² is preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer in a range of 0 to 3, and it is preferably an integer in a range of 0 to 2, more preferably 0 or 1, and particularly preferably 1. In other words, the group represented by Formula —[Y²¹—C(═O)—O]_{m″}—Y²²— is particularly preferably a group represented by Formula —Y²¹—C(═O)—O—Y²²—. Among the above, a group represented by Formula —(CH₂)_{a′}—C(═O)—O—(CH₂)_{b′}— is preferable. In the formula, a′ represents an integer in a range of 1 to 10, preferably an integer in a range of 1 to 8, more preferably an integer in a range of 1 to 5, still more preferably 1 or 2, and most preferably 1. b′ represents an integer in a range of 1 to 10, preferably an integer in a range of 1 to 8, more preferably an integer in a range of 1 to 5, still more preferably 1 or 2, and most preferably 1.

Suitable examples of the polymerizable group-containing group as W⁰ include a group represented by a chemical formula: C(R^{X11})(R^{X12})═C(R^{X13})—Ya^{x0}.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

The alkyl group having 1 to 5 carbon atoms as R^{X11}, R^{X12}, and R^{X13} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which part or all of hydrogen atoms in the alkyl group having 1 to 5 carbon atoms have been substituted with a halogen atom. The halogen atom is particularly preferably a fluorine atom.

Among these, R^{X11} and R^{X12} are each preferably a hydrogen atom, an alkyl group having 1 to 5 carbon atoms, or a fluorinated alkyl group having 1 to 5 carbon atoms, and in terms of industrial availability, a hydrogen atom or a methyl group is more preferable.

In addition, R^{X13} is preferably a hydrogen atom, an alkyl group having 1 to 5 carbon atoms, or a fluorinated alkyl group having 1 to 5 carbon atoms, and in terms of industrial availability, a hydrogen atom or a methyl group is more preferable.

The divalent linking group as Ya^{x0} is not particularly limited, and suitable examples thereof include a divalent hydrocarbon group which may have a substituent, and a divalent linking group having a hetero atom, each of which is the same as that described above.

Among the above, Ya^{x0} is preferably an ester bond [—C(═O)—O— or —O—C(═O)—], an ether bond (—O—), a linear or branched alkylene group, an aromatic hydrocarbon group, or a combination thereof, or a single bond. Among the above, Ya^{x0} is more preferably a group composed of a combination of an ester bond [—C(═O)—O— or —O—C(═O)—] and a linear alkylene group or a single bond. Specific examples of the group composed of a combination of an ester bond [—C(═O)—O—, —O—C(═O)—] and a linear alkylene group include —C(═O)—O—CH₂—.

In General Formula (a01-r), Ra⁰ represents the acid-dissociable group represented by General Formula (a01-1).

The constitutional unit (a01) is preferably a constitutional unit derived from an acrylic acid ester in which the hydrogen atom bonded to the carbon atom at the α-position may be substituted with a substituent. The constitutional unit (a01) is preferably a constitutional unit represented by General Formula (a01-1-1).

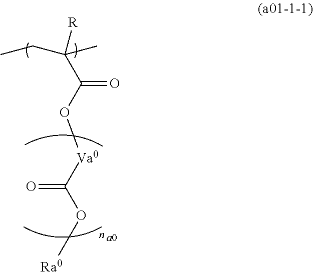

[In the formula, R represents a hydrogen atom, an alkyl group having 1 to 5 carbon atoms, or a halogenated alkyl group having 1 to 5 carbon atoms. Va⁰ represents a divalent hydrocarbon group which may have an ether bond. nₐ₀ represents an integer of 0 to 2, Ra⁰ represents the acid-dissociable group represented by General Formula (a01-r).]

In General Formula (a01-1-1), R represents a hydrogen atom, an alkyl group having 1 to 5 carbon atoms, or a halogenated alkyl group having 1 to 5 carbon atoms.

The alkyl group having 1 to 5 carbon atoms as R is preferably a linear or branched alkyl group having 1 to 5 carbon atoms. Specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which part or all of hydrogen atoms in the alkyl group having 1 to 5 carbon atoms have been substituted with a halogen atom. The halogen atom is preferably a fluorine atom.

R is preferably a hydrogen atom or a methyl group in terms of industrial availability.

In General Formula (a01-1-1), the divalent hydrocarbon group as Va⁰ may be an aliphatic hydrocarbon group or an aromatic hydrocarbon group.

The aliphatic hydrocarbon group may be saturated or unsaturated. In general, it is preferable that the aliphatic hydrocarbon group be saturated.

The aliphatic hydrocarbon group may be a linear aliphatic hydrocarbon group, may be a branched aliphatic hydrocarbon group, or may be an aliphatic hydrocarbon group containing a ring in the structure thereof.

The linear aliphatic hydrocarbon group preferably has 1 to 10 carbon atoms, more preferably has 1 to 6 carbon atoms, still more preferably has 1 to 4 carbon atoms, and particularly preferably has 1 to 3 carbon atoms.

The linear aliphatic hydrocarbon group is preferably a linear alkylene group. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group described above preferably has 2 to 10 carbon atoms, more preferably has 3 to 6 carbon atoms, still more preferably has 3 or 4 carbon atoms, and particularly preferably has 3 carbon atoms.

The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

Examples of the aliphatic hydrocarbon group containing a ring in the structure thereof include an alicyclic hydrocarbon group (a group obtained by removing two hydrogen atoms from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the linear or branched aliphatic hydrocarbon group. Examples of the linear or branched aliphatic hydrocarbon group include the same one as the above-described linear aliphatic hydrocarbon group or the above-described branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group obtained by removing two hydrogen atoms from a monocycloalkane. The monocycloalkane preferably has 3 to 6 carbon atoms. Specific examples thereof include cyclopentane and cyclohexane. The polycyclic alicyclic hydrocarbon group is preferably a group obtained by removing two hydrogen atoms from a polycycloalkane, and the polycycloalkane is preferably a group having 7 to 12 carbon atoms. Specific examples of the polycycloalkane include adamantane, norbornane, isobornane, tricyclodecane, and tetracyclododecane.

The aromatic hydrocarbon group as the divalent hydrocarbon group represented by Va⁰ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. Here, the number of carbon atoms in a substituent is not included in the number of carbon atoms in the aromatic hydrocarbon group.

Specific examples of the aromatic ring contained in the aromatic hydrocarbon group include aromatic hydrocarbon rings such as benzene, biphenyl, fluorene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom.

Examples of the aromatic hydrocarbon group include a group obtained by removing two hydrogen atoms from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group obtained by further removing one hydrogen atom from an aryl group in arylalkyl groups such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) preferably has 1 to 4 carbon atoms, more preferably has 1 or 2 carbon atoms, and still preferably has 1 carbon atom.

In General Formula (a01-1-1), nₐ₀ represents an integer in a range of 0 to 2. nₐ₀ is preferably 0 or 1 and more preferably 0.

In General Formula (a01-1-1), Ra⁰ represents the acid-dissociable group represented by General Formula (a01-r).

Specific examples of the constitutional unit (a01) are shown below. In each of the formulae shown below, R^{α} represents a hydrogen atom, a methyl group, or a trifluoromethyl group.

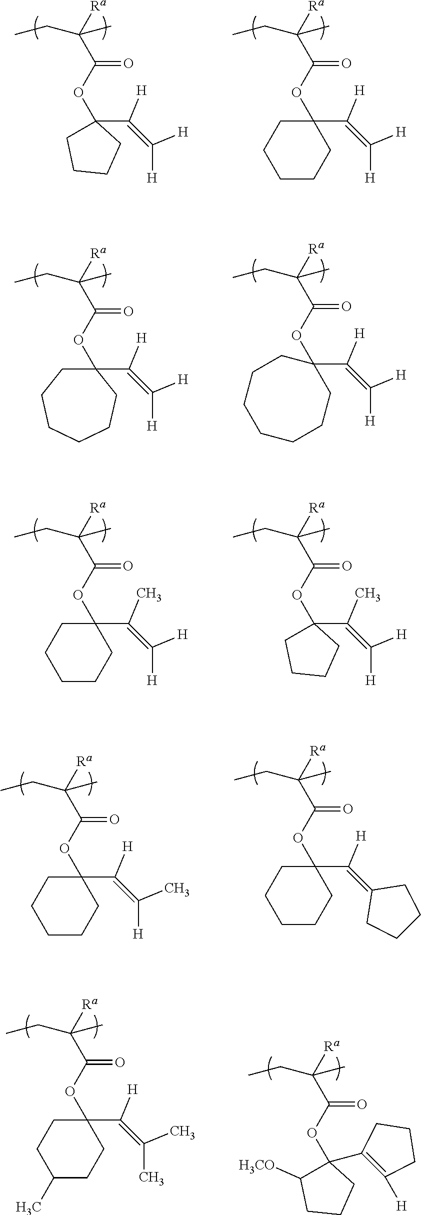

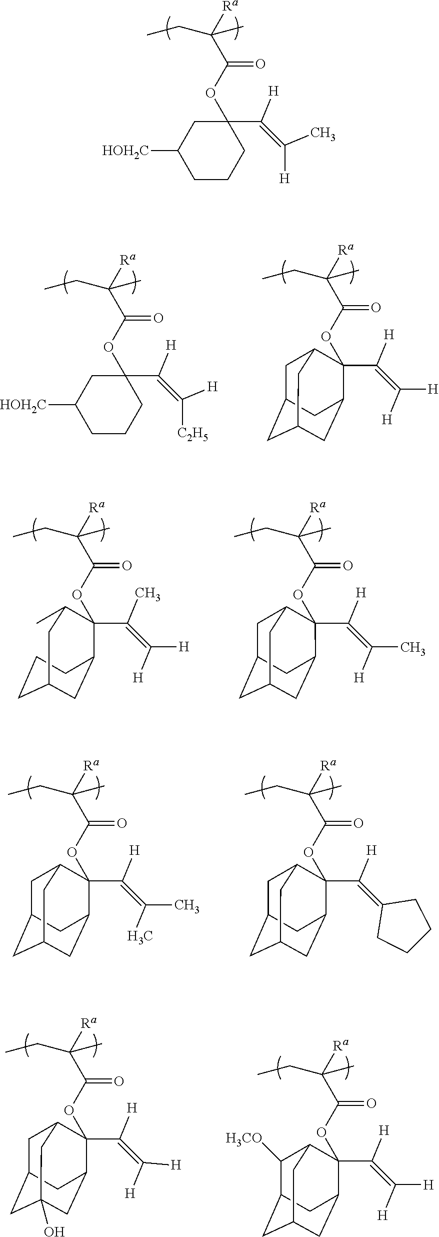

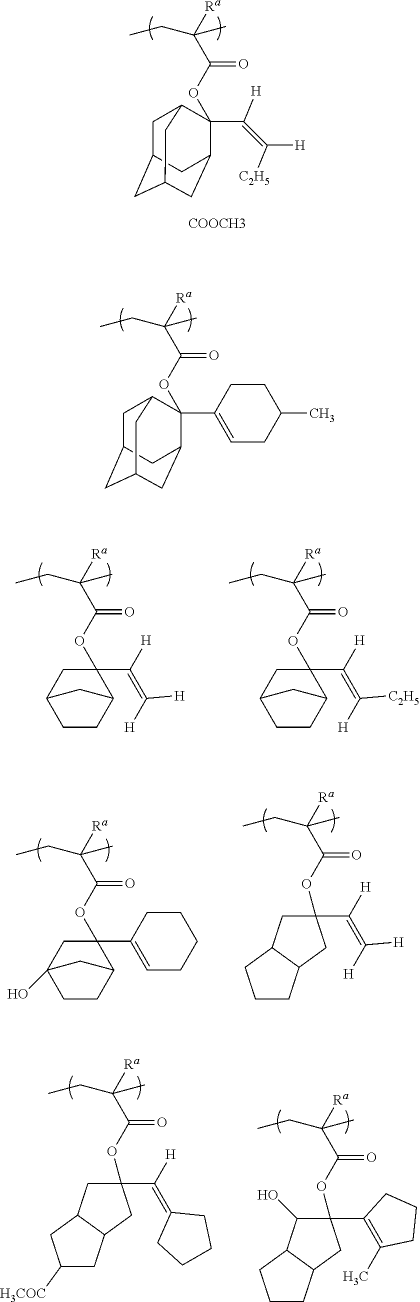

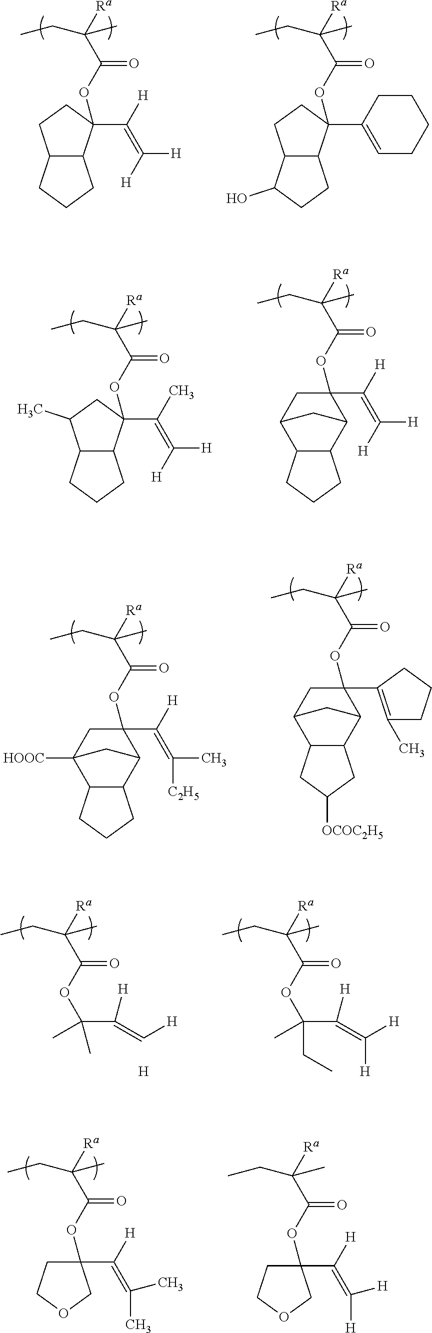

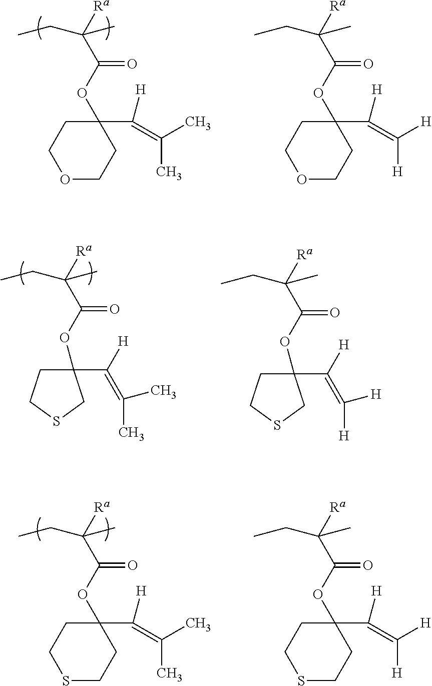

The constitutional unit (a01) may be one kind or may be two or more kinds.

The proportion of the constitutional unit (a01) in the component (A1) is preferably in a range of 10% to 80% by mole, more preferably in a range of 20% to 75% by mole, still more preferably in a range of 30% to 70% by mole, and particularly preferably in a range of 40% to 60% by mole, with respect to the total (100% by mole) of all constitutional units constituting the component (A1).

In a case where the proportion of the constitutional unit (a01) is equal to or larger than the lower limit value of the above-described preferred range, sensitivity is easily improved and roughness is easily reduced. On the other hand, in a case where the proportion of the constitutional unit (a01) is equal to or smaller than the upper limit value of the above-described preferred range, balance with other constitutional units can be obtained, and various lithography characteristics are improved.

### <<Other Constitutional Units>

The component (A1) may have other constitutional units as necessary in addition to the constitutional unit (a01) described above.

Examples of the other constitutional units include a constitutional unit (a1) containing an acid-dissociable group other than the acid-dissociable group represented by General Formula (a01-r); a constitutional unit (a2) containing a lactone-containing cyclic group, an —SO₂—-containing cyclic group, or a carbonate-containing cyclic group; a constitutional unit (a3) containing a polar group-containing aliphatic hydrocarbon group; a constitutional unit (a4) containing an acid non-dissociable aliphatic cyclic group; a constitutional unit (a10) represented by General Formula (a10-1) which will described later; and a constitutional unit (st) derived from styrene or a styrene derivative.

<Constitutional Unit (a1)>

The constitutional unit (a1) is a constitutional unit (however, a constitutional unit corresponding to the constitutional unit (a01) is excluded) containing an acid-decomposable group having a polarity which is increased under action of acid.

Examples of the acid-dissociable group include those which have been proposed so far as acid-dissociable groups for the base resin for a chemical amplification-type resist composition.

Specific examples of the acid-dissociable group of the base resin proposed for a chemical amplification-type resist composition include an “acetal-type acid-dissociable group”, a “tertiary alkyl ester-type acid-dissociable group”, and a “tertiary alkyloxycarbonyl acid-dissociable group” described below.

### Acetal-Type Acid-Dissociable Group:

Examples of the acid-dissociable group for protecting a carboxy group or a hydroxyl group as a polar group include the acid-dissociable group represented by General Formula (a1-r-1) shown below (hereinafter, also referred to as an “acetal-type acid-dissociable group”).

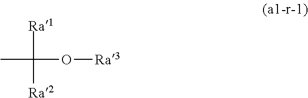

[In the formula, Ra′¹ and Ra′² represent a hydrogen atom or an alkyl group. Ra′³ represents a hydrocarbon group, and Ra′³ may be bonded to any one of Ra′¹ or Ra′² to form a ring.]

In General Formula (a1-r-1), it is preferable that at least one of Ra′¹ and Ra′² represent a hydrogen atom and more preferable that both of them represent hydrogen atoms.

In a case where Ra′¹ or Ra′² represents an alkyl group, examples of the alkyl group include the same one as the alkyl group described as the substituent which may be bonded to the carbon atom at the α-position in the description for the α-substituted acrylic acid ester, and the alkyl group preferably has 1 to 5 carbon atoms. Specific examples thereof preferably include a linear or branched alkyl group. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In General Formula (a1-r-1), examples of the hydrocarbon group as Ra′³ include a linear or branched alkyl group and a cyclic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among these, a methyl group. an ethyl group, or an n-butyl group is preferable, and a methyl group or an ethyl group is more preferable.

The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the cyclic hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane is preferably a monocycloalkane having 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as it is a cyclic conjugated system having (4n+2) π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms.

Specific examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group obtained by removing one hydrogen atom from the above-described aromatic hydrocarbon ring or aromatic heterocyclic ring (an aryl group or a heteroaryl group); a group obtained by removing one hydrogen atom from an aromatic compound having two or more aromatic rings (for example, biphenyl, fluorene); and a group obtained by substituting one hydrogen atom of the above-described aromatic hydrocarbon ring or aromatic heterocyclic ring with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring preferably has 1 to 4 carbon atoms, more preferably has 1 or 2 carbon atoms, and particularly preferably has 1 carbon atom.

The cyclic hydrocarbon group as Ra′³ may have a substituent. Examples of this substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will be also collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. In addition, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. However, part or all of hydrogen atoms contained in the chain-like saturated hydrocarbon group as R^{P1} and R^{P2}, the aliphatic cyclic saturated hydrocarbon group, and the aromatic hydrocarbon group may be substituted with a fluorine atom. In the aliphatic cyclic hydrocarbon group, one or more of the above-described substituents may be included as a single kind, or one or more of the above-described substituents may be included as a plurality of kinds.

Examples of the monovalent chain-like saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and cyclododecyl group; and polycyclic aliphatic saturated hydrocarbon groups such as a bicyclo[2.2.2]octanyl group, a tricyclo[5.2.1.02,6]decanyl group, a tricyclo [3.3.1.13,7]decanyl group, a tetracyclo[6.2.1.13,6.02,7] dodecanyl group, and an adamantyl group.

Examples of the monovalent aromatic hydrocarbon group having 6 to 30 carbon atoms include a group obtained by removing one hydrogen atom from an aromatic hydrocarbon ring such as benzene, biphenyl, fluorene, naphthalene, anthracene, or phenanthrene.

In a case where Ra′³ is bonded to any one of Ra′¹ or Ra′² to form a ring, the cyclic group is preferably a 4- to 7-membered ring, and more preferably a 4- to 6-membered ring. Specific examples of the cyclic group include a tetrahydropyranyl group and a tetrahydrofuranyl group.

### Tertiary Alkyl Ester-Type Acid-Dissociable Group:

Among the above polar groups, examples of the acid-dissociable group for protecting the carboxy group include the acid-dissociable group represented by General Formula (a1-r-2) shown below (those represented by General Formula (a01-r) are excluded).

Among the acid-dissociable groups represented by General Formula (a1-r-2), for convenience, a group which is composed of alkyl groups is referred to as a “tertiary alkyl ester-type acid-dissociable group”.

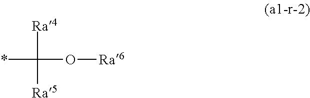

[In the formula, Ra′⁴ to Ra′⁶ each independently represents a hydrocarbon group which may have a substituent, where Ra′⁵ and Ra′⁶ may be bonded to each other to form a ring. * represents a bonding site.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (the aliphatic hydrocarbon group which is a monocyclic group, the aliphatic hydrocarbon group which is a polycyclic group, or the aromatic hydrocarbon group) as Ra′⁴ include the same ones as Ra′³ described above.

The chain-like or cyclic alkenyl group as Ra′⁴ is preferably an alkenyl group having 2 to 10 carbon atoms.

Examples of the hydrocarbon group as Ra′⁵ or Ra′⁶ include the same one as Ra′³ described above.

In a case where Ra′⁵ and Ra′⁶ are bonded to each other to form a ring, suitable examples thereof include a group represented by General Formula (a1-r2-1) and a group represented by General Formula (a1-r2-3).

On the other hand, suitable examples thereof include a group represented by General Formula (a1-r2-4) in a case where Ra′⁴ to Ra′⁶ are not bonded to each other and represent an independent hydrocarbon group.

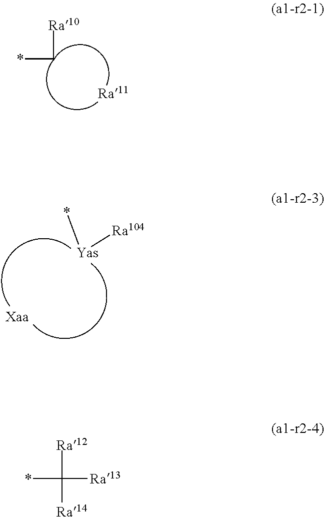

[In General Formula (a1-r2-1), Ra′¹⁰ represents a linear or branched alkyl group having 1 to 12 carbon atoms, a part of which may be substituted with a halogen atom or a hetero atom-containing group. Ra′¹¹ represents a group that forms an aliphatic cyclic group together with a carbon atom to which Ra′¹⁰ is bonded. In General Formula (a1-r2-3), Yaa represents a carbon atom. Xaa is a group that forms an aliphatic cyclic group together with Yaa. Ra¹⁰⁴ represents an aromatic hydrocarbon group which may have a substituent. In General Formula (a1-r2-4), Ra′¹² and Ra′¹³ each independently represents a monovalent chain-like saturated hydrocarbon group having 1 to 10 carbon atoms. Part or all of hydrogen atoms contained in the chain-like saturated hydrocarbon group may be substituted. Ra′¹⁴ represents a hydrocarbon group which may have a substituent. * represents a bonding site.]

In General Formula (a1-r2-1) described above, Ra′¹⁰ represents a linear or branched alkyl group having 1 to 12 carbon atoms, a part of which may be substituted with a halogen atom or a hetero atom-containing group.

The linear alkyl group as Ra′¹⁰ has 1 to 12 carbon atoms, and it preferably has 1 to 10 carbon atoms and particularly preferably has 1 to 5 carbon atoms.

Examples of the branched-chain alkyl group as Ra′¹⁰ include the same one as Ra′³.

A part of the alkyl group as Ra′¹⁰ may be substituted with a halogen atom or a hetero atom-containing group. For example, a part of hydrogen atoms constituting the alkyl group may be substituted with a halogen atom or a hetero atom-containing group. In addition, a part of carbon atoms (such as a methylene group) constituting the alkyl group may be substituted with a hetero atom-containing group.

Examples of the hetero atom referred to here include an oxygen atom, a sulfur atom, and a nitrogen atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

In General Formula (a1-r2-1), an aliphatic cyclic group that is formed by Ra′¹¹ together with a carbon atom to which Ra′¹⁰ is bonded is preferably the group described as the aliphatic hydrocarbon group (the alicyclic hydrocarbon group) which is a monocyclic group or a polycyclic group as Ra′³ in General Formula (a1-r-1). Among the above, a monocyclic alicyclic hydrocarbon group is preferable, specifically, a cyclopentyl group or a cyclohexyl group is more preferable, and a cyclopentyl group is still more preferable.

In General Formula (a1-r2-3), an aliphatic cyclic group that is formed by Xaa together with Yaa is preferably the group mentioned as the aliphatic hydrocarbon group which is a monocyclic group or a polycyclic group as Ra′³ in General Formula (a1-r-1).

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among them, Ra¹⁰⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be contained in Ra¹⁰⁴ in General Formula (a1-r2-3) include a methyl group, an ethyl group, a propyl group, a hydroxy group, a carboxy group, a halogen atom, an alkoxy group (a methoxy group, an ethoxy group, a propoxy group, a butoxy group, and the like), and an alkyloxycarbonyl group.

In General Formula (a1-r2-4), Ra′¹² and Ra′¹³ each independently represents a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ described above. Part or all of hydrogen atoms contained in the chain-like saturated hydrocarbon group may be substituted.

Among them, Ra′¹² and Ra′¹³ are preferably a hydrogen atom or an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon groups represented by Ra′¹² and Ra′¹³ are substituted, examples of the substituent include the same group as Ra^{x5} described above.

In General Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ preferably has 3 to 10 carbon atoms and more preferably has 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the cyclic hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane is preferably a monocycloalkane having 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same one as the aromatic hydrocarbon group as Ra¹⁰⁴. Among them, Ra′¹⁴ is preferably a group obtained by removing one or more hydrogen atoms from an aromatic hydrocarbon ring having 6 to 15 carbon atoms, more preferably a group obtained by removing one or more hydrogen atoms from benzene, naphthalene, anthracene, or phenanthrene, still more preferably a group obtained by removing one or more hydrogen atoms from benzene, naphthalene, or anthracene, particularly preferably a group obtained by removing one or more hydrogen atoms from naphthalene or anthracene, and most preferably a group obtained by removing one or more hydrogen atoms from naphthalene.

Examples of the substituent which may be contained in Ra′¹⁴ include the same one as the substituent which may be contained in Ra¹⁰⁴.

In a case where Ra′¹⁴ in General Formula (a1-r2-4) represents a naphthyl group, the position at which the tertiary carbon atom in General Formula (a1-r2-4) is bonded may be any of the 1-position and the 2-position of the naphthyl group.

In a case where Ra′¹⁴ in General Formula (a1-r2-4) represents an anthryl group, the position at which the tertiary carbon atom in General Formula (a1-r2-4) is bonded may be any of the 1-position, the 2-position, and 9-position of the anthryl group.

Specific examples of the group represented by General Formula (a1-r2-1) are shown below.

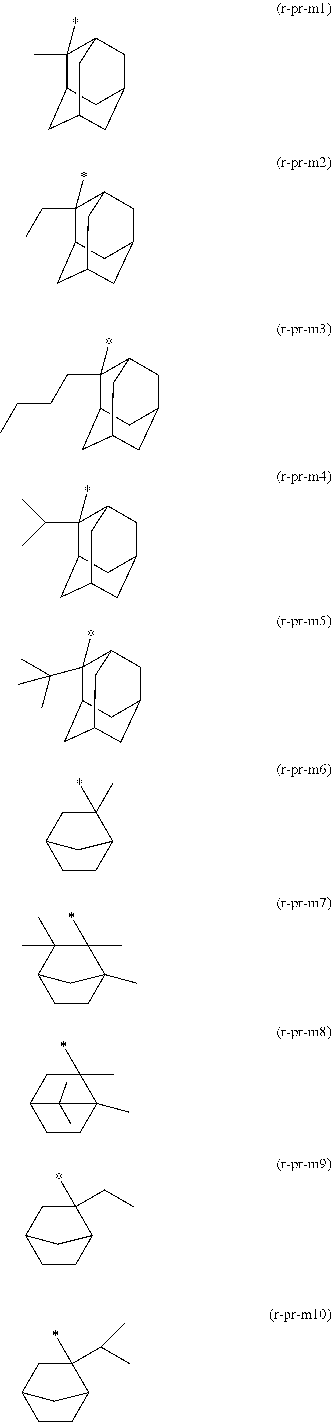

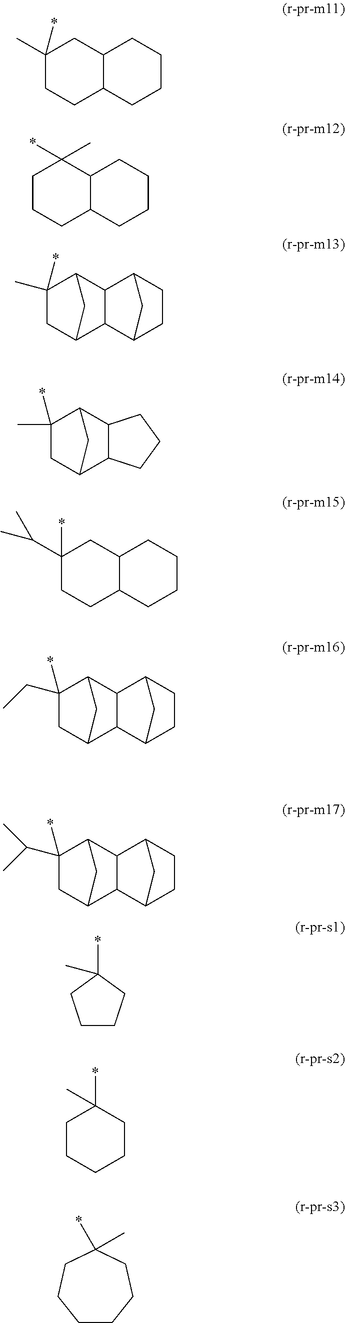

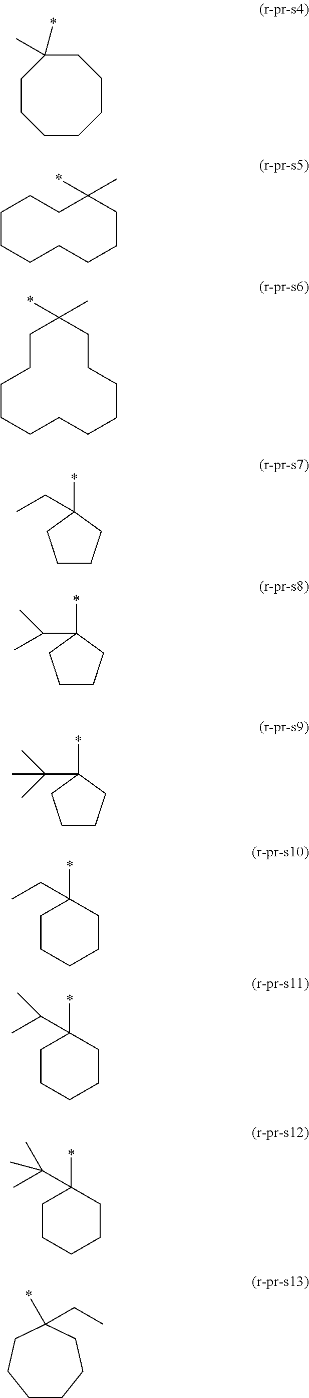

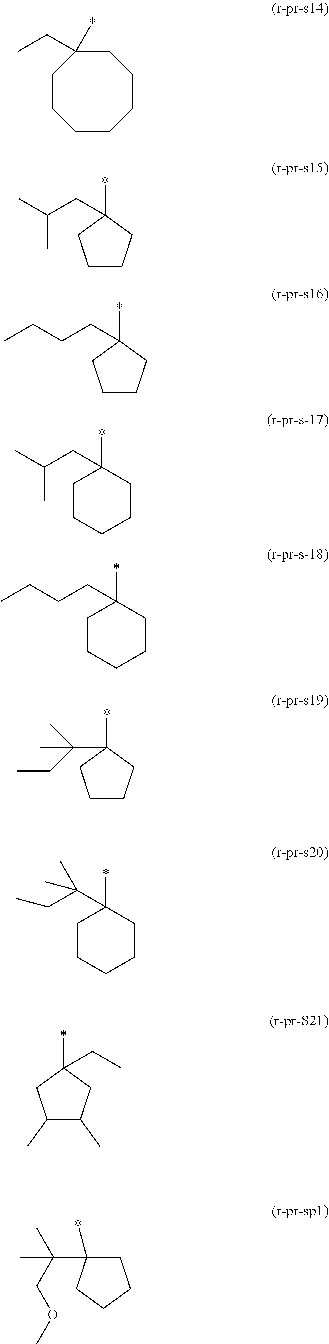

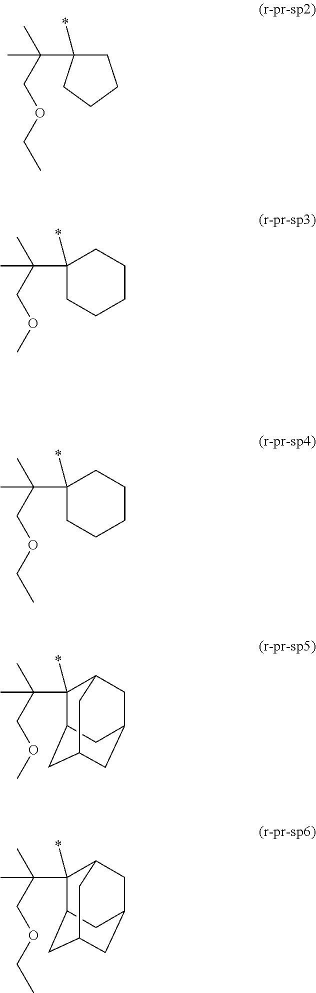

Specific examples of the group represented by General Formula (a1-r2-3) are shown below.

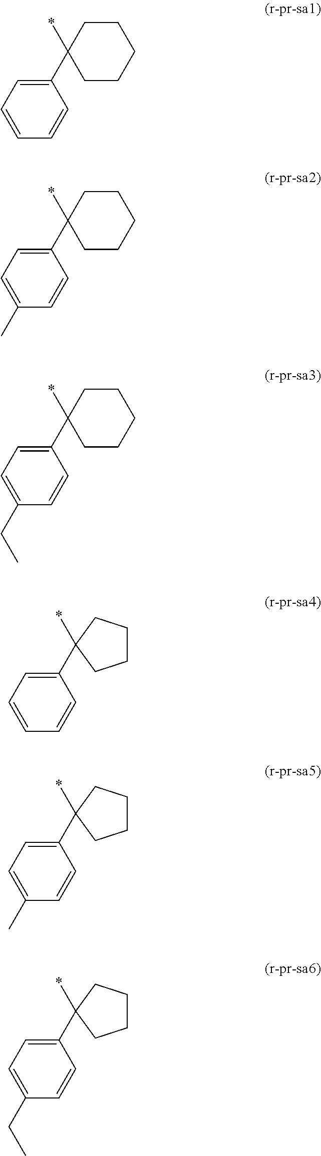

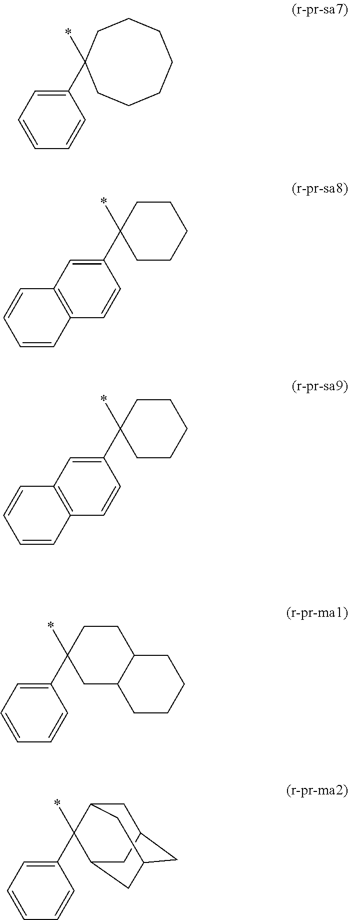

Specific examples of the group represented by General Formula (a1-r2-4) are shown below.

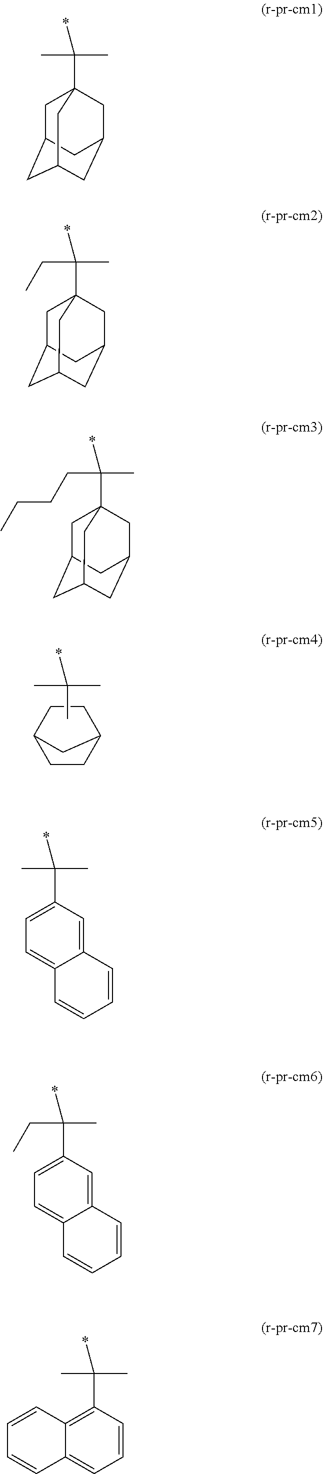

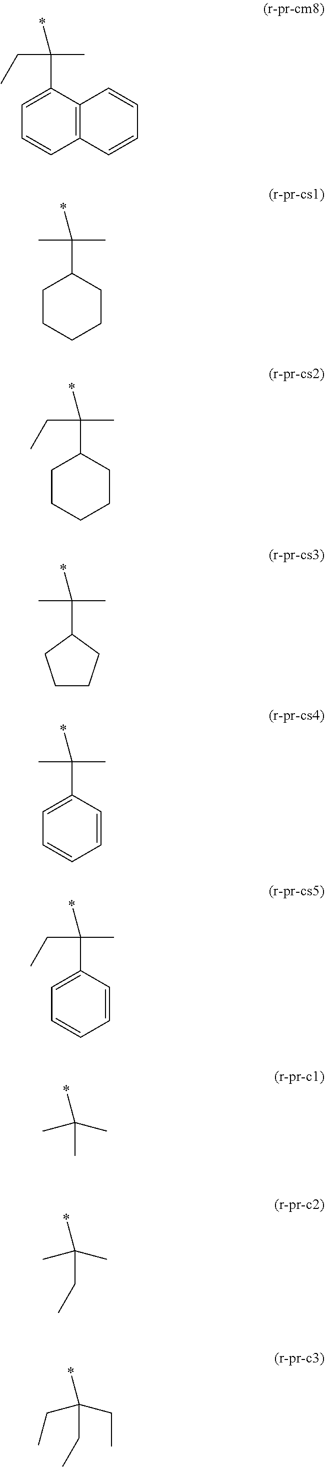

### Tertiary Alkyloxycarbonyl Acid-Dissociable Group:

Among the polar groups, examples of the acid-dissociable group for protecting a hydroxyl group include an acid-dissociable group (hereinafter, for convenience, also referred to as a “tertiary alkyloxycarbonyl acid-dissociable group”) represented by General Formula (a1-r-3) shown below.

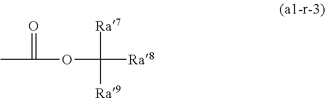

[In the formula, Ra′⁷ to Ra′⁹ each represent an alkyl group.]

In General Formula (a1-r-3), Ra′⁷ to Ra′⁹ are each preferably an alkyl group having 1 to 5 carbon atoms and more preferably an alkyl group having 1 to 3 carbon atoms.

In addition, the total number of carbon atoms in each of the alkyl groups is preferably in a range of 3 to 7, more preferably in a range of 3 to 5, and most preferably 3 or 4.

Examples of the constitutional unit (a1) include a constitutional unit derived from an acrylic acid ester in which the hydrogen atom bonded to the carbon atom at the α-position may be substituted with a substituent; a constitutional unit derived from acrylamide; a constitutional unit in which at least a part of hydrogen atoms in a hydroxyl group of a constitutional unit derived from hydroxystyrene or a hydroxystyrene derivative are protected by the substituent including an acid-decomposable group; and a constitutional unit in which at least a part of hydrogen atoms in —C(═O)—OH of a constitutional unit derived from vinylbenzoic acid or a vinylbenzoic acid derivative are protected by the substituent including an acid-decomposable group.

Among the above, the constitutional unit (a1) is preferably a constitutional unit derived from an acrylic acid ester in which the hydrogen atom bonded to the carbon atom at the α-position may be substituted with a substituent.

Preferred specific examples of such a constitutional unit (a1) include constitutional units represented by General Formula (a1-1) or (a1-2).

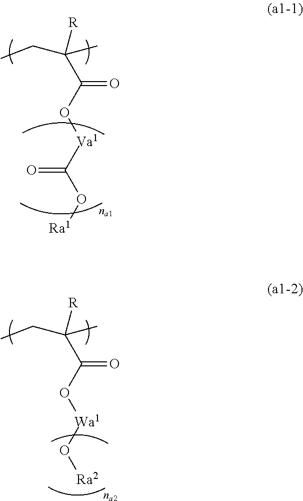

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ is the acid-dissociable group represented by General Formula (a1-r-1) or (a1-r-2). Wa¹ represents a (nₐ₂+1)-valent hydrocarbon group, nₐ₂ represents an integer in a range of 1 to 3, and Ra² represents the acid-dissociable group represented by General Formula (a1-r-1) or (a1-r-3).]

In General Formula (a1-1), R is the same as R in General Formula (a01-1-1).

In General Formula (a1-1), the divalent hydrocarbon group as Va¹ may be an aliphatic hydrocarbon group or an aromatic hydrocarbon group. Va¹ is the same as Va⁰ in General Formula (a01-1-1).

In General Formula (a1-1), nₐ₁ represents an integer in a range of 0 to 2. nₐ₁ is preferably 0 or 1.

In General Formula (a1-1), Ra¹ represents the acid-dissociable group represented by General Formula (a1-r-1) or (a1-r-2).

In General Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group be saturated. Examples of the aliphatic hydrocarbon group include a linear or branched aliphatic hydrocarbon group, an aliphatic hydrocarbon group containing a ring in the structure thereof, and a combination of the linear or branched aliphatic hydrocarbon group and the aliphatic hydrocarbon group containing a ring in the structure thereof.

The valency of (nₐ₂+1) is preferably divalent, trivalent, or tetravalent, and more preferably divalent or trivalent.

In General Formula (a1-2), Ra² represents the acid-dissociable group represented by General Formula (a1-r-1) or (a1-r-3).

Specific examples of the constitutional unit represented by General Formula (a1-1) are shown below. In each of the formulae shown below, R^{α} represents a hydrogen atom, a methyl group, or a trifluoromethyl group.

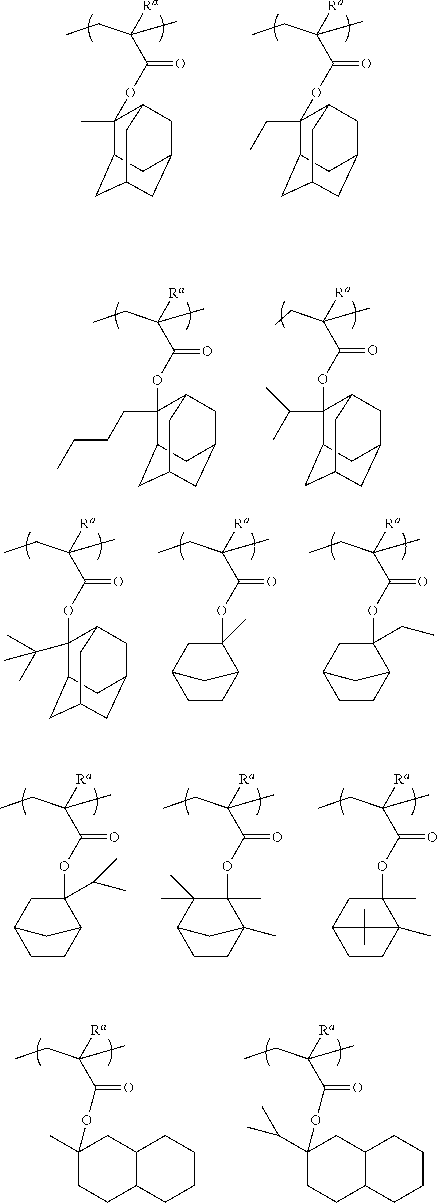

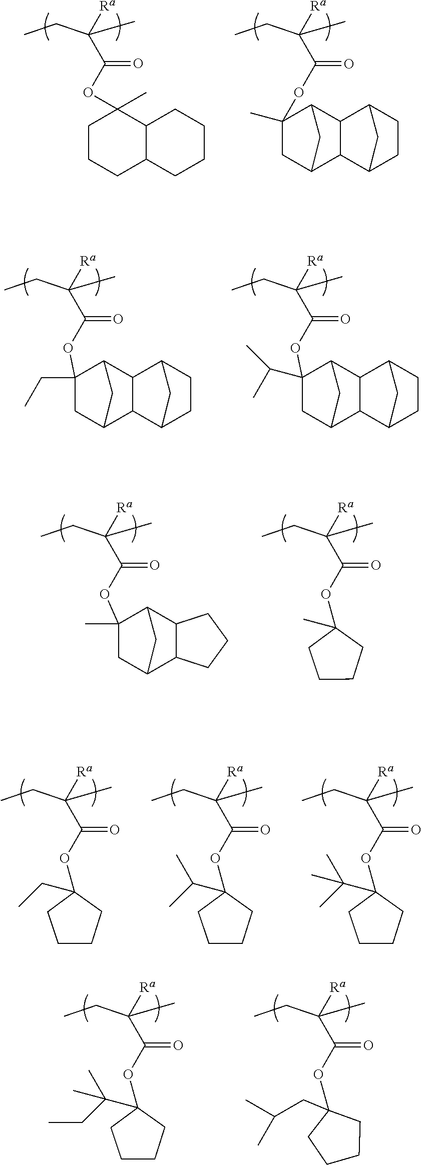

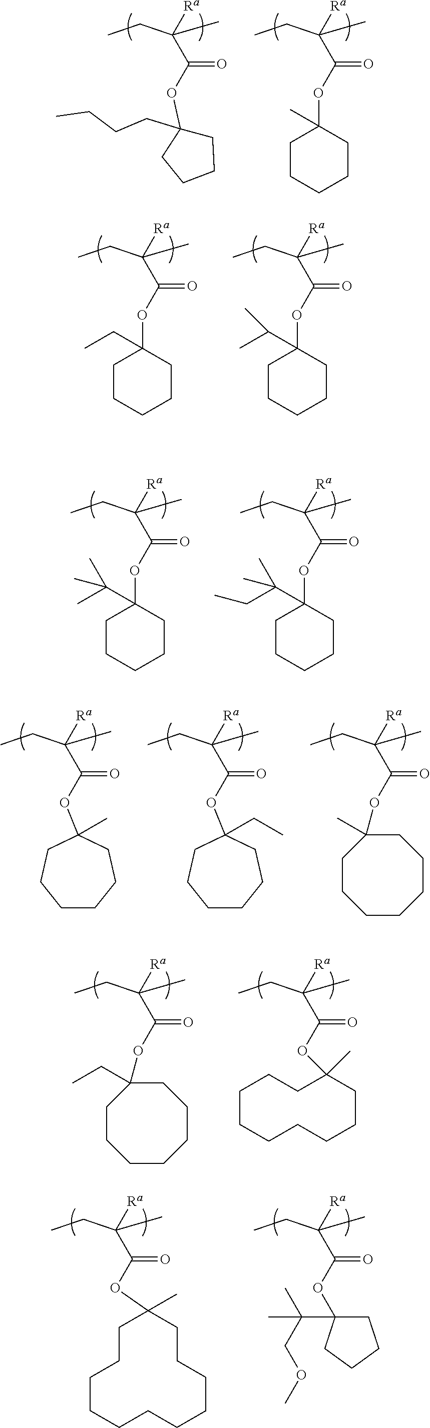

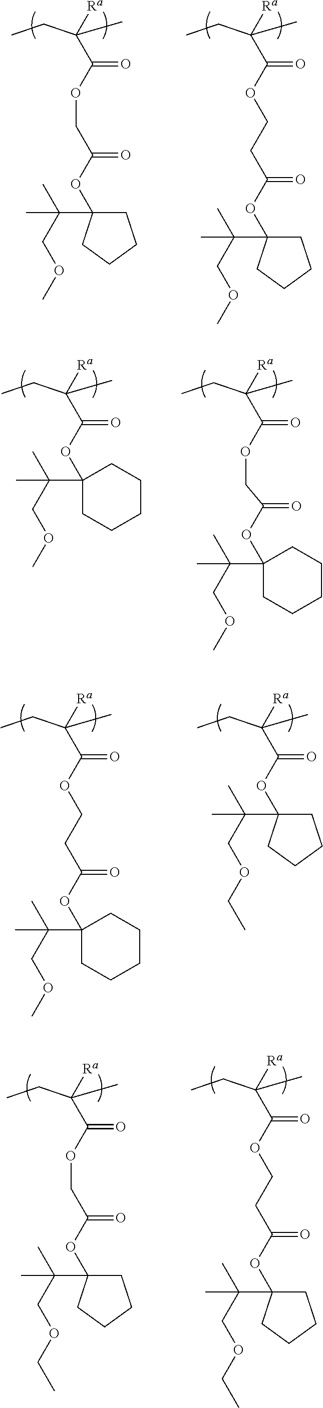

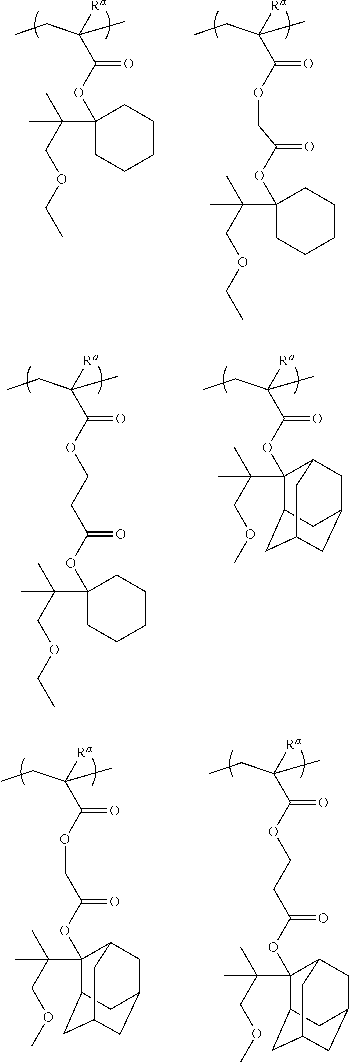

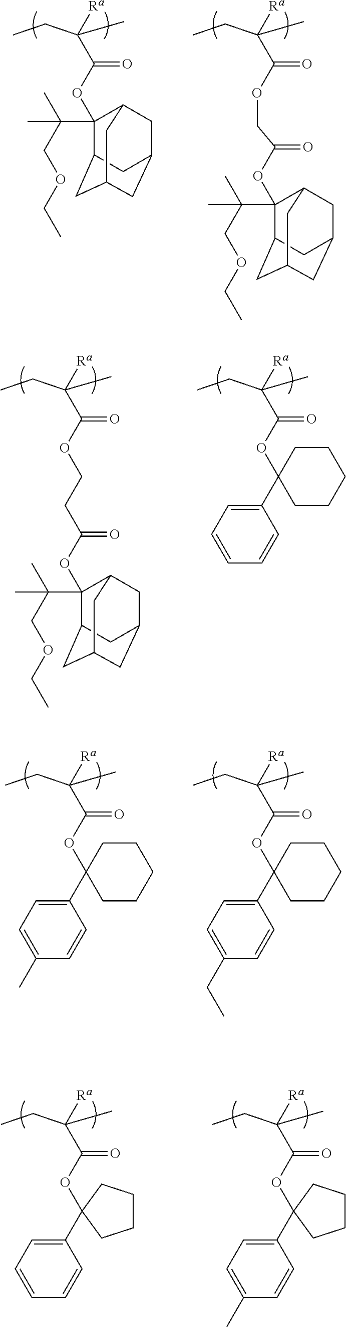

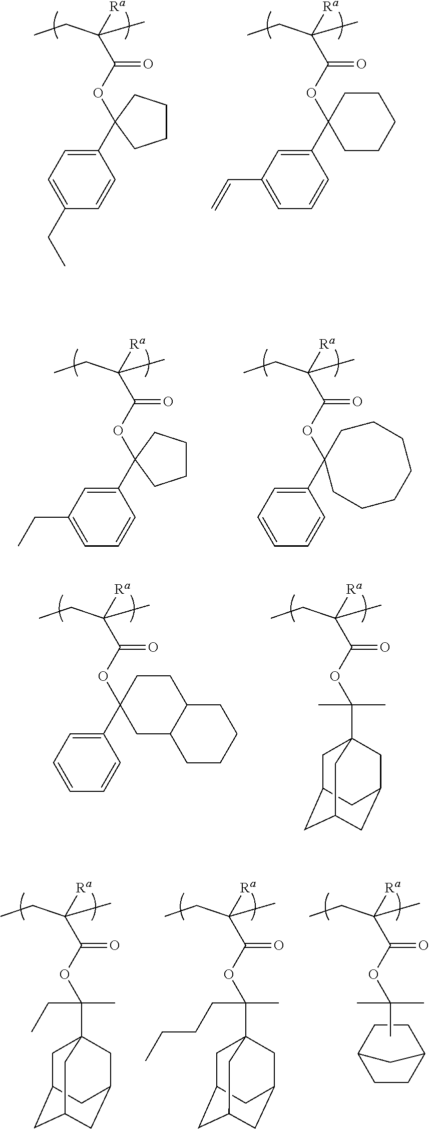

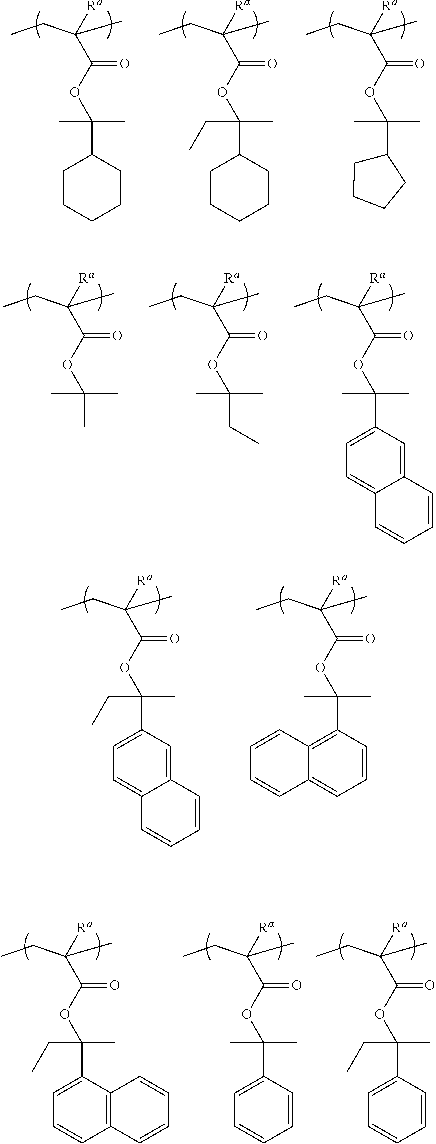

The constitutional unit (a1) contained in the component (A1) may be one kind or may be two or more kinds.

The constitutional unit (a1) is more preferably a constitutional unit represented by General Formula (a1-1) since lithography characteristics (sensitivity, shape, and the like) depending on an electron beam or EUV can be more easily increased.

The proportion of the constitutional unit (a1) in the component (A1) is preferably in a range of 0% to 50% by mole, more preferably in a range of 0% to 40% by mole, still more preferably in a range of 0% to 30% by mole, and particularly preferably in a range of 0% to 20% by mole, with respect to the total (100% by mole) of all constitutional units constituting the component (A1).

It is preferable that the component (A1) not have the constitutional unit (a1).

The proportion of the total of the constitutional unit (a01) and the constitutional unit (a1) in the component (A1) is preferably in a range of 10% to 90% by mole, more preferably in a range of 10% to 80% by mole, still more preferably in a range of 20% to 75% by mole, even still more preferably in a range of 30% to 70% by mole, and particularly preferably 40% to 60% by mole, with respect to the total amount (100% by mole) of all constitutional units constituting the component (A1).

### In Regard to Constitutional Unit (a2):

The component (A1) may further have a constitutional unit (a2) (however, a constitutional unit corresponding to the constitutional unit (a1) is excluded) containing a lactone-containing cyclic group, an —SO₂—-containing cyclic group, or a carbonate-containing cyclic group.

In a case where the component (A1) is used for forming a resist film, the lactone-containing cyclic group, the —SO₂—-containing cyclic group, or the carbonate-containing cyclic group in the constitutional unit (a2) is effective for improving the adhesiveness of the resist film to the substrate. In addition, due to having the constitutional unit (a2), lithography characteristics can be improved, for example, by the effects obtained by appropriately adjusting the acid diffusion length, increasing the adhesiveness of the resist film to the substrate, and appropriately adjusting the solubility during development.

The term “lactone-containing cyclic group” indicates a cyclic group that contains a ring (lactone ring) containing a —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group for the constitutional unit (a2) is not particularly limited, and any lactone-containing cyclic group may be used. Specific examples thereof include groups each represented by General Formulae (a2-r-1) to (a2-r-7) shown below.

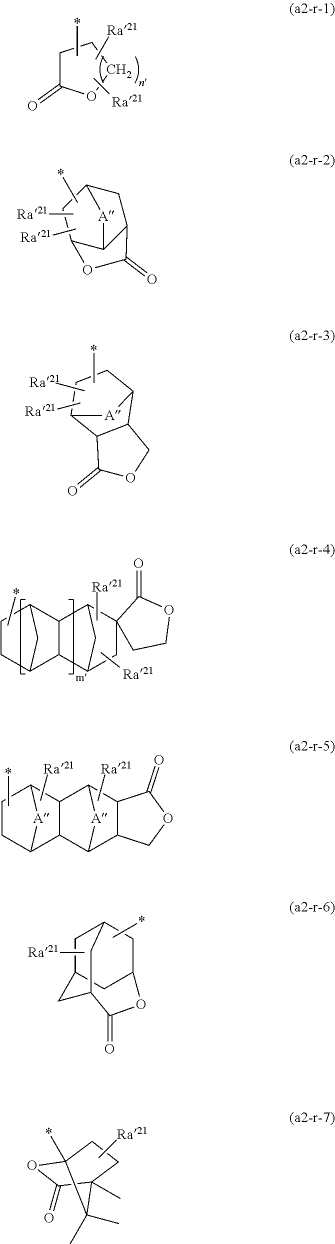

[In the formulae, each Ra′²¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or an —SO₂—-containing cyclic group; A″ represents an oxygen atom, a sulfur atom, or an alkylene group having 1 to 5 carbon atoms, which may contain an oxygen atom (—O—) or a sulfur atom (—S—); and n′ represents an integer in a range of 0 to 2, and m′ is 0 or 1.]

In General Formulae (a2-r-1) to (a2-r-7), the alkyl group as Ra′²¹ is preferably an alkyl group having 1 to 6 carbon atoms. The alkyl group is preferably a linear alkyl group or a branched alkyl group. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or an ethyl group is preferable, and a methyl group is particularly preferable.

The alkoxy group as Ra′²¹ is preferably an alkoxy group having 1 to 6 carbon atoms. Further, the alkoxy group is preferably a linear or branched alkoxy group. Specific examples of the alkoxy groups include a group formed by linking the above-described alkyl group mentioned as the alkyl group represented by Ra′²¹ to an oxygen atom (—O—).

The halogen atom as Ra′²¹ is preferably a fluorine atom.

Examples of the halogenated alkyl group as Ra′²¹ include a group obtained by substituting part or all of hydrogen atoms in the above-described alkyl group as Ra′²¹ with the above-described halogen atoms. The halogenated alkyl group is preferably a fluorinated alkyl group and particularly preferably a perfluoroalkyl group.

In —COOR″ and —OC(═O)R″ as Ra′²¹, R″ represents a hydrogen atom, an alkyl group, a lactone-containing cyclic group, a carbonate-containing cyclic group, or an —SO₂—-containing cyclic group.

The alkyl group as R″ may be linear, branched, or cyclic, and preferably has 1 to 15 carbon atoms.

In a case where R″ represents a linear or branched alkyl group, it is preferably an alkyl group having 1 to 10 carbon atoms, more preferably an alkyl group having 1 to 5 carbon atoms, and particularly preferably a methyl group or an ethyl group.

In a case where R″ represents a cyclic alkyl group, the cyclic alkyl group preferably has 3 to 15 carbon atoms, more preferably has 4 to 12 carbon atoms, and particularly preferably has 5 to 10 carbon atoms. Specific examples thereof include a group obtained by removing one or more hydrogen atoms from a monocycloalkane. which may or may not be substituted with a fluorine atom or a fluorinated alkyl group; and a group obtained by removing one or more hydrogen atoms from a polycycloalkane such as bicycloalkane, tricycloalkane, or tetracycloalkane. More specific examples thereof include a group obtained by removing one or more hydrogen atoms from a monocycloalkane such as cyclopentane or cyclohexane; and a group obtained by removing one or more hydrogen atoms from a polycycloalkane such as adamantane, norbornane, isobornane, tricyclodecane, or tetracyclododecane.

Examples of the lactone-containing cyclic group as R″ include the same ones as the groups each represented by General Formulae (a2-r-1) to (a2-r-7).

The carbonate-containing cyclic group as R″ is the same as the carbonate-containing cyclic group described below. Specific examples of the carbonate-containing cyclic group include groups each represented by General Formulae (ax3-r-1) to (ax3-r-3).

The —SO₂—-containing cyclic group as R″ is the same as the —SO₂—-containing cyclic group described below. Specific examples thereof include groups each represented by General Formulae (a5-r-1) to (a5-r-4).

The hydroxyalkyl group as Ra′²¹ preferably has 1 to 6 carbon atoms, and specific examples thereof include a group in which at least one hydrogen atom in the alkyl group as Ra′²¹ has been substituted with a hydroxyl group.

Among the above, each Ra′²¹ is independently preferably a hydrogen atom or a cyano group.

In General Formulae (a2-r-2), (a2-r-3), and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, examples of which include —O—CH₂—, —CH₂—O—CH₂—, —S—CH₂—, and —CH₂—S—CH₂—. A″ is preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

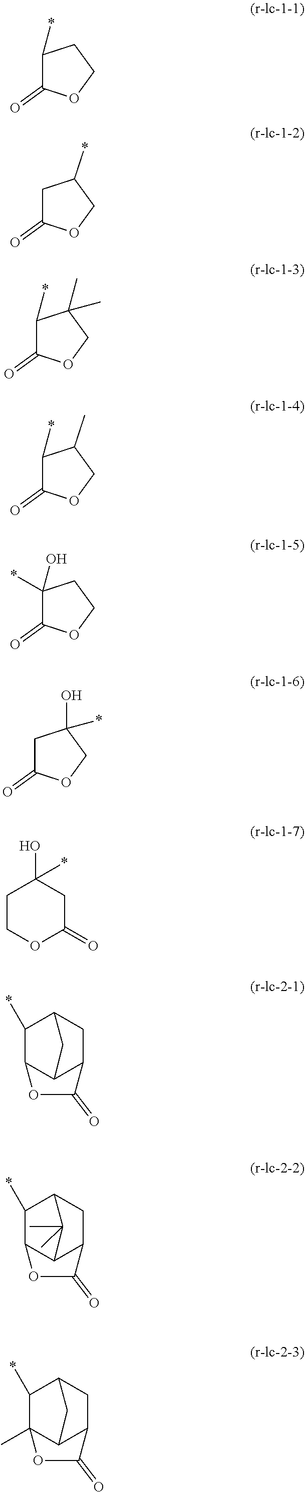

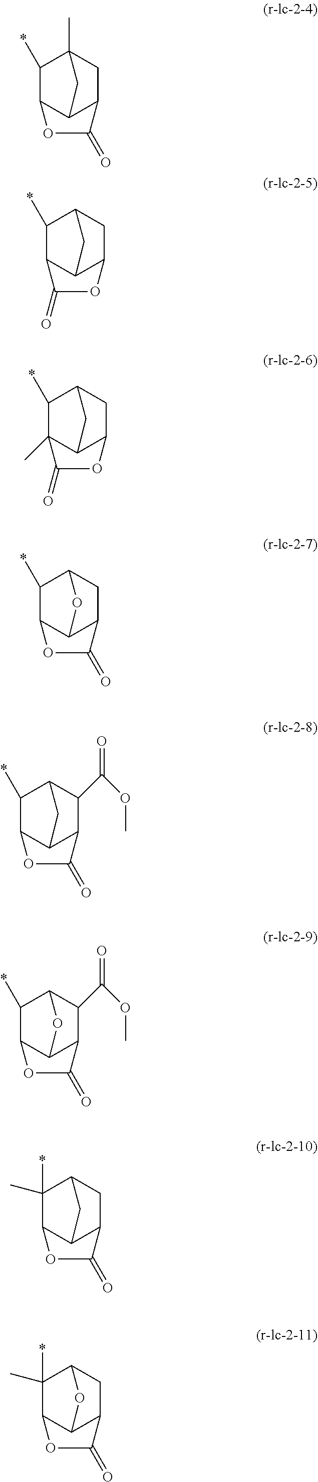

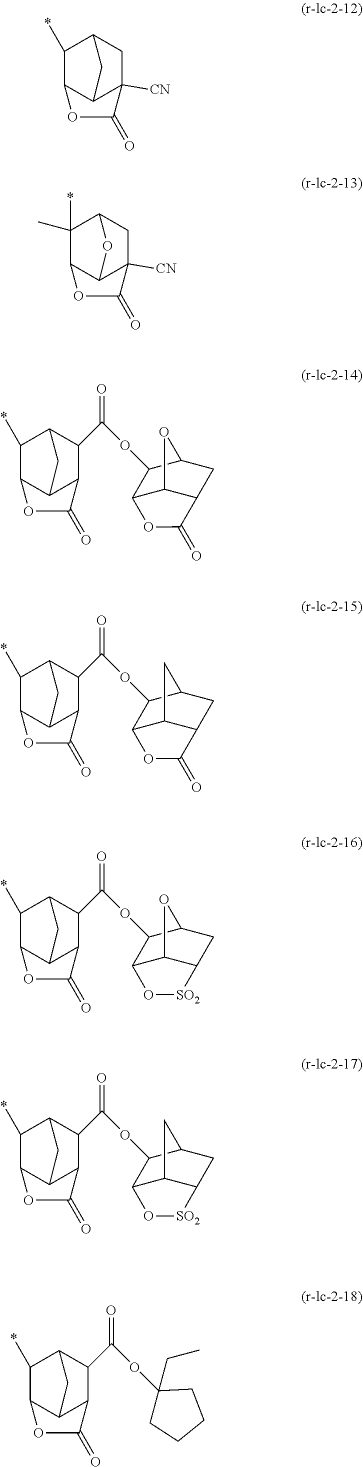

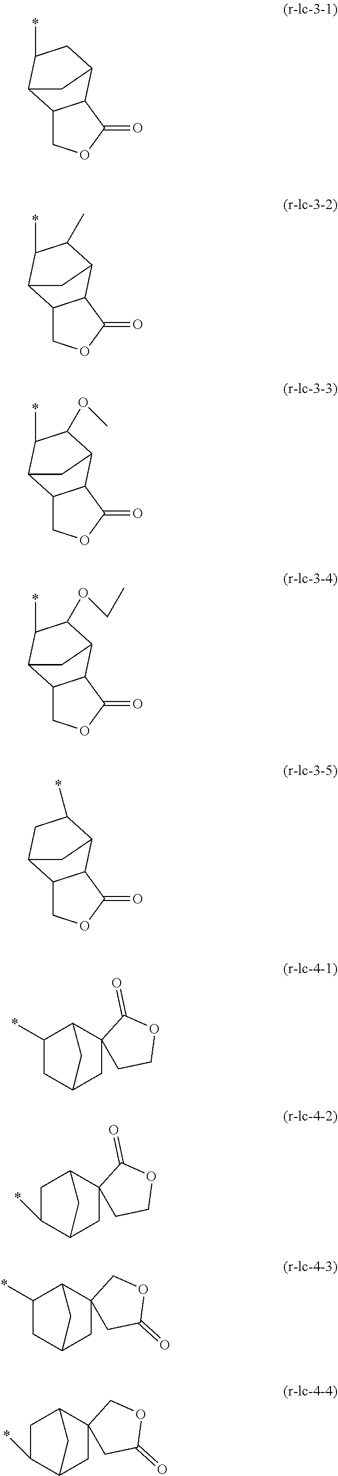

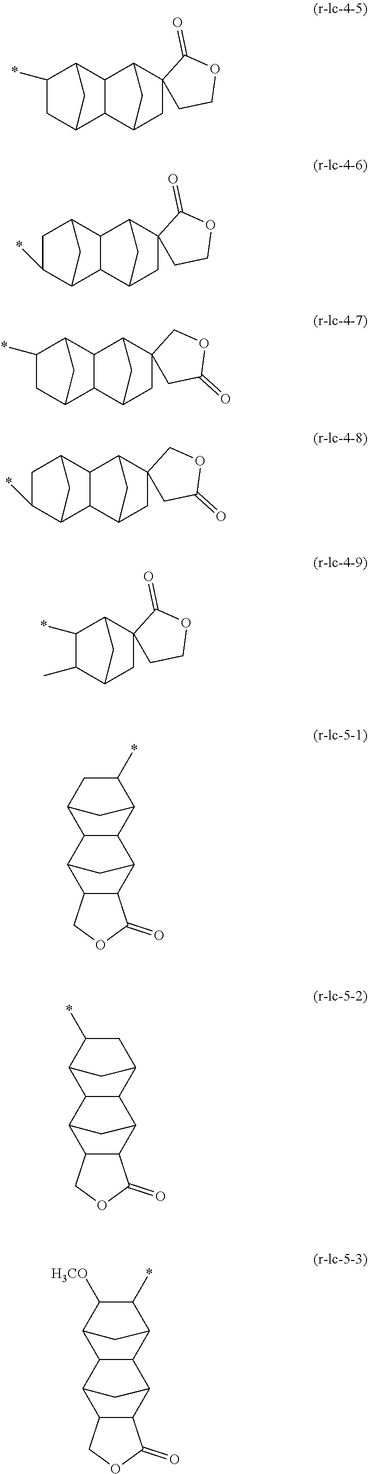

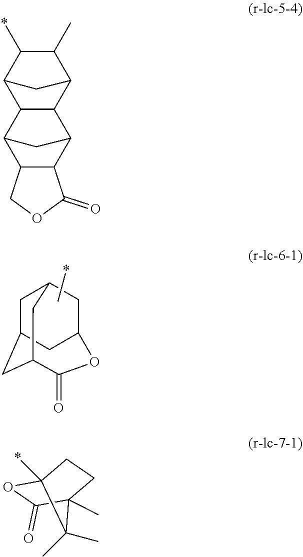

The term “—SO₂—-containing cyclic group” indicates a cyclic group having a ring containing —SO₂— in the ring skeleton thereof, and specifically, is a cyclic group in which the sulfur atom (S) in —SO₂— forms part of the ring skeleton of the cyclic group. In a case where the ring containing —SO₂— in the ring skeleton thereof is counted as the first ring and the group contains only the ring, the group is referred to as a monocyclic group. In a case where the group further has other ring structures, the group is referred to as a polycyclic group regardless of the structures. The —SO₂—-containing cyclic group may be a monocyclic group or a polycyclic group.

The —SO₂—-containing cyclic group is preferably a cyclic group containing —O—SO₂— in the ring skeleton thereof, in other words, a cyclic group containing a sultone ring in which —O—S— in the —O—SO₂— group forms part of the ring skeleton thereof. More specific examples of the —SO₂—-containing cyclic group include groups each represented by General Formulae (a5-r-1) to (a5-r-4) shown below.

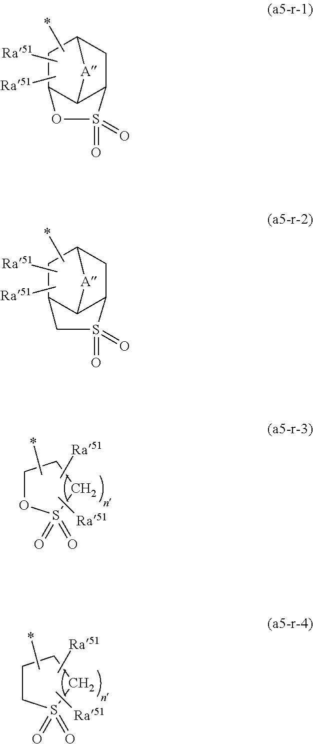

[In the formulae, each Ra′⁵¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or an —SO₂—-containing cyclic group; A″ represents an oxygen atom, a sulfur atom, or an alkylene group having 1 to 5 carbon atoms, which may contain an oxygen atom or a sulfur atom; and n′ represents an integer in a range of 0 to 2.]

In General Formulae (a5-r-1) and (a5-r-2), A″ is the same as A″ in General Formulae (a2-r-2), (a2-r-3), and (a2-r-5).

Examples of the alkyl group, the alkoxy group, the halogen atom, the halogenated alkyl group, —COOR″, —OC(═O)R″, and the hydroxyalkyl group as Ra′⁵¹ include the same as those described in the description for Ra′²¹ in General Formulae (a2-r-1) to (a2-r-7).

Specific examples of the groups each represented by General Formulae (a5-r-1) to (a5-r-4) are shown below. In the formulae shown below, “Ac” represents an acetyl group.

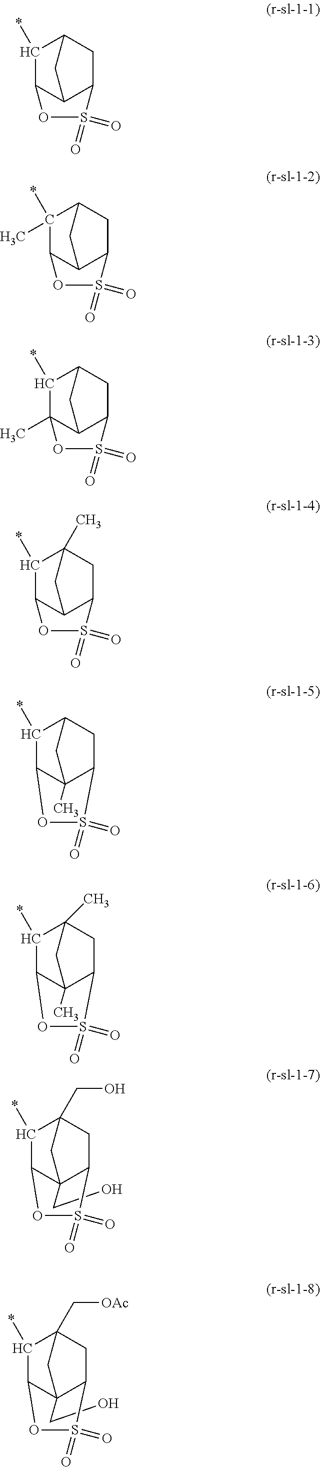

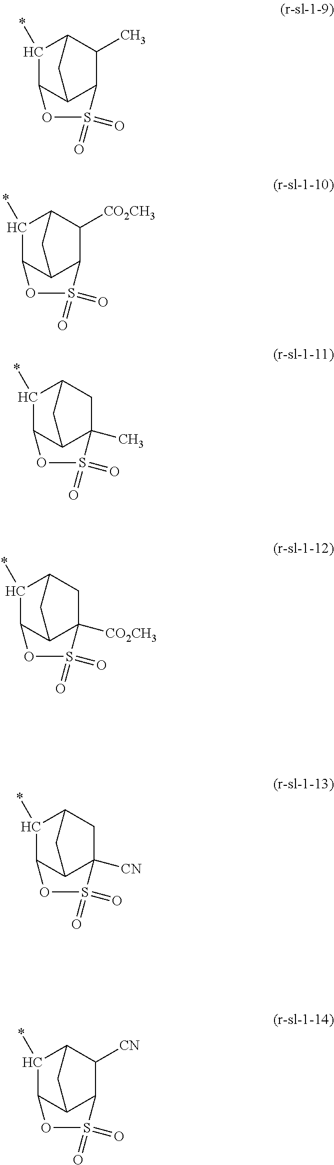

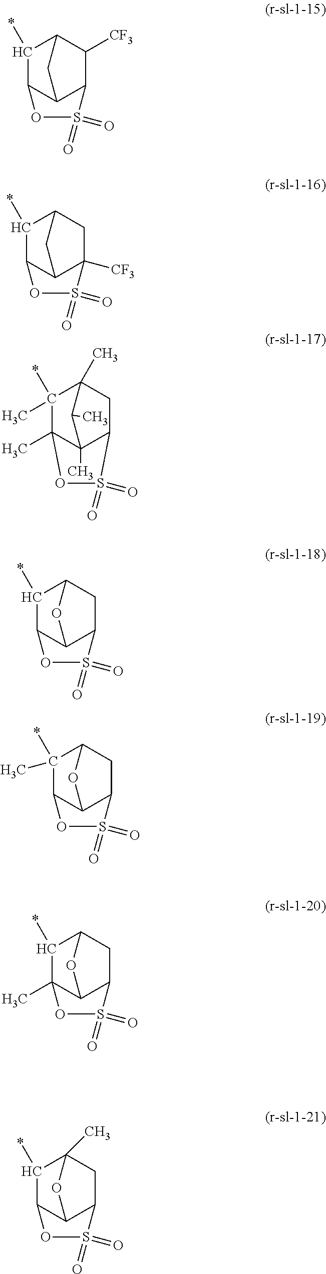

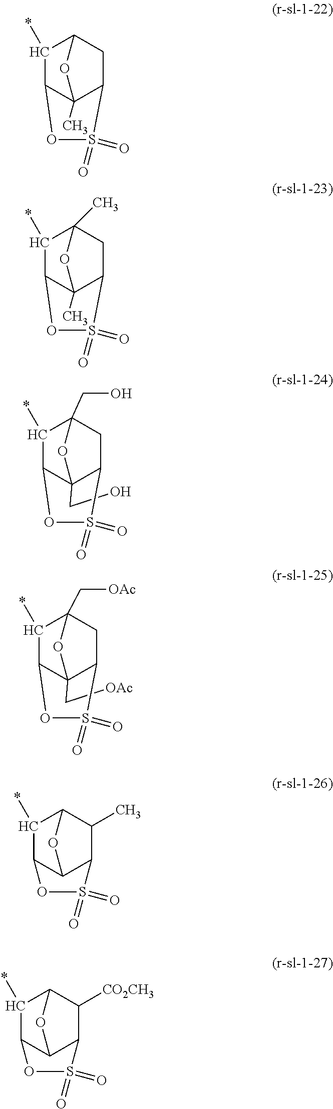

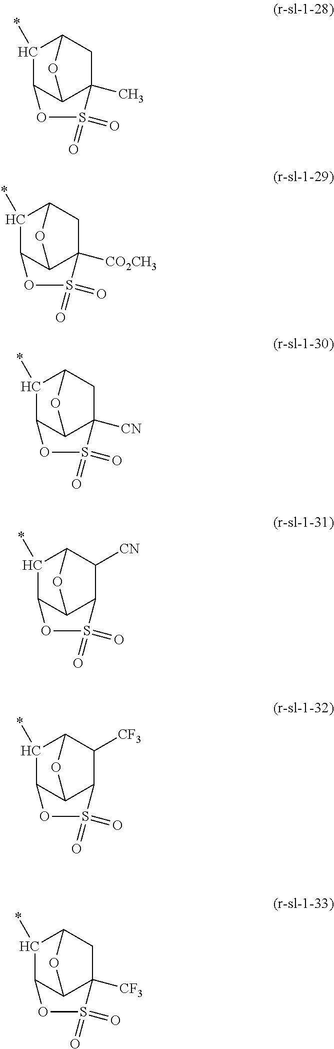

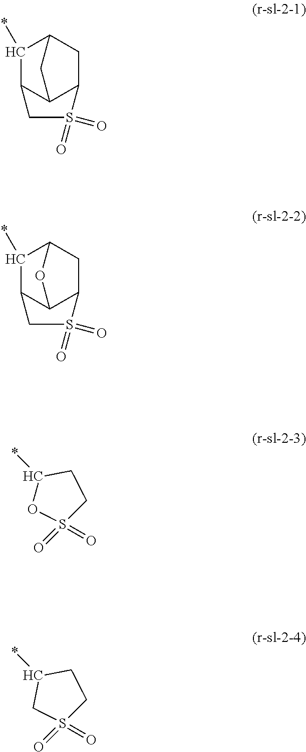

The term “carbonate-containing cyclic group” indicates a cyclic group having a ring (a carbonate ring) containing —O—C(═O)—O— in the ring skeleton thereof. In a case where the carbonate ring is counted as the first ring and the group contains only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The carbonate-containing cyclic group is not particularly limited, and any carbonate-containing cyclic group may be used. Specific examples thereof include groups each represented by General Formulae (ax3-r-1) to (ax3-r-3) shown below.

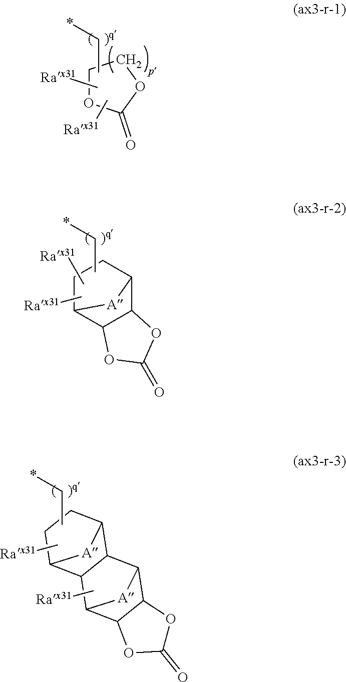

[In the formulae, each Ra′^{x31} independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or an —SO₂—-containing cyclic group; A″ represents an oxygen atom, a sulfur atom, or an alkylene group having 1 to 5 carbon atoms, which may contain an oxygen atom or a sulfur atom; and p′ represents an integer in a range of 0 to 3, and q′ is 0 or 1.]

In General Formulae (ax3-r-2) and (ax3-r-3), A″ is the same as A″ in General Formulae (a2-r-2), (a2-r-3), and (a2-r-5).

Examples of the alkyl group, the alkoxy group, the halogen atom, the halogenated alkyl group, —COOR″, —OC(═O)R″, and the hydroxyalkyl group as Ra′³¹ include the same as those described in the description for Ra′²¹ in General Formulae (a2-r-1) to (a2-r-7).

Specific examples of the groups each represented by General Formulae (ax3-r-1) to (ax3-r-3) are shown below.

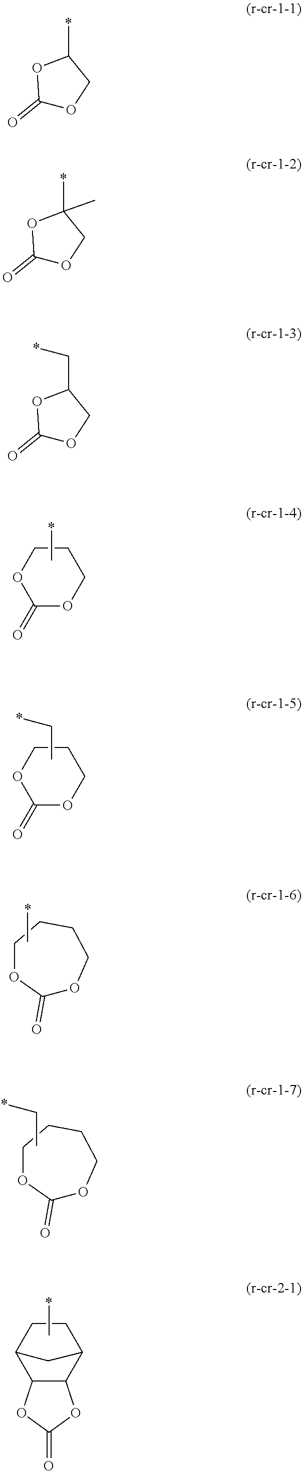

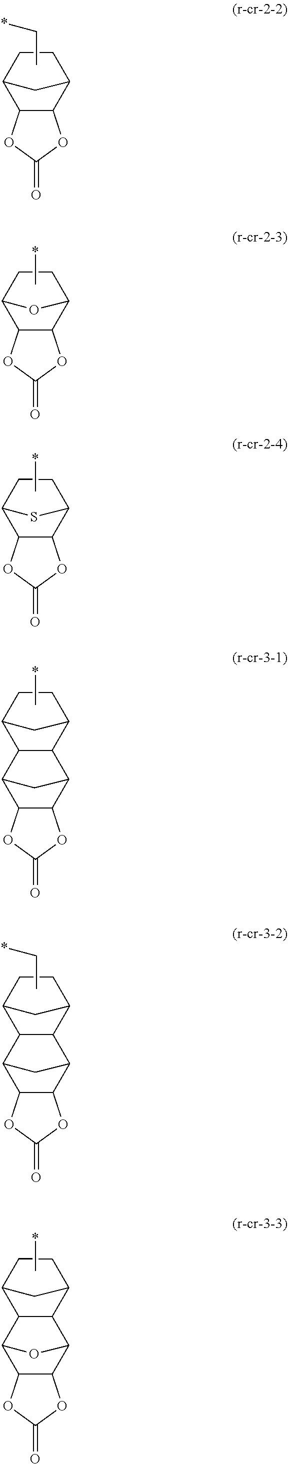

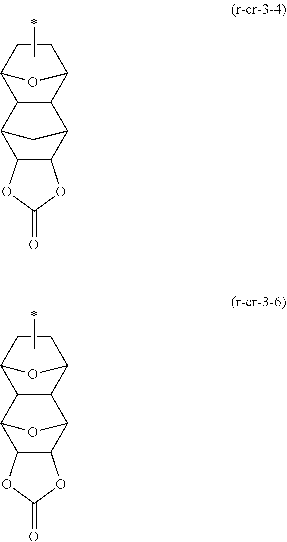

Among them, the constitutional unit (a2) is preferably a constitutional unit derived from an acrylic acid ester in which the hydrogen atom bonded to the carbon atom at the α-position may be substituted with a substituent.

The constitutional unit (a2) is preferably a constitutional unit represented by General Formula (a2-1).

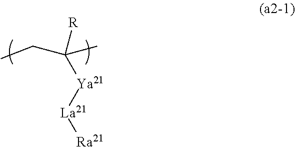

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO— or —CONHCS—, and R′ represents a hydrogen atom or a methyl group. However, in a case where La²¹ represents —O—, Ya²¹ does not represent —CO—. Ra²¹ represents a lactone-containing cyclic group, a carbonate-containing cyclic group, or an —SO₂—-containing cyclic group.]

In General Formula (a2-1), R has the same definition as described above. R is preferably a hydrogen atom, an alkyl group having 1 to 5 carbon atoms, or a fluorinated alkyl group having 1 to 5 carbon atoms, and particularly preferably a hydrogen atom or a methyl group in terms of industrial availability.

In General Formula (a2-1), the divalent linking group as Ya²¹ is not particularly limited, and suitable examples thereof include a divalent hydrocarbon group which may have a substituent, and a divalent linking group having a hetero atom.

Divalent Hydrocarbon Group which May have Substituent:

In a case where Ya²¹ represents a divalent hydrocarbon group which may have a substituent, the hydrocarbon group may be an aliphatic hydrocarbon group or an aromatic hydrocarbon group.

### Aliphatic Hydrocarbon Group as Ya²¹

The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group, and an aliphatic hydrocarbon group containing a ring in the structure thereof.

### Linear or Branched Aliphatic Hydrocarbon Group

The linear aliphatic hydrocarbon group described above preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group described above preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

The linear or branched aliphatic hydrocarbon group may have a substituent or may not have a substituent. Examples of the substituent include a fluorine atom, a fluorinated alkyl group having 1 to 5 carbon atoms, which has been substituted with a fluorine atom, and a carbonyl group.

### Aliphatic Hydrocarbon Group Containing Ring in Structure Thereof

Examples of the aliphatic hydrocarbon group containing a ring in the structure thereof include a cyclic aliphatic hydrocarbon group which may have a substituent containing a hetero atom in the ring structure thereof (a group obtained by removing two hydrogen atoms from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in a linear or branched aliphatic hydrocarbon group. Examples of the linear or branched aliphatic hydrocarbon group include the same as those described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane is preferably a monocycloalkane having 3 to 6 carbon atoms, and specific examples thereof include cyclopentane and cyclohexane. The polycyclic alicyclic hydrocarbon group is preferably a group obtained by removing two hydrogen atoms from a polycycloalkane, and the polycycloalkane preferably has 7 to 12 carbon atoms. Specific examples of the polycycloalkane include adamantane, norbornane, isobornane, tricyclodecane, and tetracyclododecane.

The cyclic aliphatic hydrocarbon group may have or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and a methyl group, an ethyl group, a propyl group, an n-butyl group, or a tert-butyl group is more preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as the substituent is preferably a fluorine atom.

Examples of the halogenated alkyl group as the substituent include groups in which part or all of hydrogen atoms in the above-described alkyl groups have been substituted with the above-described halogen atoms.

In the cyclic aliphatic hydrocarbon group, a part of carbon atoms constituting the ring structure thereof may be substituted with a substituent containing a hetero atom. The substituent containing a hetero atom is preferably —O—, —C(═O)—O—, —S—, —S(═O)₂—, or —S(═O)₂—O—.

### Aromatic Hydrocarbon Group as Ya²¹

The aromatic hydrocarbon group is a hydrocarbon group having at least one aromatic ring.

The aromatic ring is not particularly limited as long as it is a cyclic conjugated system having (4n+2) π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms in a substituent is not included in the number of carbon atoms in the aromatic ring.

Specific examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group obtained by removing two hydrogen atoms from the above-described aromatic hydrocarbon ring or aromatic heterocyclic ring (an arylene group or a heteroarylene group); a group obtained by removing two hydrogen atoms from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group in which one hydrogen atom further has been removed from an aryl group in arylalkyl groups such as a benzyl group, a phenethyl group, a 1-naphthylmethyl group, a 2-naphthylmethyl group, a 1-naphthylethyl group, or a 2-naphthylethyl group). The alkylene group bonded to the aryl group or the heteroaryl group preferably has 1 to 4 carbon atoms, more preferably has 1 or 2 carbon atoms, and particularly preferably has 1 carbon atom.

In the aromatic hydrocarbon group, the hydrogen atom contained in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and a methyl group, an ethyl group, a propyl group, an n-butyl group, or a tert-butyl group is more preferable.

Examples of the alkoxy group, the halogen atom, and the halogenated alkyl group, as the substituent, include those exemplified as the substituent that is substituted for a hydrogen atom contained in the cyclic aliphatic hydrocarbon group.

### Divalent Linking Group Containing Hetero Atom

In a case where Ya²¹ represents a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group, an acyl group, or the like), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²², or —Y²¹—S(═O)₂—O—Y²²— [in the formulae, Y²¹ and Y²² each independently represents a divalent hydrocarbon group which may have a substituent, O represents an oxygen atom, and m″ represents an integer in a range of 0 to 3].

In a case where the divalent linking group containing a hetero atom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) preferably has 1 to 10 carbon atoms, more preferably has 1 to 8 carbon atoms, and particularly preferably has 1 to 5 carbon atoms. In Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²², or —Y²¹—S(═O)₂—O—Y²², Y²¹ and Y²² each independently represents a divalent hydrocarbon group which may have a substituent. Examples of the divalent hydrocarbon group include the same one as “the divalent hydrocarbon group which may have a substituent” described in the explanation of the above-described divalent linking group as Ya²¹.

Y²¹ is preferably a linear aliphatic hydrocarbon group, more preferably a linear alkylene group, still more preferably a linear alkylene group having 1 to 5 carbon atoms, and particularly preferably a methylene group or an ethylene group.

Y²² is preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²², m″ represents an integer of 0 to 3, preferably an integer of 0 to 2, more preferably 0 or 1, and particularly preferably 1. In other words, the group represented by Formula —[Y²¹—C(═O)—O]_{m″}—Y²²— is particularly preferably a group represented by Formula —Y²¹—C(═O)—O—Y²²—. Among the above, a group represented by Formula —(CH₂)_{a′}—C(═O)—O—(CH₂)_{b′}— is preferable. In the formula, a′ represents an integer in a range of 1 to 10, preferably an integer in a range of 1 to 8, more preferably an integer in a range of 1 to 5, still more preferably 1 or 2, and most preferably 1. b′ represents an integer in a range of 1 to 10, preferably an integer in a range of 1 to 8, more preferably an integer in a range of 1 to 5, still more preferably 1 or 2, and most preferably 1.

Among the above, Ya²¹ is preferably a single bond, an ester bond [—C(═O)—O—], an ether bond (—O—), a linear or branched alkylene group, or a combination thereof.

In General Formula (a2-1), Ra²¹ represents a lactone-containing cyclic group, an —SO₂—-containing cyclic group, or a carbonate-containing cyclic group.

Suitable examples of the lactone-containing cyclic group, the —SO₂—-containing cyclic group, and the carbonate-containing cyclic group as Ra²¹ include groups each represented by General Formulae (a2-r-1) to (a2-r-7), groups each represented by General Formulae (a5-r-1) to (a5-r-4), and groups each represented by General Formulae (ax3-r-1) to (ax3-r-3) described above.

Among them, a lactone-containing cyclic group or an —SO₂—-containing cyclic group is preferable, groups each represented by General Formula (a2-r-1), (a2-r-2), (a2-r-6), or (a5-r-1) are more preferable, and groups each represented by General Formula (a2-r-2) or (a5-r-1) are still more preferable. Specifically, any one of groups each represented by Chemical Formulae (r-lc-1-1) to (r-lc-1-7), (r-lc-2-1) to (r-lc-2-18), (r-lc-6-1). (r-sl-1-1), or (r-sl-1-18), is preferable, any one of groups each represented by Chemical Formulae (r-lc-2-1) to (r-lc-2-18), or (r-sl-1-1) is more preferable, and any one of groups each represented by Chemical Formula (r-lc-2-1), (r-lc-2-12), or (r-sl-1-1) is still more preferable.

The constitutional unit (a2) contained in the component (A1) may be one kind or may be two or more kinds.

In a case where the component (A1) has the constitutional unit (a2), the proportion of the constitutional unit (a2) is preferably in a range of 5% to 60% by mole, more preferably in a range of 10% to 60% by mole, still more preferably in a range of 20% to 60% by mole, and particularly preferably in a range of 30% to 60% by mole with respect to the total (100% by mole) of all constitutional units constituting the component (A1).

In a case where the proportion of the constitutional unit (a2) is equal to or larger than the lower limit value of the preferred range, the effect obtained by allowing the constitutional unit (a2) to be contained can be sufficiently achieved by the effect described above. In a case where the proportion of the constitutional unit (a2) is equal to or smaller than the upper limit value of the preferred range, balance with other constitutional units can be obtained, and various lithography characteristics are improved.

### In Regard to Constitutional Unit (a3):

The component (A1) may further have a constitutional unit (a3) (however, a constitutional unit corresponding to the constitutional unit (a1) or the constitutional unit (a2) is excluded) containing a polar group-containing aliphatic hydrocarbon group. In a case where the component (A1) has the constitutional unit (a3), the hydrophilicity of the component (A) is increased, which contributes to an improvement in resolution. In addition, acid diffusion length can be properly adjusted.

Examples of the polar group include a hydroxyl group, a cyano group, a carboxy group, or a hydroxyalkyl group in which a part of hydrogen atoms of the alkyl group have been substituted with a fluorine atom, and the polar group is particularly preferably a hydroxyl group.

Examples of the aliphatic hydrocarbon group include a linear or branched hydrocarbon group (preferably an alkylene group) having 1 to 10 carbon atoms, and a cyclic aliphatic hydrocarbon group (a cyclic group). The cyclic group may be a monocyclic group or a polycyclic group. For example, these cyclic groups can be appropriately selected from a large number of groups that have been proposed in resins for a resist composition for an ArF excimer laser.

In a case where the cyclic group is a monocyclic group, the monocyclic group preferably has 3 to 10 carbon atoms. Among them, a constitutional unit derived from an acrylic acid ester that contains an aliphatic monocyclic group containing a hydroxyl group, a cyano group, a carboxy group, or a hydroxyalkyl group in which a part of hydrogen atoms of the alkyl group have been substituted with a fluorine atom is particularly preferable. Examples of the monocyclic group include a group obtained by removing two or more hydrogen atoms from a monocycloalkane. Specific examples of the monocyclic group include a group obtained by removing two or more hydrogen atoms from a monocycloalkane such as cyclopentane, cyclohexane, or cyclooctane. Among these monocyclic groups, a group obtained by removing two or more hydrogen atoms from cyclopentane or a group obtained by removing two or more hydrogen atoms from cyclohexane are industrially preferable.

In a case where the cyclic group is a polycyclic group, the polycyclic group preferably has 7 to 30 carbon atoms. Among them, a constitutional unit derived from an acrylic acid ester that contains an aliphatic polycyclic group containing a hydroxyl group, a cyano group, a carboxy group, or a hydroxyalkyl group in which a part of hydrogen atoms of the alkyl group have been substituted with a fluorine atom is particularly preferable. Examples of the polycyclic group include groups obtained by removing two or more hydrogen atoms from a bicycloalkane, tricycloalkane, tetracycloalkane, or the like. Specific examples thereof include a group obtained by removing two or more hydrogen atoms from a polycycloalkane such as adamantane, norbornane, isobornane, tricyclodecane, or tetracyclododecane. Among these polycyclic groups, a group obtained by removing two or more hydrogen atoms from adamantane, a group obtained by removing two or more hydrogen atoms from norbornane, or a group obtained by removing two or more hydrogen atoms from tetracyclododecane are industrially preferable.

The constitutional unit (a3) is not particularly limited, and any constitutional unit may be used as long as the constitutional unit contains a polar group-containing aliphatic hydrocarbon group.

The constitutional unit (a3) is a constitutional unit derived from an acrylic acid ester in which the hydrogen atom bonded to the carbon atom at the α-position may be substituted with a substituent, and a constitutional unit containing a polar group-containing aliphatic hydrocarbon group is preferable.

In a case where the hydrocarbon group in the polar group-containing aliphatic hydrocarbon group is a linear or branched hydrocarbon group having 1 to 10 carbon atoms, the constitutional unit (a3) is preferably a constitutional unit derived from a hydroxyethyl ester of acrylic acid.

In addition, as the constitutional unit (a3), in a case where the hydrocarbon group in the polar group-containing aliphatic hydrocarbon group is a polycyclic group, a constitutional unit represented by General Formula (a3-1), a constitutional unit represented by General Formula (a3-2), and a constitutional unit represented by General Formula (a3-3) are preferable, and in a case where the hydrocarbon group is a monocyclic group, a constitutional unit represented by General Formula (a3-4) is preferable.

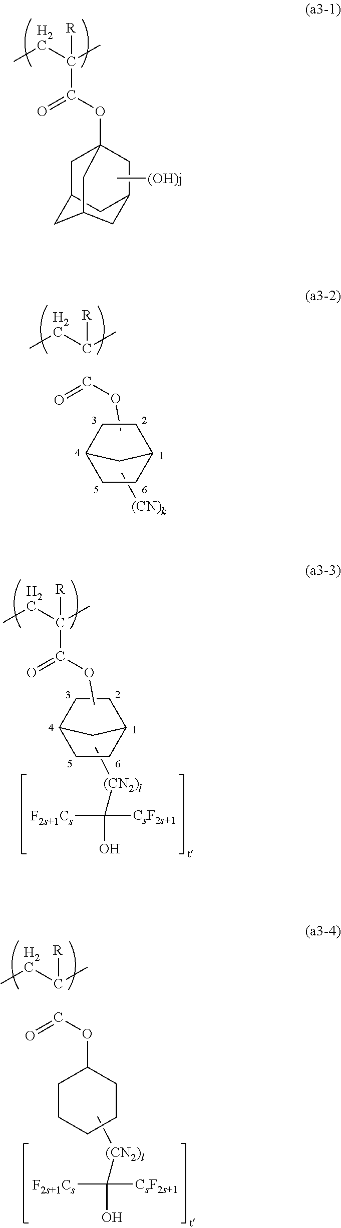

[In the formulae, R has the same definition as described above, j represents an integer in a range of 1 to 3, k represents an integer in a range of 1 to 3, t′ represents an integer in a range of 1 to 3, l represents an integer in a range of 0 to 5, and s represents an integer in a range of 1 to 3.]

In General Formula (a3-1), j is preferably 1 or 2 and more preferably 1. In a case where j represents 2, it is preferable that the hydroxyl groups be bonded to the 3- and 5-positions of the adamantyl group. In a case where j represents 1, it is preferable that the hydroxyl group be bonded to the 3-position of the adamantyl group.

It is preferable that j represent 1, and it is particularly preferable that the hydroxyl group be bonded to the 3-position of the adamantyl group.

In General Formula (a3-2), k is preferably 1. The cyano group is preferably bonded to the 5- or 6-position of the norbornyl group.

In General Formula (a3-3), it is preferable that t′ represent 1. It is preferable that l represent 1. It is preferable that s represent 1. Further, it is preferable that a 2-norbornyl group or 3-norbornyl group be bonded to the terminal of the carboxy group of the acrylic acid. It is preferable that the fluorinated alkyl alcohol be bonded to the 5-position or 6-position of the norbornyl group.

In General Formula (a3-4), it is preferable that t′ represent 1 or 2. It is preferable that l represent 0 or 1. It is preferable that s represent 1. It is preferable that the fluorinated alkyl alcohol be bonded to the 3- or 5-position of the cyclohexyl group.

The constitutional unit (a3) contained in the component (A1) may be one kind or may be two or more kinds.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole, with respect to the total (100% by mole) of all constitutional units constituting the component (A1).

In a case where the proportion of the constitutional unit (a3) is equal to or larger than the lower limit value of the preferred range, the effect obtained by allowing the constitutional unit (a3) to be contained can be sufficiently achieved by the effect described above. In a case where the proportion of the constitutional unit (a3) is equal to or smaller than the upper limit value of the preferred range, balance with other constitutional units can be obtained, and various lithography characteristics are improved.

### In Regard to Constitutional Unit (a4):

The component (A1) may further have a constitutional unit (a4) containing an acid non-dissociable aliphatic cyclic group.

In a case where the component (A1) has the constitutional unit (a4), the dry etching resistance of the formed resist pattern is improved. In addition, the hydrophobicity of the component (A) increases. The improvement in hydrophobicity contributes to the improvement in resolution, a resist pattern shape, and the like, particularly in the case of a solvent developing process.

The term “acid non-dissociable cyclic group” in the constitutional unit (a4) is a cyclic group that remains in the constitutional unit without being dissociated even when an acid acts in a case where the acid is generated in the resist composition by exposure (for example, in a case where acid is generated from the constitutional unit that generates acid upon exposure, or the component (B)).

Examples of the constitutional unit (a4) preferably include a constitutional unit derived from an acrylic acid ester including an acid non-dissociable aliphatic cyclic group. As the cyclic group, a large number of cyclic groups known in the related art as cyclic groups used as a resin component of a resist composition for ArF excimer laser, KrF excimer laser (preferably ArF excimer laser), or the like can be used.

The cyclic group is particularly preferably at least one selected from a tricyclodecyl group, an adamantyl group, a tetracyclododecyl group, an isobornyl group, and a norbornyl group, from the viewpoint of industrial availability. These polycyclic groups may have, as a substituent, a linear or branched alkyl group having 1 to 5 carbon atoms.

Specific examples of the constitutional unit (a4) include constitutional units each represented by General Formulae (a4-1) to (a4-7).

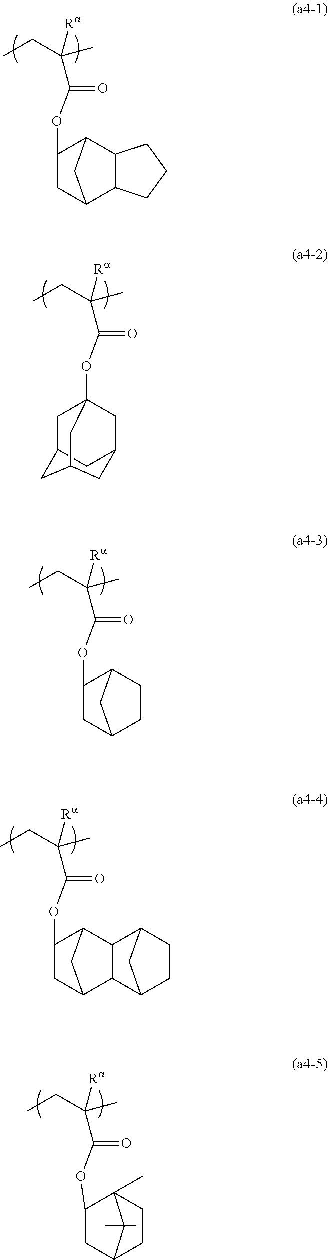

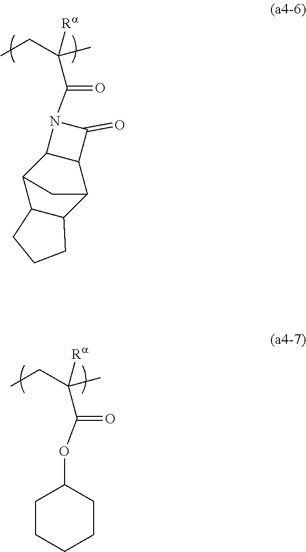

[In the formulae, R^{α} has the same definition as described above.]

The constitutional unit (a4) contained in the component (A1) may be one kind or may be two or more kinds.

In a case where the component (A1) has the constitutional unit (a4), the proportion of the constitutional unit (a4) is preferably in a range of 1% to 40% by mole and more preferably in a range of 5% to 20% by mole, with respect to the total (100% by mole) of all constitutional units constituting the component (A1).

In a case where the proportion of the constitutional unit (a4) is equal to or larger than the lower limit value of the preferred range, the effect that is obtained by allowing the constitutional unit (a4) to be contained can be sufficiently achieved. In a case where the proportion of the constitutional unit (a4) is equal to or smaller than the upper limit value of the preferred range, the balance with other constitutional units is obtained easily.

In regard to constitutional unit (a10):

The constitutional unit (a10) is a constitutional unit represented by General Formula (a10-1).

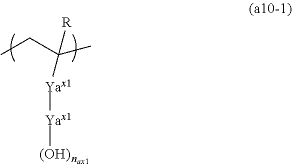

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or more.]

In General Formula (a10-1), R represents a hydrogen atom, an alkyl group having 1 to 5 carbon atoms, or a halogenated alkyl group having 1 to 5 carbon atoms. R is the same as R in General Formula (a01-1-1).

In General Formula (a10-1), Ya^{x1} represents a single bond or a divalent linking group.

In the chemical formulae described above, the divalent linking group as Ya^{x1} is not particularly limited, and suitable examples thereof include a divalent hydrocarbon group which may have a substituent, and a divalent linking group having hetero atoms. Examples of the divalent linking group as Ya^{x1} include the same as those described as the divalent linking group as Ya²¹ in General Formula (a2-1).

Among the above, Ya^{x1} is preferably a single bond, an ester bond [—C(═O)—O—, —O—C(═O)—], an ether bond (—O—), a linear or branched alkylene group, or a combination thereof, and more preferably a single bond or an ester bond [—C(═O)—O—, —O—C(═O)—].

In General Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as it is a cyclic conjugated system having (4n+2) π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Specific examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among the above, Wa^{x1} is preferably a group obtained by removing (nₐₓ₁+1) hydrogen atoms from benzene, naphthalene, anthracene, or biphenyl, more preferably a group obtained by removing (nₐₓ₁+1) hydrogen atoms from benzene or naphthalene, and still more preferably a group obtained by removing (nₐₓ₁+1) hydrogen atoms from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include the same as those described as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. The aromatic hydrocarbon group as Wa^{x1} preferably has no substituent.

In General Formula (a10-1), nₐₓ₁ represents an integer of 1 or more, preferably an integer in a range of 1 to 10, more preferably an integer in a range of 1 to 5, still more preferably 1, 2, or 3, and particularly preferably 1 or 2.

Specific examples of the constitutional unit (a10) represented by General Formula (a10-1) are shown below.

In the formulae shown below, R^{α} represents a hydrogen atom, a methyl group, or a trifluoromethyl group.

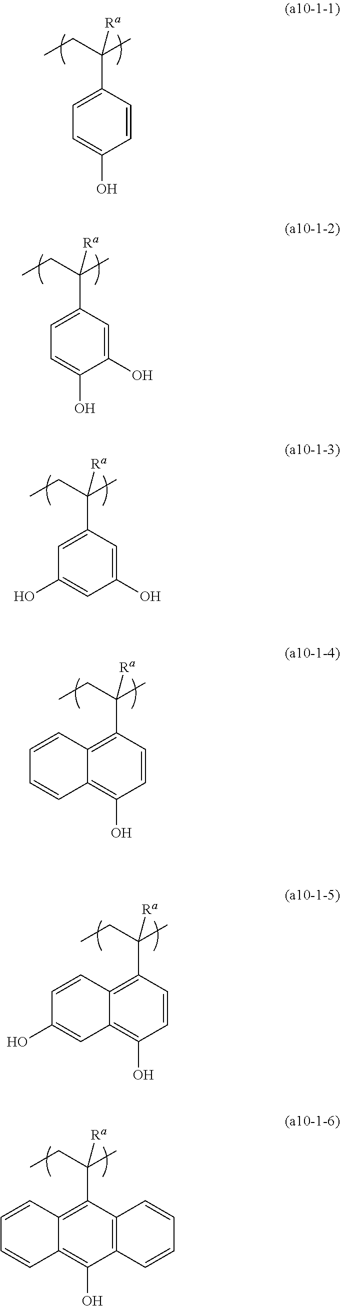

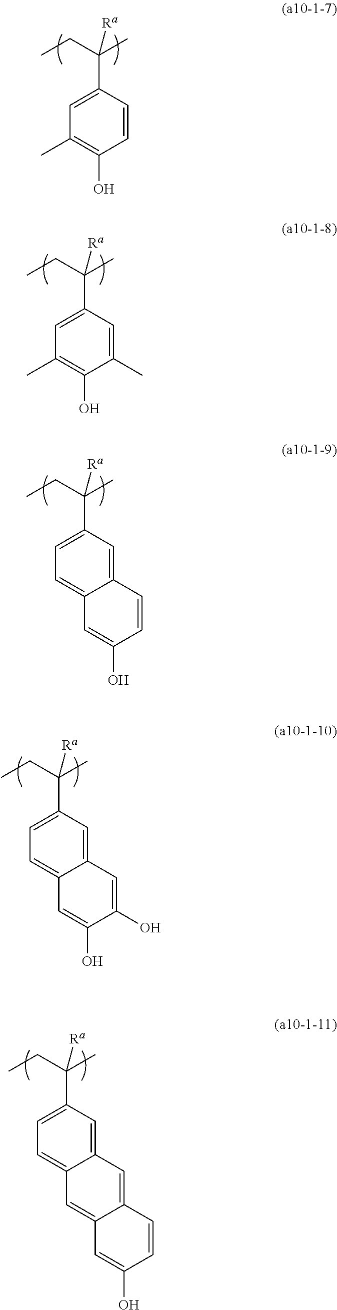

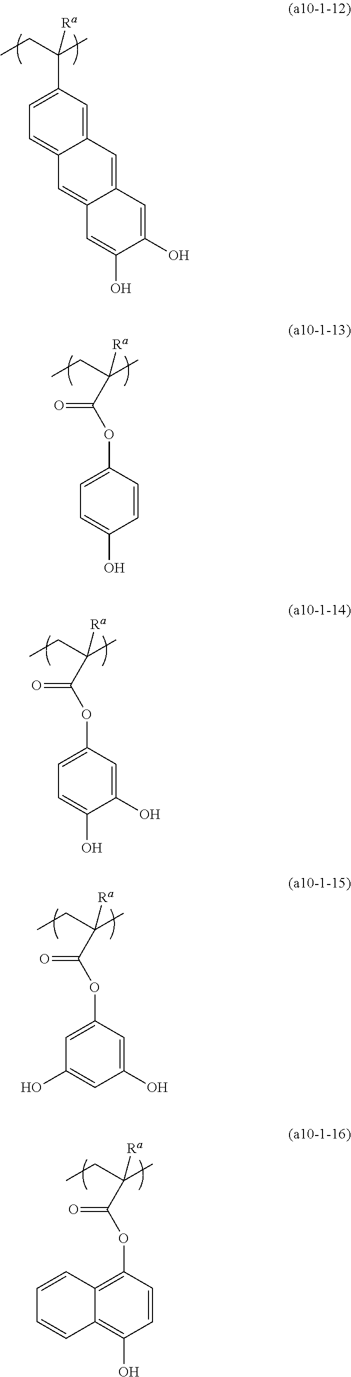

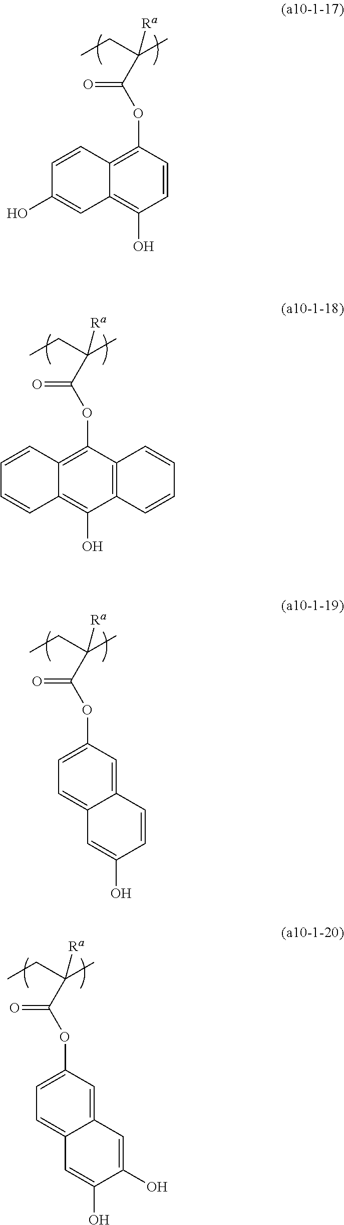

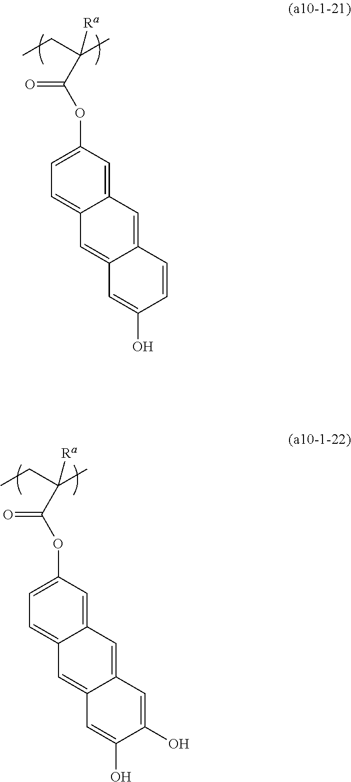

The constitutional unit (a10) contained in the component (A1) may be one kind or may be two or more kinds.

In a case where the component (A1) has the constitutional unit (a10), the proportion of the constitutional unit (a10) in the component (A1) is preferably in a range of 5% to 80% by mole, more preferably in a range of 5% to 70% by mole, still more preferably in a range of 10% to 60% by mole, and particularly preferably in a range of 20% to 50% by mole, with respect to the total (100% by mole) of all constitutional units constituting the component (A1).

In a case where the proportion of the constitutional unit (a10) is equal to or larger than the lower limit value of the above-described preferred range, the sensitivity is more easily increased. In a case where the proportion of the constitutional unit (a10) is equal to or smaller than the upper limit value of the above-described preferred range, the balance between the constitutional unit (a10) and other constitutional units is easily achieved.

### In Regard to Constitutional Unit (st):

The constitutional unit (st) is a constitutional unit derived from styrene or a styrene derivative. The term “constitutional unit derived from styrene” means a constitutional unit that is formed by the cleavage of an ethylenic double bond of styrene. The term “constitutional unit derived from a styrene derivative” means a constitutional unit formed by the cleavage of an ethylenic double bond of a styrene derivative.

The term “styrene derivative” means a compound in which at least a part of hydrogen atoms of styrene are substituted with a substituent. Examples of the styrene derivative include a derivative in which the hydrogen atom at the α-position of styrene is substituted with a substituent, a derivative in which one or more hydrogen atoms of the benzene ring of styrene are substituted with a substituent, and a derivative in which the hydrogen atom at the α-position of styrene and one or more hydrogen atoms of the benzene ring are substituted with a substituent.

Examples of the substituent that is substituted for the hydrogen atom at the α-position of styrene include an alkyl group having 1 to 5 carbon atoms or a halogenated alkyl group having 1 to 5 carbon atoms.

The alkyl group having 1 to 5 carbon atoms is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group obtained by substituting part or all of hydrogen atoms in the alkyl group having 1 to 5 carbon atoms with a halogen atom. The halogen atom is particularly preferably a fluorine atom.

The substituent that is substituted for the hydrogen atom at the α-position of styrene is preferably an alkyl group having 1 to 5 carbon atoms or a fluorinated alkyl group having 1 to 5 carbon atoms, more preferably an alkyl group having 1 to 3 carbon atoms or a fluorinated alkyl group having 1 to 3 carbon atoms, and still more preferably a methyl group from the viewpoint of industrial availability.

Examples of the substituent that is substituted for the hydrogen atom of the benzene ring of styrene include an alkyl group, an alkoxy group, a halogen atom, and a halogenated alkyl group.

The alkyl group as the substituent is preferably an alkyl group having 1 to 5 carbon atoms, and a methyl group, an ethyl group, a propyl group, an n-butyl group, or a tert-butyl group is more preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as the substituent is preferably a fluorine atom.

Examples of the halogenated alkyl group as the substituent include groups in which part or all of hydrogen atoms in the above-described alkyl groups have been substituted with the above-described halogen atoms.

The substituent that is substituted for the hydrogen atom of the benzene ring of styrene is preferably an alkyl group having 1 to 5 carbon atoms, more preferably a methyl group or an ethyl group, and still more preferably a methyl group.

The constitutional unit (st) is preferably a constitutional unit derived from styrene or a constitutional unit derived from a styrene derivative in which the hydrogen atom at the α-position of styrene is substituted with an alkyl group having 1 to 5 carbon atoms or a halogenated alkyl group having 1 to 5 carbon atoms, more preferably a constitutional unit derived from styrene, or a constitutional unit derived from a styrene derivative in which the hydrogen atom at the α-position of styrene is substituted with a methyl group, and still more preferably a constitutional unit derived from styrene.

The constitutional unit (st) contained in the component (A1) may be one kind or may be two or more kinds.

In a case where the component (A1) has the constitutional unit (st), the proportion of the constitutional unit (st) is preferably in a range of 1% to 30% by mole and more preferably in a range of 3% to 20% by mole with respect to the total (100% by mole) of all constitutional units constituting the component (A1).

The component (A1) may be used alone or in combination of two or more kinds thereof.

Examples of the component (A1) include a polymeric compound having a repeating structure of the constitutional unit (a01), where the component (A1) is preferably a polymeric compound having a repeating structure of the constitutional unit (a01) and the constitutional unit (a10).

Among the above, the component (A1) is more preferably a polymeric compound consisting of a repeating structure of the constitutional unit (a01); or a polymeric compound consisting of a repeating structure of the constitutional unit (a01) and the constitutional unit (a10).

In the polymeric compound having a repeating structure of the constitutional unit (a01) and the constitutional unit (a10), the proportion of the constitutional unit (a01) is preferably in a range of 10% to 90% by mole, more preferably in a range of 20% to 80% by mole, still more preferably in a range of 30% to 70% by mole, and particularly preferably in a range of 40% to 70% by mole with respect to the total (100% by mole) of all constitutional units constituting the polymeric compound.

The proportion of the constitutional unit (a10) in the polymeric compound described above is preferably in a range of 10% to 80% by mole, more preferably in a range of 20% to 70% by mole, still more preferably in a range of 30% to 60% by mole, and particularly preferably in a range of 30% to 50% by mole, with respect to the total (100% by mole) of all constitutional units constituting the polymeric compound.

The component (A1) can be produced by dissolving, in a polymerization solvent, each monomer from which the constitutional unit is derived, adding thereto a radical polymerization initiator such as azobisisobutyronitrile (AIBN) or dimethyl azobisisobutyrate (for example, V-601) to carry out polymerization, and then carrying out a deprotection reaction.

A —C(CF₃)₂—OH group may be introduced into the terminal thereof during the polymerization by using a chain transfer agent such as HS—CH₂—CH₂—CH₂—C(CF₃)₂—OH in combination. A copolymer into which a hydroxyalkyl group, formed by substitution of a part of hydrogen atoms in the alkyl group with fluorine atoms, has been introduced is effective for reducing development defects and reducing line edge roughness (LER: uneven irregularities of a line side wall).

The weight-average molecular weight (Mw) (in terms of the polystyrene equivalent value determined by gel permeation chromatography (GPC)) of the component (A1), which is not particularly limited, is preferably in a range of 1,000 to 50,000, more preferably in a range of 2,000 to 30.000, and still more preferably in a range of 3,000 to 20,000. The Mw of the component (A1) is more preferably in a range of 4,000 to 15,000 and particularly preferably in a range of 5,000 to 10,000.

In a case where the Mw of the component (A1) is equal to or smaller than the upper limit value of the above-described preferred range, solubility in a resist solvent sufficient for use as a resist is likely to be obtained. In a case where the Mw of the component (A1) is equal to or larger than the lower limit value of the above-described preferred range, the dry etching resistance and the cross-sectional shape of the resist pattern are good.

The polydispersity (Mw/Mn) of the component (A1) is not particularly limited; however, it is preferably in a range of 1.0 to 4.0, more preferably in a range of 1.0 to 3.0, and particularly preferably in a range of 1.0 to 2.0. In addition, Mn indicates the number-average molecular weight.

### In Regard to Component (A2)

In the resist composition according to the present embodiment, a base material component (hereinafter, referred to as a “component (A2)”) that exhibits changed solubility in a developing solution under action of acid, which does not correspond to the component (A1), may be used in combination as the component (A).

The component (A2) is not particularly limited and may be freely selected and used from a large number of base material components for the chemical amplification-type resist composition known in the related art.

As the component (A2), a polymeric compound or a low-molecular-weight compound may be used alone or in a combination of two or more kinds thereof.

The proportion of the component (A1) in the component (A) is preferably 25% by mass or more, more preferably 50% by mass or more, still more preferably 75% by mass or more, and may be 100% by mass with respect to the total mass of the component (A). In a case where the proportion of the component (A1) is 25% by mass or more, a resist pattern having various excellent lithography characteristics such as high sensitivity, resolution, and roughness amelioration can be easily formed.

The content of the component (A) in the resist composition according to the present embodiment may be adjusted depending on the resist film thickness to be formed.

### <Acid Generator Component (B)>

The component (B) in the resist composition according to the present embodiment contains a compound (B0) represented by General Formula (b0) (hereinafter, also referred to as a “component (B0)”).

### <<Compound (B0)>>

The component (B0) is a compound represented by General Formula (b0).

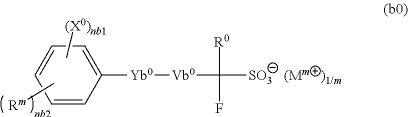

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. Yb⁰ represents a divalent linking group or a single bond. Vb⁰ represents a single bond, an alkylene group, or a fluorinated alkylene group. R⁰ represents a hydrogen atom, a fluorinated alkyl group having 1 to 5 carbon atoms, or a fluorine atom. M^{m+} represents an m-valent organic cation, where m represents an integer of 1 or more.]

### {Anion Moiety of Component (B0)}

In General Formula (b0), X⁰ represents a bromine atom or an iodine atom and is preferably an iodine atom.

In General Formula (b0), R^{m} represents a hydroxy group, an alkyl group, a fluorine atom, or a chlorine atom. The alkyl group as R^{m} is preferably an alkyl group having 1 to 5 carbon atoms and more preferably a methyl group or an ethyl group.

In General Formula (b0), nb1 represents an integer in a range of 1 to 5, and nb2 represents an integer in a range of 0 to 4, where 1≤nb1+nb2≤5 is satisfied.

nb1 is preferably an integer in a range of 1 to 3, more preferably 2 or 3, and still more preferably 3.

nb2 is preferably an integer in a range of 0 to 3, more preferably 0 or 1, and still more preferably 0.

In General Formula (b0), Yb⁰ represents a divalent linking group or a single bond. Suitable examples of the divalent linking group as Yb⁰ include a divalent linking group containing an oxygen atom.

In a case where Yb⁰ represents a divalent linking group containing an oxygen atom, Yb⁰ may contain an atom other than the oxygen atom. Examples of the atom other than the oxygen atom include a carbon atom, a hydrogen atom, a sulfur atom, and a nitrogen atom.

Examples of divalent linking groups containing an oxygen atom include non-hydrocarbon-based oxygen atom-containing linking groups such as an oxygen atom (an ether bond; —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon-based oxygen atom-containing linking groups with an alkylene group. A sulfonyl group (—SO₂—) may be further linked to this combination.

In General Formula (b0). Vb⁰ represents an alkylene group, a fluorinated alkylene group, or a single bond.

The alkylene group and the fluorinated alkylene group as Vb⁰ each preferably has 1 to 4 carbon atoms and more preferably has 1 to 3 carbon atoms. Examples of the fluorinated alkylene group as Vb⁰ include a group obtained by substituting part or all of hydrogen atoms in the alkylene group with a fluorine atom. Among them, Vb⁰ is preferably an alkylene group having 1 to 4 carbon atoms, a fluorinated alkylene group having 1 to 4 carbon atoms, or a single bond, and more preferably a group obtained by substituting a part of hydrogen atoms of an alkylene group having 1 to 3 carbon atoms with a fluorine atom, or a single bond, and still more preferably —CH(CF₃)— or a single bond.

In General Formula (b0), R⁰ represents a hydrogen atom, a fluorinated alkyl group having 1 to 5 carbon atoms, or a fluorine atom. R⁰ is preferably a fluorine atom or a perfluoroalkyl group having 1 to 5 carbon atoms and more preferably a fluorine atom.

In the present embodiment, the anion moiety of the component (B0) is preferably an anion represented by General Formula (b0-an0).

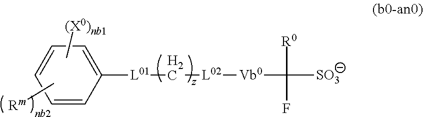

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}). R^{a}'s each independently represents a hydrogen atom or an alkyl group. z represents an integer in a range of 0 to 10. Vb⁰ represents a single bond, an alkylene group, or a fluorinated alkylene group. R⁰ represents a hydrogen atom, a fluorinated alkyl group having 1 to 5 carbon atoms, or a fluorine atom.]

X⁰, R^{m}, nb1, nb2, Vb⁰, and R⁰ in General Formula (b0-an0) are each the same as X⁰, R^{m}, nb1, nb2, Vb⁰, and R⁰ in General Formula (b0) described above.

In General Formula (b0-an0), L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}). R^{a}'s each independently represents a hydrogen atom or an alkyl group.

The alkylene group as L⁰¹ and L⁰² and the alkyl group as R^{a} each preferably has 1 to 4 carbon atoms and more preferably 1 to 3 carbon atoms.

In General Formula (b0-an0), among the above, it is preferable that any one of L⁰¹ and L⁰² be —OCO— or —COO—, and it is more preferable that L⁰¹ be —OCO— or —COO and L⁰² be a single bond, —OCO—, or —COO—.

More specifically, in General Formula (b0-an0), -L⁰¹-(CH₂)z-L⁰²-Vb⁰- is preferably —COO—Vb⁰-, —OCO—Vb⁰-, or —COO—(CH₂), —COO—Vb⁰-.

In General Formula (b0-an0), z represents an integer in a range of 0 to 10, preferably an integer of 0 to 5, and more preferably an integer in a range of 0 to 3.

Specific examples of the anion moiety of the component (B0) are shown below.

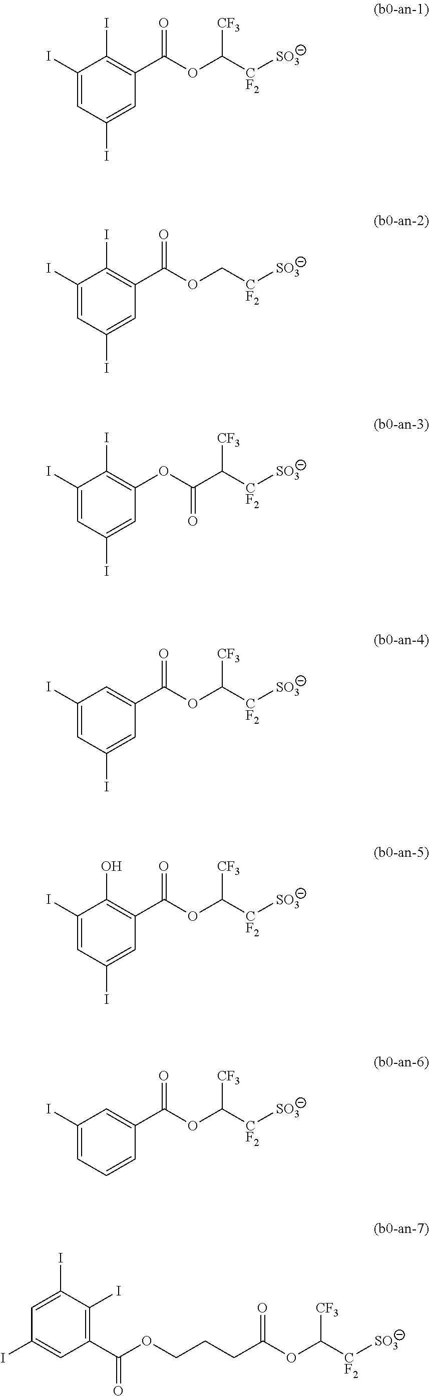

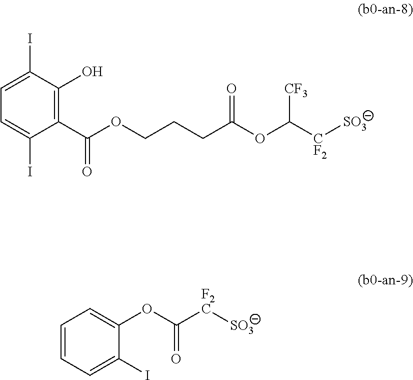

### {Cation Moiety of Component (B0)}

In General Formula (b0), M^{m+} represents an m-valent organic cation. Among them, a sulfonium cation and an iodonium cation are preferable.

m represents an integer of 1 or more.

Examples of the preferred cation moiety (M^{m+})_{1/m}) include an organic cation represented by each of General Formulae (ca-1) to (ca-5).

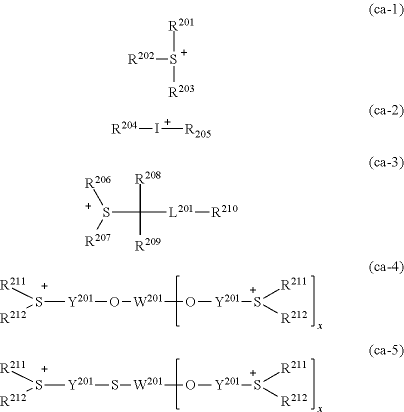

[In the formula, R²⁰¹ to R²⁰⁷, R²¹¹, and R²¹² each independently represents an aryl group, an alkyl group, or an alkenyl group, each of which may have a substituent. R²⁰¹ to R²⁰³, R²⁰⁶ and R²⁰⁷, and R²¹¹ and R²¹² may be bonded to each other to form a ring together with the sulfur atom in the formula. R²⁰⁸ and R²⁰⁹ each independently represents a hydrogen atom or an alkyl group having 1 to 5 carbon atoms. R²¹⁰ represents an aryl group which may have a substituent, an alkyl group which may have a substituent, an alkenyl group which may have a substituent, or an —SO₂—-containing cyclic group which may have a substituent. L²⁰¹ represents —C(═O)— or —C(═O)—O—.] Y²⁰¹'s each independently represents an arylene group, an alkylene group, or an alkenylene group. x represents 1 or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms, and a phenyl group or a naphthyl group is preferable.

The alkyl group as R²⁰¹ to R²⁰⁷, R²¹¹, and R²¹² is preferably a chain-like or cyclic alkyl group, where the number of carbon atoms thereof is in a range of 1 to 30.

It is preferable that the alkenyl group as R²⁰¹ to R²⁰⁷, R²¹¹, and R²¹² have 2 to 10 carbon atoms.

Examples of the substituent which may be contained in R²⁰¹ to R²⁰⁷ and R²¹⁰ to R²¹² include an alkyl group, a halogen atom, a halogenated alkyl group, a carbonyl group, a cyano group, an amino group, an aryl group, and groups each represented by General Formulae (ca-r-1) to (ca-r-7).

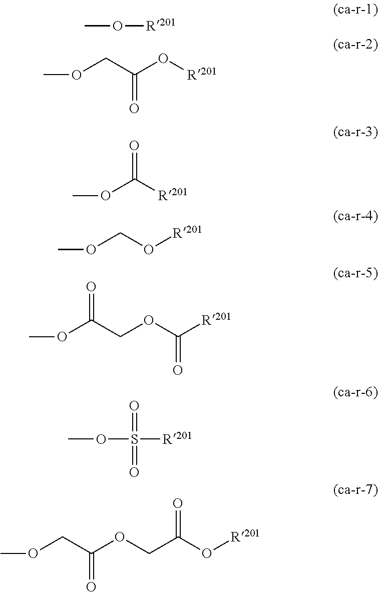

[In the formulae, each R′²⁰¹ independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

The aromatic hydrocarbon group as R′²⁰¹ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms in a substituent is not included in the number of carbon atoms in the aromatic hydrocarbon group.

Specific examples of the aromatic ring which the aromatic hydrocarbon group has as R′²⁰¹ include benzene, fluorene, naphthalene, anthracene, phenanthrene, biphenyl, and an aromatic heterocyclic ring obtained by substituting part of carbon atoms constituting any one of these aromatic rings with a hetero atom. Examples of the hetero atom in the aromatic heterocyclic rings include an oxygen atom, a sulfur atom, and a nitrogen atom.

Specific examples of the aromatic hydrocarbon group as R′²⁰¹ include a group (an aryl group such as a phenyl group or a naphthyl group) obtained by removing one hydrogen atom from the above-described aromatic ring and a group (an arylalkyl group such as a benzyl group, a phenethyl group, a 1-naphthylmethyl group, a 2-naphthylmethyl group, 1-naphthylethyl group, or a 2-naphthylethyl group) obtained by substituting one hydrogen atom in the aromatic ring with an alkylene group. The alkylene group (an alkyl chain in the arylalkyl group) preferably has 1 to 4 carbon atoms, more preferably has 1 or 2 carbon atoms, and particularly preferably has 1 carbon atom.

Examples of the cyclic aliphatic hydrocarbon group as R′²⁰¹ include aliphatic hydrocarbon groups containing a ring in the structure thereof.

Examples of the aliphatic hydrocarbon group containing a ring in the structure thereof include an alicyclic hydrocarbon group (a group obtained by removing one hydrogen atom from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group preferably has 3 to 20 carbon atoms and more preferably 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane is preferably a monocycloalkane having 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among the above, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

Among them, the cyclic aliphatic hydrocarbon group as R′²⁰¹ is preferably a group obtained by removing one or more hydrogen atoms from a monocycloalkane or a polycycloalkane, more preferably a group obtained by removing one hydrogen atom from a polycycloalkane, particularly preferably an adamantyl group or a norbornyl group, and most preferably an adamantyl group.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

The cyclic hydrocarbon group as R′²⁰¹ may contain a hetero atom such as a heterocyclic ring. Specific examples thereof include the lactone-containing cyclic groups each represented by General Formulae (a2-r-1) to (a2-r-7), the —SO₂—-containing cyclic groups each represented by General Formulae (a5-r-1) to (a5-r-4), and other heterocyclic groups each represented by Chemical Formulae (r-hr-1) to (r-hr-16).

Examples of the substituent of the cyclic group as R′²⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as the substituent is preferably a fluorine atom.

Examples of the above-described halogenated alkyl group as the substituent include a group in which part or all of hydrogen atoms in an alkyl group having 1 to 5 carbon atoms such as a methyl group, an ethyl group, a propyl group, an n-butyl group, or a tert-butyl group have been substituted with the above-described halogen atom.

The carbonyl group as the substituent is a group that is substituted for a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

Such a chain-like alkenyl group as R′²⁰¹ may be linear or branched, preferably has 2 to 10 carbon atoms, more preferably has 2 to 5 carbon atoms, still more preferably has 2 to 4 carbon atoms, and particularly preferably has 3 carbon atoms. Examples of the linear alkenyl group include a vinyl group, a propenyl group (an allyl group), and a butynyl group. Examples of the branched alkenyl group include a 1-methylvinyl group. a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent in the chain-like alkyl group or alkenyl group as R′²⁰¹, an alkoxy group, a halogen atom, a halogenated alkyl group, a hydroxyl group, a carbonyl group, a nitro group, an amino group, a cyclic group as R′²⁰¹ or the like may be used.

As the cyclic group which may have a substituent, the chain-like alkyl group which may have a substituent, or the chain-like alkenyl group which may have a substituent, as R′²⁰¹, a group that is the same as the acid-dissociable group represented by above-described General Formula (a1-r-2) can be mentioned as the same one as the cyclic group which may have a substituent or the chain-like alkyl group which may have a substituent, in addition to the groups described above.

Among them, R′²⁰¹ is preferably a cyclic group which may have a substituent, and more preferably a cyclic hydrocarbon group which may have a substituent. More specific examples thereof preferably include a phenyl group, a naphthyl group, a group obtained by removing one or more hydrogen atoms from a polycycloalkane, lactone-containing cyclic groups each represented by any one of General Formulae (a2-r-1) to (a2-r-7), and —SO₂—-containing cyclic groups each represented by any one of General Formulae (a5-r-1) to (a5-r-4).

In Formulae (ca-1) to (ca-5), in a case where R²⁰¹ to R²⁰³, R²⁰⁶ and R²⁰⁷, and R²¹¹ and R²¹² are bonded to each other to form a ring together with the sulfur atom in the formula, these groups may be bonded to each other through a hetero atom such as a sulfur atom, an oxygen atom, or a nitrogen atom, or a functional group such as a carbonyl group, —SO—, —SO₂—, —SO₃—, —COO—, —CONH—, or —N(R_{N})— (here, R_{N} represents an alkyl group having 1 to 5 carbon atoms). Regarding the ring to be formed, it is preferable that a ring containing the sulfur atom in the formula in the ring skeleton thereof be a 3- to 10-membered ring, and it is particularly preferable that it be a 5- to 7-membered ring, in a case where the sulfur atom is included. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represents a hydrogen atom or an alkyl group having 1 to 5 carbon atoms and are preferably a hydrogen atom or an alkyl group having 1 to 3 carbon atoms. In a case of representing an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or an —SO₂—-containing cyclic group which may have a substituent.

Examples of the aryl group as R²¹⁰ include an unsubstituted aryl group having 6 to 20 carbon atoms, and a phenyl group or a naphthyl group is preferable.

The alkyl group as R²¹⁰ is preferably a chain-like or cyclic alkyl group which has 1 to 30 carbon atoms.

The alkenyl group as R²¹⁰ preferably has 2 to 10 carbon atoms.

The —SO₂—-containing cyclic group which may have a substituent, as R²¹⁰, is preferably a “—SO₂—-containing polycyclic group”, and more preferably a group represented by General Formula (a5-r-1).

Y²⁰¹'s each independently represents an arylene group, an alkylene group, or an alkenylene group.

Examples of the arylene group as Y²⁰¹ include groups obtained by removing one hydrogen atom from an aryl group mentioned as the aromatic hydrocarbon group represented by R¹⁰¹ in General Formula (b-1) described above.

Examples of the alkylene group and alkenylene group as Y²⁰¹ include groups obtained by removing one hydrogen atom from the chain-like alkyl group or the chain-like alkenyl group as R¹⁰¹ in General Formula (b-1) described above.

In General Formula (ca-4), x represents 1 or 2.

W²⁰¹ represents an (x+1)-valent linking group, that is, a divalent or trivalent linking group.

The divalent linking group as W²⁰¹ is preferably a divalent hydrocarbon group which may have a substituent, and examples thereof include the same divalent hydrocarbon group, which may have a substituent, as Ya²¹ in General Formula (a2-1) described above. The divalent linking group as W²⁰¹ may be linear, branched, or cyclic, and it is preferably cyclic. Among these, an arylene group having both terminals at which two carbonyl groups are combined is preferable. Examples of the arylene group include a phenylene group and a naphthylene group, and a phenylene group is particularly preferable.

Examples of the trivalent linking group as W²⁰¹ include a group obtained by removing one hydrogen atom from the above-described divalent linking group as W²⁰¹ and a group obtained by bonding the divalent linking group to another divalent linking group. The trivalent linking group as W²⁰¹ is preferably a group obtained by bonding two carbonyl groups to an arylene group.

Specific examples of the suitable cation represented by General Formula (ca-1) include cations each represented by Chemical Formulae (ca-1-1) to (ca-1-72) shown below.

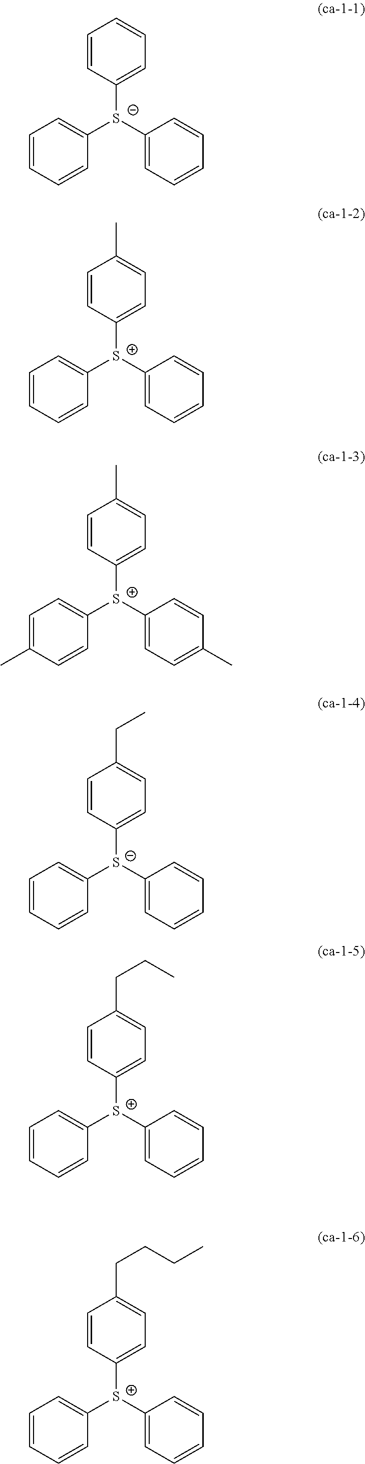

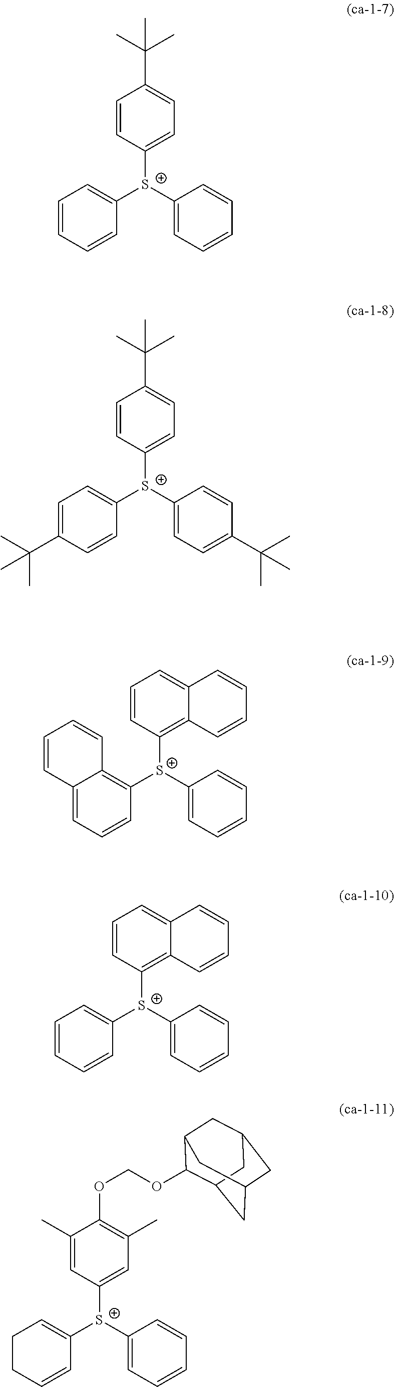

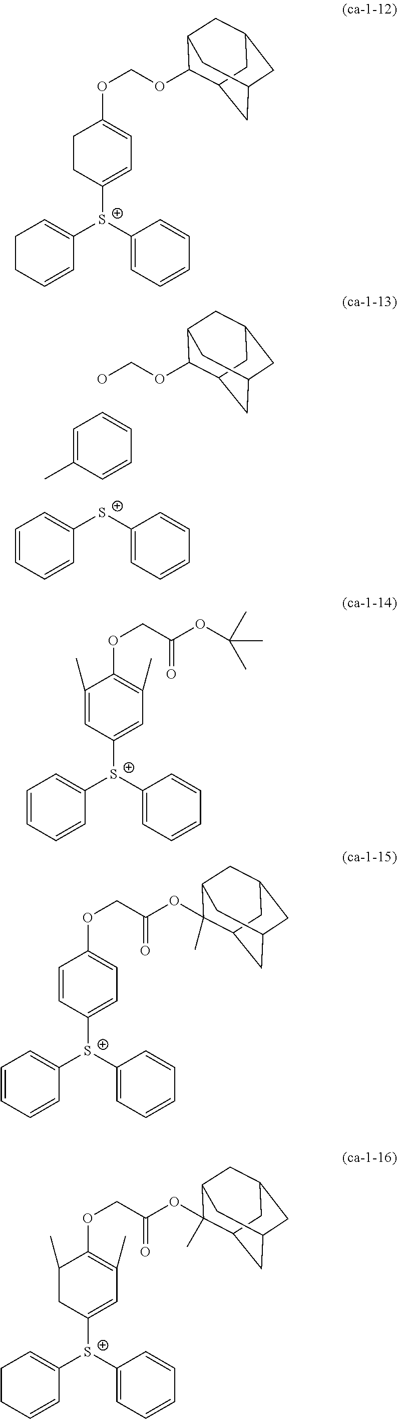

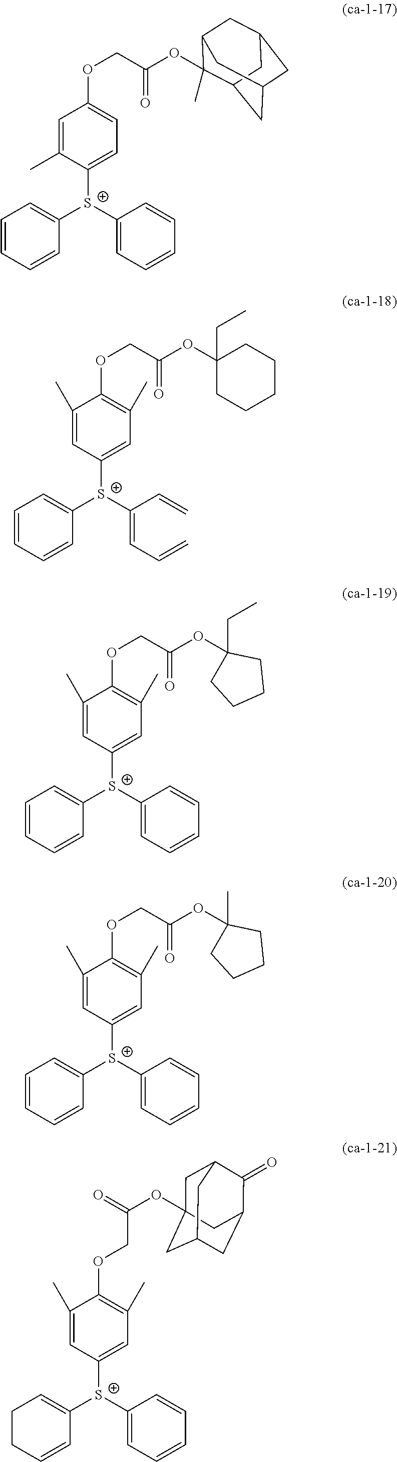

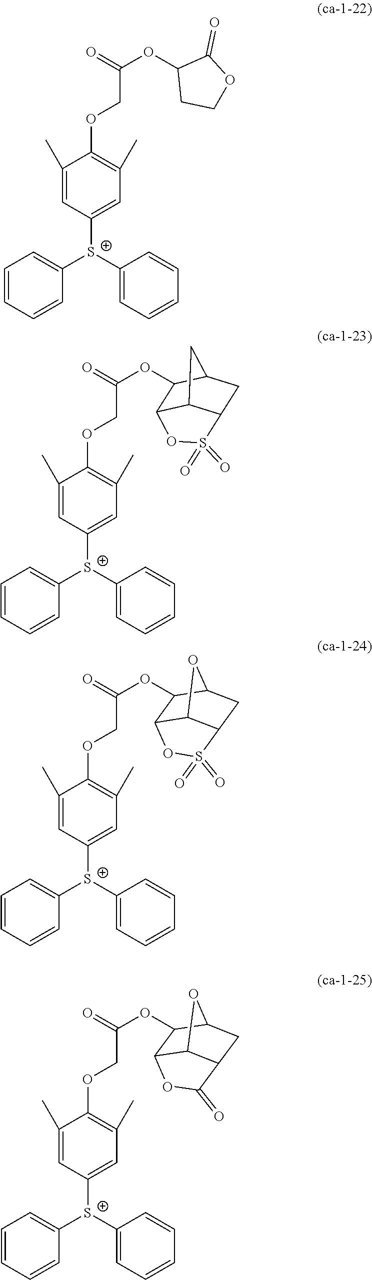

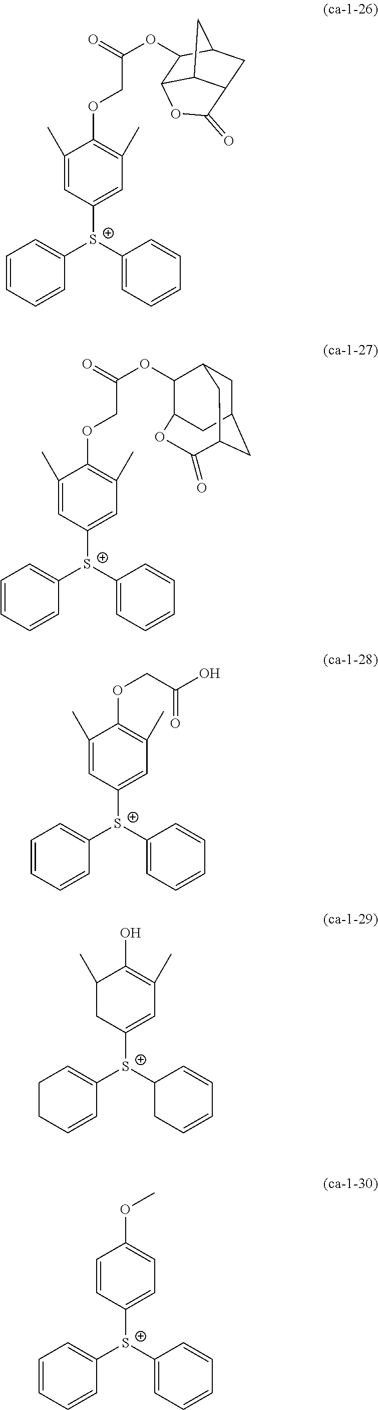

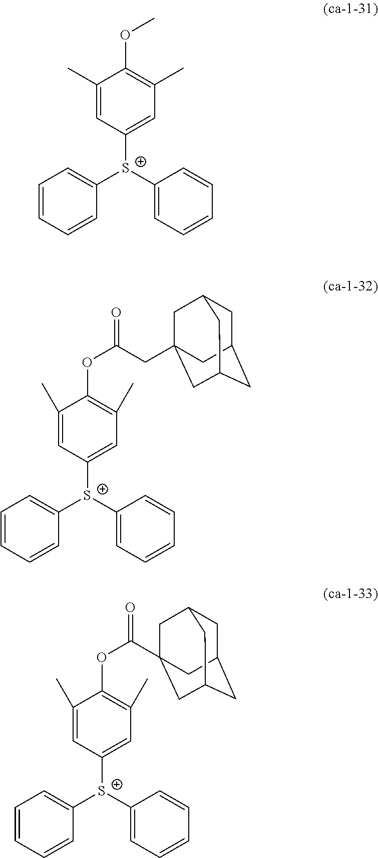

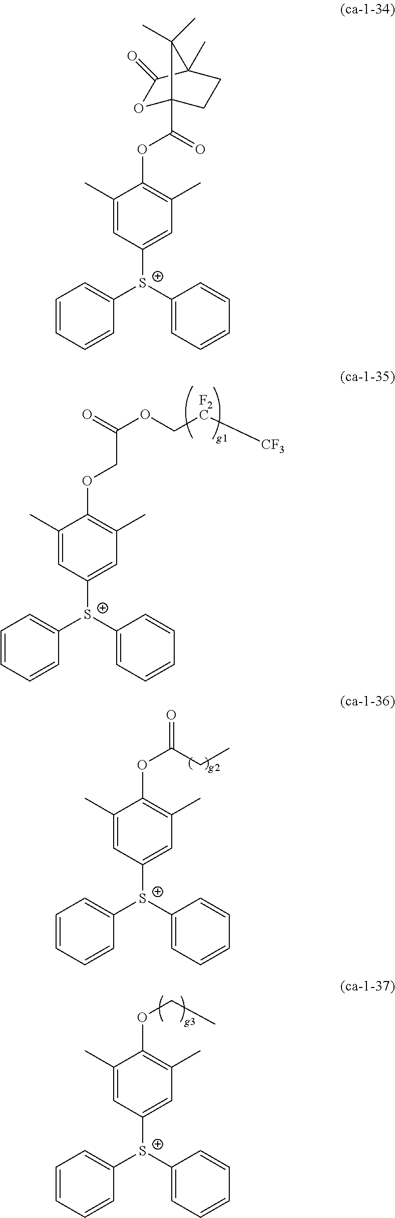

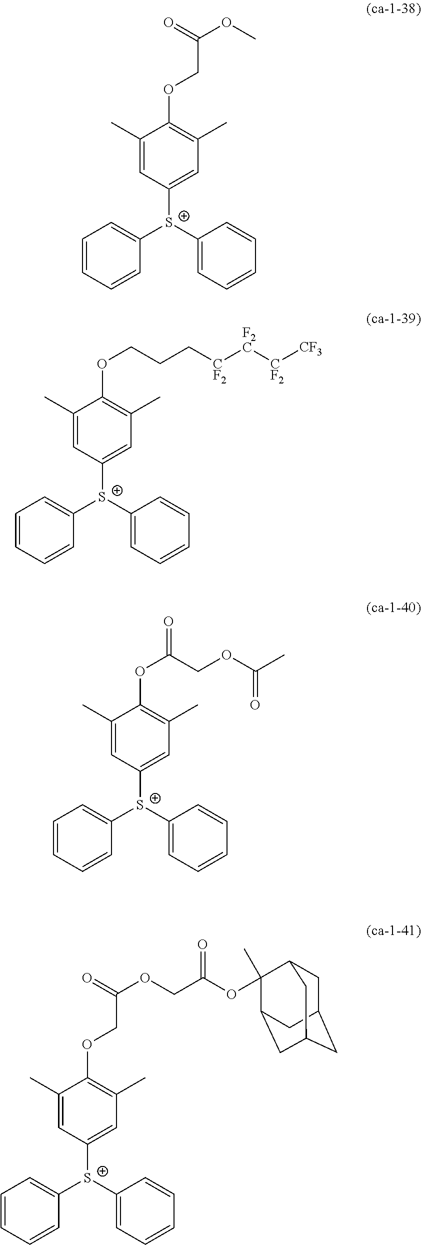

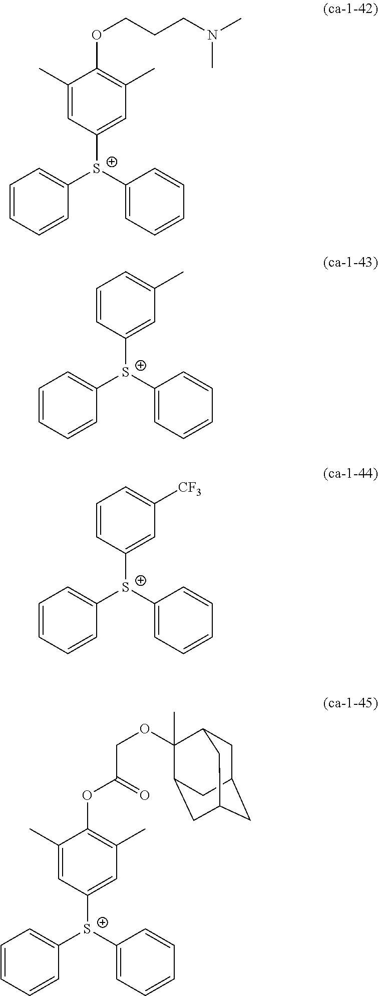

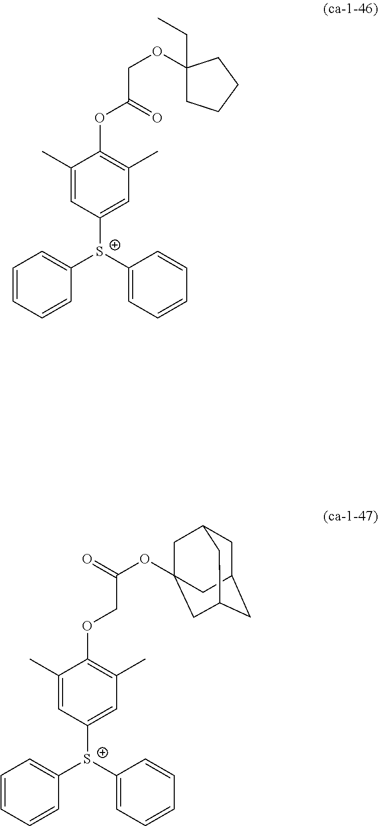

[In the formulae, g1, g2, and g3 represent the numbers of repetitions, g1 is an integer in a range of 1 to 5, g2 is an integer in a range of 0 to 20, and g3 is an integer in a range of 0 to 20.]

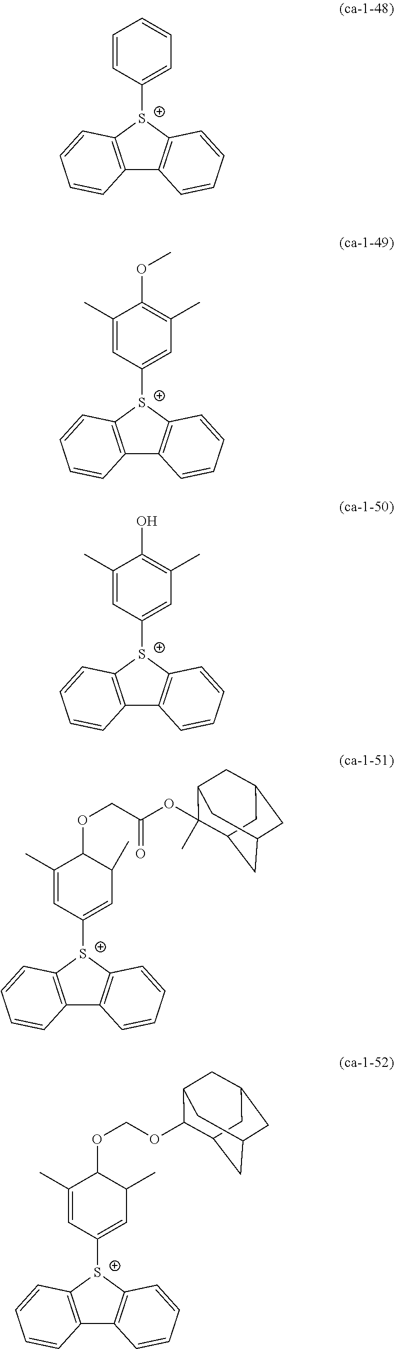 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

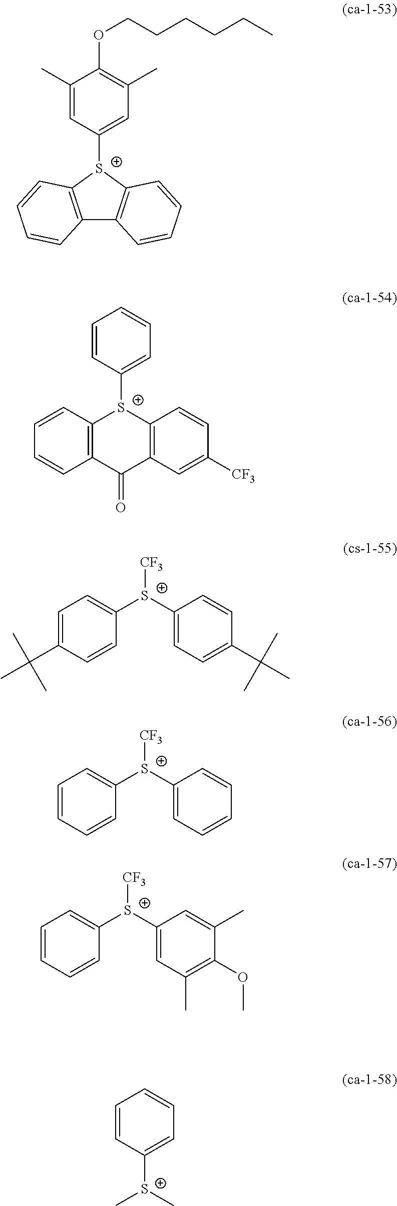

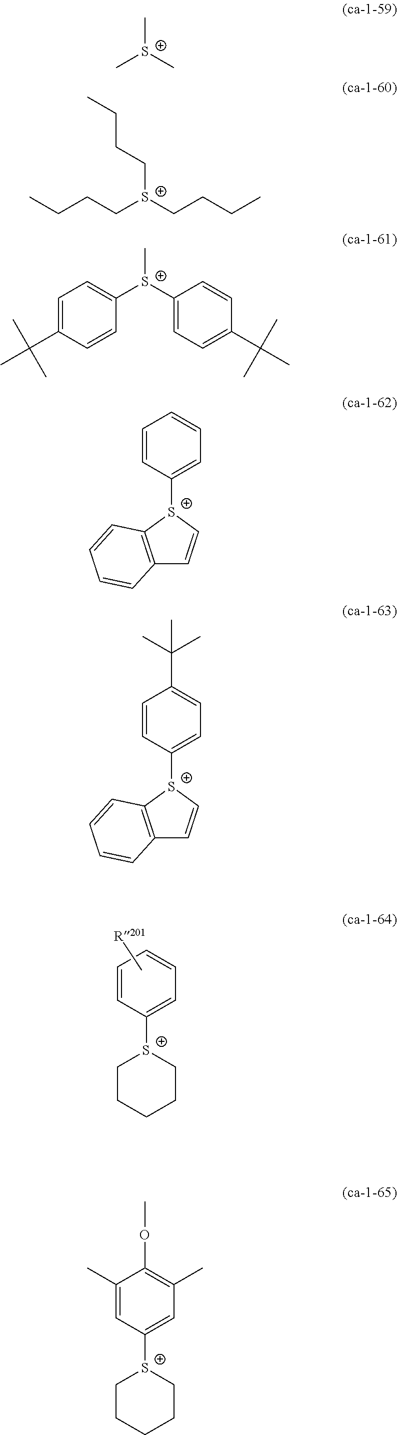

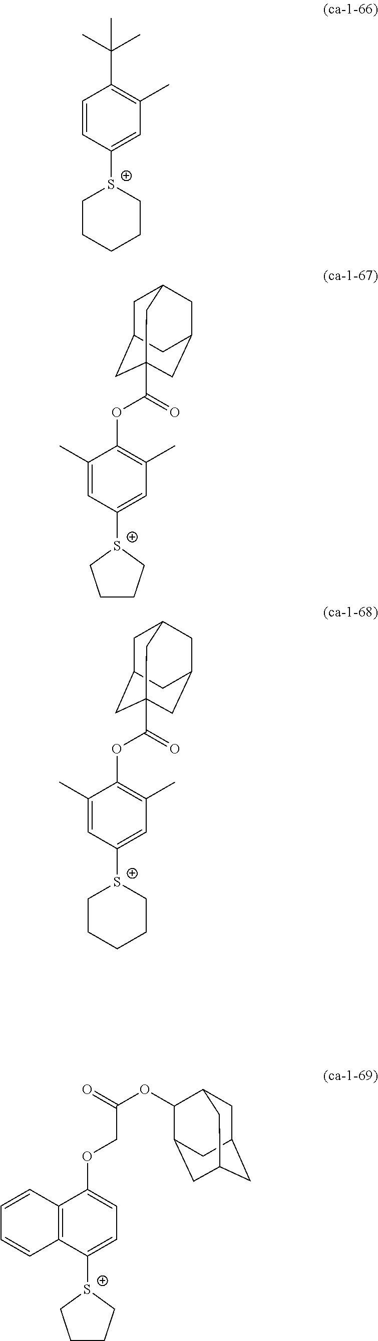 [SMILES: Cc1cc([S+]2CCCCC2)ccc1C(C)(C)C ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

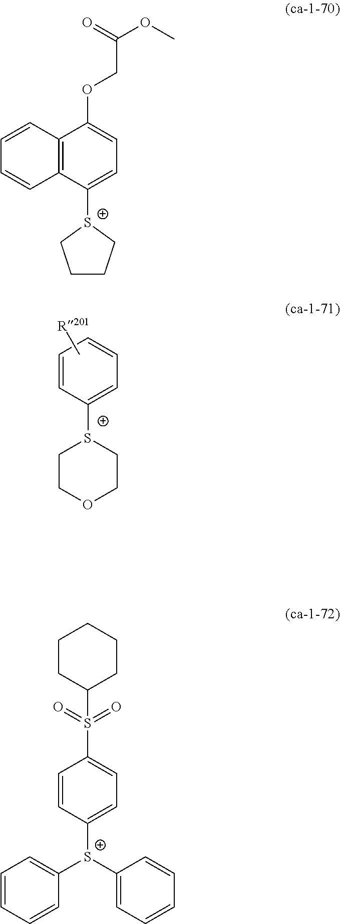

[In the formulae, R″²⁰¹ represents a hydrogen atom or a substituent, and examples of the substituent include the same as those exemplified as the substituent which may be contained in R²⁰¹ to R²⁰⁷ and R²¹⁰ to R²¹².]

Specific examples of the suitable cation represented by General Formula (ca-2) include a diphenyliodonium cation and a bis(4-tert-butylphenyl)iodonium cation.

Specific examples of the suitable cation represented by General Formula (ca-3) include cations each represented by General Formulae (ca-3-1) to (ca-3-6).

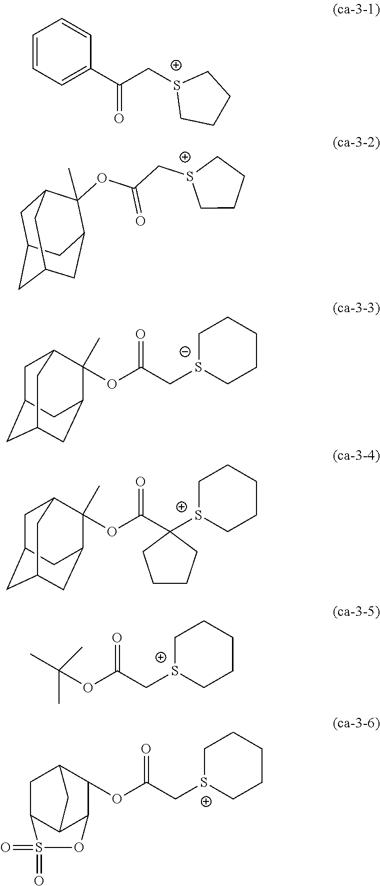

Specific examples of the suitable cations each represented by General Formula (ca-4) include cations each represented by General Formulae (ca-4-1) and (ca-4-2) shown below.

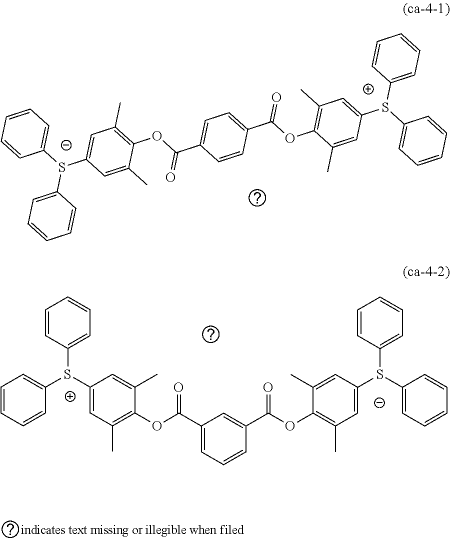

Specific examples of the suitable cations each represented by General Formula (ca-5) include cations each represented by General Formulae (ca-5-1) to (ca-5-3) shown below.

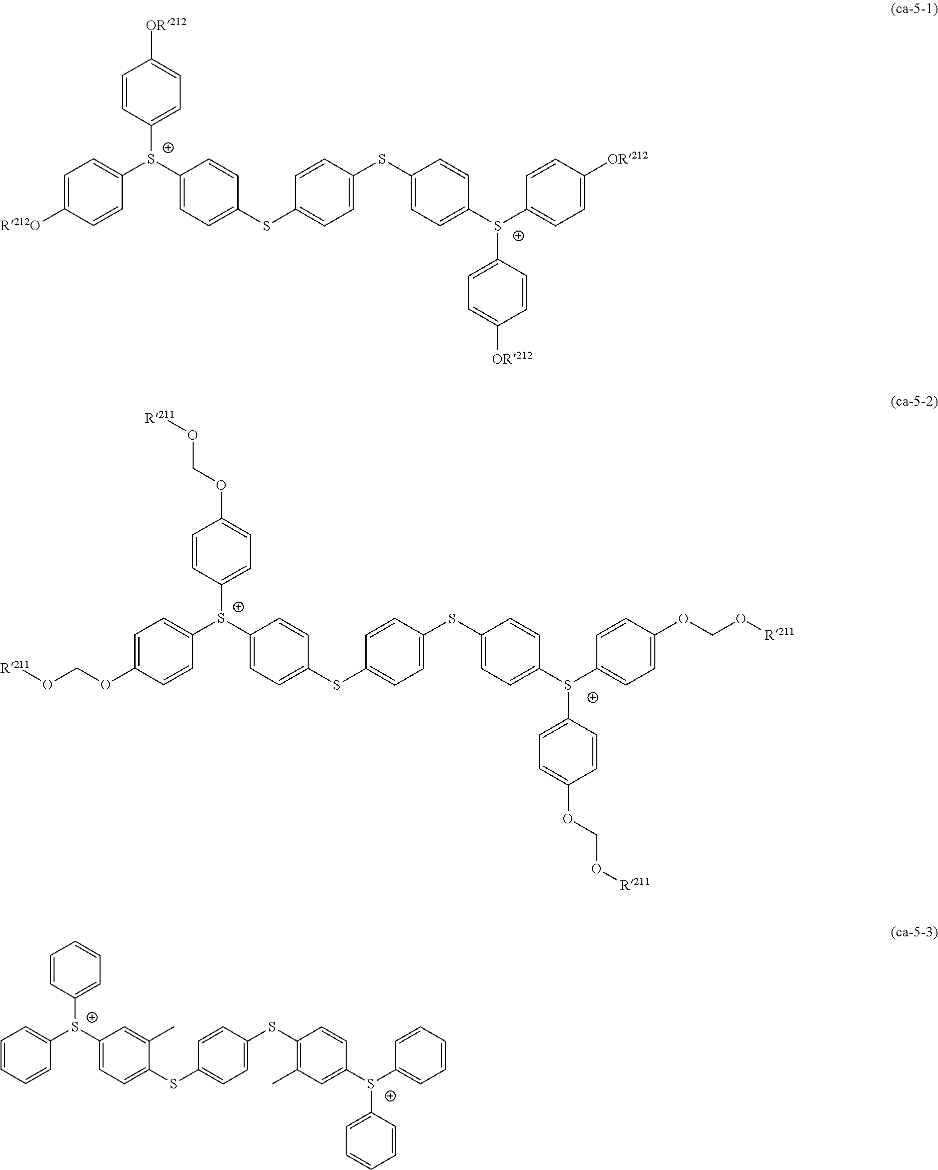

Among the above, the cation moiety (M^{m+})_{1/m}) is preferably a cation represented by General Formula (ca-1) or (ca-2), and more preferably a cation represented by General Formulae (ca-1).

In the resist composition according to the present embodiment, among the above, the component (B0) is preferably a compound represented by General Formula (b0-1).

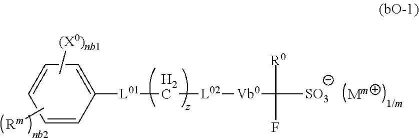

[In the formula, X⁰ represents a bromine atom or an iodine atom. R^{m} represents a hydroxy group, an alkyl group, a fluorine atom, or a chlorine atom. nb1 represents an integer in a range of 1 to 5, and nb2 represents an integer in a range of 0 to 4, where 1≤nb1+nb2≤5 is satisfied. L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}). R^{a}'s each independently represents a hydrogen atom or an alkyl group. z represents an integer in a range of 0 to 10. Vb⁰ represents a single bond, an alkylene group, or a fluorinated alkylene group. R⁰ represents a hydrogen atom, a fluorinated alkyl group having 1 to 5 carbon atoms, or a fluorine atom. M^{m+} represents an m-valent organic cation, where m represents an integer of 1 or more.]

The anion moiety of the compound represented by General Formula (b0-1) is the same as the anion represented by General Formula (b0-an0).

The cation moiety of the compound represented by General Formula (b0-1) is the same as the cation moiety of the compound represented by General Formula (b0).

Specific examples of the component (B0) are shown below but are not limited thereto.

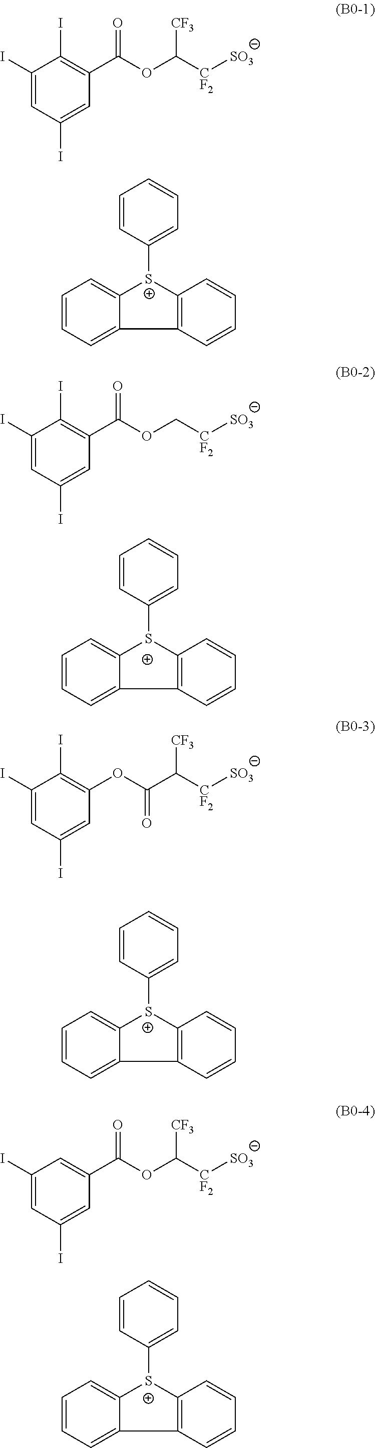

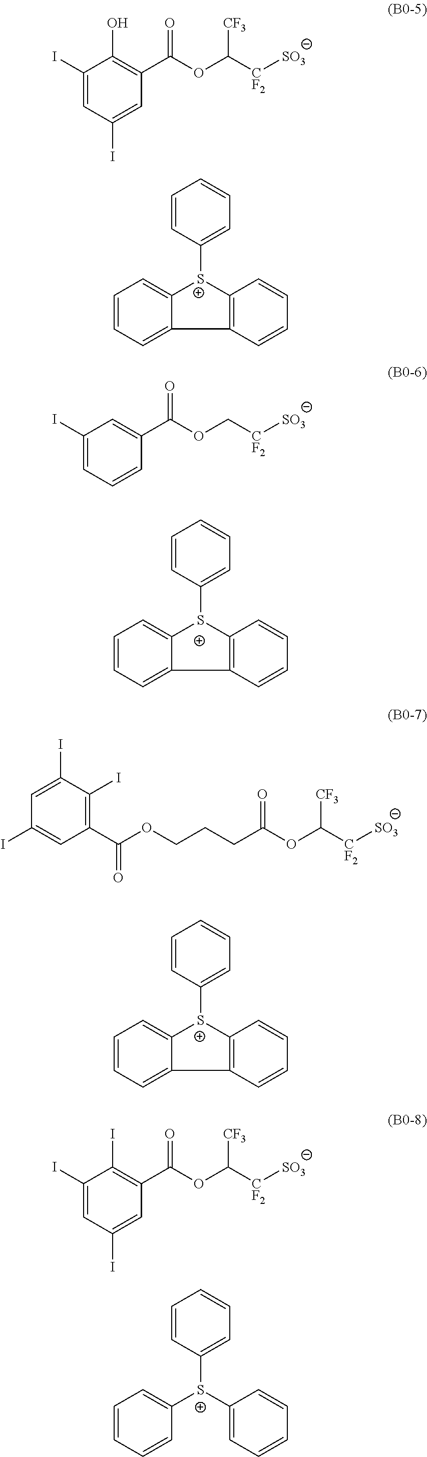

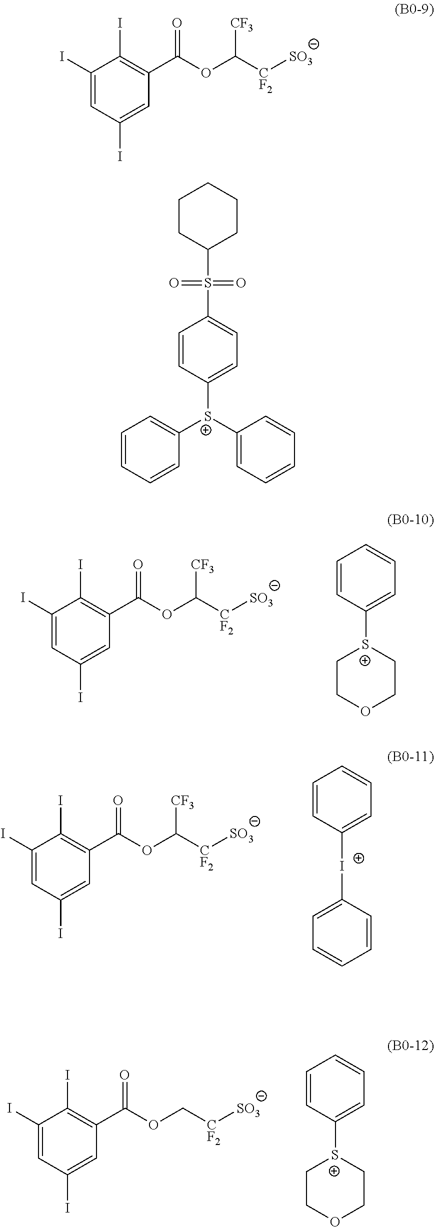

In the resist composition according to the present embodiment, the component (B0) may be used alone or may be used in a combination of two or more kinds thereof.

In the resist composition according to the present embodiment, the content of the component (B0) is preferably in a range of 5 to 40 parts by mass, more preferably in a range of 10 to 40 parts by mass, still more preferably in a range of 15 to 40 parts by mass, and particularly preferably in a range of 20 to 35 parts by mass, with respect to 100 parts by mass of the component (A).

In a case where the content of the component (B0) is equal to or larger than the lower limit value of the above-described preferred range, lithography characteristics such as sensitivity, reduction of linewidth roughness (LWR), and an exposure margin are further improved in the resist pattern formation. On the other hand, in a case where the content thereof is equal to or smaller than the upper limit value of the preferred range, a homogeneous solution is easily obtained when each of the components of the resist composition is dissolved in an organic solvent, and the storage stability as a resist composition is further improved.

The proportion of the component (B0) in the total component (B) contained in the resist composition according to the present embodiment is, for example, 50% by mass or more, preferably 70% by mass or more, and more preferably 95% by mass or more. The proportion of the component (B0) in the total component (B) may be 100% by mass.

The component (B) in the resist composition according to the present embodiment may contain an acid generator component (B1) (hereinafter, also referred to as a “component (B1)”) other than the above-described component (B0).

### <<Component (B1)>>

Examples of the component (B1) are numerous and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators; iminosulfonate-based acid generators; and disulfonate-based acid generators.

Examples of the onium salt-based acid generator include a compound represented by General Formula (b-1) (hereinafter, also referred to as a “component (b-1)”), a compound represented by General Formula (b-2) (hereinafter, also referred to as a “component (b-2)”), and a compound represented by General Formula (b-3) (hereinafter, also referred to as a “component (b-3)”).

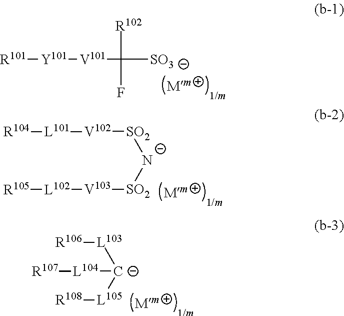

[In the formulae, R¹⁰¹ and R¹⁰⁴ to R¹⁰⁸ each independently represents a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group containing an oxygen atom or a single bond. V¹⁰¹ to V¹⁰³ each independently represents a single bond, an alkylene group, or a fluorinated alkylene group. L¹⁰¹ and L¹⁰² each independently represents a single bond or an oxygen atom. L¹⁰³ to L¹⁰⁵ each independently represents a single bond, —CO—, or —SO₂—. M′^{m+} represents an m-valent onium cation, where m represents an integer of 1 or more.]

### {Anion Moiety}

Anion in Component (b-1)

In General Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

The aromatic hydrocarbon group as R¹⁰¹ is a hydrocarbon group having an aromatic ring. The aromatic hydrocarbon group preferably has 3 to 30 carbon atoms, more preferably has 5 to 30, still more preferably has 5 to 20, particularly preferably has 6 to 15, and most preferably has 6 to 10. However, the number of carbon atoms in a substituent is not included in the number of carbon atoms in the aromatic hydrocarbon group.

Specific examples of the aromatic ring which the aromatic hydrocarbon group has as R¹⁰¹ include benzene, fluorene, naphthalene, anthracene, phenanthrene, biphenyl, and an aromatic heterocyclic ring obtained by substituting part of carbon atoms constituting any one of these aromatic rings with a hetero atom. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the above-described aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group. 1-naphthylethyl group, or a 2-naphthylethyl group). The alkylene group (an alkyl chain in the arylalkyl group) preferably has 1 to 4 carbon atoms, more preferably has 1 or 2 carbon atoms, and particularly preferably has 1 carbon atom.

Examples of the cyclic aliphatic hydrocarbon group as R¹⁰¹ include aliphatic hydrocarbon groups containing a ring in the structure thereof.

Examples of the aliphatic hydrocarbon group containing a ring in the structure thereof include an alicyclic hydrocarbon group (a group obtained by removing one hydrogen atom from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group preferably has 3 to 20 carbon atoms and more preferably 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane is preferably a monocycloalkane having 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among the above, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

Among the above, the cyclic aliphatic hydrocarbon group as R¹⁰¹ is preferably a group obtained by removing one or more hydrogen atoms from a monocycloalkane or a polycycloalkane, more preferably a group obtained by removing one hydrogen atom from a polycycloalkane, still more preferably an adamantyl group or a norbornyl group, and particularly preferably an adamantyl group.

The linear aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and most preferably has 1 to 3 carbon atoms. The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group preferably has 2 to 10 carbon atoms, more preferably has 3 to 6 carbon atoms, still more preferably has 3 or 4 carbon atoms, and most preferably has 3 carbon atoms. The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

The cyclic hydrocarbon group as R¹⁰¹ may contain a hetero atom such as a heterocyclic ring. Specific examples thereof include the lactone-containing cyclic groups each represented by General Formulae (a2-r-1) to (a2-r-7), the —SO₂—-containing cyclic groups each represented by General Formulae (a5-r-1) to (a5-r-4), and other heterocyclic groups each represented by Chemical Formulae (r-hr-1) to (r-hr-16). In the formulae, * represents a bonding site for bonding to Y¹⁰¹ in General Formula (b-1).

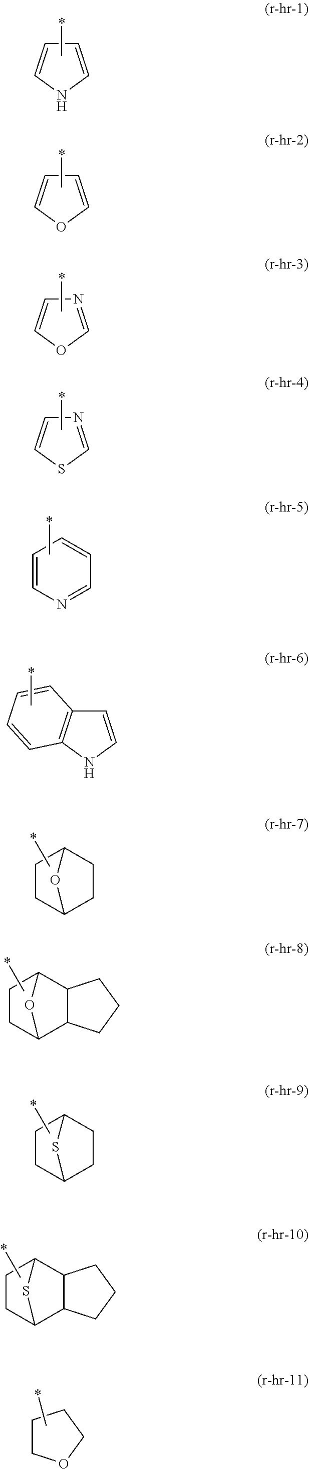

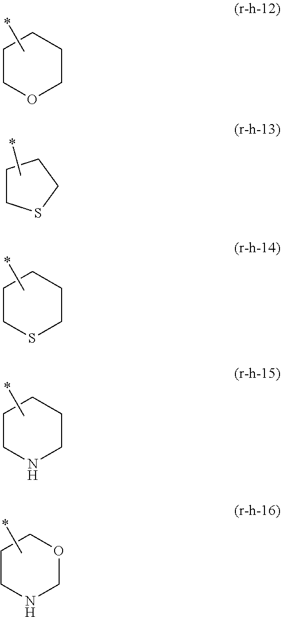

Examples of the substituent of the cyclic group as R¹⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include a group obtained by substituting part or all of hydrogen atoms in an alkyl group having 1 to 5 carbon atoms such as a methyl group, an ethyl group, a propyl group, an n-butyl group, or a tert-butyl group, with the above-described halogen atom.

The carbonyl group as the substituent is a group that is substituted for a methylene group (—CH₂—) constituting the cyclic hydrocarbon group.

The cyclic hydrocarbon group as R¹⁰¹ may be a condensed ring-type group containing a condensed ring in which an aliphatic hydrocarbon ring and an aromatic ring are condensed. Examples of the condensed ring include a condensed ring in which one or more aromatic rings are condensed with a polycycloalkane having a bridged ring-based polycyclic skeleton. Specific examples of the bridged ring-based polycycloalkane include bicycloalkanes such as bicyclo[2.2.1]heptane (norbornane) and bicyclo[2.2.2]octane. The condensed ring type is preferably a group containing a condensed ring, in which two or three aromatic rings are condensed with a bicycloalkane, and more preferably a group containing a condensed ring in which two or three aromatic rings are condensed with bicyclo[2.2.2]octane. Specific examples of the condensed ring-type group as R¹⁰¹ include groups represented by General Formulae (r-br-1) and (r-br-2). In the formulae. * represents a bonding site for bonding to Y¹⁰¹ in General Formula (b-1).

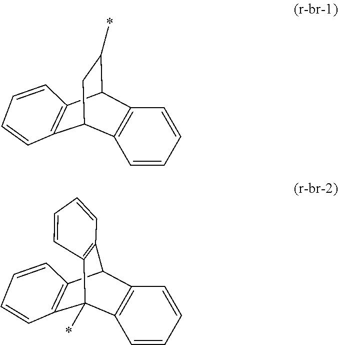

Examples of the substituent which may be contained in the condensed ring-type group as R¹⁰¹ include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed ring-type group include the same as those described as the substituent of the cyclic group as R¹⁰¹.

Examples of the aromatic hydrocarbon group as the substituent of the condensed ring-type group include a group obtained by removing one hydrogen atom from the above-described aromatic ring (an aryl group; for example, a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and heterocyclic groups each represented by General Formulae (r-hr-1) to (r-hr-6).

Examples of the alicyclic hydrocarbon group as the substituent of the condensed ring-type group include a group obtained by removing one hydrogen atom from a monocycloalkane such as cyclopentane or cyclohexane; a group obtained by removing one hydrogen atom from a polycycloalkane such as adamantane, norbornane, isobornane, tricyclodecane, or tetracyclododecane; the lactone-containing cyclic groups each represented by General Formulae (a2-r-1) to (a2-r-7); the —SO₂—-containing cyclic groups each represented by General Formulae (a5-r-1) to (a5-r-4); and the heterocyclic groups each represented by General Formulae (r-hr-7) to (r-hr-16).

Chain-Like Alkyl Group which May have Substituent:

The chain-like alkyl group as R¹⁰¹ may be linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms in the chain-like alkenyl group is preferably 2 to 10, more preferably 2 to 5, still more preferably 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent in the chain-like alkyl group or alkenyl group as R¹⁰¹, an alkoxy group, a halogen atom, a halogenated alkyl group, a hydroxyl group, a carbonyl group, a nitro group, an amino group, a cyclic group as R¹⁰¹ or the like may be used.

Among them, R¹⁰¹ is preferably a cyclic group which may have a substituent, and more preferably a cyclic hydrocarbon group which may have a substituent. More specifically, the cyclic hydrocarbon group is preferably a group obtained by removing one or more hydrogen atoms from a phenyl group, a naphthyl group, a polycycloalkane; the lactone-containing cyclic groups each represented by General Formulae (a2-r-1) to (a2-r-7); or the —SO₂—-containing cyclic groups each represented by General Formulae (a5-r-1) to (a5-r-4), more preferably a group obtained by removing one or more hydrogen atoms from a polycycloalkane, or the —SO₂—-containing cyclic groups each represented by General Formulae (a5-r-1) to (a5-r-4), and still more preferably an adamantyl group or the —SO₂—-containing cyclic groups represented by General Formula (a5-r-1).

In a case where the cyclic hydrocarbon group has a substituent, the substituent is preferably a hydroxyl group.

In General Formula (b-1), Y¹⁰¹ represents a single bond or a divalent linking group containing an oxygen atom.

In a case where Y¹⁰¹ represents a divalent linking group containing an oxygen atom. Y¹⁰¹ may contain an atom other than the oxygen atom. Examples of the atom other than the oxygen atom include a carbon atom, a hydrogen atom, a sulfur atom, and a nitrogen atom.

Examples of divalent linking groups containing an oxygen atom include non-hydrocarbon-based oxygen atom-containing linking groups such as an oxygen atom (an ether bond; —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon-based oxygen atom-containing linking groups with an alkylene group. A sulfonyl group (—SO₂—) may be further linked to this combination. Examples of such a divalent linking group containing an oxygen atom include linking groups each represented by General Formulae (y-al-1) to (y-al-7) shown below. In General Formulae (y-al-1) to (y-al-7), the one that is bonded to R¹⁰¹ in General Formula (b-1) is V′¹⁰¹ in General Formulae (y-al-1) to (y-al-7).

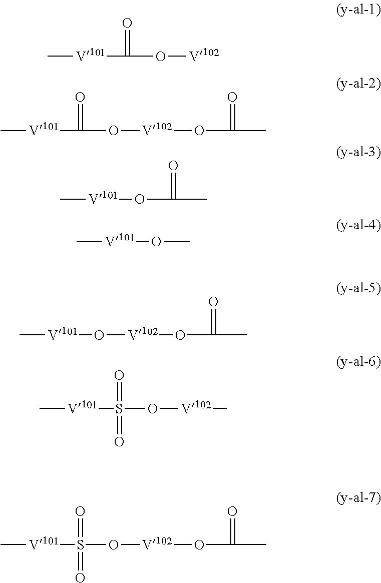

[In the formulae, V′¹⁰¹ represents a single bond or an alkylene group having 1 to 5 carbon atoms, and V′¹⁰² represents a divalent saturated hydrocarbon group having 1 to 30 carbon atoms.]

The divalent saturated hydrocarbon group as V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

In addition, a part of methylene groups in the alkylene group as V′¹⁰¹ or V′¹⁰² may be substituted with a divalent aliphatic cyclic group having 5 to 10 carbon atoms. The aliphatic cyclic group is preferably a divalent group obtained by removing one hydrogen atom from the cyclic aliphatic hydrocarbon group (a monocyclic aliphatic hydrocarbon group or a polycyclic aliphatic hydrocarbon group) as Ra′³ in General Formula (a1-r-1), and a cyclohexylene group, a 1,5-adamantylene group, or a 2,6-adamantylene group is more preferable.

Y¹⁰¹ is preferably a divalent linking group containing an ester bond or a divalent linking group containing an ether bond and more preferably linking groups each represented by General Formulae (y-al-1) to (y-al-5).

In General Formula (b-1), V¹⁰¹ represents a single bond, an alkylene group, or a fluorinated alkylene group. The alkylene group and the fluorinated alkylene group as V¹⁰¹ preferably have 1 to 4 carbon atoms. Examples of the fluorinated alkylene group as V¹⁰¹ include a group obtained substituting part or all of hydrogen atoms in the alkylene group as V¹⁰¹ with a fluorine atom. Among the above, V¹⁰¹ is preferably a single bond or a fluorinated alkylene group having 1 to 4 carbon atoms.

In General Formula (b-1), R¹⁰² represents a fluorine atom or a fluorinated alkyl group having 1 to 5 carbon atoms. R¹⁰² is preferably a fluorine atom or a perfluoroalkyl group having 1 to 5 carbon atoms and more preferably a fluorine atom.

In a case where Y¹⁰¹ represents a single bond, specific examples of the anion moiety represented by General Formula (b-1) include a fluorinated alkylsulfonate anion such as a trifluoromethanesulfonate anion or a perfluorobutanesulfonate anion; and in a case where Y¹⁰¹ represents a divalent linking group containing an oxygen atom, specific examples thereof include an anion represented by any one of General Formulae (an-1) to (an-3) shown below.

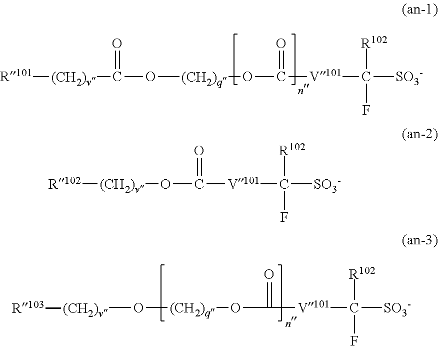

[In the formula, R″¹⁰¹ represents an aliphatic cyclic group which may have a substituent, monovalent heterocyclic groups each represented by Chemical Formulae (r-hr-1) to (r-hr-6), a condensed ring-type group represented by General Formula (r-br-1) or (r-br-2), and a chain-like alkyl group which may have a substituent. R″¹⁰² is an aliphatic cyclic group which may have a substituent, the condensed ring-type group represented by General Formula (r-br-1) or (r-br-2), the lactone-containing cyclic groups each represented by General Formulae (a2-r-1), (a2-r-3) to (a2-r-7), or the —SO₂—-containing cyclic groups each represented by General Formulae (a5-r-1) to (a5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer in a range of 0 to 3, each q″ independently represents an integer in a range of 0 to 20, and n″ represents 0 or 1.]

The aliphatic cyclic group as R″¹⁰¹, R″¹⁰², and R″¹⁰³, which may have a substituent, is preferably the group exemplified as the cyclic aliphatic hydrocarbon group as R¹⁰¹ in General Formula (b-1). Examples of the substituent include the same one as the substituent that may be substituted for the cyclic aliphatic hydrocarbon group as R¹⁰¹ in General Formula (b-1).

The aromatic cyclic group which may have a substituent, as R″¹⁰³, is preferably the group exemplified as the aromatic hydrocarbon group for the cyclic hydrocarbon group as R¹⁰¹ in General Formula (b-1). Examples of the substituent include the same one as the substituent that may be substituted for the aromatic hydrocarbon group as R¹⁰¹ in General Formula (b-1).

The chain-like alkyl group as R″¹⁰¹, which may have a substituent, is preferably the group exemplified as the chain-like alkyl group as R¹⁰¹ in General Formula (b-1).

The chain-like alkenyl group as R″¹⁰³, which may have a substituent, is preferably the group exemplified as the chain-like alkenyl group as R¹⁰¹ in General Formula (b-1).

Anion in Component (b-2)

In General Formula (b-2), R¹⁰⁴ and R¹⁰⁵ each independently represents a cyclic group which may have a substituent, a chain-like alkyl group which may have a substituent, or a chain-like alkenyl group which may have a substituent, and examples thereof each include the same one as R¹⁰¹ in General Formula (b-1). However, R¹⁰⁴ and R¹⁰⁵ may be bonded to each other to form a ring.

R¹⁰⁴ and R¹⁰⁵ are preferably a chain-like alkyl group which may have a substituent, and more preferably a linear or branched alkyl group or a linear or branched fluorinated alkyl group.

The chain-like alkyl group preferably has 1 to 10 carbon atoms, more preferably has 1 to 7 carbon atoms, and still more preferably has 1 to 3 carbon atoms. It is preferable that the number of carbon atoms in the chain-like alkyl group as R¹⁰⁴ and R¹⁰⁵ be small since the solubility in a resist solvent is also excellent in this range of the number of carbon atoms. In addition, in the chain-like alkyl group as R¹⁰⁴ and R¹⁰⁵, it is preferable that the number of hydrogen atoms substituted with a fluorine atom be large since the acid strength increases and the transparency to high energy radiation of 250 nm or less or electron beams is improved. The proportion of fluorine atoms in the chain-like alkyl group, that is, the fluorination rate is preferably in a range of 70% to 100% and more preferably in a range of 90% to 100%, and it is most preferable that the chain-like alkyl group be a perfluoroalkyl group in which all hydrogen atoms be substituted with a fluorine atom.

In General Formula (b-2), V¹⁰² and V¹⁰³ each independently represents a single bond, an alkylene group, or a fluorinated alkylene group, and examples thereof each include the same one as V¹⁰¹ in General Formula (b-1).

In General Formula (b-2), L¹⁰¹ and L¹⁰² each independently represents a single bond or an oxygen atom.

Anion in Component (b-3)

In General Formula (b-3), R¹⁰⁶ to R¹⁰⁸ each independently represents a cyclic group which may have a substituent, a chain-like alkyl group which may have a substituent, or a chain-like alkenyl group which may have a substituent, and examples thereof each include the same one as R¹⁰¹ in General Formula (b-1).

In Formula (b-3), L¹⁰³ to L¹⁰⁵ each independently represents a single bond, —CO—, or —SO₂—.

Among the above, the anion moiety of the component (B) is preferably an anion of the component (b-1). Among these, an anion represented by any one of General Formulae (an-1) to (an-3) is more preferable, an anion represented by any one of General Formula (an-1) or (an-2) is still more preferable, and an anion represented by General Formula (an-2) is particularly preferable.

### {Cation Moiety}

In Formulae (b-1), (b-2), and (b-3) described above, M′^{m+} represents an m-valent onium cation. Among them, a sulfonium cation and an iodonium cation are preferable.

m represents an integer of 1 or more.

Preferred examples of the cation moiety ((M′^{m+})_{1/m}) include organic cations each represented by General Formulae (ca-1) to (ca-5).

In the resist composition according to the present embodiment, the component (B1) may be used alone or in a combination of two or more kinds thereof.

In a case where the resist composition contains the component (B1), the content of the component (B1) in the resist composition is preferably less than 40 parts by mass, more preferably in a range of 1 to 30 parts by mass, and still more preferably in a range of 1 to 20 parts by mass with respect to 100 parts by mass of the component (A).

In a case where the content of the component (B1) is set to be in the preferred range described above, pattern formation can be satisfactorily carried out. In addition, in a case where each component of the resist composition is dissolved in an organic solvent, the above range is preferable since a homogeneous solution is easily obtained and the storage stability of the resist composition is improved.

It is preferable that the resist composition according to the present embodiment not contain the component (B1).

### <Other Components>

The resist composition according to the present embodiment may further contain other components in addition to the component (A) and the component (B) described above. Examples of the other components include a component (D), a component (E), a component (F), and a component (S), which are described below.

### <<Base Component (D)>>

It is preferable that the resist composition according to the present embodiment further contain a base component (a component (D)) that traps (that is, controls the acid diffusion) acid that is generated upon exposure, in addition to the component (A) and the component (B). The component (D) acts as a quencher (an acid diffusion controlling agent) which traps the acid generated in the resist composition upon exposure.

Examples of the component (D) include a photodecomposable base (D1) having acid diffusion controllability (hereinafter, referred to as a “component (D1)”) which is lost by the decomposition upon exposure and a nitrogen-containing organic compound (D2) (hereinafter, referred to as a “component (D2)”) which does not correspond to the component (D1). Among these, the photodecomposable base (the component (D1)) is preferable since it is easy to enhance the roughness reducing property. In addition, in a case where the component (D1) is contained, it becomes easy to enhance both the characteristics of increasing the sensitivity and suppressing the occurrence of coating defects.

### In Regard to Component (D1)

In a case where a resist composition containing the component (D1) is obtained, the contrast between exposed portions and unexposed portions of the resist film can be further improved at the time of the formation of a resist pattern.

The component (D1) is not particularly limited as long as it is decomposed upon exposure and loses the acid diffusion controllability. The component (D1) is preferably one or more compounds selected from the group consisting of a compound represented by General Formula (d1-1) (hereinafter, referred to as a “component (d1-1)”), a compound represented by General Formula (d1-2) (hereinafter, referred to as a “component (d1-2)”), and a compound represented by General Formula (d1-3) (hereinafter, referred to as a “component (d1-3)”).

At exposed portions of the resist film, the components (d1-1) to (d1-3) are decomposed and then lose the acid diffusion controllability (basicity), and thus they cannot act as a quencher, whereas they act as a quencher at unexposed portions of the resist film.

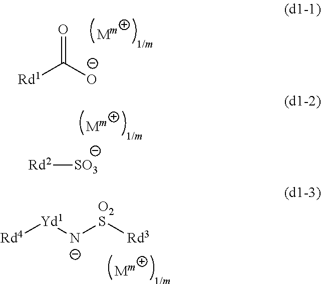

[In the formulae, Rd¹ to Rd⁴ represent cyclic groups which may have a substituent, chain-like alkyl groups which may have a substituent, or chain-like alkenyl groups which may have a substituent. Here, the carbon atom adjacent to the S atom in Rd² in General Formula (d1-2) has no fluorine atom bonded thereto. Yd¹ represents a single bond or a divalent linking group. m represents an integer of 1 or more, and each M^{m+} independently represents an m-valent organic cation.]

{Component (d1-1)}

### Anion Moiety

In General Formula (d1-1), Rd¹ represents a cyclic group which may have a substituent, a chain-like alkyl group which may have a substituent, or a chain-like alkenyl group which may have a substituent, and examples thereof include the same one as R′²⁰¹

Among these, Rd¹ is preferably an aromatic hydrocarbon group which may have a substituent, an aliphatic cyclic group which may have a substituent, or a chain-like alkyl group which may have a substituent. Examples of the substituent which may be contained in these groups include a hydroxyl group, an oxo group, an alkyl group, an aryl group, a fluorine atom, a fluorinated alkyl group, lactone-containing cyclic groups each represented by any of General Formulae (a2-r-1) to (a2-r-7), an ether bond, an ester bond, and a combination thereof. In a case where an ether bond or an ester bond is included as the substituent, it may be bonded through an alkylene group, and the substituent in this case is preferably a linking group represented by each of General Formulae (y-al-1) to (y-al-5). It should be noted that in a case where the aromatic hydrocarbon group, the aliphatic cyclic group, or the chain-like alkyl group, as Rd¹, has a linking group represented by each of General Formulae (y-al-1) to (y-al-7) as a substituent, in General Formulae (y-al-1) to (y-al-7), the group that is bonded to a carbon atom constituting the aromatic hydrocarbon group, the aliphatic cyclic group, or the chain-like alkyl group, as Rd¹, in General Formula (d3-1) is V′¹⁰¹ in General Formulae (y-al-1) to (y-al-7).

Suitable examples of the aromatic hydrocarbon group include a phenyl group, a naphthyl group, and a polycyclic structure (a polycyclic structure consisting of a bicyclooctane skeleton and a ring structure other than the bicyclooctane skeleton) including a bicyclooctane skeleton.

The aliphatic cyclic group is preferably a group obtained by removing one or more hydrogen atoms from a polycycloalkane such as adamantane, norbornane, isobornane, tricyclodecane, or tetracyclododecane.

The chain-like alkyl group preferably has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group, and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group preferably has 1 to 11 carbon atoms, more preferably has 1 to 8 carbon atoms, and still more preferably has 1 to 4 carbon atoms. The fluorinated alkyl group may contain an atom other than a fluorine atom. Examples of the atom other than a fluorine atom include an oxygen atom, a sulfur atom, and a nitrogen atom.

In General Formula (d1-1), Rd¹ is preferably, among the above, a chain-like alkyl group which may have a substituent, more preferably a chain-like alkyl group having at least a fluorine atom as a substituent, and still more preferably a chain-like alkyl group having a fluorine atom and a hydroxy group as a substituent.

Specific examples of the preferred anion moiety for the component (d1-1) are shown below.

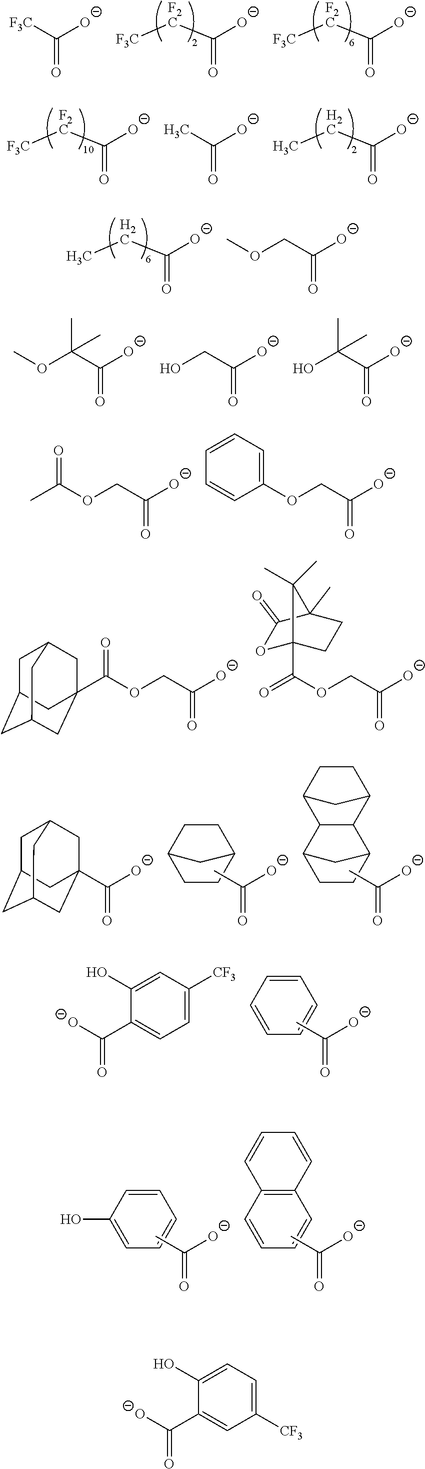

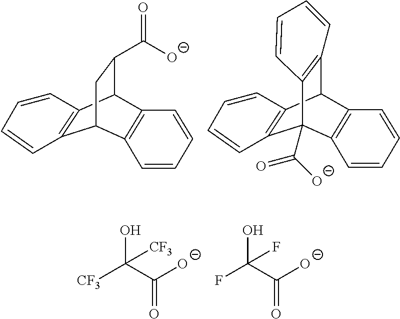 [SMILES: O=C([O-])C1CC2c3ccccc3C1c1ccccc12 ; O=C([O-])C12c3ccccc3C(c3ccccc31)c1ccccc12 ; O=C([O-])C(O)(C(F)(F)F)C(F)(F)F ; O=C([O-])C(O)(F)F]

### Cation Moiety

In General Formula (d1-1), M^{m+} represents an m-valent organic cation.

The suitable examples of the organic cation as M^{m+} include the same cations as the cations each represented by General Formulae (ca-1) to (ca-5), the cations each represented by General Formula (ca-1) are preferable, and cations each represented by General Formulae (ca-1-1) to (ca-1-78) are more preferable.

The component (d1-1) may be used alone or in a combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same one as R′²⁰¹

Here, the carbon atom adjacent to the S atom in Rd² has no fluorine atom bonded thereto (the carbon atom adjacent to the S atom in Rd² is not substituted with a fluorine atom). As a result, the anion of the component (d1-2) becomes an appropriately weak acid anion, thereby improving the quenching ability of the component (D).

Rd² is preferably a chain-like alkyl group which may have a substituent or an aliphatic cyclic group which may have a substituent, and more preferably an aliphatic cyclic group which may have a substituent.

The chain-like alkyl group preferably has 1 to 10 carbon atoms and more preferably has 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent. Examples of the substituent include the same ones as the substituents which may be contained in the hydrocarbon group (the aromatic hydrocarbon group, the aliphatic cyclic group, or the chain-like alkyl group) as Rd¹ in General Formula (d1-1).

Among the above, the anion moiety of the component (d1-2) is preferably a camphorsulfonic acid anion.

Specific examples of the preferred anion moiety for the component (d1-2) are shown below.

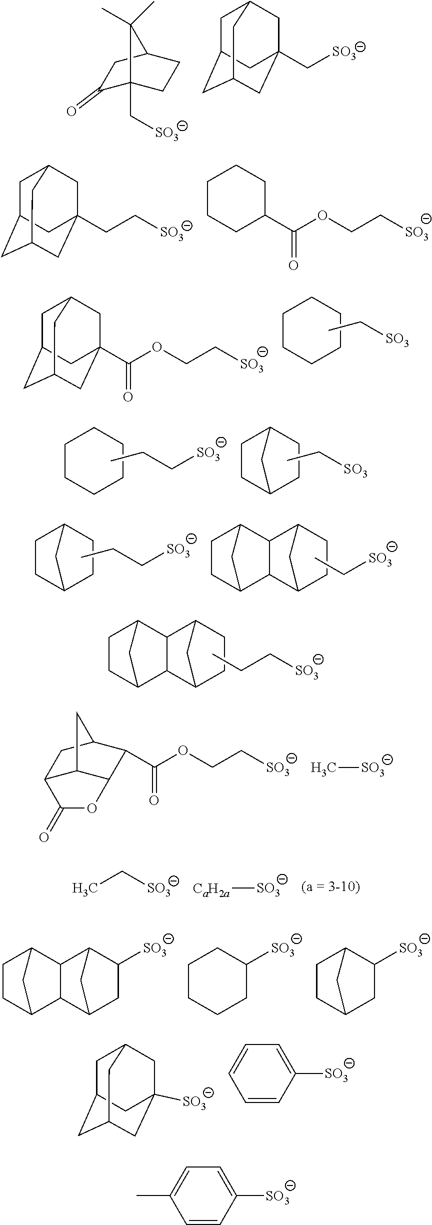

### Cation Moiety

In General Formula (d1-2), M^{m+} represents an m-valent organic cation and is the same as M^{m+} in General Formula (d1-1).

The component (d1-2) may be used alone or in a combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same one as R′²⁰¹. Among these, a cyclic group having a fluorine atom, a chain-like alkyl group, or a chain-like alkenyl group is preferable. Among them, a fluorinated alkyl group is preferable, and the same one as the fluorinated alkyl group as Rd¹ is more preferable.

In General Formula (d1-3), Rd⁴ represents a cyclic group which may have a substituent, a chain-like alkyl group which may have a substituent, or a chain-like alkenyl group which may have a substituent, and examples thereof include the same one as R′²⁰¹

Among them, an alkyl group which may have a substituent, an alkoxy group which may have a substituent, an alkenyl group which may have a substituent, or a cyclic group which may have a substituent is preferable.

The alkyl group as Rd⁴ is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. A part of hydrogen atoms in the alkyl group as Rd⁴ may be substituted with a hydroxyl group, a cyano group, or the like.

The alkoxy group as Rd⁴ is preferably an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among the above, a methoxy group or an ethoxy group is preferable.

Examples of the alkenyl group as Rd⁴ include the same one as R′²⁰¹, and a vinyl group, a propenyl group (an allyl group), a 1-methylpropenyl group, or a 2-methylpropenyl group is preferable. These groups may have an alkyl group having 1 to 5 carbon atoms or a halogenated alkyl group having 1 to 5 carbon atoms as a substituent.

Examples of the cyclic group as Rd⁴ include the same one as the cyclic group as R′²⁰¹, and an alicyclic group obtained by removing one or more hydrogen atoms from a cycloalkane such as cyclopentane, cyclohexane, adamantane, norbornane, isobornane, tricyclodecane, or tetracyclododecane, or an aromatic group such as a phenyl group or a naphthyl group is preferable. In a case where Rd⁴ represents an alicyclic group, the resist composition can be satisfactorily dissolved in an organic solvent, thereby improving the lithography characteristics. In a case where Rd⁴ represents an aromatic group, the resist composition is excellent in light absorption efficiency and thus has good sensitivity and lithography characteristics in the lithography using EUV or the like as a light source for exposure.

In General Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group containing a hetero atom. Each divalent linking group includes the same as those described in the description for the divalent hydrocarbon group which may have a substituent and the divalent linking group containing a hetero atom described above as the divalent linking group as Ya²¹ in General Formula (a2-1).

Yd¹ is preferably a carbonyl group, an ester bond, an amide bond, an alkylene group, or a combination thereof. The alkylene group is more preferably a linear or branched alkylene group, and still more preferably a methylene group or an ethylene group.

Specific examples of the preferred anion moiety for the component (d1-3) are shown below.

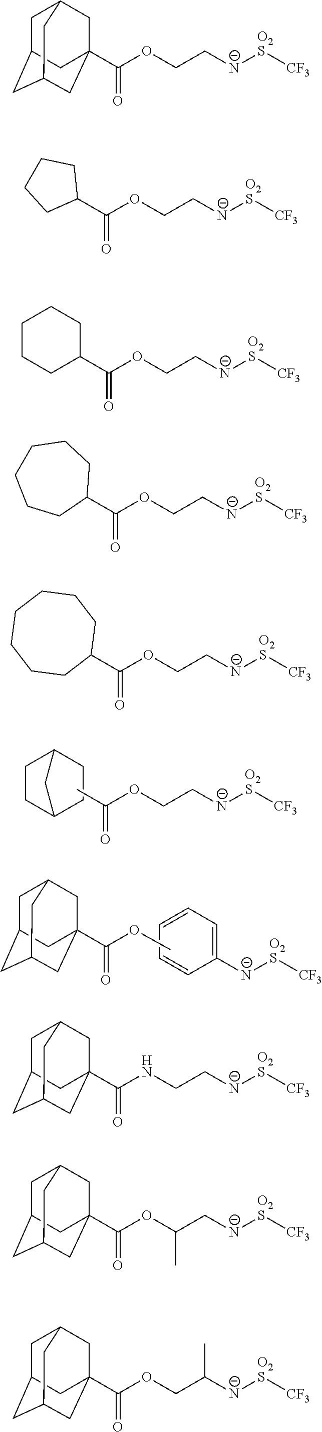

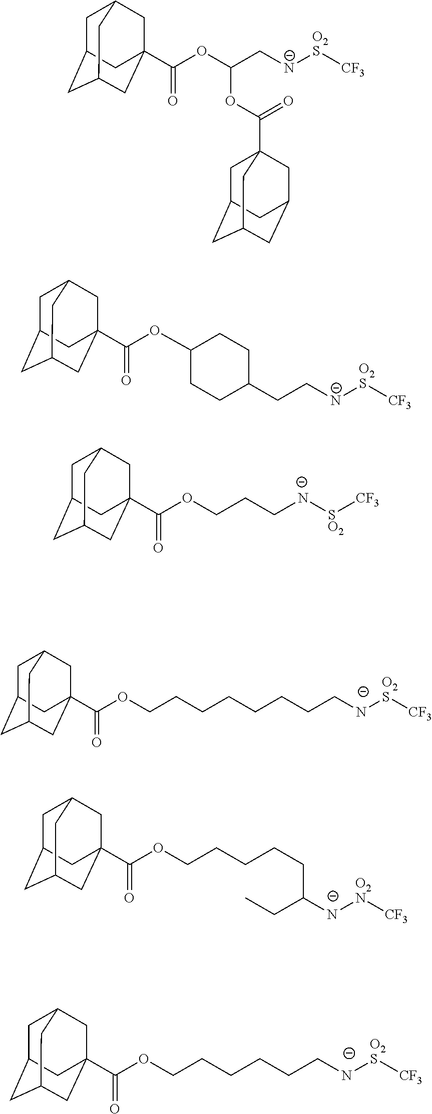

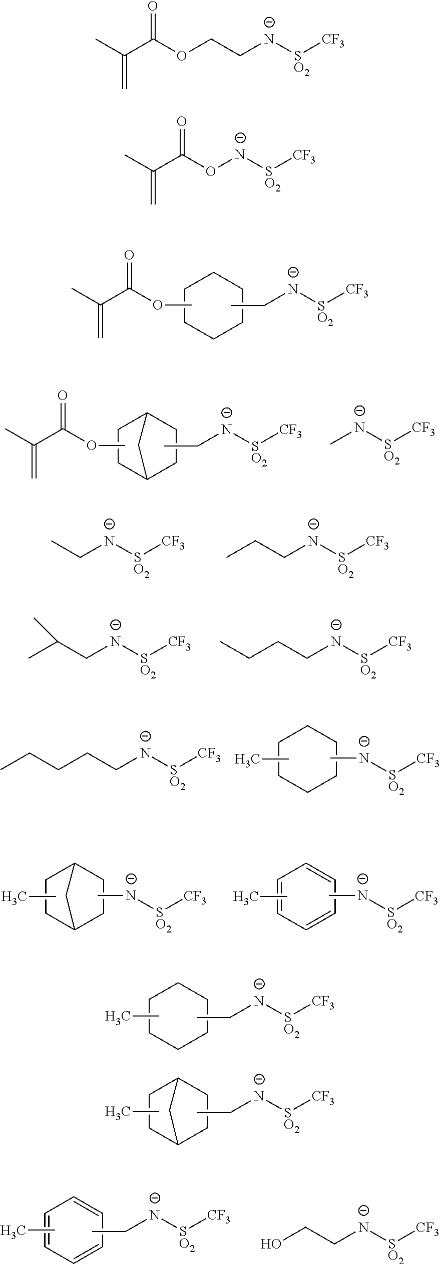

### Cation Moiety

In General Formula (d1-3), M^{m+} represents an m-valent organic cation and is the same as M^{m+} in General Formula (d1-1).

The component (d1-3) may be used alone or in a combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 20 parts by mass, more preferably in a range of 1 to 15 parts by mass, and still more preferably in a range of 3 to 10 parts by mass with respect to 100 parts by mass of the component (A1).

In a case where the content of the component (D1) is equal to or larger than the preferred lower limit value, excellent lithography characteristics and an excellent resist pattern shape are easily obtained. On the other hand, in a case where the content thereof is equal to or smaller than the upper limit value, the sensitivity can be maintained satisfactorily, and the throughput is also excellent.

In the resist composition according to the present embodiment, the component (D1) preferably contains the component (d1-1).

The content of the component (d1-1) in the total component (D) contained in the resist composition according to the present embodiment is preferably 50% by mass or more, preferably 70% by mass or more, and still more preferably 90% by mass or more, and the component (D) may consist of only a compound for the component (d1-1).

### Production Method for Component (D1):

The production methods for the components (d1-1) and (d1-2) described above are not particularly limited, and the components (d1-1) and (d1-2) can be produced by conventionally known methods.

In addition, the production method for the component (d1-3) is not particularly limited, and the component (d1-3) can be produced, for example, in the same manner as disclosed in United States Patent Application, Publication No. 2012-0149916.

### In Regard to Component (D2)

The component (D) may contain a nitrogen-containing organic compound component (hereinafter, referred to as a “component (D2)”) which does not correspond to the above-described component (D1).

The component (D2) is not particularly limited as long as it acts as an acid diffusion controlling agent and does not correspond to the component (D1), and any known compound may be used. Among the above, aliphatic amines are preferable, and among the aliphatic amines, a secondary aliphatic amine or a tertiary aliphatic amine is more preferable.

The aliphatic amine is an amine having one or more aliphatic groups, and the aliphatic groups preferably have 1 to 12 carbon atoms.

Examples of these aliphatic amines include an amine in which at least one hydrogen atom of ammonia (NH₃) has been substituted with an alkyl group or hydroxyalkyl group having 12 or fewer carbon atoms (alkyl amines or alkyl alcohol amines) and a cyclic amine.

Specific examples of the alkyl amine and the alkyl alcohol amine include monoalkyl amines such as n-hexyl amine, n-heptyl amine, n-octyl amine, n-nonyl amine, and n-decyl amine; dialkyl amines such as diethyl amine, di-n-propyl amine, di-n-heptyl amine, di-n-octyl amine, and dicyclohexyl amine; trialkyl amines such as trimethyl amine, triethyl amine, tri-n-propyl amine, tri-n-butyl amine, tri-n-pentyl amine, tri-n-hexyl amine, tri-n-heptyl amine, tri-n-octyl amine, tri-n-nonyl amine, tri-n-decyl amine, and tri-n-dodecyl amine; and alkyl alcohol amines such as diethanol amine, triethanol amine, diisopropanol amine, triisopropanol amine, di-n-octanol amine, and tri-n-octanol amine. Among these, trialkyl amines of 5 to 10 carbon atoms are preferable, and tri-n-pentyl amine and tri-n-octyl amine are particularly preferable.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1, 5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris {2-(2-methoxyethoxymethoxy)ethyl}amine, tris {2-(1-methoxyethoxy)ethyl}amine, tris {2-(1-ethoxyethoxy)ethyl}amine, tris{2-(1-ethoxypropoxy)ethyl}amine, tris[2-{2-(2-hydroxyethoxy)ethoxy}ethyl]amine and triethanol amine triacetate, and triethanol amine triacetate is preferable.

In addition, as the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, and N-tert-butoxycarbonylpyrrolidine.

Among those described above, the component (D2) is preferably an alkyl amine and more preferably a trialkyl amine having 5 to 10 carbon atoms.

The component (D2) may be used alone or in a combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is equal to or larger than the preferred lower limit value, excellent lithography characteristics and an excellent resist pattern shape are easily obtained. On the other hand, in a case where the content thereof is equal to or smaller than the upper limit value, the sensitivity can be maintained satisfactorily and the throughput is also excellent.

<<At Least One Compound (E) Selected from the Group Consisting of Organic Carboxylic Acid, Phosphorus Oxo Acid, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as a “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid, and among them, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivative include phosphoric acid esters such as di-n-butyl phosphate and diphenyl phosphate.

Examples of the phosphonic acid derivative include phosphonic acid esters such as dimethyl phosphonate, di-n-butyl phosphonate, phenylphosphonic acid, diphenyl phosphonate, and dibenzyl phosphonate.

Examples of the phosphinic acid derivative include phosphinic acid esters and phenylphosphinic acid.

In the resist composition according to the present embodiment, one kind of the component (E) may be used alone, or two or more kinds thereof may be used in combination.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

### <<Fluorine Additive Component (F)>>

The resist composition according to the present embodiment may further include a fluorine additive component (hereinafter, referred to as a “component (F)”) in order to impart water repellency to the resist film or to improve lithography characteristics.

As the component (F), a fluorine-containing polymeric compound described in Japanese Unexamined Patent Application, First Publication No. 2010-002870, Japanese Unexamined Patent Application, First Publication No. 2010-032994, Japanese Unexamined Patent Application, First Publication No. 2010-277043, Japanese Unexamined Patent Application, First Publication No. 2011-13569, and Japanese Unexamined Patent Application, First Publication No. 2011-128226 can be mentioned.

Specific examples of the component (F) include polymers having a constitutional unit (f1) represented by General Formula (f1-1) shown below. This polymer is preferably a polymer (a homopolymer) consisting only of a constitutional unit (f1) represented by General Formula (f1-1); a copolymer of the constitutional unit (f1) and the constitutional unit (a1); a copolymer of the constitutional unit (f1), a constitutional unit derived from acrylic acid or methacrylic acid, and the constitutional unit (a1), and more preferably a copolymer of the constitutional unit (f1) and the constitutional unit (a1). The constitutional unit (a1) to be copolymerized with the constitutional unit (f1) is preferably a constitutional unit derived from 1-ethyl-1-cyclooctyl (meth)acrylate or a constitutional unit derived from 1-methyl-1-adamantyl (meth)acrylate, and more preferably a constitutional unit derived from 1-ethyl-1-cyclooctyl (meth)acrylate.

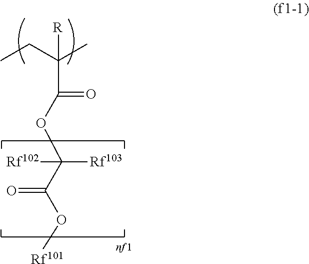

[In the formula, R is the same as that described above. Rf¹⁰² and Rf¹⁰³ each independently represents a hydrogen atom, a halogen atom, an alkyl group having 1 to 5 carbon atoms, or a halogenated alkyl group having 1 to 5 carbon atoms, and Rf¹⁰² and Rf¹⁰³ may be the same or different from each other. nf¹ represents an integer in a range of 0 to 5, and Rf¹⁰¹ represents an organic group containing a fluorine atom.]

In General Formula (f1-1), R bonded to the carbon atom at the α-position is the same as that described above. R is preferably a hydrogen atom or a methyl group.

In General Formula (f1-1), the halogen atom of Rf¹⁰² and Rf¹⁰³ is preferably a fluorine atom. Examples of the alkyl group having 1 to 5 carbon atoms as Rf¹⁰² and Rf¹⁰³ include the same one as the alkyl group having 1 to 5 carbon atoms as R, where a methyl group or an ethyl group is preferable. Specific examples of the halogenated alkyl group having 1 to 5 carbon atoms as Rf¹⁰² and Rf¹⁰³ include groups in which part or all of hydrogen atoms of the above-described alkyl groups of 1 to 5 carbon atoms have been substituted with a halogen atom. The halogen atom is preferably a fluorine atom. Among the above, Rf¹⁰² and Rf¹⁰³ are preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In General Formula (f1-1), nf¹ represents an integer in a range of 0 to 5, preferably an integer in a range of 0 to 3, and more preferably an integer of 1 or 2.

In General Formula (f1-1), Rf¹⁰¹ represents an organic group containing a fluorine atom and is preferably a hydrocarbon group containing a fluorine atom.

The hydrocarbon group containing a fluorine atom may be linear, branched, or cyclic, and preferably has 1 to 20 carbon atoms, more preferably has 1 to 15 carbon atoms, and particularly preferably has 1 to 10 carbon atoms.

In addition, in the hydrocarbon group containing a fluorine atom, 25% or more of the hydrogen atoms in the hydrocarbon group are preferably fluorinated, more preferably 50% or more are fluorinated, and particularly preferably 60% or more are fluorinated since the hydrophobicity of the resist film during immersion exposure increases.

Among the above, Rf¹⁰¹ is more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of the polystyrene equivalent value determined by gel permeation chromatography) of the component (F) is preferably in a range of 1,000 to 50,000, more preferably in a range of 5,000 to 40,000, and most preferably in a range of 10,000 to 30,000. In a case where the weight-average molecular weight is equal to or smaller than the upper limit value of this range, the resist composition exhibits sufficient solubility in a resist solvent to be used as a resist. On the other hand, in a case where the weight-average molecular weight is equal to or larger than the lower limit value of this range, the water repellency of the resist film is excellent.

Further, the polydispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in a combination of two or more kinds thereof.

In a case where the resist composition contains the component (F), the content of the component (F) in the resist composition is preferably in a range of 0.5 to 10 parts by mass and more preferably in a range of 1 to 10 parts by mass with respect to 100 parts by mass of the component (A).

### <<Organic Solvent Component (S)>>

The resist composition according to the present embodiment may be produced by dissolving the resist materials in an organic solvent component (hereinafter, referred to as a “component (S)”).

The component (S) may be any organic solvent which can dissolve each of the components to be used to obtain a homogeneous solution, and optional organic solvent can be appropriately selected from those which are conventionally known in the related art as solvents for a chemical amplification-type resist composition and then used.

Examples of the component (S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols, such as ethylene glycol, diethylene glycol, propylene glycol and dipropylene glycol; compounds having an ester bond, such as ethylene glycol monoacetate, diethylene glycol monoacetate, propylene glycol monoacetate, and dipropylene glycol monoacetate; polyhydric alcohol derivatives including compounds having an ether bond, such as a monoalkyl ether (such as monomethyl ether, monoethyl ether, monopropyl ether or monobutyl ether) or monophenyl ether of any of these polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzyl ether, cresylmethyl ether, diphenyl ether, dibenzyl ether, phenetole, butylphenyl ether, ethyl benzene, diethyl benzene, pentyl benzene, isopropyl benzene, toluene, xylene, cymene and mesitylene; and dimethylsulfoxide (DMSO).

In the resist composition according to the present embodiment, the component (S) may be used alone or as a mixed solvent of two or more kinds thereof. Among these, PGMEA, PGME, γ-butyrolactone, EL, or cyclohexanone is preferable.

In addition, a mixed solvent obtained by mixing PGMEA with a polar solvent is also preferable as the component (S). The blending ratio (mass ratio) of the mixed solvent can be appropriately determined, taking into consideration the compatibility of the PGMEA with the polar solvent, but is preferably in a range of 1:9 to 9:1 and more preferably in a range of 2:8 to 8:2.

More specifically, in a case where EL or cyclohexanone is blended as the polar solvent, the PGMEA:EL or cyclohexanone mass ratio is preferably in a range of 1:9 to 9:1 and more preferably in a range of 2:8 to 8:2. Alternatively, in a case where PGME is blended as the polar solvent, the PGMEA:PGME mass ratio is preferably in a range of 1:9 to 9:1, more preferably in a range of 2:8 to 8:2, and still more preferably in a range of 3:7 to 7:3. Furthermore, a mixed solvent of PGMEA, PGME, and cyclohexanone is also preferable.

In addition, the component (S) is also preferably a mixed solvent of at least one selected from PGMEA and EL and γ-butyrolactone. In this case, as the mixing ratio, the mass ratio of the former to the latter is preferably in a range of 70:30 to 95:5.

The amount of the component (S) to be used is not particularly limited and is appropriately set, depending on a thickness of a film to be coated, to a concentration at which the component (S) can be applied onto a substrate or the like. Generally, the component (S) is used such that the solid content concentration of the resist composition is in a range of 0.1% to 20% by mass and preferably in a range of 0.2% to 15% by mass.

As desired, other miscible additives can be also added to the resist composition according to the present embodiment. For example, for improving the performance of the resist film, an additive resin, a dissolution inhibitor, a plasticizer, a stabilizer, a colorant, a halation prevention agent, and a dye can be appropriately contained therein.

After dissolving the resist material in the component (S), the resist composition according to the present embodiment may be subjected to the removal of impurities and the like by using a porous polyimide membrane, a porous polyamideimide membrane, or the like. For example, the resist composition may be filtered using a filter consisting of a porous polyimide membrane, a filter consisting of a porous polyamideimide membrane, or a filter consisting of a porous polyimide membrane and a porous polyamideimide membrane. Examples of the porous polyimide membrane and the porous polyamideimide membrane include those described in Japanese Unexamined Patent Application, First Publication No. 2016-155121.

The resist composition according to the present embodiment described above contains a resin component (A1) having the constitutional unit (a01) and the compound (B0) (the component (B0)). In the resist composition in the related art, there is a trade-off relationship between sensitivity and roughness, and thus there is a tendency that in a case where one of them is improved, the other is deteriorated. However, in the resist composition according to the present embodiment, it is possible to eliminate the trade-off relationship and favorably maintain both the sensitivity and the roughness. Further, the exposure margin is also improved. The reason both the sensitivity and the roughness are favorably maintained in the resist composition according to the present embodiment while a wide exposure margin is ensured is presumed to be as follows.

The component (B0) contains an iodine atom in the anion moiety, whereby the sensitivity to EUV, EB, and the like is improved.

In the constitutional unit (a01), the deprotection rate is improved, and the sensitivity is improved by introducing a carbon-carbon double bond into the acid-dissociable group. In addition, the carbon-carbon double bond introduced into the acid-dissociable group improves the balance between the hydrophilicity and the hydrophobicity of the resist composition, which reduces the roughness.

Further, a wide exposure margin can be ensured due to the synergistic effect of the component (A1) having the constitutional unit (a01) and the component (B0). As a result, it is conceived that it is possible to achieve high sensitivity and low roughness while ensuring a wide exposure margin.

### [Resist Composition According to Second Aspect]

### <Component (A)>

### In Regard to Component (A1)

In the resist composition according to the second aspect, the component (A1) has a constitutional unit (a02) that contains a lactone-containing cyclic group containing an acid-dissociable group, an —SO₂—-containing cyclic group containing an acid-dissociable group, or a carbonate-containing cyclic group containing an acid-dissociable group.

<Constitutional Unit (a02)>

The constitutional unit (a02) is a constitutional unit that contains a lactone-containing cyclic group containing an acid-dissociable group, an —SO₂—-containing cyclic group containing an acid-dissociable group, or a carbonate-containing cyclic group containing an acid-dissociable group.

The description of the lactone-containing cyclic group is the same as that in the first aspect.

The lactone-containing cyclic group for the constitutional unit (a02) is not particularly limited, and any lactone-containing cyclic group may be used. Specific examples thereof include groups each represented by General Formulae (a02-r1-1) to (a02-r1-7) shown below.

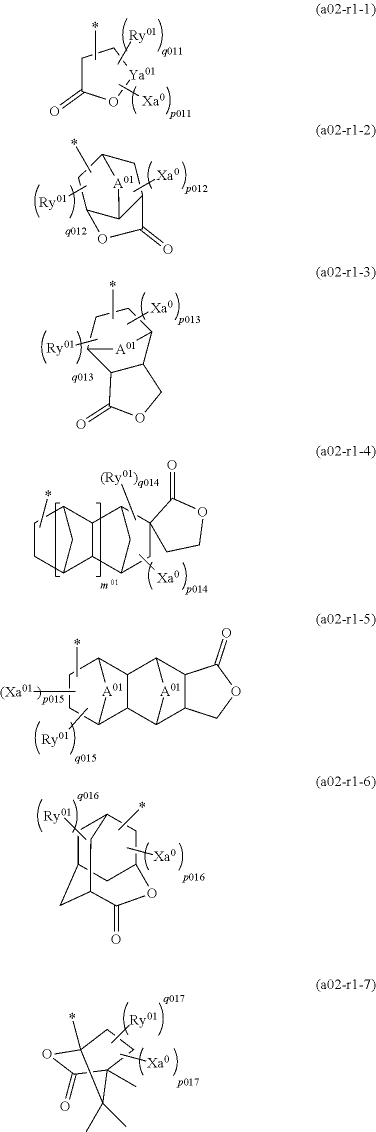

[In the formula, Xa⁰'s each independently represents a group containing an acid-dissociable group. Ya⁰¹ represents a single bond or a linear alkylene group having n⁰¹ carbon atoms, where n⁰¹ represents 1 or 2. Ry⁰¹'s each independently represents an alkyl group, an alkoxy group, a halogen atom, a halogenated alkyl group, a hydroxyl group, —COOR⁰″, —OC(═O)R⁰″, a hydroxyalkyl group, or a cyano group. R⁰″ represents a hydrogen atom, an alkyl group, a lactone-containing cyclic group, a carbonate-containing cyclic group, or an —SO₂—-containing cyclic group. A⁰¹'s each independently represents an oxygen atom, a sulfur atom, or an alkylene group having 1 to 5 carbon atoms, which may contain an oxygen atom, a sulfur atom. m⁰¹ represents 0 or 1. p⁰¹¹ represents an integer in a range of 1 to (3+2n⁰¹), and q⁰¹¹ represents an integer in a range of 0 to (2+2n⁰¹), where p⁰¹¹+q⁰¹¹≤3+2n⁰¹ is satisfied (here, n⁰¹=0 in a case where Ya⁰¹ is a single bond). p⁰¹² represents an integer in a range of 1 to 7, and q⁰¹² represents an integer in a range of 0 to 6, where p⁰¹²+q⁰¹²≤7 is satisfied. p⁰¹³ represents an integer in a range of 1 to 7, and q⁰¹³ represents an integer in a range of 0 to 6, where p⁰¹³+q⁰¹³≤7 is satisfied. p⁰¹⁴ represents an integer in a range of 1 to 8, and q⁰¹⁴ represents an integer in a range of 0 to 7, where p⁰¹⁴+q⁰¹⁴≤8 is satisfied. p⁰¹⁵ represents an integer in a range of 1 to 10, and q⁰¹⁵ represents an integer in a range of 0 to 9, where p⁰¹⁵+q⁰¹⁵≤10 is satisfied. p⁰¹⁶ represents an integer in a range of 1 to 8, and q⁰¹⁶ represents an integer in a range of 0 to 7, where p⁰¹⁶+q⁰¹⁶≤8 is satisfied. p⁰¹⁷ represents an integer in a range of 1 to 4, and q⁰¹⁷ represents an integer in a range of 0 to 3, where p⁰¹⁷+q⁰¹⁷≤4 is satisfied. * represents a bonding site.]

In General Formulae (a02-r1-1) to (a02-r1-7), Ry⁰¹'s each independently represents an alkyl group, an alkoxy group, a halogen atom, a halogenated alkyl group, a hydroxyl group, —COOR⁰″, —OC(═O)R⁰″, a hydroxyalkyl group, or a cyano group. R⁰″ represents a hydrogen atom, an alkyl group, a lactone-containing cyclic group, a carbonate-containing cyclic group, or an —SO₂—-containing cyclic group.

The alkyl group as Ry⁰¹ is preferably an alkyl group having 1 to 6 carbon atoms. It is preferable that the alkyl group be linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or an ethyl group is preferable, and a methyl group is more preferable.

The alkoxy group as Ry⁰¹ is preferably an alkoxy group having 1 to 6 carbon atoms. It is preferable that the alkoxy group be linear or branched. Specific examples of the alkoxy groups include a group obtained by linking the above-described alkyl group mentioned as the alkyl group represented by Ry⁰¹ to an oxygen atom (—O—).

The halogen atom as Ry⁰¹ is preferably a fluorine atom.

Examples of the halogenated alkyl group as Ry⁰¹ include a group obtained by substituting part or all of hydrogen atoms in the above-described alkyl group as Ry⁰¹ with the above-described halogen atoms. The halogenated alkyl group is preferably a fluorinated alkyl group and more preferably a perfluoroalkyl group.

In —COOR⁰″ and —OC(═O)R⁰″, each R⁰″ independently represents a hydrogen atom, an alkyl group, a lactone-containing cyclic group, a carbonate-containing cyclic group, or an —SO₂—-containing cyclic group.

The alkyl group as R⁰″ may be linear, branched, or cyclic. The alkyl group as R⁰″ preferably has 1 to 15 carbon atoms.

In a case where R⁰″ represents a linear alkyl group, the linear alkyl group preferably has 1 to 10 carbon atoms and more preferably has 1 to 5 carbon atoms, and it is still more preferably a methyl group or an ethyl group. In a case where R⁰″ represents a branched alkyl group, the branched alkyl group preferably has 3 to 10 carbon atoms, more preferably has 3 to 5 carbon atoms, and still more preferably has 3 or 4 carbon atoms.

In a case where R⁰″ represents a cyclic alkyl group, the cyclic alkyl group preferably has 3 to 15 carbon atoms, more preferably has 4 to 12 carbon atoms, and still more preferably has 5 to 10 carbon atoms. Specific examples thereof include a group obtained by removing one or more hydrogen atoms from a monocycloalkane which may be substituted with a fluorine atom or a fluorinated alkyl group; and a group obtained by removing one or more hydrogen atoms from a polycycloalkane such as bicycloalkane, tricycloalkane, or tetracycloalkane. More specific examples thereof include a group obtained by removing one or more hydrogen atoms from a monocycloalkane such as cyclopentane or cyclohexane; and a group obtained by removing one or more hydrogen atoms from a polycycloalkane such as adamantane, norbornane, isobornane, tricyclodecane, or tetracyclododecane.

Examples of the lactone-containing cyclic group as R₀″ include groups each represented by General Formulae (a2-r-1) to (a2-r-7) which will be described later.

Examples of the carbonate-containing cyclic group as R⁰″ include groups each represented by General Formulae (ax3-r-1) to (ax3-r-3) which will be described later.

Examples of the —SO₂—-containing cyclic group as R⁰″ include groups each represented by General Formulae (a5-r-1) to (a5-r-4) which will be described later.

The hydroxyalkyl group as Ry⁰¹ preferably has 1 to 6 carbon atoms. Specific examples thereof include a group obtained by substituting at least one hydrogen atom in the alkyl group as Ry⁰¹ with a hydroxyl group.

Among the above, Ry⁰¹ is preferably a hydrogen atom or a cyano group.

In General Formula (a02-r1-1), Ya⁰¹ represents a single bond or a linear alkylene group having n⁰¹ carbon atoms. n⁰¹ represents 1 or 2. Ya⁰¹ is preferably a linear alkylene group having n⁰¹ carbon atoms and more preferably a linear alkylene group having one carbon atom (a methylene group).

In General Formulae (a02-r1-2), (a02-r1-3), and (a02-r1-5), A⁰¹'s each independently represents an oxygen atom, a sulfur atom, or an alkylene group having 1 to 5 carbon atoms, which may contain an oxygen atom, a sulfur atom.

The alkylene group having 1 to 5 carbon atoms as A⁰¹ is preferably a linear or branched alkylene group, and examples thereof include a methylene group, an ethylene group, an n-propylene group, and an isopropylene group. In a case where the alkylene group as A⁰¹ contains an oxygen atom or a sulfur atom, examples thereof include a group in which —O— or —S— is interposed at the terminal of the alkylene group or between the carbon atoms of the alkylene group. Specific examples thereof include —O—CH₂—, —CH₂— O—CH₂—, —S—CH₂—, and —CH₂—S—CH₂—. A⁰¹ is preferably an alkylene group having 1 to 5 carbon atoms or an oxygen atom (—O—), more preferably an alkylene group having 1 to 5 carbon atoms, and still more preferably a methylene group.

In General Formulae (a02-r1-1) to (a02-r1-7), Xa⁰'s each independently represents a group containing an acid-dissociable group. Xa⁰ will be described later.

In General Formula (a02-r1-4), m⁰¹ represents 0 or 1. m⁰¹ is preferably 0.

In General Formula (a02-r1-4), Ra⁰¹ and Xa⁰¹ may be bonded to any position of the polycycloalkane ring bonded to the γ-butyrolactone ring. In General Formula (a02-r1-5). Ra⁰¹ and Xa⁰¹ may be bonded to any position of the polycycloalkane ring condensed with the γ-butyrolactone ring.

In General Formula (a02-r1-1), p⁰¹¹ represents an integer in a range of 1 to (3+2n⁰¹), and q⁰¹¹ represents an integer in a range of 0 to (2+2n⁰¹), where p⁰¹¹+q⁰¹¹≤3+2n⁰¹ is satisfied (here, n⁰¹=0 in a case where Ya is a single bond). p⁰¹¹ is preferably 1 to 3, more preferably 1 or 2, and still more preferably 1. q⁰¹¹ is preferably 0 to 3, more preferably 0 to 2, and still more preferably 0 or 1.

In General Formula (a02-r1-2), p⁰¹² represents an integer in a range of 1 to 7, and q⁰¹² represents an integer in a range of 0 to 6, where p⁰¹²+q⁰¹²≤7 is satisfied. p⁰¹² is preferably 1 to 3, more preferably 1 or 2, and still more preferably 1. q⁰¹² is preferably 0 to 3, more preferably 0 to 2, and still more preferably 0 or 1. In General Formula (a02-r1-3), p⁰¹³ represents an integer in a range of 1 to 7, and q⁰¹³ represents an integer in a range of 0 to 6, where p⁰¹³+q⁰¹³≤7 is satisfied. p⁰¹³ is preferably 1 to 3, more preferably 1 or 2, and still more preferably 1. q⁰¹³ is preferably 0 to 3, more preferably 0 to 2, and still more preferably 0 or 1.

In General Formula (a02-r1-4), p⁰¹⁴ represents an integer in a range of 1 to 8, and q⁰¹⁴ represents an integer in a range of 0 to 7, where p⁰¹⁴+q⁰¹⁴≤8 is satisfied. p⁰¹⁴ is preferably 1 to 3, more preferably 1 or 2, and still more preferably 1. q⁰¹⁴ is preferably 0 to 3, more preferably 0 to 2, and still more preferably 0 or 1.

In General Formula (a02-r1-5), p⁰¹⁵ represents an integer in a range of 1 to 10, and q⁰¹⁵ represents an integer in a range of 0 to 9, where p⁰¹⁵+q⁰¹⁵≤10 is satisfied. p⁰¹⁵ represents an integer in a range of 1 to 8, and q⁰¹⁵ represents an integer in a range of 0 to 7, where p⁰¹⁶+q⁰¹⁵≤8 is satisfied. p⁰¹⁵ is preferably 1 to 3, more preferably 1 or 2, and still more preferably 1. q⁰¹⁵ is preferably 0 to 3, more preferably 0 to 2, and still more preferably 0 or 1.

In General Formula (a02-r1-6), p⁰¹⁶ represents an integer in a range of 1 to 8, and q⁰¹⁶ represents an integer in a range of 0 to 7, where p⁰¹⁶+q⁰¹⁶≤8 is satisfied. p⁰¹⁶ is preferably 1 to 3, more preferably 1 or 2, and still more preferably 1. q⁰¹⁶ is preferably 0 to 3, more preferably 0 to 2, and still more preferably 0 or 1.

In General Formula (a02-r1-7), p⁰¹⁷ represents an integer in a range of 1 to 4, and q⁰¹³ represents an integer in a range of 0 to 3, where p⁰¹³+q⁰¹³≤4 is satisfied. p⁰¹⁷ is preferably 1 to 3, more preferably 1 or 2, and still more preferably 1. q⁰¹⁷ is preferably 0 to 3, more preferably 0 to 2, and still more preferably 0 or 1.

Specific examples of the groups each represented by General Formulae (a02-r1-1) to (a02-r1-7) are shown below. In the following formulae, Xa⁰ represents a group containing an acid-dissociable group. * represents a bonding site.

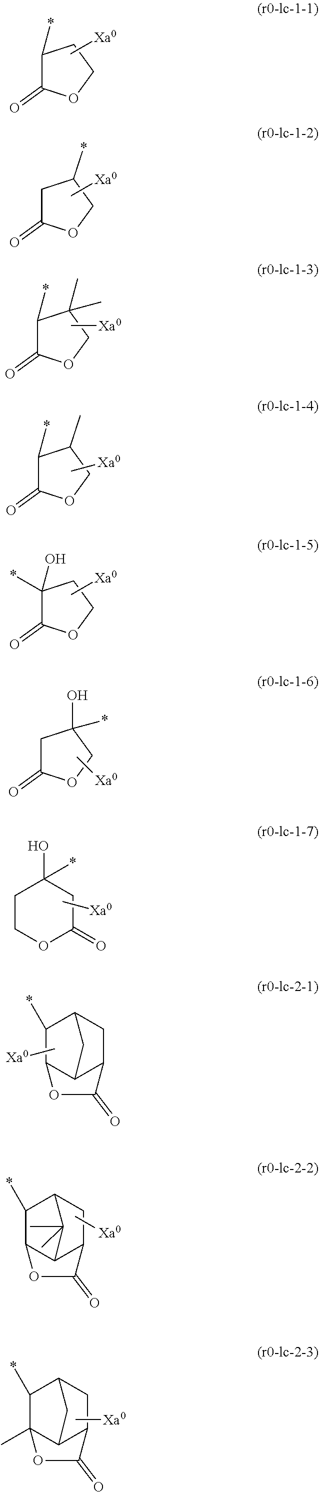

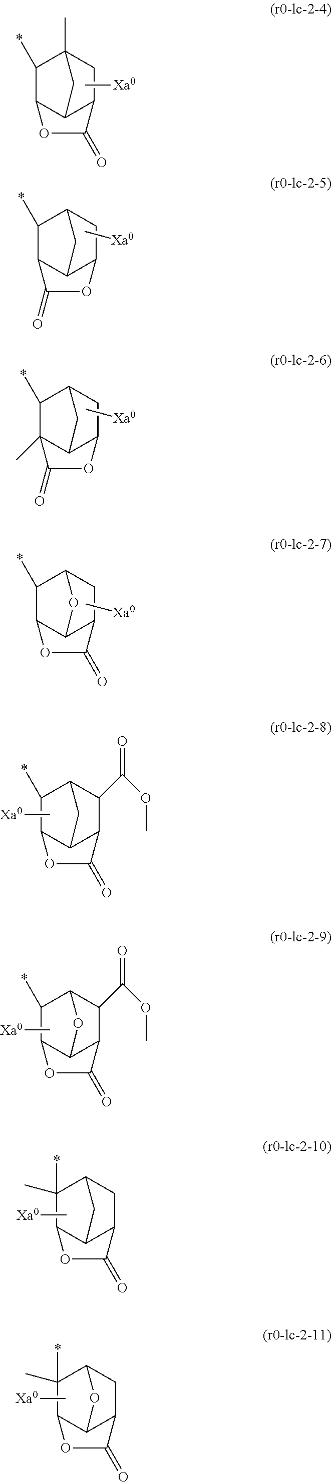

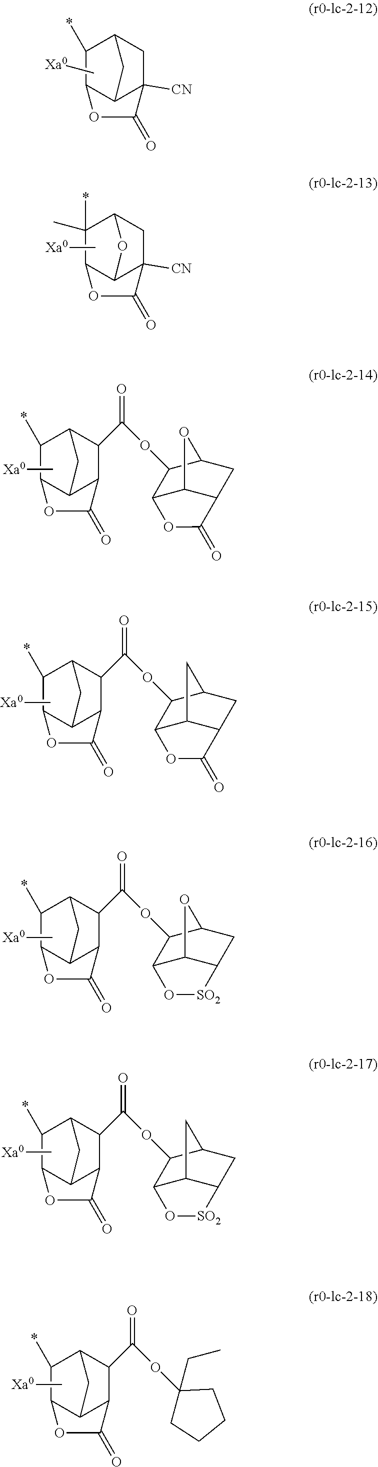

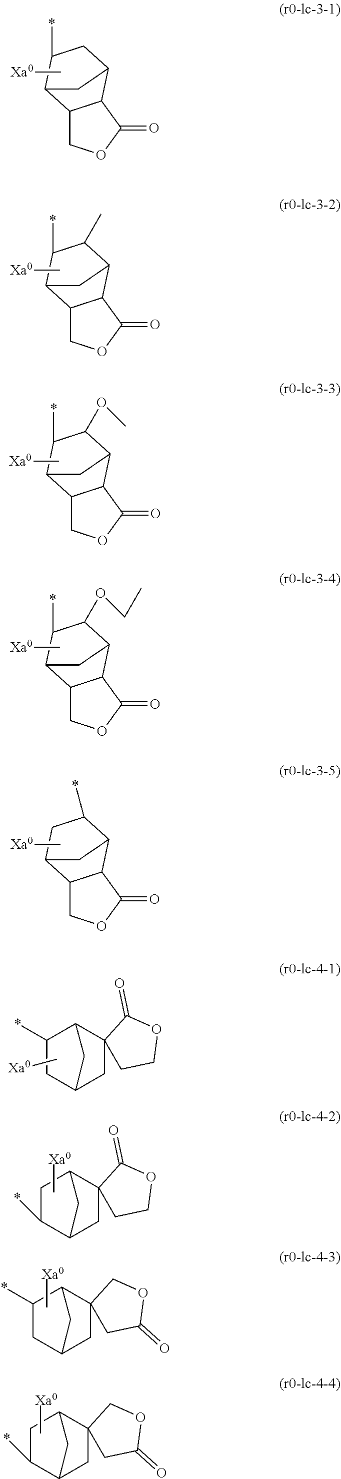

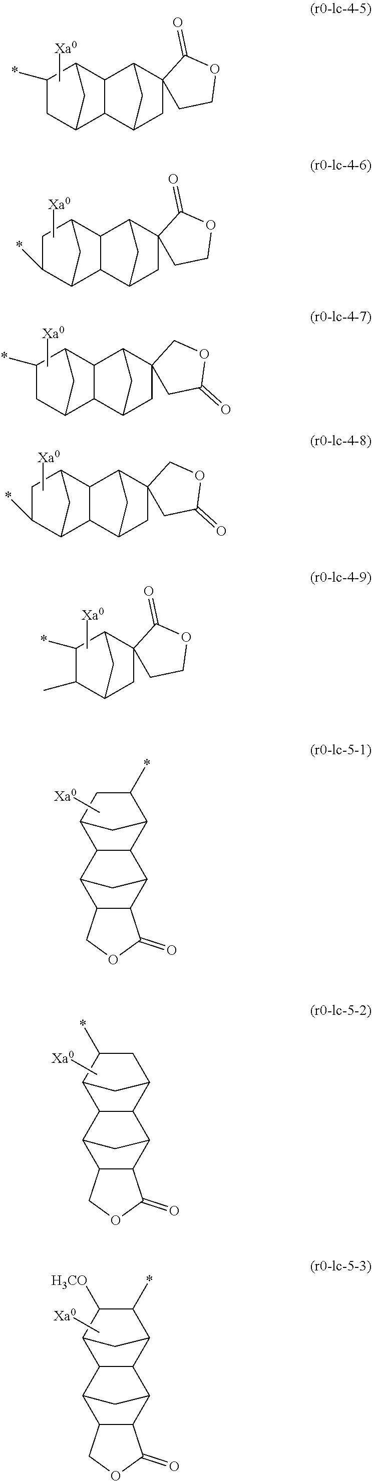

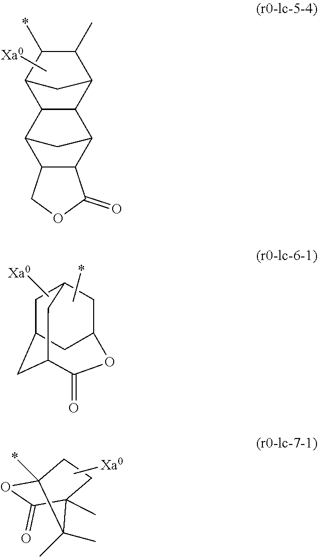

The explanation of the —SO₂—-containing cyclic group is the same as that in the first aspect.

Examples of the —SO₂—-containing cyclic group in the constitutional unit (a02) include groups each represented by General Formulae (a02-r5-1) to (a02-r5-4) shown below.

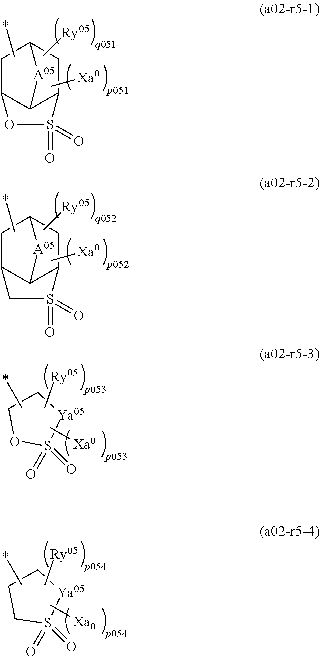

[In the formula, Xa⁰'s each independently represents a group containing an acid-dissociable group. Ry⁰⁵'s each independently represents an alkyl group, an alkoxy group, a halogen atom, a halogenated alkyl group, a hydroxyl group, —COOR⁰″, —OC(═O)R⁰″, a hydroxyalkyl group, or a cyano group. R⁰″ represents a hydrogen atom, an alkyl group, a lactone-containing cyclic group, a carbonate-containing cyclic group, or an —SO₂—-containing cyclic group. A⁰⁵ represents an alkylene group having 1 to 5 carbon atoms which may contain an oxygen atom or a sulfur atom, an oxygen atom, or a sulfur atom. Ya⁰⁵ represents a single bond or a linear alkylene group having n⁰⁵ carbon atoms, where n⁰¹ represents 1 or 2. p⁰⁵¹ represents an integer in a range of 1 to 7, and q⁰⁵¹ represents an integer in a range of 0 to 6, where p⁰⁵¹+q⁰⁵¹≤7 is satisfied. p⁰⁵² represents an integer in a range of 1 to 7, and q⁰⁵² represents an integer in a range of 0 to 6, where p⁰⁵²+q⁰⁵²≤7 is satisfied. p⁰⁵³ represents an integer in a range of 1 to (3+2n⁰¹), and q⁰⁵³ represents an integer in a range of 0 to (2+2n⁰¹), where p⁰⁵³+q⁰⁵³≤3+2n⁰¹ is satisfied (here, n⁰¹=0 in a case where Ya⁰⁵ is a single bond). p⁰⁵⁴ represents an integer in a range of 1 to (5+2n⁰¹), and q⁰⁵⁴ represents an integer in a range of 0 to (4+2n⁰¹), where p⁰⁵⁴+q⁰⁵⁴≤5+2n⁰¹ is satisfied (here, n⁰¹=0 in a case where Ya⁰⁵ is a single bond.]

In General Formulae (a02-r5-1) and (a02-r5-2), A⁰⁵ represents an alkylene group having 1 to 5 carbon atoms which may contain an oxygen atom or a sulfur atom, an oxygen atom, or a sulfur atom. A⁰⁵ is the same as A⁰¹ in General Formulae (a02-r1-2), (a02-r1-3), and (a02-r1-5).

In General Formulae (a02-r5-1) and (a02-r5-2), Ry⁰⁵'s each independently represents an alkyl group, an alkoxy group, a halogen atom, a halogenated alkyl group, a hydroxyl group, —COOR⁰″, —OC(═O)R⁰″, a hydroxyalkyl group, or a cyano group. R⁰″ represents a hydrogen atom, an alkyl group, a lactone-containing cyclic group, a carbonate-containing cyclic group, or an —SO₂—-containing cyclic group. Examples of the alkyl group, the alkoxy group, the halogen atom, the halogenated alkyl group, —COOR⁰″, —OC(═O)R⁰″, and the hydroxyalkyl group as Ry⁰⁵ include the same as those described in the description for Ry⁰¹ in General Formulae (a02-r1-1) to (a02-r1-7).

In General Formulae (a02-r5-3) and (a02-r5-4), Ya⁰⁵ represents a single bond or a linear alkylene group having n⁰¹ carbon atoms. n⁰¹ represents 1 or 2. Ya⁰⁵ is preferably a linear alkylene group having n⁰¹ carbon atoms and more preferably a linear alkylene group having one carbon atom (a methylene group).

In General Formulae (a02-r5-1) to (a02-r5-4), Xa⁰'s each independently represents a group containing an acid-dissociable group. Xa⁰ will be described later.

In General Formula (a02-r5-1), p⁰⁵¹ represents an integer in a range of 1 to 7, and q⁰⁵¹ represents an integer in a range of 0 to 6, where p⁰⁵¹+q⁰⁵¹≤7 is satisfied. p⁰⁵¹ is preferably 1 to 3, more preferably 1 or 2, and still more preferably 1. q⁰⁵¹ is preferably 0 to 3, more preferably 0 to 2, and still more preferably 0 or 1.

In General Formula (a02-r5-2), p⁰⁵² represents an integer in a range of 1 to 7, and q⁰⁵² represents an integer in a range of 0 to 6, where p⁰⁵²+q⁰⁵²≤7 is satisfied. p⁰⁵² is preferably 1 to 3, more preferably 1 or 2, and still more preferably 1. q⁰⁵² is preferably 0 to 3, more preferably 0 to 2, and still more preferably 0 or 1.

In General Formula (a02-r5-3), p⁰⁵³ represents an integer in a range of 1 to (3+2n⁰¹), and q⁰⁵³ represents an integer in a range of 0 to (2+2n⁰¹), where p⁰⁵³+q⁰⁵³≤3+2n⁰¹ is satisfied (here, n⁰¹=0 in a case where Ya⁰⁵ is a single bond). p⁰⁵³ is preferably 1 to 3, more preferably 1 or 2, and still more preferably 1. q⁰⁵³ is preferably 0 to 3, more preferably 0 to 2, and still more preferably 0 or 1.

In General Formula (a02-r5-4), p⁰⁵⁴ represents an integer in a range of 1 to (5+2n⁰¹), and q⁰⁵⁴ represents an integer in a range of 0 to (4+2n⁰¹), where p⁰⁵⁴+q⁰⁵⁴≤5+2n⁰¹ is satisfied (here, n⁰¹=0 in a case where Ya⁰⁵ is a single bond). p⁰⁵⁴ is preferably 1 to 3, more preferably 1 or 2, and still more preferably 1. q⁰⁵⁴ is preferably 0 to 3, more preferably 0 to 2, and still more preferably 0 or 1.

Specific examples of the groups each represented by General Formulae (a02-r5-1) to (a02-r5-4) are shown below. In the following formulae, “Ac” represents an acetyl group. Xa⁰ represents a group containing an acid-dissociable group. * represents a bonding site.

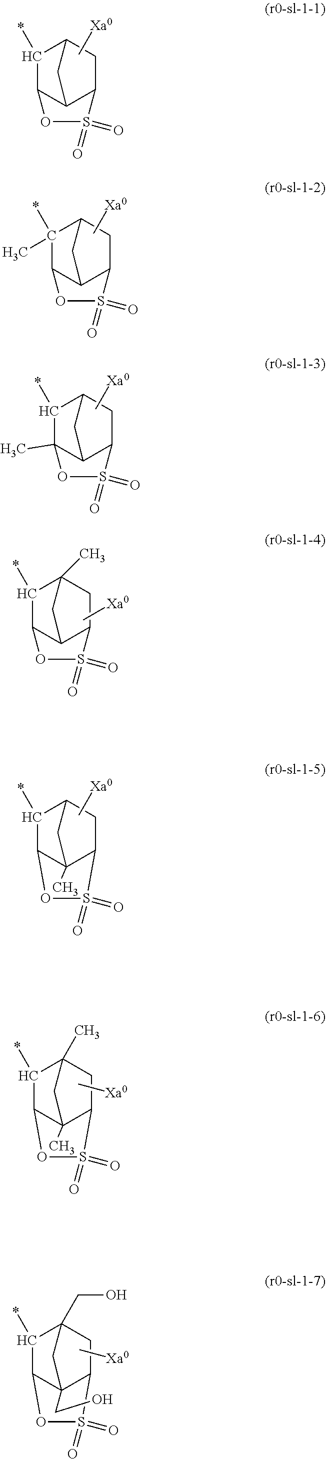

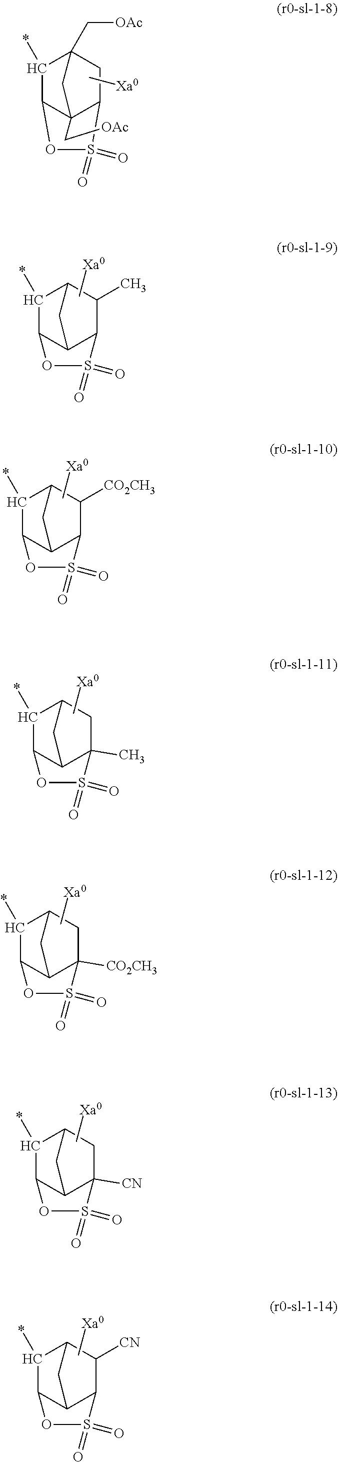

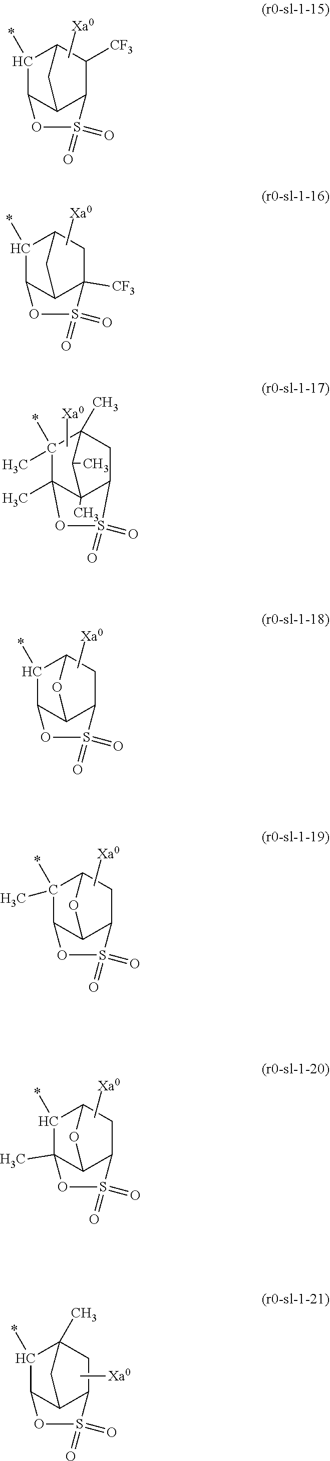

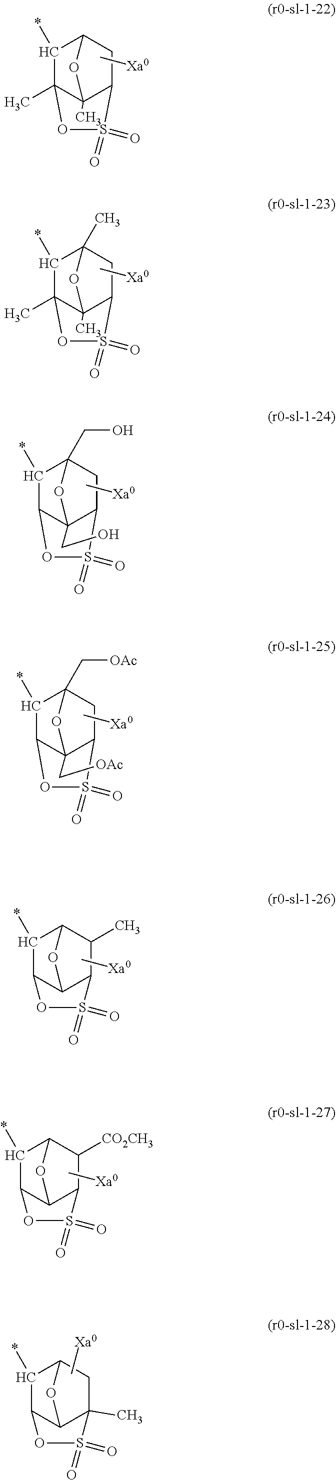

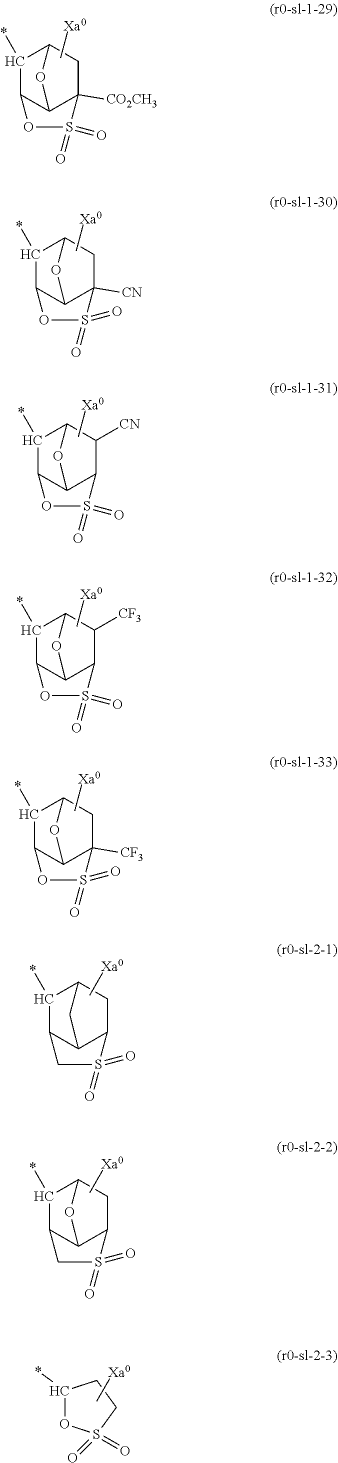

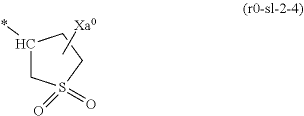

The explanation of the carbonate-containing cyclic group is the same as that in the first aspect.

Examples of the carbonate-containing cyclic group in the constitutional unit (a02) include groups each represented by General Formulae (a02-rx3-1) to (a02-rx3-3) shown below.

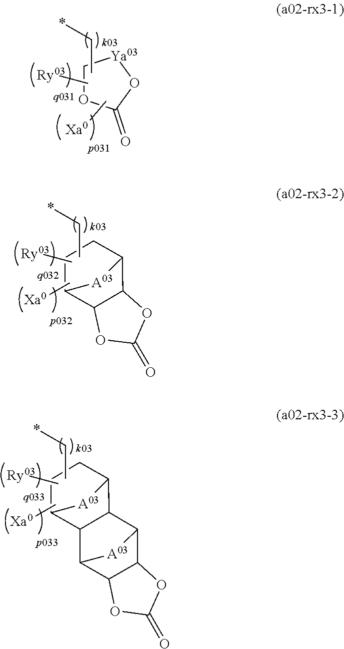

[In the formula, Xa⁰'s each independently represents a group containing an acid-dissociable group. Ya⁰³ represents a single bond or a linear alkylene group having n⁰³ carbon atoms, where n⁰³ represents an integer of 1 or 3. Ry⁰³'s each independently represents an alkyl group, an alkoxy group, a halogen atom, a halogenated alkyl group, a hydroxyl group, —COOR⁰″, —OC(═O)R⁰″, a hydroxyalkyl group, or a cyano group. R⁰″ represents a hydrogen atom, an alkyl group, a lactone-containing cyclic group, a carbonate-containing cyclic group, or an —SO₂—-containing cyclic group. A03 represents an alkylene group having 1 to 5 carbon atoms which may contain an oxygen atom or a sulfur atom, an oxygen atom, or a sulfur atom. k⁰³ represents 0 or 1. p⁰³¹ represents an integer in a range of 1 to (1+2n⁰³), q⁰³¹ represents an integer in a range of 0 to 2n⁰³, where p⁰³¹+q⁰³¹≤1+2n⁰³ is satisfied. p⁰³² represents an integer in a range of 1 to 7, and q⁰³² represents an integer in a range of 0 to 6, where p⁰³²+q⁰³²≤7 is satisfied. p⁰³³ represents an integer in a range of 1 to 11, and q⁰³³ represents an integer in a range of 0 to 10, where p⁰³³+q⁰³³≤11 is satisfied. * represents a bonding site.]

In General Formulae (a02-rx3-2) and (a02-rx3-3), A03 represents an alkylene group having 1 to 5 carbon atoms which may contain an oxygen atom or a sulfur atom, an oxygen atom, or a sulfur atom. A03 is the same as A01 in General Formulae (a02-r1-2) and (a02-r1-3).

In General Formulae (a02-rx3-1) to (a02-rx3-3), Ry⁰³'s each independently represents an alkyl group, an alkoxy group, a halogen atom, a halogenated alkyl group, a hydroxyl group, —COOR⁰″, —OC(═O)R₀″, a hydroxyalkyl group, or a cyano group. R⁰″ represents a hydrogen atom, an alkyl group, a lactone-containing cyclic group, a carbonate-containing cyclic group, or an —SO₂—-containing cyclic group. Examples of the alkyl group, the alkoxy group, the halogen atom, the halogenated alkyl group, —COOR⁰″, —OC(═O)R⁰″, and the hydroxyalkyl group as Ry⁰³ include the same as those described in the description for Ry⁰¹ in General Formulae (a02-r1-1) to (a02-r1-7).

In General Formula (a02-rx3-1), Ya⁰³ represents a single bond or a linear alkylene group having n⁰³ carbon atoms. n⁰³ represents an integer in a range of 1 to 3. n⁰³ is preferably 1 or 2 and more preferably 1. Ya⁰³ is preferably a linear alkylene group having n⁰³ carbon atoms and more preferably a linear alkylene group having one carbon atom (a methylene group).

In General Formulae (a02-rx3-1) to (a02-rx3-3), Xa⁰'s each independently represents a group containing an acid-dissociable group. Xa⁰ will be described later.

In General Formulae (a02-rx3-1) to (a02-rx3-3), k⁰³ represents 0 or 1. k⁰³ is preferably 0.

In General Formula (a02-rx3-1), p⁰³¹ represents an integer in a range of 1 to (1+2n⁰³), q⁰³¹ represents an integer in a range of 0 to 2n⁰³, where p⁰³¹+q⁰³¹≤1+2n⁰³ is satisfied. p⁰³¹ is preferably 1 to 3, more preferably 1 or 2, and still more preferably 1. q⁰³¹ is preferably 0 to 3, more preferably 0 to 2, and still more preferably 0 or 1. In General Formula (a02-rx3-2), p⁰³² represents an integer in a range of 1 to 7, and q⁰³² represents an integer in a range of 0 to 6, where p⁰³²+q⁰³²≤7 is satisfied. p⁰³² is preferably 1 to 3, more preferably 1 or 2, and still more preferably 1. q⁰³² is preferably 0 to 3, more preferably 0 to 2, and still more preferably 0 or 1.

In General Formula (a02-rx3-3), p⁰³³ represents an integer in a range of 1 to 11, q⁰³³ represents an integer in a range of 0 to 10, where p⁰³³+q⁰³³≤11 is satisfied. p⁰³³ is preferably 1 to 3, more preferably 1 or 2, and still more preferably 1. q⁰³³ is preferably 0 to 3, more preferably 0 to 2, and still more preferably 0 or 1.

Specific examples of the groups each represented by General Formulae (a02-rx3-1) to (a02-rx3-3) are shown below. In the formulae, Xa⁰ represents a group containing an acid-dissociable group. * represents a bonding site.

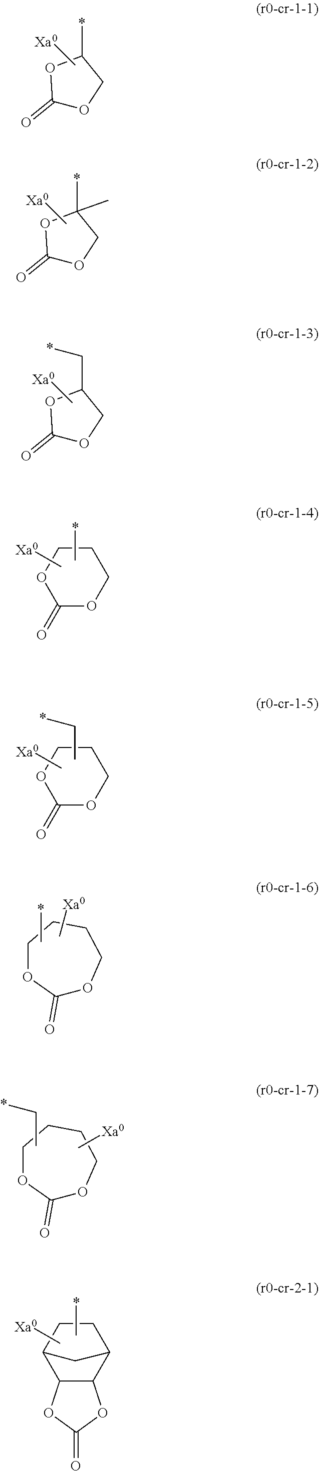

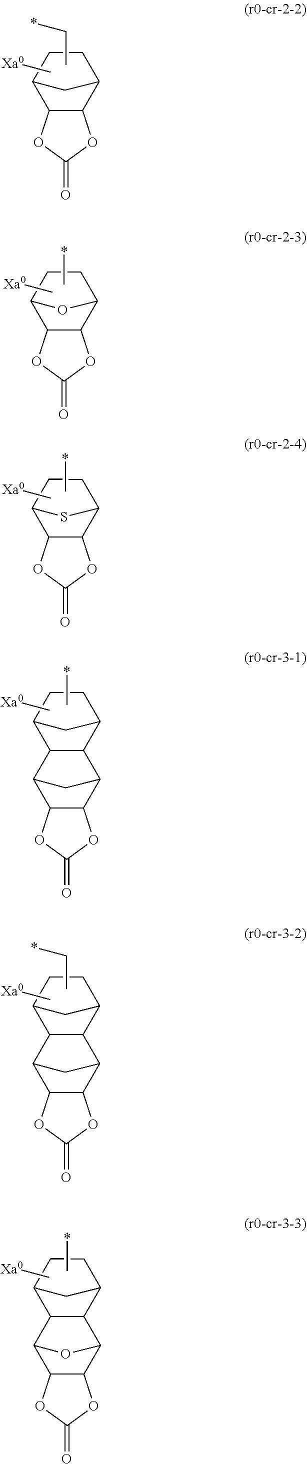

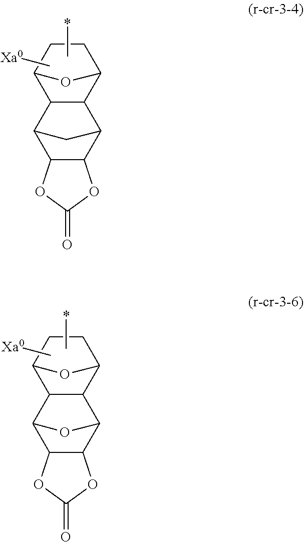

### <<Group Containing Acid-Dissociable Group: Xa^{0>>}

In General Formulae (a02-r1-1) to (a02-r1-7), General Formulae (a02-r5-1) to (a02-r5-4), and General Formulae (a02-rx3-1) to (a02-rx3-3), Xa⁰'s each independently represents a group containing an acid-dissociable group.

Examples of the acid-dissociable group include those which have been proposed so far as acid-dissociable groups for the base resin for a chemical amplification-type resist composition. Examples thereof include “the acetal-type acid-dissociable group”, “the tertiary alkyl ester-type acid-dissociable group”, and “the tertiary alkyloxycarbonyl acid-dissociable group”, which are described in the first aspect.

### Acetal-Type Acid-Dissociable Group:

Examples of the acetal-type acid-dissociable group include the acid-dissociable group represented by General Formula (a1-r-1).

### Tertiary Alkyl Ester-Type Acid-Dissociable Group:

Examples of the tertiary alkyl ester-type acid-dissociable group include the acid-dissociable group represented by General Formula (a1-r-2).

In General Formula (a1-r-2), in a case where Ra′⁵ to Ra′⁶ are bonded to each other to form a ring, suitable examples of the ring include a group represented by General Formula (a1-r2-1), a group represented by General Formula (a1-r2-3), and a group represented by General Formula (a1-r2-2).

On the other hand, suitable examples thereof include the group represented by General Formula (a1-r2-4) in a case where Ra′⁴ to Ra′⁶ are not bonded to each other and represent an independent hydrocarbon group.

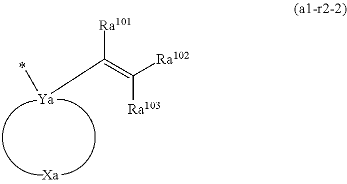

[In General Formula (a1-r2-2), Ya represents a carbon atom. Xa represents a group that forms a cyclic hydrocarbon group together with Ya. Part or all of hydrogen atoms contained in the cyclic hydrocarbon group may be substituted. Ra¹⁰¹ to Ra¹⁰³ each independently represents a hydrogen atom, a monovalent chain-like saturated hydrocarbon group having 1 to 10 carbon atoms, or a monovalent aliphatic cyclic saturated hydrocarbon group having 3 to 20 carbon atoms. Part or all of hydrogen atoms contained in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. * represents a bonding site.]

The linear alkyl group as Ra′¹⁰ in General Formula (a1-r2-1) preferably has 1 to 12 carbon atoms, more preferably has 1 to 10 carbon atoms, still more preferably has 1 to 5 carbon atoms, and particularly preferably has 1 to 3 carbon atoms. Specific examples thereof include a methyl group, an ethyl group, an n-propyl group, an n-butyl group, and an n-pentyl group. Among them, a methyl group, an ethyl group, or an n-butyl group is preferable, and a methyl group or an ethyl group is more preferable.

The branched alkyl group as Ra′¹⁰ preferably has 3 to 12 carbon atoms, more preferably 3 to 10 carbon atoms, still more preferably 3 to 5 carbon atoms, and particularly preferably 3 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among them, an isopropyl group is preferable.

A part of the alkyl group as Ra′¹⁰ may be substituted with a halogen atom or a hetero atom-containing group. For example, a part of hydrogen atoms constituting the alkyl group may be substituted with a halogen atom or a hetero atom-containing group. Alternatively, a part of carbon atoms (such as a methylene group) constituting the alkyl group may be substituted with a hetero atom-containing group.

Examples of the hetero atom include an oxygen atom, a sulfur atom, and a nitrogen atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

Ra′¹⁰ is preferably a linear or branched alkyl group having 1 to 5 carbon atoms, more preferably a linear or branched alkyl group having 1 to 3 carbon atoms, still more preferably a methyl group, an ethyl group, or an isopropyl group, and particularly preferably a methyl group.

In General Formula (a1-r2-1), Ra′¹¹ represents a group that forms an aliphatic cyclic group together with a carbon atom to which Ra′¹⁰ is bonded. The aliphatic cyclic group may be a polycyclic group or may be a monocyclic group.

The aliphatic hydrocarbon group which is a monocyclic group is preferably a group obtained by removing one hydrogen atom from a monocycloalkane. The monocycloalkane is preferably a monocycloalkane having 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, and tetracyclododecane.

Among them, the aliphatic cyclic group is preferably a cyclopentyl group, a cyclohexyl group, or an adamantyl group, and more preferably a cyclopentyl group or an adamantyl group.

In General Formula (a1-r2-2), examples of the cyclic hydrocarbon group that is formed by Xa together with Ya include a group in which one or more hydrogen atoms are further removed from a cyclic monovalent hydrocarbon group (an aliphatic hydrocarbon group) as Ra′³ in General Formula (a1-r-1).

The cyclic hydrocarbon group that is formed by Xa together with Ya may have a substituent. Examples of this substituent include the same one as the substituent which may be contained in the cyclic hydrocarbon group as Ra′³.

In General Formula (a1-r2-2), examples of the monovalent chain-like saturated hydrocarbon group having 1 to 10 carbon atoms, as Ra¹⁰¹ to Ra¹⁰³, include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms, as Ra¹⁰¹ to Ra¹⁰³, include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7] dodecanyl group, and an adamantyl group.

Among them, Ra¹⁰¹ to Ra¹⁰³ are preferably a hydrogen atom or a monovalent chain-like saturated hydrocarbon group having 1 to 10 carbon atoms, and among them, a hydrogen atom, a methyl group, and an ethyl group are more preferable, and a hydrogen atom is particularly preferable from the viewpoint of easy synthesis.

Examples of the substituent contained in the chain-like saturated hydrocarbon group represented by Ra¹⁰¹ to Ra¹⁰³ or the aliphatic cyclic saturated hydrocarbon group include the same groups as Ra^{x5} described above.

Examples of the group containing a carbon-carbon double bond generated by bonding two or more of Ra¹⁰¹ to Ra¹⁰³ to each other to form a cyclic structure include a cyclopentenyl group, a cyclohexenyl group, a methylcyclopentenyl group, a methylcyclohexenyl group, a cyclopentylideneethenyl group, and a cyclohexylideneethenyl group. Among these, a cyclopentenyl group, a cyclohexenyl group, or a cyclopentylideneethenyl group is preferable from the viewpoint of easy synthesis.

Specific examples of the group represented by General Formula (a1-r2-1) include an acid-dissociable group represented by any one of General Formulae (r-pr-m1) to (r-pr-m17), General Formulae (r-pr-s1) to (r-pr-s21), and General Formulae (r-pr-sp1) to (r-pr-sp6).

Specific examples of the group represented by General Formula (a1-r2-2) include an acid-dissociable group represented by any one of General Formulae (r-pr-sv1) to (r-pr-sv12) and General Formulae (r-pr-mv1) to (r-pr-mv21).

Specific examples of the group represented by General Formula (a1-r2-3) include an acid-dissociable group represented by any one of General Formulae (r-pr-sa1) to (r-pr-sa9) and General Formulae (r-pr-ma1) and (r-pr-ma2).

Specific examples of the group represented by General Formula (a1-r2-4) include an acid-dissociable group represented by any one of General Formulae (r-pr-cm1) to (r-pr-cm8), General Formulae (r-pr-cs1) to (r-pr-cs5), and General Formulae (r-pr-c1) to (r-pr-c3).

### Tertiary Alkyloxycarbonyl Acid-Dissociable Group:

Examples of the tertiary alkyloxycarbonyl acid-dissociable group include the acid-dissociable group represented by General Formula (a1-r-3).

The acid-dissociable group contained in Xa⁰ is preferably a tertiary alkyl ester-type acid-dissociable group, more preferably an acid-dissociable group represented by General Formula (a1-r-2), still more preferably an acid-dissociable group represented by General Formula (a1-r2-1) or (a1-r2-4), and particularly preferably an acid-dissociable group represented by General Formula (a1-r2-1). Among the above, it is preferably acid-dissociable groups represented by General Formulae (r-pr-m1) to (r-pr-m17) and General Formulae (r-pr-s1) to (r-pr-s20), and more preferably acid-dissociable groups represented by General Formulae (r-pr-m1) to (r-pr-m5) and General Formulae (r-pr-s1) to (r-pr-s20).

Xa⁰ may be a group composed of only an acid-dissociable group or may be a group composed of an acid-dissociable group and a group other than the acid-dissociable group. Examples of the group other than the acid-dissociable group include a divalent hydrocarbon group which may have a substituent and a divalent linking group containing a hetero atom.

Divalent Hydrocarbon Group which May have Substituent:

In a case where the group other than the acid-dissociable group is a divalent hydrocarbon group which may have a substituent, the hydrocarbon group may be an aliphatic hydrocarbon group or an aromatic hydrocarbon group.

### Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group, and an aliphatic hydrocarbon group containing a ring in the structure thereof.

### Linear or Branched Aliphatic Hydrocarbon Group

The linear aliphatic hydrocarbon group described above preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—]. a tetramethylene group [—(CH₂)₄—], and a pentamethylene group [—(CH₂)₅—].

The branched aliphatic hydrocarbon group described above preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group. Specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH)CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂—, and —CH₂CH(CH₃)CH₂CH₂—. The alkyl group in the alkylalkylene group is preferably a linear alkyl group having 1 to 5 carbon atoms.

The linear or branched aliphatic hydrocarbon group may have a substituent or may not have a substituent. Examples of the substituent include a fluorine atom, a fluorinated alkyl group having 1 to 5 carbon atoms, which has been substituted with a fluorine atom, and a carbonyl group.

### Aliphatic Hydrocarbon Group Containing Ring in Structure Thereof

Examples of the aliphatic hydrocarbon group containing a ring in the structure thereof include a cyclic aliphatic hydrocarbon group which may have a substituent containing a hetero atom in the ring structure thereof (a group obtained by removing two hydrogen atoms from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in a linear or branched aliphatic hydrocarbon group. Examples of the linear or branched aliphatic hydrocarbon group include the same as those described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The monocycloalkane is preferably a monocycloalkane having 3 to 6 carbon atoms, and specific examples thereof include cyclopentane and cyclohexane. The polycyclic alicyclic hydrocarbon group is preferably a group obtained by removing two hydrogen atoms from a polycycloalkane, and the polycycloalkane preferably has 7 to 12 carbon atoms. Specific examples of the polycycloalkane include adamantane, norbornane, isobornane, tricyclodecane, and tetracyclododecane.

The cyclic aliphatic hydrocarbon group may have or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and a methyl group, an ethyl group, a propyl group, an n-butyl group, or a tert-butyl group is more preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as the substituent is preferably a fluorine atom.

Examples of the halogenated alkyl group as the substituent include groups in which part or all of hydrogen atoms in the above-described alkyl groups have been substituted with the above-described halogen atoms.

In the cyclic aliphatic hydrocarbon group, a part of carbon atoms constituting the ring structure thereof may be substituted with a substituent containing a hetero atom. The substituent containing a hetero atom is preferably —O—, —C(═O)—O—, —S—, —S(═O)₂—, or —S(═O)₂—O—.

### Aromatic Hydrocarbon Group

The aromatic hydrocarbon group is a hydrocarbon group having at least one aromatic ring.

The aromatic ring is not particularly limited as long as it is a cyclic conjugated system having (4n+2) π electrons, and it may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms in a substituent is not included in the number of carbon atoms in the aromatic ring.

Specific examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group obtained by removing two hydrogen atoms from the above-described aromatic hydrocarbon ring or aromatic heterocyclic ring (an arylene group or a heteroarylene group); a group obtained by removing two hydrogen atoms from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group in which one hydrogen atom further has been removed from an aryl group in arylalkyl groups such as a benzyl group, a phenethyl group, a 1-naphthylmethyl group, a 2-naphthylmethyl group, a 1-naphthylethyl group, or a 2-naphthylethyl group). The alkylene group bonded to the aryl group or the heteroaryl group preferably has 1 to 4 carbon atoms, more preferably has 1 or 2 carbon atoms, and particularly preferably has 1 carbon atom.

In the aromatic hydrocarbon group, the hydrogen atom contained in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and a methyl group, an ethyl group, a propyl group, an n-butyl group, or a tert-butyl group is more preferable.

Examples of the alkoxy group, the halogen atom, and the halogenated alkyl group, as the substituent, include those exemplified as the substituent that is substituted for a hydrogen atom contained in the cyclic aliphatic hydrocarbon group.

### Divalent Linking Group Containing Hetero Atom

In a case where the group other than the acid-dissociable group is a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group, an acyl group, or the like), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²², —Y²¹—O—C(═O)—Y²², or —Y²¹—S(═O)₂—O—Y²²— [in the formulae, Y²¹ and Y²² each independently represents a divalent hydrocarbon group which may have a substituent, O represents an oxygen atom, and m″ represents an integer in a range of 0 to 3].

In a case where the divalent linking group containing a hetero atom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) preferably has 1 to 10 carbon atoms, more preferably has 1 to 8 carbon atoms, and particularly preferably has 1 to 5 carbon atoms.

In Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²², Y²¹ and Y²² each independently represents a divalent hydrocarbon group which may have a substituent. Examples of the divalent hydrocarbon group include the same as those for “the divalent hydrocarbon group which may have a substituent” described in the explanation of the above-described divalent linking group as Ya²¹.

Y²¹ is preferably a linear aliphatic hydrocarbon group, more preferably a linear alkylene group, still more preferably a linear alkylene group having 1 to 5 carbon atoms, and particularly preferably a methylene group or an ethylene group.

Y²² is preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. In other words, the group represented by Formula —[Y²¹—C(═O)—O]_{m″}—Y²²— is particularly preferably a group represented by Formula —Y²¹—C(═O)—O—Y²²—. Among the above, a group represented by Formula —(CH₂)_{a′}—C(═O)—O—(CH₂)_{b′}— is preferable. In the formula, a′ represents an integer in a range of 1 to 10, preferably an integer in a range of 1 to 8, more preferably an integer in a range of 1 to 5, still more preferably 1 or 2, and most preferably 1. b′ represents an integer in a range of 1 to 10, preferably an integer in a range of 1 to 8, more preferably an integer in a range of 1 to 5, still more preferably 1 or 2, and most preferably 1.

Among the above, the group other than the acid-dissociable group is preferably a single bond, an ester bond [—C(═O)—O—], an ether bond (—O—), a linear or branched alkylene group, or a combination thereof.

Xa⁰ is preferably a group represented by General Formula (Xa0-1).

*—Ya⁰-Xa⁰⁰ (xa0-1)

[In the formula, Ya⁰ represents a divalent linking group containing a hetero atom, and Xa⁰⁰ represents an acid-dissociable group. * represents a bonding site.]

In General Formula (Xa0-1), Ya⁰ represents a divalent linking group containing a hetero atom. Examples of the divalent linking group including a hetero atom include those exemplified above. Among the above, Ya⁰ is preferably an ester bond [—C(═O)—O—], an ether bond (—O—), a linear or branched alkylene group, or a combination thereof.

In General Formula (Xa0-1), Xa⁰⁰ represents an acid-dissociable group. The acid-dissociable group is preferably an acid-dissociable group represented by General Formula (a1-r-2), more preferably an acid-dissociable group represented by General Formula (a1-r2-1) or (a1-r2-4), and still more preferably an acid-dissociable group represented by General Formula (a1-r2-1). Among the above, it is preferably acid-dissociable groups represented by General Formulae (r-pr-m1) to (r-pr-m17) and General Formulae (r-pr-s1) to (r-pr-s20), and more preferably acid-dissociable groups represented by General Formulae (r-pr-m1) to (r-pr-m5) and General Formulae (r-pr-s1) to (r-pr-s20).

Xa⁰ is more preferably a group represented by General Formula (Xa0-1-1).

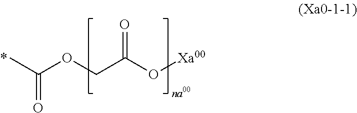

[In the formula, na⁰⁰ represents 0 or 1, and Xa⁰⁰ represents an acid-dissociable group. * represents a bonding site.]

The constitutional unit (a02) preferably contains a lactone-containing cyclic group containing an acid-dissociable group, more preferably contains a lactone-containing cyclic group represented by any one of General Formula (a02-r1-1) to (a02-r1-7), and still more preferably contains a lactone-containing cyclic group represented by General Formula (a02-r1-1) or (a02-r1-2).

Specific examples of the lactone-containing cyclic group represented by General Formula (a02-r1-1) or (a02-r1-2) are shown below. * represents a bonding site.

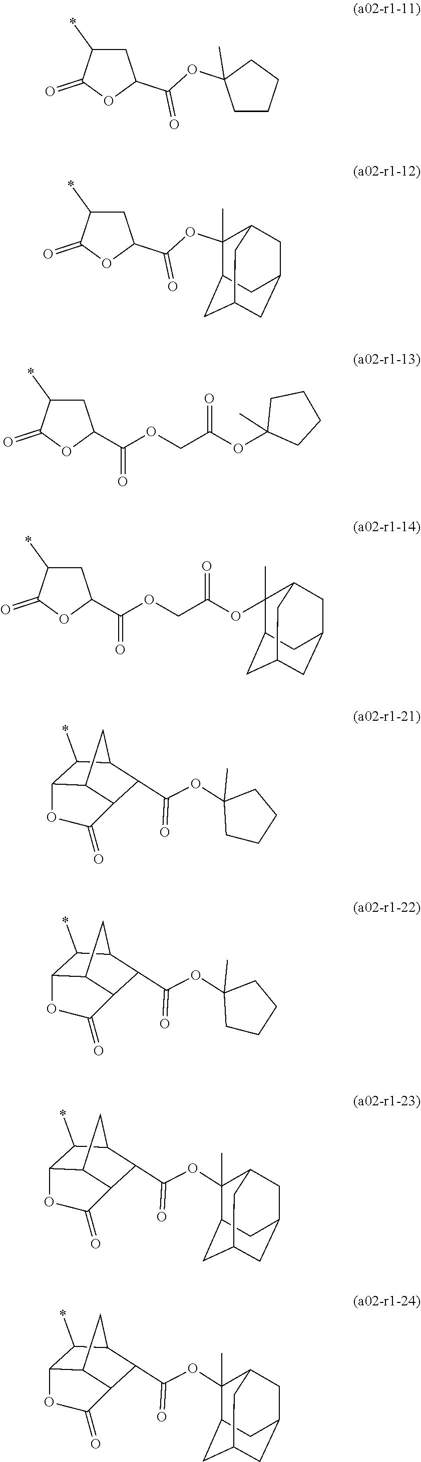

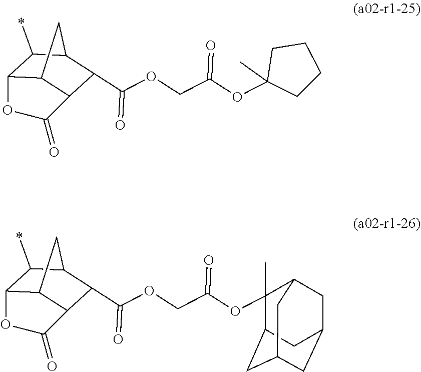

The constitutional unit (a02) is preferably a constitutional unit derived from an acrylic acid ester in which the hydrogen atom bonded to the carbon atom at the α-position may be substituted with a substituent.

The constitutional unit (a02) is preferably a constitutional unit represented by General Formula (a02-1).

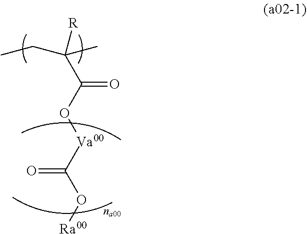

[In the formula, R represents a hydrogen atom, an alkyl group having 1 to 5 carbon atoms, or a halogenated alkyl group having 1 to 5 carbon atoms. Va⁰⁰ represents a divalent hydrocarbon group which may have an ether bond. nₐ₀₀ represents an integer in a range of 0 to 2. R⁰⁰ represents a lactone-containing cyclic group containing an acid-dissociable group, an —SO₂—-containing cyclic group containing an acid-dissociable group, or a carbonate-containing cyclic group containing an acid-dissociable group.]

In General Formula (a02-1), R is the same as R in General Formula (a01-1-1).

In General Formula (a02-1), the divalent hydrocarbon group as Va⁰⁰ may be an aliphatic hydrocarbon group or an aromatic hydrocarbon group. In General Formula (a02-1), examples of Va⁰⁰ include the same one as Va⁰ in General Formula (a01-1-1).

Va⁰⁰ is preferably a linear or branched aliphatic hydrocarbon group, more preferably a linear or branched alkylene group, still more preferably a linear or branched alkylene group having 1 to 4 carbon atoms, and even still more preferably a methylene group or an ethylene group.

In General Formula (a02-1), nₐ₀₀ represents an integer in a range of 0 to 2. nₐ₀₀ is preferably 0 or 1 and more preferably 0.

In General Formula (a02-1), Ra⁰⁰ represents a lactone-containing cyclic group containing an acid-dissociable group, an —SO₂—-containing cyclic group containing an acid-dissociable group, or a carbonate-containing cyclic group containing an acid-dissociable group. Ra⁰⁰ is preferably a lactone-containing cyclic group containing an acid-dissociable group, more preferably a lactone-containing cyclic group represented by any one of General Formula (a02-r-1) to (a02-r1-7), and still more preferably a lactone-containing cyclic group represented by General Formula (a02-r-1) or (a02-r-2).

Specific examples of the constitutional unit (a02) are shown below. R^{α} represents a hydrogen atom, a methyl group, or a trifluoromethyl group.

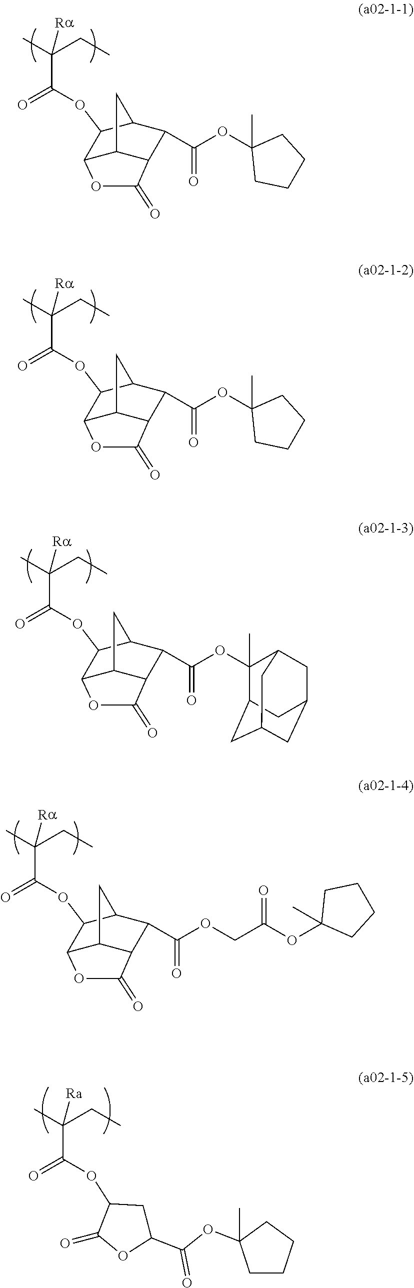

The constitutional unit (a02) which is contained in the component (A1) may be one kind or may be two or more kinds.

The proportion of the constitutional unit (a02) in the component (A1) is preferably in a range of 5% to 80% by mole, more preferably in a range of 10% to 75% by mole, still more preferably in a range of 30% to 70% by mole, and particularly preferably in a range of 40% to 60% by mole, with respect to the total (100% by mole) of all constitutional units constituting the component (A1).

In a case where the proportion of the constitutional unit (a02) is equal to or larger than the lower limit value of the above-described preferred range, lithography characteristics such as sensitivity, resolution, and roughness amelioration are improved. On the other hand, in a case where it is equal to or smaller than the upper limit value of the above-described preferred range, balance with other constitutional units can be obtained, and various lithography characteristics are improved.

### <<Other Constitutional Units>>

The component (A1) may have other constitutional units as necessary in addition to the constitutional unit (a02) described above.

Examples of the other constitutional units include a constitutional unit (a1) containing an acid-decomposable group having a polarity which is increased under action of acid (however, a constitutional unit corresponding to the constitutional unit (a02) is excluded) or the constitutional unit (a2) described above (however, a constitutional unit corresponding to the constitutional unit (a02) is excluded); the constitutional unit (a3) described above; the constitutional unit (a4) described above; the constitutional unit (a10) represented by General Formula (a10-1) described above, and the constitutional unit (st) described above.

<Constitutional Unit (a1)>

The constitutional unit (a1) is a constitutional unit (however, a constitutional unit corresponding to the constitutional unit (a02) is excluded) containing an acid-decomposable group having a polarity which is increased under action of acid.

Examples of the acid-dissociable group include those which have been proposed so far as acid-dissociable groups for the base resin for a chemical amplification-type resist composition. Examples thereof include “the acetal-type acid-dissociable group”, “the tertiary alkyl ester-type acid-dissociable group”, and “the tertiary alkyloxycarbonyl acid-dissociable group”, which are described in the first aspect.

Preferred specific examples of the constitutional unit (a1) include the constitutional unit represented by General Formula (a1-1) or (a1-2).

Suitable examples of the acid-dissociable group represented by General Formula (a1-r-2) as Ra¹ in General Formula (a1-1), include a group represented by General Formula (a1-r2-1), a group represented by General Formula (a1-r2-2), a group represented by General Formula (a1-r2-3), and a group represented by General Formula (a1-r2-4).

Specific examples of the constitutional unit represented by General Formula (a1-1) include the same as those described above in the first aspect.

The constitutional unit (a1) contained in the component (A1) may be one kind or may be two or more kinds.

The constitutional unit (a1) is more preferably a constitutional unit represented by General Formula (a1-1) since lithography characteristics (sensitivity, shape, and the like) depending on an electron beam or EUV can be more easily increased.

The proportion of the constitutional unit (a1) in the component (A1) is preferably in a range of 0% to 50% by mole, more preferably in a range of 0% to 40% by mole, still more preferably in a range of 0% to 30% by mole, and particularly preferably in a range of 0% to 20% by mole, with respect to the total (100% by mole) of all constitutional units constituting the component (A1).

It is preferable that the component (A1) not have the constitutional unit (a1).

The proportion of the total of the constitutional unit (a02) and the constitutional unit (a1) in the component (A1) is preferably in a range of 10% to 90% by mole, more preferably in a range of 10% to 80% by mole, still more preferably in a range of 20% to 75% by mole, even still more preferably in a range of 30% to 70% by mole, and particularly preferably 40% to 60% by mole, with respect to the total amount (100% by mole) of all constitutional units constituting the component (A1).

### In Regard to Constitutional Unit (a2):

The component (A1) may further have a constitutional unit (a2) (however, constitutional units corresponding to the constitutional units (a02) and (a1) are excluded) containing a lactone-containing cyclic group, an —SO₂—-containing cyclic group, or a carbonate-containing cyclic group.

The description for the constitutional unit (a2) is the same as the description of the constitutional unit (a2) in the first aspect.

The constitutional unit (a2) contained in the component (A1) may be one kind or may be two or more kinds.

In a case where the component (A1) has the constitutional unit (a2), the proportion of the constitutional unit (a2) is preferably in a range of 1% to 50% by mole, more preferably in a range of 3% to 40% by mole, still more preferably in a range of 5% to 30% by mole, and particularly preferably in a range of 5% to 25% by mole with respect to the total (100% by mole) of all constitutional units constituting the component (A1).

In a case where the proportion of the constitutional unit (a2) is equal to or larger than the lower limit value of the preferred range, the effect obtained by allowing the constitutional unit (a2) to be contained can be sufficiently achieved by the effect described above. In a case where the proportion of the constitutional unit (a2) is equal to or smaller than the upper limit value of the preferred range, balance with other constitutional units can be obtained, and various lithography characteristics are improved.

### In Regard to Constitutional Unit (a3):

The component (A1) may further have a constitutional unit (a3) (however, constitutional units corresponding to the constitutional unit (a02), the constitutional unit (a1), and the constitutional unit (a2) are excluded) containing a polar group-containing aliphatic hydrocarbon group.

The description for the constitutional unit (a3) is the same as the description of the constitutional unit (a3) in the first aspect.

The constitutional unit (a3) contained in the component (A1) may be one kind or may be two or more kinds.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole, with respect to the total (100% by mole) of all constitutional units constituting the component (A1).

In a case where the proportion of the constitutional unit (a3) is equal to or larger than the lower limit value of the preferred range. the effect obtained by allowing the constitutional unit (a3) to be contained can be sufficiently achieved by the effect described above. In a case where the proportion of the constitutional unit (a3) is equal to or smaller than the upper limit value of the preferred range, balance with other constitutional units can be obtained, and various lithography characteristics are improved.

### In Regard to Constitutional Unit (a4):

The component (A1) may further have a constitutional unit (a4) containing an acid non-dissociable aliphatic cyclic group.

The description for the constitutional unit (a4) is the same as the description of the constitutional unit (a4) in the first aspect.

The constitutional unit (a4) contained in the component (A1) may be one kind or may be two or more kinds.

In a case where the component (A1) has the constitutional unit (a4), the proportion of the constitutional unit (a4) is preferably in a range of 1% to 40% by mole and more preferably in a range of 5% to 20% by mole, with respect to the total (100% by mole) of all constitutional units constituting the component (A1).

In a case where the proportion of the constitutional unit (a4) is equal to or larger than the lower limit value of the preferred range, the effect that is obtained by allowing the constitutional unit (a4) to be contained can be sufficiently achieved. In a case where the proportion of the constitutional unit (a4) is equal to or smaller than the upper limit value of the preferred range, the balance with other constitutional units is obtained easily.

In Regard to Constitutional Unit (a10):

The constitutional unit (a10) is a constitutional unit represented by General Formula (a10-1).

The description for the constitutional unit (a10) is the same as the description of the constitutional unit (a10) in the first aspect.

The constitutional unit (a10) contained in the component (A1) may be one kind or may be two or more kinds.

In a case where the component (A1) has the constitutional unit (a10), the proportion of the constitutional unit (a10) in the component (A1) is preferably in a range of 10% to 80% by mole, more preferably in a range of 20% to 70% by mole, still more preferably in a range of 30% to 60% by mole, and particularly preferably in a range of 40% to 60% by mole, with respect to the total (100% by mole) of all constitutional units constituting the component (A1).

In a case where the proportion of the constitutional unit (a10) is equal to or larger than the lower limit value of the above-described preferred range, the sensitivity is more easily increased. In a case where the proportion of the constitutional unit (a10) is equal to or smaller than the upper limit value of the above-described preferred range, the balance between the constitutional unit (a10) and other constitutional units is easily achieved.

### In Regard to Constitutional Unit (st):

The constitutional unit (st) is a constitutional unit derived from styrene or a styrene derivative.

The description for the constitutional unit (st) is the same as the description of the constitutional unit (st) in the first aspect.

The constitutional unit (st) contained in the component (A1) may be one kind or may be two or more kinds.

In a case where the component (A1) has the constitutional unit (st), the proportion of the constitutional unit (st) is preferably in a range of 1% to 30% by mole and more preferably in a range of 3% to 20% by mole with respect to the total (100% by mole) of all constitutional units constituting the component (A1).

The component (A1) may be used alone or in combination of two or more kinds thereof.

Examples of the component (A1) include a polymeric compound having a repeating structure of the constitutional unit (a02), where the component (A1) is preferably a polymeric compound having a repeating structure of the constitutional unit (a02) and the constitutional unit (a10).

Among the above, the component (A1) is more preferably a polymeric compound consisting of a repeating structure of the constitutional unit (a02); a polymeric compound consisting of a repeating structure of the constitutional unit (a02) and the constitutional unit (a10); a polymeric compound consisting of a repeating structure of a constitutional unit (a02), a constitutional unit (a10), and a constitutional unit (a3); or a polymeric compound consisting of a repeating structure of a constitutional unit (a02), a constitutional unit (a10), and a constitutional unit (a2).

In the polymeric compound having a repeating structure of the constitutional unit (a02) and the constitutional unit (a10), the proportion of the constitutional unit (a02) is preferably in a range of 10% to 90% by mole, more preferably in a range of 20% to 80% by mole, still more preferably in a range of 30% to 70% by mole, and particularly preferably in a range of 40% to 60% by mole with respect to the total (100% by mole) of all constitutional units constituting the polymeric compound.

The proportion of the constitutional unit (a10) in the polymeric compound described above is preferably in a range of 10% to 90% by mole, more preferably in a range of 20% to 80% by mole, still more preferably in a range of 30% to 70% by mole, and particularly preferably in a range of 40% to 60% by mole, with respect to the total (100% by mole) of all constitutional units constituting the polymeric compound.

In the polymeric compound having a repeating structure of the constitutional unit (a02), the constitutional unit (a10), and the constitutional unit (a3), the proportion of the constitutional unit (a02) is preferably in a range of 10% to 90% by mole, more preferably in a range of 20% to 80% by mole, still more preferably in a range of 30% to 70% by mole, and particularly preferably in a range of 40% to 60% by mole with respect to the total (100% by mole) of all constitutional units constituting the polymeric compound.

In addition, the proportion of the constitutional unit (10) in the polymeric compound described above is preferably in a range of 10% to 90% by mole, more preferably in a range of 20% to 80% by mole, still more preferably in a range of 30% to 70% by mole, and particularly preferably in a range of 40% to 60% by mole, with respect to the total (100% by mole) of all constitutional units constituting the polymeric compound.

In addition, the proportion of the constitutional unit (a3) in the polymeric compound described above is preferably in a range of 1% to 30% by mole, more preferably in a range of 5% to 25% by mole, still more preferably in a range of 5% to 20% by mole, and particularly preferably in a range of 5% to 15% by mole, with respect to the total (100% by mole) of all constitutional units constituting the polymeric compound.

In the polymeric compound having a repeating structure of the constitutional unit (a02), the constitutional unit (a10), and the constitutional unit (a2), the proportion of the constitutional unit (a02) is preferably in a range of 10% to 90% by mole, more preferably in a range of 20% to 80% by mole, still more preferably in a range of 30% to 70% by mole, and particularly preferably in a range of 40% to 60% by mole with respect to the total (100% by mole) of all constitutional units constituting the polymeric compound.

In addition, the proportion of the constitutional unit (10) in the polymeric compound described above is preferably in a range of 10% to 90% by mole, more preferably in a range of 20% to 80% by mole, still more preferably in a range of 30% to 70% by mole, and particularly preferably in a range of 40% to 60% by mole, with respect to the total (100% by mole) of all constitutional units constituting the polymeric compound.

In addition, the proportion of the constitutional unit (a3) in the polymeric compound described above is preferably in a range of 1% to 30% by mole, more preferably in a range of 3% to 25% by mole, still more preferably in a range of 3% to 20% by mole, and particularly preferably in a range of 5% to 20% by mole, with respect to the total (100% by mole) of all constitutional units constituting the polymeric compound.

The molar ratio of the constitutional unit (a02) to the constitutional unit (a10) in the polymeric compound (the constitutional unit (a02):the constitutional unit (a10)) is preferably in a range of 2:8 to 8:2, more preferably in a range of 3:7 to 7:3, and still more preferably in a range of 4:6 to 6:4.

The component (A1) can be produced by the same method as the production method for the component (A1) described in the first aspect.

The weight-average molecular weight (Mw) (in terms of the polystyrene equivalent value determined by gel permeation chromatography (GPC)) of the component (A1), which is not particularly limited, is preferably in a range of 1,000 to 50,000, more preferably in a range of 2,000 to 30,000, and still more preferably in a range of 3,000 to 20,000. The Mw of the component (A1) is more preferably in a range of 4,000 to 15,000 and particularly preferably in a range of 5,000 to 10,000.

In a case where the Mw of the component (A1) is equal to or smaller than the upper limit value of the above-described preferred range, solubility in a resist solvent sufficient for use as a resist is likely to be obtained. In a case where the Mw of the component (A1) is equal to or larger than the lower limit value of the above-described preferred range, the dry etching resistance and the cross-sectional shape of the resist pattern are good.

The polydispersity (Mw/Mn) of the component (A1) is not particularly limited; however, it is preferably in a range of 1.0 to 4.0, more preferably in a range of 1.0 to 3.0, and particularly preferably in a range of 1.0 to 2.0. In addition, Mn indicates the number-average molecular weight.

### in Regard to Component (A2)

In the resist composition according to the present embodiment, a base material component (hereinafter, referred to as a “component (A2)”) that exhibits changed solubility in a developing solution under action of acid, which does not correspond to the component (A1), may be used in combination as the component (A).

The component (A2) is not particularly limited and may be freely selected and used from a large number of base material components for the chemical amplification-type resist composition known in the related art.

As the component (A2), a polymeric compound or a low molecular weight compound may be used alone or in a combination of two or more kinds thereof.

The proportion of the component (A1) in the component (A) is preferably 25% by mass or more, more preferably 50% by mass or more, still more preferably 75% by mass or more, and may be 100% by mass with respect to the total mass of the component (A). In a case where the proportion of the component (A1) is 25% by mass or more, a resist pattern having various excellent lithography characteristics such as high sensitivity, resolution, and roughness amelioration can be easily formed.

The content of the component (A) in the resist composition according to the present embodiment may be adjusted depending on the resist film thickness to be formed.

### <Acid Generator Component (B)>

The component (B) in the resist composition according to the present embodiment contains the compound (B0) represented by General Formula (b0) (the component (B0)).

The description for the component (B0) is the same as the description of the component (B0) in the first aspect.

In the resist composition according to the present embodiment, the component (B0) may be used alone or may be used in a combination of two or more kinds thereof.

In the resist composition according to the present embodiment, the content of the component (B0) is preferably in a range of 5 to 40 parts by mass, more preferably in a range of 10 to 40 parts by mass, still more preferably in a range of 15 to 40 parts by mass, and particularly preferably in a range of 20 to 35 parts by mass, with respect to 100 parts by mass of the component (A).

In a case where the content of the component (B0) is equal to or larger than the lower limit value of the above-described preferred range, lithography characteristics such as sensitivity, reduction of linewidth roughness (LWR), and an exposure margin are further improved in the resist pattern formation. On the other hand, in a case where the content thereof is equal to or smaller than the upper limit value of the preferred range, a homogeneous solution is easily obtained when each of the components of the resist composition is dissolved in an organic solvent, and the storage stability as a resist composition is further improved.

The proportion of the component (B0) in the total component (B) contained in the resist composition according to the present embodiment is, for example, 50% by mass or more, preferably 70% by mass or more, and more preferably 95% by mass or more. The proportion of the component (B0) in the total component (B) may be 100% by mass.

The component (B) in the resist composition according to the present embodiment may contain an acid generator component (B1) (a component (B1)) other than the above-described component (B0). The description for the component (B1) is the same as the description of the component (B1) in the first aspect.

It is preferable that the resist composition according to the present embodiment not contain the component (B1).

### <Other Components>

The resist composition according to the present embodiment may further contain other components in addition to the component (A) and the component (B) described above. Examples of the other components include the component (D), the component (E), the component (F), and the component (S), which are described above.

### <<Base Component (D)>>

It is preferable that the resist composition according to the present embodiment further contain a base component (a component (D)) that traps (that is, controls the acid diffusion) acid that is generated upon exposure, in addition to the component (A) and the component (B).

The description for the component (D) is the same as the description of the component (D) in the first aspect.

<<At Least One Compound (E) Selected from the Group Consisting of Organic Carboxylic Acid, Phosphorus Oxo Acid, and Derivatives Thereof>>

The resist composition according to the present embodiment may contain the compound (E) (the component (E)) described above.

The description for the component (E) is the same as the description of the component (E) in the first aspect.

### <<Fluorine Additive Component (F)>>

The resist composition according to the present embodiment may contain a fluorine additive component (a component (F)).

The description for the component (F) is the same as the description of the component (F) in the first aspect.

### <<Organic Solvent Component (S)>>

The resist composition according to the present embodiment may be produced by dissolving the resist materials in the organic solvent component (the component (S)).

The description for the component (S) is the same as the description of the component (S) in the first aspect.

As desired, other miscible additives can be also added to the resist composition according to the present embodiment. For example, for improving the performance of the resist film, an additive resin, a dissolution inhibitor, a plasticizer, a stabilizer, a colorant, a halation prevention agent, and a dye can be appropriately contained therein.

After dissolving the resist material in the component (S), the resist composition according to the present embodiment may be subjected to the removal of impurities and the like by using a porous polyimide membrane, a porous polyamideimide membrane, or the like. For example, the resist composition may be filtered using a filter consisting of a porous polyimide membrane, a filter consisting of a porous polyamideimide membrane, or a filter consisting of a porous polyimide membrane and a porous polyamideimide membrane. Examples of the porous polyimide membrane and the porous polyamideimide membrane include those described in Japanese Unexamined Patent Application, First Publication No. 2016-155121.

The resist composition according to the present embodiment described above contains a resin component (A1) having the constitutional unit (a02) and the compound (B0) (the component (B0)). In the resist composition in the related art, there is a trade-off relationship between sensitivity and roughness, and thus there is a tendency that in a case where one of them is improved, the other is deteriorated. However, in the resist composition according to the present embodiment, it is possible to eliminate the trade-off relationship and favorably maintain both the sensitivity and the roughness. Further, the resolution is also improved. The reason both the roughness and the resolution are maintained favorably in the resist composition according to the present embodiment while the high sensitivity is maintained is presumed to be as follows.

The component (B0) contains an iodine atom in the anion moiety, whereby the sensitivity to EUV, EB, and the like is improved.

The constitutional unit (a02) contains a lactone-containing cyclic group containing an acid-dissociable group, an —SO₂—-containing cyclic group containing an acid-dissociable group, or a carbonate-containing cyclic group containing an acid-dissociable group, whereby the hydrophilicity of the resist film after deprotection is improved. As a result, the affinity for the developing solution is improved, and the roughness is reduced. In addition, in a case where the component (B0) contains an iodine atom in the anion moiety, the amount of acid generated by exposure increases, and the deprotection rate of the acid-dissociable group contained in the constitutional unit (a02) is increased. As a result, the dissolution contrast is improved, and the resolution is improved.

As a result, it is conceived that it is possible to achieve low roughness and improvement of resolution while maintaining high sensitivity.

### [Resist Composition According to Third Aspect]

### <Component (A)>

### In Regard to Component (A1)

In the resist composition according to the third aspect, the component (A1) has the constitutional unit (a031) containing an acid-dissociable group represented by General Formula (a03-r1), and the constitutional unit (a032) containing an acid-dissociable group represented by General Formula (a03-r2).

<Constitutional Unit (a031)>

The constitutional unit (a031) is a constitutional unit containing the acid-dissociable group which is represented by General Formula (a03-r1).

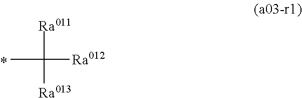

[In General Formula (a03-r1), Ra⁰¹¹ to Ra⁰¹³ each independently represents a saturated aliphatic hydrocarbon group which may have a substituent, where Ra⁰¹² and Ra⁰¹³ may be bonded to each other to form a ring. * represents a bonding site.]

In General Formula (a03-r1), Ra⁰¹¹ to Ra⁰¹³ each independently represents a saturated aliphatic hydrocarbon group which may have a substituent. Ra⁰¹² and Ra⁰¹³ may be bonded to each other to form a ring.

Examples of the saturated aliphatic hydrocarbon group as Ra⁰¹¹ to Ra⁰¹³ include a linear, branched, or cyclic alkyl group.

Examples of the linear alkyl group include a linear alkyl group having 1 to 15 carbon atoms. The linear alkyl group preferably has 1 to 12 carbon atoms, more preferably has 1 to 10 carbon atoms, still more preferably has 1 to 6 carbon atoms, and particularly preferably has 1 to 3 carbon atoms. Examples of the linear alkyl group include a methyl group, an ethyl group, an n-propyl group, an n-butyl group, and an n-pentyl group.

Examples of the branched alkyl group include a branched alkyl group having 3 to 15 carbon atoms. The branched alkyl group preferably has 3 to 12 carbon atoms, more preferably has 3 to 10 carbon atoms, still more preferably has 3 to 6 carbon atoms, and particularly preferably has 3 to 5 carbon atoms. Examples of the branched alkyl group include an isopropyl group, an isobutyl group, a tert-butyl group, an isopentyl group, a neopentyl group a 1,1-diethylpropyl group, and a 2,2-dimethylbutyl group.

The linear or branched alkyl group as Ra⁰¹¹ to Ra⁰¹³ may have a substituent. Examples of the substituent include a halogen atom, a hydroxy group, a carboxy group, and an alkoxy group. In the linear or branched alkyl group as Ra⁰¹¹ to Ra⁰¹³, part of methylene groups constituting the alkyl chain may be substituted with a hetero atom-containing group. Examples of the hetero atom include an oxygen atom, a nitrogen atom, and a sulfur atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

The cyclic alkyl group (the cycloalkyl group) may be a monocyclic group or may be a polycyclic group. Examples of the cyclic alkyl group include a cyclic alkyl group having 3 to 15 carbon atoms. The cyclic alkyl group preferably has 3 to 12 carbon atoms, more preferably has 3 to 10 carbon atoms, still more preferably has 3 to 6 carbon atoms, and particularly preferably has 5 or 6 carbon atoms.

Specific examples of the monocyclic cycloalkyl group include a cyclopropyl group, a cyclobutyl group, a cyclopentyl group, a cyclohexyl group, a cycloheptyl group, a cyclooctyl group, a cyclodecyl group, and a cyclododecyl group.

Examples of the polycyclic cycloalkyl group include a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, an adamantyl group, a norbornyl group, and an isobornyl group.

The cyclic alkyl group as Ra⁰¹¹ to Ra⁰¹³ may have a substituent. Examples of the substituent include a linear or branched alkyl group, a halogen atom, a hydroxy group, a carboxy group, and a linear or branched alkoxy group. The linear alkyl group or the linear alkoxy group, as the substituent, preferably has 1 to 5 carbon atoms, more preferably has 1 to 3 carbon atoms, and still more preferably has 1 or 2 carbon atoms. The branched alkyl group or the branched alkoxy group, as the substituent, preferably has 3 to 6 carbon atoms, more preferably has 3 to 5 carbon atoms, and still more preferably has 3 or 4 carbon atoms.

Ra⁰¹² and Ra⁰¹³ may be bonded to each other to form a ring. Examples of the ring that is formed by bonding Ra⁰¹² and Ra⁰¹³ to each other include an aliphatic saturated hydrocarbon group having 3 to 15 carbon atoms. Specific examples thereof include the same as those described as the cyclic alkyl group. The ring that is formed by bonding Ra⁰¹² and Ra⁰¹³ to each other is preferably a monocyclic aliphatic saturated hydrocarbon group.

In a case where Ra⁰¹² and Ra⁰¹³ are bonded to each other to form a ring, examples of the acid-dissociable group represented by General Formula (a03-r1) include an acid-dissociable group represented by General Formula (a03-r1-1).

In a case where Ra⁰¹² and Ra⁰¹³ are not bonded to each other and represent an independent saturated aliphatic hydrocarbon group, examples of the acid-dissociable group represented by General Formula (a03-r1) include an acid-dissociable group represented by General Formula (a03-r1-2).

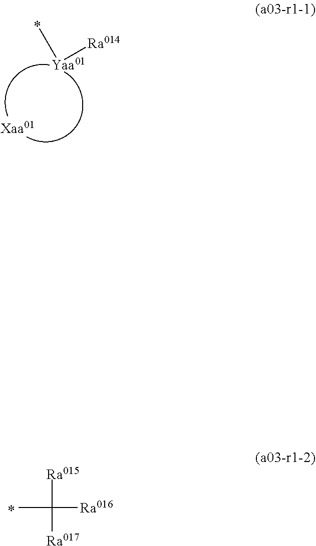

[In General Formula (a03-r1-1), Yaa⁰¹ represents a carbon atom. Xaa⁰¹ represents a group that forms an aliphatic cyclic group together with Yaa⁰¹. Ra⁰¹⁴ represents a linear, branched, or cyclic alkyl group which may have a substituent.

In General Formula (a03-r1-2), Ra⁰¹⁵ and Ra⁰¹⁶ each independently represents a linear or branched alkyl group which may have a substituent. Ra⁰¹⁷ represents a saturated aliphatic hydrocarbon group which may have a substituent.

* represents a bonding site.]

In General Formula (a03-r1-1), Yaa⁰¹ represents a carbon atom.

In Formula (a03-r1-1), Xaa⁰¹ represents a group that forms an aliphatic cyclic group together with Yaa⁰¹. Examples of the aliphatic cyclic group include an aliphatic cyclic group having 3 to 15 carbon atoms. The aliphatic cyclic group may be monocyclic or polycyclic. The aliphatic cyclic group preferably has 3 to 12 carbon atoms, more preferably has 3 to 10 carbon atoms, still more preferably has 3 to 6 carbon atoms, and particularly preferably has 5 or 6 carbon atoms. Specific examples thereof include the same as those described as the cyclic alkyl group as Ra⁰¹¹ to Ra⁰¹³. The aliphatic cyclic group that is formed by Xaa⁰¹ together with Yaa⁰¹ is preferably a monocyclic monocycloalkyl group. Among the above, it is preferably a cyclopentyl group or a cyclohexyl group, and more preferably a cyclopentyl group.

The aliphatic cyclic group may have a substituent. Examples of the substituent include the same as those described as the substituent of the cyclic alkyl group as Ra⁰¹¹ to Ra⁰¹³. Among them, the substituent is preferably a linear or branched alkyl group having 1 to 5 carbon atoms, more preferably a linear alkyl group having 1 to 3 carbon atoms, and still more preferably a methyl group or an ethyl group.

In General Formula (a03-r1-1), Ra⁰¹⁴ represents a linear, branched, or cyclic alkyl group which may have a substituent. Examples of the linear, branched, or cyclic alkyl group as Ra⁰¹⁴ include the same one as the linear, branched, or cyclic alkyl group as Ra⁰¹¹ to Ra⁰¹³. Among the above, Ra⁰¹⁴ is preferably a methyl group, an ethyl group, a propyl group, or an isopropyl group.

Examples of the substituent which may be contained in the linear or branched alkyl group as Ra⁰¹⁴ include the same as those described as the substituent of the linear or branched alkyl group as Ra⁰¹¹ to Ra⁰¹³.

In the linear or branched alkyl group as Ra⁰¹⁴, a part of methylene groups constituting the alkyl chain may be substituted with a hetero atom-containing group. Examples of the hetero atom include an oxygen atom, a nitrogen atom, and a sulfur atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

Examples of the substituent which may be contained in the cyclic alkyl group as Ra⁰¹⁴ include the same as those described as the substituent of the cyclic alkyl group as Ra⁰¹¹ to Ra⁰¹³.

The linear, branched, or cyclic alkyl group as Ra⁰¹⁴ preferably has no substituent.

In General Formula (a03-r1-2), Ra⁰¹⁵ and Ra⁰¹⁶ each independently represents a linear or branched alkyl group which may have a substituent.

Examples of the linear or branched alkyl group as Ra⁰¹⁵ and Ra⁰¹⁶ include the same one as the linear or branched alkyl group as Ra⁰¹¹ to Ra⁰¹³.

Examples of the substituent which may be contained in the linear or branched alkyl group as Ra⁰¹⁵ and Ra⁰¹⁶ include the same as those described as the substituent of the linear or branched alkyl group as Ra⁰¹¹ to Ra⁰¹³.

Ra⁰¹⁵ and Ra⁰¹⁶ are preferably a linear or branched alkyl group having 1 to 5 carbon atoms, more preferably a linear or branched alkyl group having 1 to 3 carbon atoms, and still more preferably an ethyl group or a methyl group.

Ra⁰¹⁷ represents a saturated aliphatic hydrocarbon group which may have a substituent. Examples of the saturated aliphatic hydrocarbon group as Ra⁰¹¹ to Ra⁰¹³ include a linear, branched, or cyclic alkyl group.

Examples of the linear, branched, or cyclic alkyl group as Ra⁰¹⁷ include the same one as the linear, branched, or cyclic alkyl group as Ra⁰¹¹ to Ra⁰¹³. Among the above, Ra⁰¹⁷ is preferably a linear or branched alkyl group, more preferably a methyl group, an ethyl group, a propyl group, or an isopropyl group, still more preferably a methyl group, an ethyl group, or a propyl group, and particularly preferably an ethyl group.

Examples of the substituent which may be contained in the linear or branched alkyl group as Ra⁰¹⁴ include the same as those described as the substituent of the linear or branched alkyl group as Ra⁰¹¹ to Ra⁰¹³.

Examples of the substituent which may be contained in the cyclic alkyl group as Ra⁰¹⁴ include the same as those described as the substituent of the cyclic alkyl group as Ra⁰¹¹ to Ra⁰¹³.

The linear, branched, or cyclic alkyl group as Ra⁰¹⁴ preferably has no substituent.

Specific examples of the acid-dissociable group represented by General Formula (a03-r1-1) include an acid-dissociable group represented by any one of General Formulae (r-pr-m1) to (r-pr-m17), General Formulae (r-pr-s1) to (r-pr-s21), General Formulae (r-pr-sp1) to (r-pr-sp6), and General Formulae (r-pr-ss1) to (r-pr-ss5).

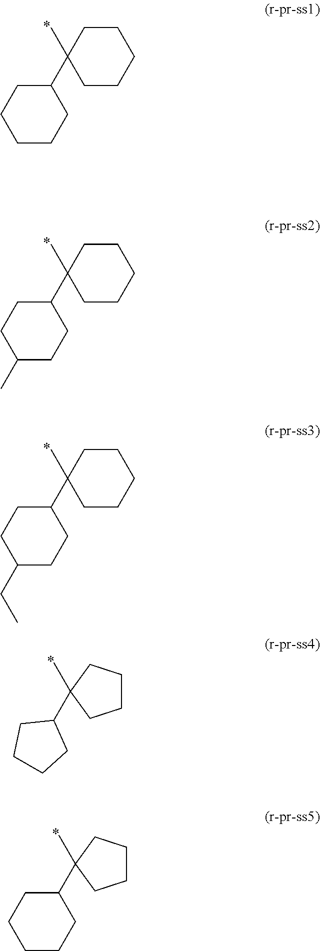

Specific examples of the acid-dissociable group represented by General Formula (a03-r1-2) include an acid-dissociable group represented by any one of General Formulae (r-pr-cm1) to (r-pr-cm4), General Formulae (r-pr-cs1) to (r-pr-cs3), and General Formulae (r-pr-c1) to (r-pr-c3).

Examples of the constitutional unit (a031) include a constitutional unit derived from an acrylic acid ester in which the hydrogen atom bonded to the carbon atom at the α-position may be substituted with a substituent; a constitutional unit derived from acrylamide; a constitutional unit in which at least a part of hydrogen atoms in a hydroxyl group of a constitutional unit derived from hydroxystyrene or a hydroxystyrene derivative are protected by the substituent including an acid-decomposable group; and a constitutional unit in which at least a part of hydrogen atoms in —C(═O)—OH of a constitutional unit derived from vinylbenzoic acid or a vinylbenzoic acid derivative are protected by the substituent including an acid-decomposable group.

The constitutional unit (a031) is preferably a constitutional unit derived from an acrylic acid ester in which the hydrogen atom bonded to the carbon atom at the α-position may be substituted with a substituent.

Examples of the constitutional unit (a031) include a constitutional unit represented by General Formula (a031-1).

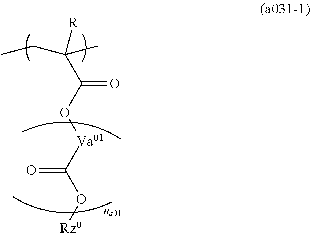

[In the formula, R represents a hydrogen atom, an alkyl group having 1 to 5 carbon atoms, or a halogenated alkyl group having 1 to 5 carbon atoms. Va⁰¹ represents a divalent hydrocarbon group which may have an ether bond. nₐ₀₁ represents an integer in a range of 0 to 2. Rz⁰¹ represents the acid-dissociable group represented by General Formula (a03-r1).]

In General Formula (a031-1), R is the same as R in General Formula (a01-1-1).

In General Formula (a031-1), the divalent hydrocarbon group as Va⁰¹ may be an aliphatic hydrocarbon group or an aromatic hydrocarbon group. In General Formula (a031-1), examples of Va⁰¹ include the same one as Va⁰ in General Formula (a01-1-1).

Va⁰¹ is preferably a linear or branched aliphatic hydrocarbon group, more preferably a linear or branched alkylene group, still more preferably a linear or branched alkylene group having 1 to 4 carbon atoms, and even still more preferably a methylene group or an ethylene group.

In General Formula (a031-1), nₐ₀₁ represents an integer in a range of 0 to 2. n₀₁ is preferably 0 or 1 and more preferably 0.

In General Formula (a031-1), Rz⁰¹ represents the acid-dissociable group represented by General Formula (a03-r1).

Specific examples of the constitutional unit represented by General Formula (a031-1) are shown below. In each of the formulae shown below, R^{α} represents a hydrogen atom, a methyl group, or a trifluoromethyl group.

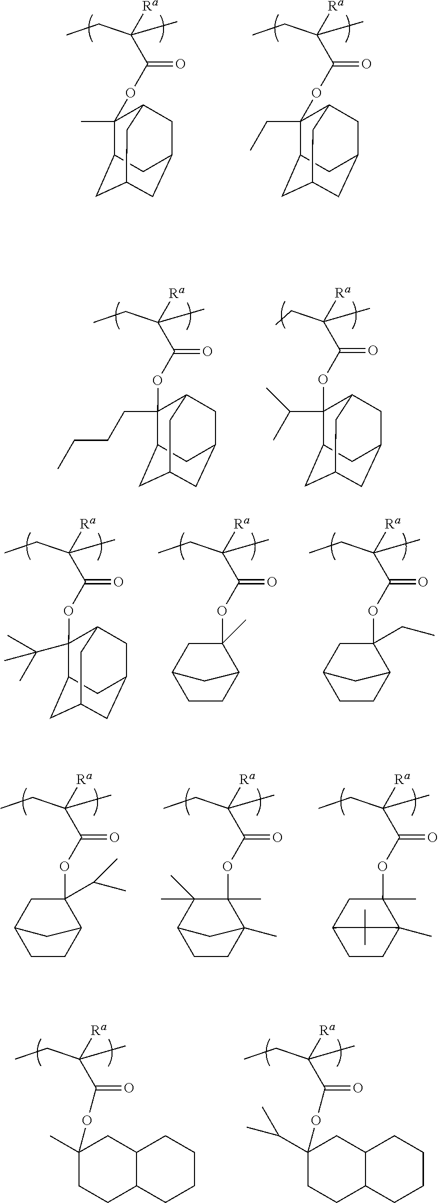

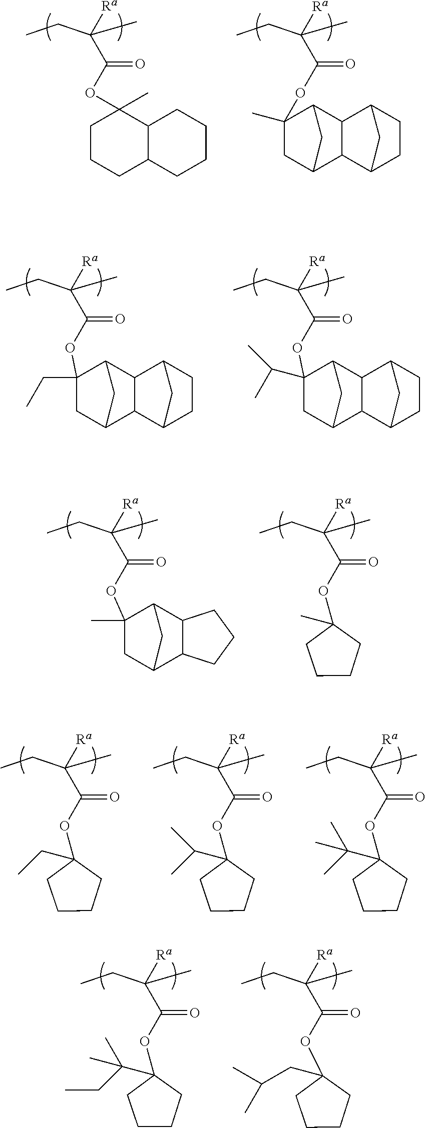

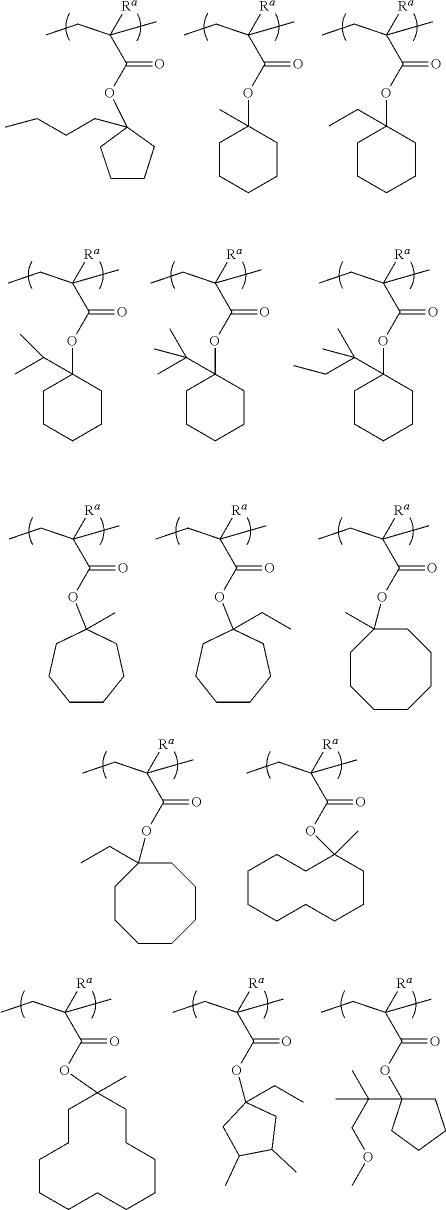

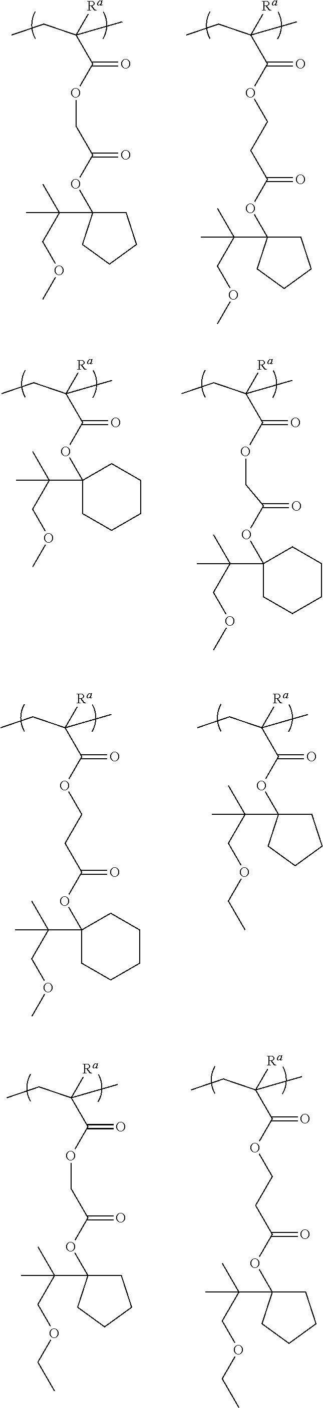

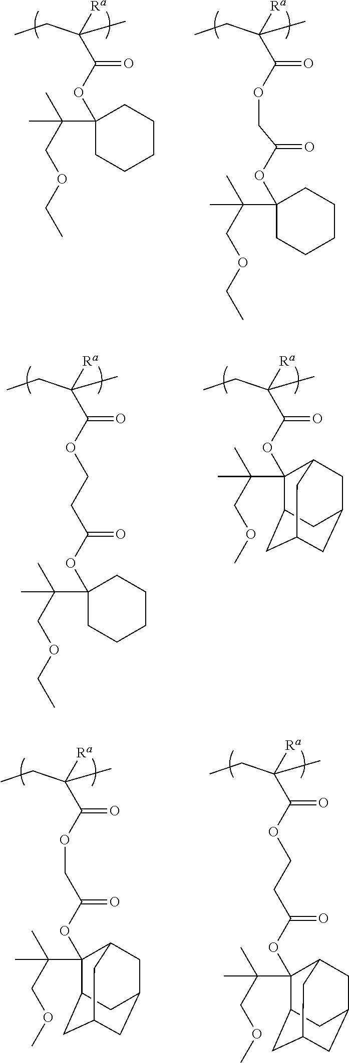

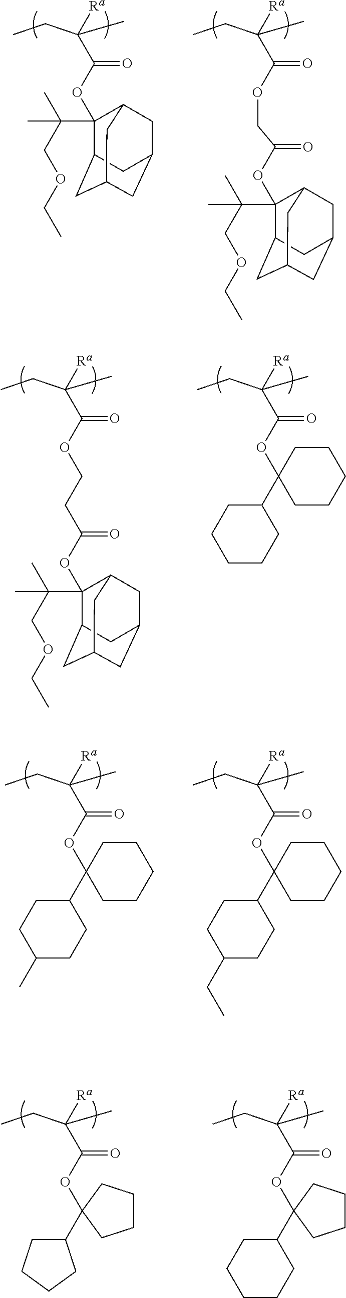

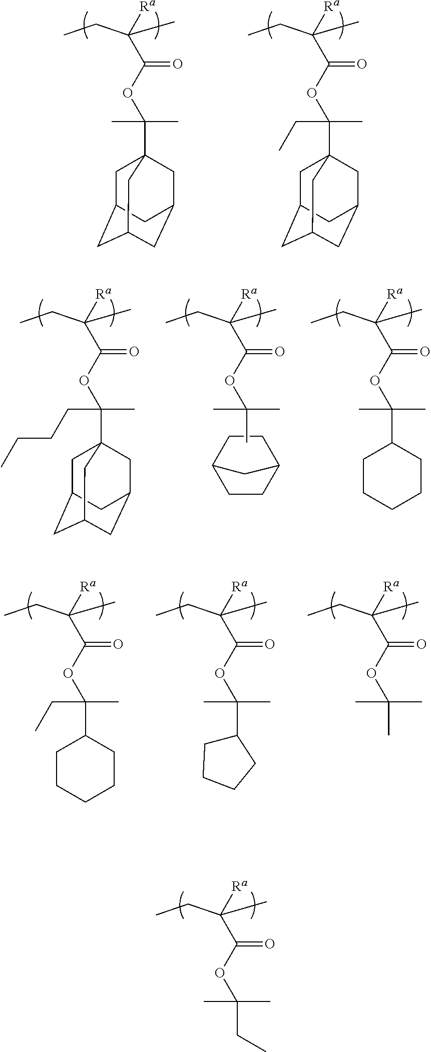

The constitutional unit (a031) may be one kind or may be two or more kinds.

The proportion of the constitutional unit (a031) in the component (A1) is preferably in a range of 5% to 80% by mole, more preferably in a range of 10% to 70% by mole, still more preferably in a range of 20% to 60% by mole, and particularly preferably in a range of 20% to 50% by mole, with respect to the total (100% by mole) of all constitutional units constituting the component (A1).

In a case where the proportion of the constitutional unit (a031) is equal to or larger than the lower limit value of the above-described preferred range, lithography characteristics such as roughness and a pattern shape are improved. In addition, in a case where the proportion of the constitutional unit (a031) is equal to or smaller than the upper limit value of the above-described preferred range, balance with other constitutional units can be obtained, and various lithography characteristics are improved.

<Constitutional Unit (a032)>

The constitutional unit (a032) is a constitutional unit (a032) containing the acid-dissociable group which is represented by General Formula (a03-r2).

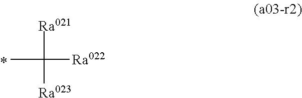

[In General Formula (a03-r2), Ra⁰²¹ represents a hydrocarbon group containing an aromatic ring, which may have a substituent, and Ra⁰²² and Ra⁰²³ each independently represents a hydrocarbon group which may have a substituent, where Ra⁰²² and Ra⁰²³ may be bonded to each other to form a ring. * represents a bonding site.]

In General Formula (a03-r2), Ra⁰²¹ represents a hydrocarbon group containing an aromatic ring, which may have a substituent.

The hydrocarbon group containing an aromatic ring, as Ra⁰²¹, preferably has 5 to 30 carbon atoms, more preferably has 6 to 15 carbon atoms, and still more preferably has 6 to 10 carbon atoms.

The aromatic ring is not particularly limited as long as it is a cyclic conjugated system having (4n+2) π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. The aromatic ring may be an aromatic hydrocarbon ring or may be an aromatic heterocyclic ring. Examples of the aromatic hydrocarbon ring include benzene, naphthalene, anthracene, and phenanthrene. Examples of the aromatic heterocyclic ring include an aromatic heterocyclic ring obtained by substituting a part of carbon atoms constituting the aromatic hydrocarbon ring with a heteroatom. Examples of the hetero atom include an oxygen atom, a sulfur atom, and a nitrogen atom. Examples of the aromatic heterocyclic ring include a pyridine ring and a thiophene ring.

The hydrocarbon group containing an aromatic ring, as Ra⁰²¹, may have a substituent. Examples of the substituent include a linear or branched alkyl group, a halogen atom, a hydroxy group, a carboxy group, and a linear or branched alkoxy group. The linear alkyl group or the linear alkoxy group, as the substituent, preferably has 1 to 5 carbon atoms, more preferably has 1 to 3 carbon atoms, and still more preferably has 1 or 2 carbon atoms. The branched alkyl group or the branched alkoxy group, as the substituent, preferably has 3 to 6 carbon atoms, more preferably has 3 to 5 carbon atoms, and still more preferably has 3 or 4 carbon atoms.

Specific examples of Ra⁰²¹ include a group obtained by removing one hydrogen atom from the above-described aromatic hydrocarbon ring or aromatic heterocyclic ring (an aryl group or a heteroaryl group); a group obtained by removing one hydrogen atom from an aromatic compound having two or more aromatic rings (biphenyl, fluorene or the like); and a group obtained by substituting one hydrogen atom of the above-described aromatic hydrocarbon ring or aromatic heterocyclic ring with an alkylene group (an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring preferably has 1 to 4 carbon atoms, more preferably has 1 or 2 carbon atoms, and particularly preferably has 1 carbon atom.

Among them, Ra⁰²¹ is preferably a group obtained by removing one hydrogen atom from an aromatic hydrocarbon ring having 6 to 15 carbon atoms, more preferably a group obtained by removing one hydrogen atom from benzene, naphthalene, anthracene, or phenanthrene, still more preferably a group obtained by removing one hydrogen atom from benzene, naphthalene, or anthracene, particularly preferably a group (a phenyl group, 1-naphthyl group, 2-naphthyl group, or the like) obtained by removing one hydrogen atom from benzene or naphthalene, and most preferably a group (a phenyl group) obtained by removing one hydrogen atom from benzene.

The hydrocarbon group containing an aromatic ring, as Ra⁰²¹, may have a substituent. Examples of the substituent include a methyl group, an ethyl group, a propyl group, a hydroxy group, a carboxy group, a halogen atom, an alkoxy group (a methoxy group, an ethoxy group, a propoxy group, a butoxy group, and the like), and an alkyloxycarbonyl group.

In General Formula (a03-r2), Ra⁰²² and Ra⁰²³ each independently represents a hydrocarbon group which may have a substituent, where Ra⁰²² and Ra⁰²³ may be bonded to each other to form a ring.

Examples of the hydrocarbon groups as Ra⁰²² and Ra⁰²³ include a linear aliphatic hydrocarbon group, a branched aliphatic hydrocarbon group, and a cyclic hydrocarbon group.

The linear aliphatic hydrocarbon group may be saturated or unsaturated; however, it is preferably saturated. Examples of the linear aliphatic hydrocarbon group include a linear alkyl group. The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferable, and a methyl group or an ethyl group is more preferable.

The branched aliphatic hydrocarbon group may be saturated or unsaturated; however, it is preferably saturated. Examples of the branched aliphatic hydrocarbon group include a branched alkyl group. The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among them, an isopropyl group is preferable.

The linear or branched aliphatic hydrocarbon group as Ra⁰²² and Ra⁰²³ may have a substituent. Examples of the substituent include a halogen atom, a hydroxy group, a carboxy group, and an alkoxy group. In the linear or branched aliphatic hydrocarbon group as Ra⁰²² and Ra⁰²³, a part of methylene groups constituting the aliphatic hydrocarbon chain may be substituted with a hetero atom-containing group. Examples of the hetero atom include an oxygen atom, a nitrogen atom, and a sulfur atom. Examples of the hetero atom-containing group include —O—, —C(═O)—O—, —O—C(═O)—, —C(═O)—, —O—C(═O)—O—, —C(═O)—NH—, —NH—, —S—, —S(═O)₂—, and —S(═O)₂—O—.

The cyclic hydrocarbon group as Ra⁰²² and Ra⁰²³ may be a cyclic aliphatic hydrocarbon group or may be an aromatic hydrocarbon group. The cyclic hydrocarbon group as Ra⁰²² and Ra⁰²³ may be a polycyclic group or may be a monocyclic group.

Examples of the cyclic aliphatic hydrocarbon group include a cyclic alkyl group (a cycloalkyl group). The cyclic alkyl group may be a monocyclic group or may be a polycyclic group. Examples of the cyclic alkyl group include a cyclic alkyl group having 3 to 15 carbon atoms. The cyclic alkyl group preferably has 3 to 12 carbon atoms, more preferably has 3 to 10 carbon atoms, still more preferably has 3 to 6 carbon atoms, and particularly preferably has 5 or 6 carbon atoms.

Specific examples of the monocyclic cycloalkyl group include a cyclopropyl group, a cyclobutyl group, a cyclopentyl group, a cyclohexyl group, a cycloheptyl group, a cyclooctyl group, a cyclodecyl group, and a cyclododecyl group.

Examples of the polycyclic cycloalkyl group include a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, an adamantyl group, a norbornyl group, and an isobornyl group.

The aromatic hydrocarbon group as Ra⁰²² and Ra⁰²³ is a hydrocarbon group having at least one aromatic ring. The aromatic ring preferably has 5 to 30 carbon atoms, more preferably has 5 to 20 carbon atoms, still more preferably has 6 to 15 carbon atoms, and particularly preferably has 6 to 12 carbon atoms.

Examples of the aromatic ring include aromatic hydrocarbon rings such as benzene, naphthalene, anthracene, and phenanthrene; and an aromatic heterocyclic ring obtained by substituting a part of carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group include the same as those described as the hydrocarbon group containing an aromatic ring, as Ra⁰²¹.

The cyclic hydrocarbon group as Ra⁰²² and Ra⁰²³ may have a substituent. Examples of the substituent include a methyl group, an ethyl group, a propyl group, a hydroxy group, a carboxy group, a halogen atom, an alkoxy group (a methoxy group, an ethoxy group, a propoxy group, a butoxy group, and the like), and an alkyloxycarbonyl group.

Ra⁰²² and Ra⁰²³ may be bonded to each other to form a ring. Examples of the ring that is formed by bonding Ra⁰²² and Ra⁰²³ to each other include the same as those described as the cyclic hydrocarbon group. The ring that is formed by bonding Ra⁰²² and Ra⁰²³ to each other is preferably a monocyclic or polycyclic aliphatic hydrocarbon group and more preferably a monocyclic aliphatic hydrocarbon group.

In a case where Ra⁰²² and Ra⁰²³ are bonded to each other to form a ring, examples of the acid-dissociable group represented by General Formula (a03-r2) include an acid-dissociable group represented by General Formula (a03-r2-1).

In a case where Ra⁰²² and Ra⁰²³ are not bonded to each other and represent an independent hydrocarbon group, examples of the acid-dissociable group represented by General Formula (a03-r2) include an acid-dissociable group represented by General Formula (a03-r2-2).

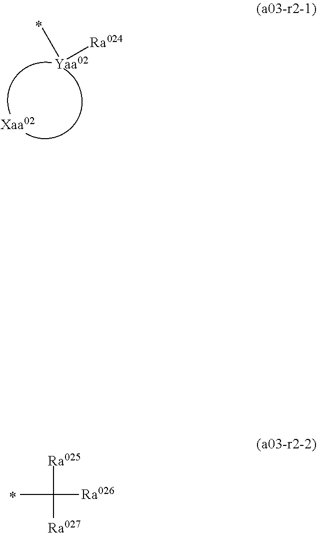

[In General Formula (a03-r2-1), Yaa⁰² represents a carbon atom. Xaa⁰² represents a group that forms an aliphatic cyclic group together with Yaa⁰². Ra⁰²⁴ represents an aromatic hydrocarbon group which may have a substituent.

In General Formula (a03-r2-2), Ra⁰²⁵ and Ra⁰²⁶ each independently represents a linear or branched alkyl group which may have a substituent, and Ra⁰²⁷ represents an aromatic hydrocarbon group which may have a substituent.

* represents a bonding site.]

In General Formula (a03-r2-1), Yaa⁰² represents a carbon atom.

In Formula (a03-r2-1), Xaa⁰² represents a group that forms an aliphatic cyclic group together with Yaa⁰². Examples of the aliphatic cyclic group that is formed by Xaa⁰² in General Formula (a03-r1-1) together with Yaa⁰² include the same as those described as the group in which the Xaa⁰² forms an aliphatic cyclic group together with Yaa⁰¹. Among them, a cyclopentyl group or a cyclohexyl group is preferable.

In General Formula (a03-r2-1), Ra⁰²⁴ represents an aromatic hydrocarbon group which may have a substituent. Examples of the aromatic hydrocarbon group include a group obtained by removing one hydrogen atom from an aromatic hydrocarbon ring having 5 to 30 carbon atoms. Among them, Ra⁰²⁴ is preferably a group obtained by removing one hydrogen atom from an aromatic hydrocarbon ring having 6 to 15 carbon atoms, more preferably a group obtained by removing one hydrogen atom from benzene, naphthalene, anthracene, or phenanthrene, still more preferably a group obtained by removing one hydrogen atom from benzene, naphthalene, or anthracene, particularly preferably a group (a phenyl group, 1-naphthyl group, or 2-naphthyl group) obtained by removing one hydrogen atom from benzene or naphthalene, and most preferably a group (a phenyl group) obtained by removing one hydrogen atom from benzene.

In a case where Ra⁰²⁴ represents a naphthyl group, the position at which Yaa⁰² is bonded may be any one of the 1-position or the 2-position of the naphthyl group. In a case where Ra⁰²⁴ represents an anthryl group, the position at which Yaa⁰² is bonded may be any one of the 1-position, the 2-position, or the 9-position of the anthryl group.

The aromatic hydrocarbon group as Ra⁰²⁴ may have a substituent. Examples of the substituent include a methyl group, an ethyl group, a propyl group, a hydroxy group, a carboxy group, a halogen atom, an alkoxy group (a methoxy group, an ethoxy group, a propoxy group, a butoxy group, and the like), and an alkyloxycarbonyl group.

In General Formula (a03-r2-2), Ra⁰²⁵ and Ra⁰²⁶ each independently represents a linear or branched alkyl group which may have a substituent. Examples of Ra⁰²⁵ and Ra⁰²⁶ include the same ones as Ra⁰¹⁵ and Ra⁰¹⁶ in General Formula (a03-r1-2).

Ra⁰²⁵ and Ra⁰²⁶ are preferably a linear or branched alkyl group having 1 to 5 carbon atoms, more preferably a linear or branched alkyl group having 1 to 3 carbon atoms, and still more preferably an ethyl group or a methyl group.

In General Formula (a03-r2-2), Ra⁰²⁷ represents an aromatic hydrocarbon group which may have a substituent. Examples of Ra⁰²⁷ include the same one as Ra⁰²⁴. Among them, Ra⁰²⁷ is preferably a group obtained by removing one hydrogen atom from an aromatic hydrocarbon ring having 6 to 15 carbon atoms, more preferably a group obtained by removing one hydrogen atom from benzene, naphthalene, anthracene, or phenanthrene, still more preferably a group obtained by removing one hydrogen atom from benzene, naphthalene, or anthracene, and particularly preferably a group (a phenyl group, a 1-naphthyl group, or a 2-naphthyl group) obtained by removing one hydrogen atom from benzene or naphthalene.

In a case where Ra⁰²⁷ represents a naphthyl group, the position at which the tertiary carbon atom in General Formula (a03-r2-2) is bonded may be any one of the 1-position or the 2-position of the naphthyl group.

In a case where Ra⁰²⁷ represents an anthryl group, the position at which the tertiary carbon atom in General Formula (a03-r2-2) is bonded may be any one of the 1-position, the 2-position, or the 9-position of the anthryl group.

Specific examples of the acid-dissociable group represented by General Formula (a03-r2-1) include an acid-dissociable group represented by any one of General Formulae (r-pr-sa1) to (r-pr-sa9) and General Formulae (r-pr-ma1) and (r-pr-ma2).

Specific examples of the acid-dissociable group represented by General Formula (a03-r2-2) include an acid-dissociable group represented by any one of General Formulae (r-pr-cm5) to (r-pr-cm8) and General Formulae (r-pr-cs4) and (r-pr-cs5).

Examples of the constitutional unit (a032) include a constitutional unit derived from an acrylic acid ester in which the hydrogen atom bonded to the carbon atom at the α-position may be substituted with a substituent; a constitutional unit derived from acrylamide; a constitutional unit in which at least a part of hydrogen atoms in a hydroxyl group of a constitutional unit derived from hydroxystyrene or a hydroxystyrene derivative are protected by the substituent including an acid-decomposable group; and a constitutional unit in which at least a part of hydrogen atoms in —C(═O)—OH of a constitutional unit derived from vinylbenzoic acid or a vinylbenzoic acid derivative are protected by the substituent including an acid-decomposable group.

The constitutional unit (a032) is preferably a constitutional unit derived from an acrylic acid ester in which the hydrogen atom bonded to the carbon atom at the α-position may be substituted with a substituent.

Examples of the constitutional unit (a032) include a constitutional unit represented by General Formula (a032-1).

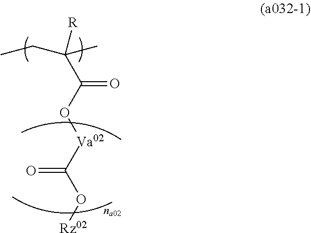

[In the formula, R represents a hydrogen atom, an alkyl group having 1 to 5 carbon atoms, or a halogenated alkyl group having 1 to 5 carbon atoms. Va⁰² represents a divalent hydrocarbon group which may have an ether bond. na⁰² represents an integer in a range of 0 to 2. Rz⁰² represents the acid-dissociable group represented by General Formula (a03-r2).]

In General Formula (a032-1), R is the same as R in General Formula (a01-1-1).

In General Formula (a032-1), the divalent hydrocarbon group as Va⁰² may be an aliphatic hydrocarbon group or an aromatic hydrocarbon group. Va⁰² is the same as Va⁰¹ in General Formula (a031-1).

In General Formula (a032-1), nₐ₀₂ represents an integer in a range of 0 to 2. nₐ₀₂ is preferably 0 or 1 and more preferably 0.

In General Formula (a032-1), Rz⁰² represents the acid-dissociable group represented by General Formula (a03-r2).

Specific examples of the constitutional unit represented by General Formula (a032-1) are shown below. In each of the formulae shown below, R^{α} represents a hydrogen atom, a methyl group, or a trifluoromethyl group.

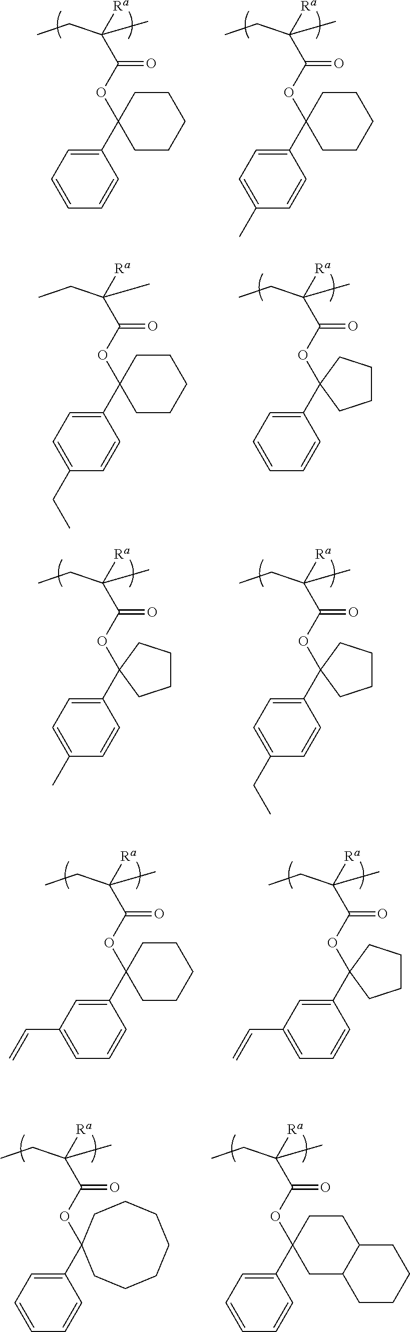

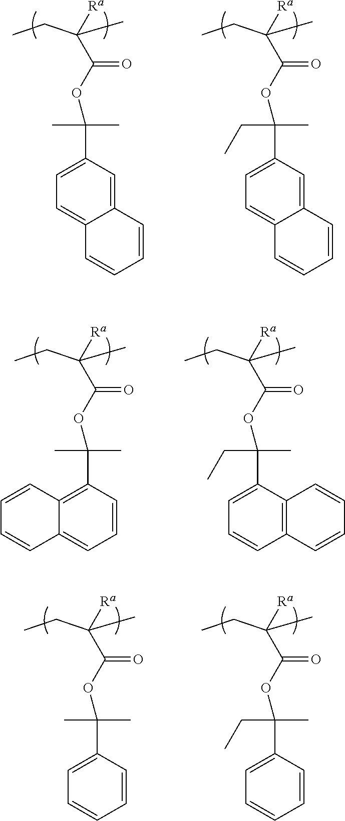

The constitutional unit (a032) may be one kind or may be two or more kinds.

The proportion of the constitutional unit (a032) in the component (A1) is preferably in a range of 5% to 70% by mole, more preferably in a range of 10% to 60% by mole, still more preferably in a range of 15% to 50% by mole, and particularly preferably in a range of 15% to 40% by mole, with respect to the total (100% by mole) of all constitutional units constituting the component (A1).

In a case where the proportion of the constitutional unit (a032) is equal to or larger than the lower limit value of the above-described preferred range, sensitivity is improved. On the other hand, in a case where the proportion of the constitutional unit (a032) is equal to or smaller than the upper limit value of the above-described preferred range, balance with other constitutional units can be obtained, and various lithography characteristics are improved.

the molar ratio of the constitutional unit (a031) to the constitutional unit (a032) (the constitutional unit (a031):the constitutional unit (a032)) is preferably in a range of 20:80 to 80:20, more preferably in a range of 30:70 to 70:30, still more preferably in a range of 40:60 to 60:40, and particularly preferably in a range of 50:50 to 60:40.

In a case where the molar ratio of the constitutional unit (a031) to the constitutional unit (a032) is within the above-described preferred range, all of the sensitivity, the roughness, and the pattern shape are likely to be improved.

The proportion of the total of the constitutional unit (a031) and the constitutional unit (a032) in the component (A1) is preferably in a range of 10% to 90% by mole, more preferably in a range of 20% to 80% by mole, still more preferably in a range of 30% to 70% by mole, and particularly preferably 40% to 60% by mole, with respect to the total amount (100% by mole) of all constitutional units constituting the component (A1).

### <<Other Constitutional Units>>

The component (A1) may have other constitutional units as necessary in addition to the constitutional unit (a031) and the constitutional unit (a032) described above.

Examples of the other constitutional units include the constitutional unit (a1) containing an acid-dissociable group, other than the acid-dissociable group represented by General Formula (a03-r1) and the acid-dissociable group represented by General Formula (a03-r2); the constitutional unit (a2) described above; the constitutional unit (a3) described above; the constitutional unit (a4) described above; the constitutional unit (a10) described above; and the constitutional unit (st) described above.

<Constitutional Unit (a1)>

The constitutional unit (a1) is a constitutional unit (however, constitutional units corresponding to the constitutional unit (a031) and the constitutional unit (a032) are excluded) containing an acid-decomposable group having a polarity which is increased under action of acid.

Examples of the acid-dissociable group include those which have been proposed so far as acid-dissociable groups for the base resin for a chemical amplification-type resist composition. Examples thereof include “the acetal-type acid-dissociable group”, “the tertiary alkyl ester-type acid-dissociable group”, and “the tertiary alkyloxycarbonyl acid-dissociable group”, which are described in the first aspect.

Examples of the tertiary alkyl ester-type acid-dissociable group include the acid-dissociable group represented by General Formula (a1-r-2) (a group represented by General Formula (a03-r1) or (a03-r2) is excluded).

Examples of the acid-dissociable group represented by General Formula (a1-r-2) include a group represented by General Formula (a1-r2-2).

Preferred specific examples of the constitutional unit (a1) include the constitutional unit represented by General Formula (a1-1) or (a1-2).

In Formula (a1-1), suitable examples of the acid-dissociable group as Ra′, which is represented by General Formula (a1-r-2), include the group represented by General Formula (a1-r2-4).

Specific examples of the constitutional unit represented by General Formula (a1-1) are shown below. In each of the formulae shown below, Ra represents a hydrogen atom, a methyl group, or a trifluoromethyl group.

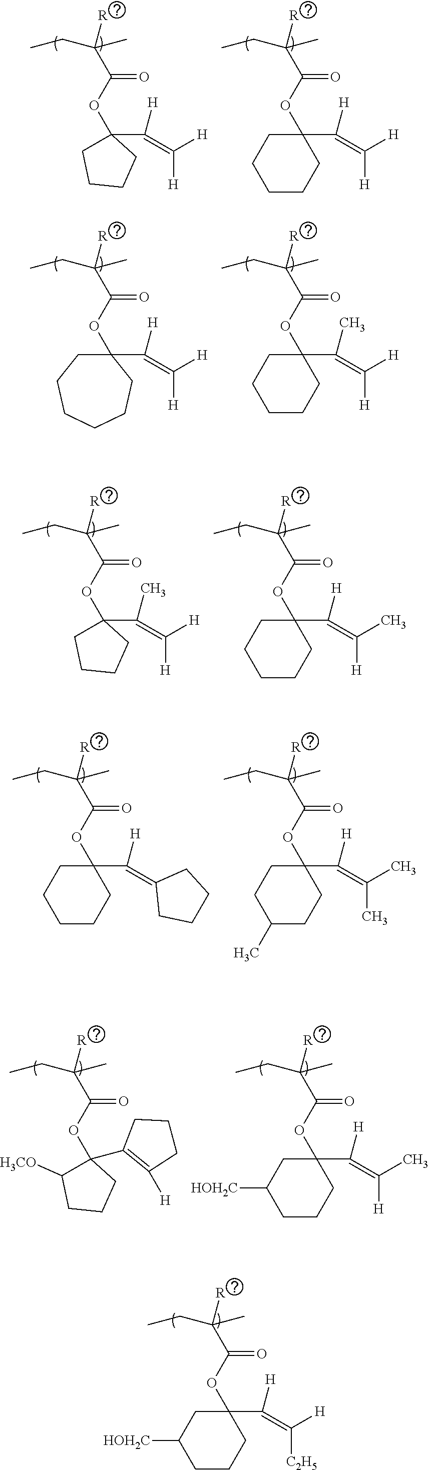

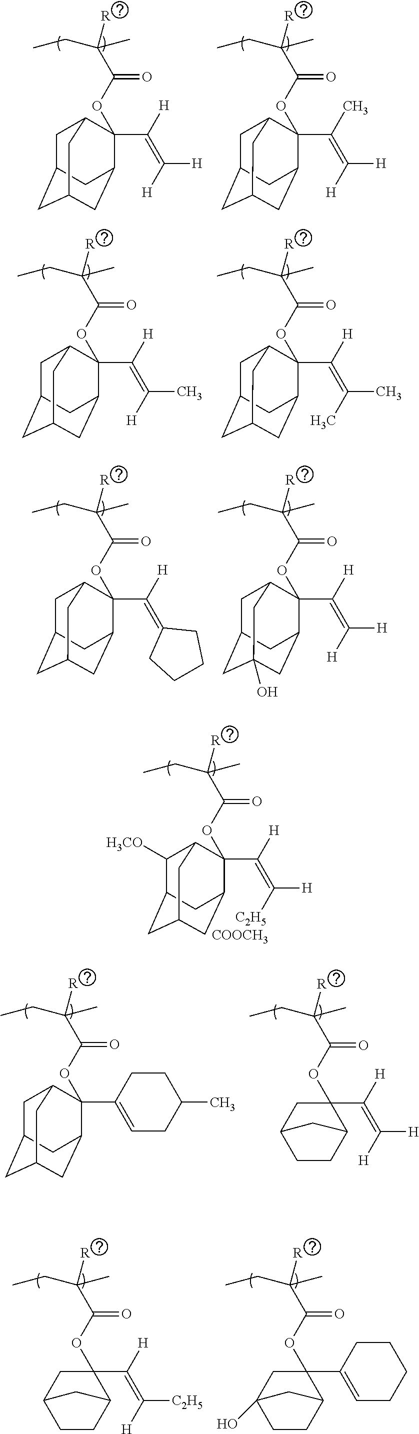

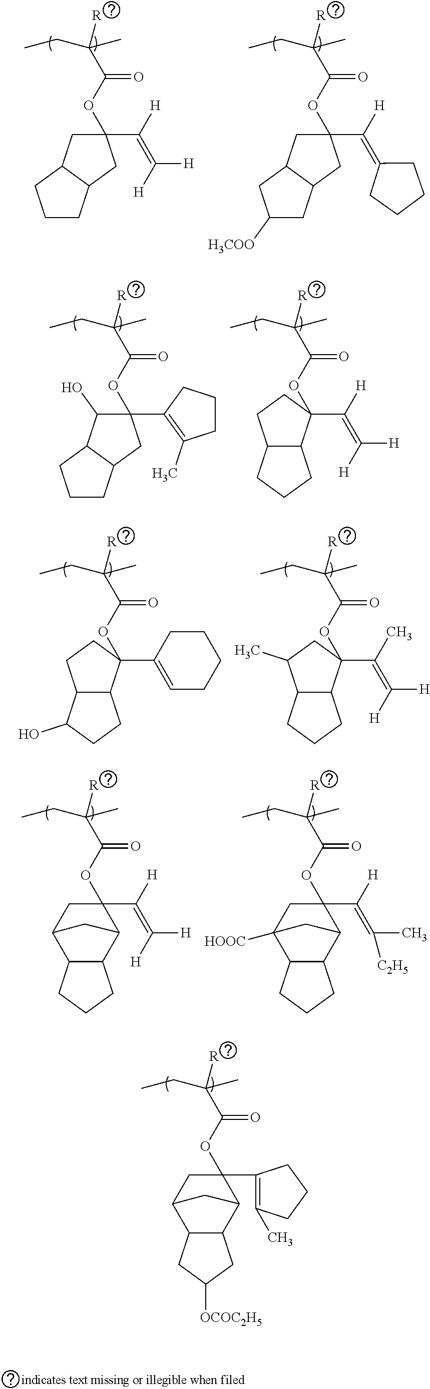

The constitutional unit (a1) contained in the component (A1) may be one kind or may be two or more kinds.

The constitutional unit (a1) is more preferably a constitutional unit represented by General Formula (a1-1) since lithography characteristics (sensitivity, shape, and the like) depending on an electron beam or EUV can be more easily increased.

The proportion of the constitutional unit (a1) in the component (A1) is preferably in a range of 0% to 50% by mole, more preferably in a range of 0% to 40% by mole, still more preferably in a range of 0% to 30% by mole, and particularly preferably in a range of 0% to 60% by mole, with respect to the total (100% by mole) of all constitutional units constituting the component (A1).

It is preferable that the component (A1) not have the constitutional unit (a1).

The proportion of the total of the constitutional unit (a031), the constitutional unit (a032), and the constitutional unit (a1) in the component (A1) is preferably in a range of 10% to 90% by mole, more preferably in a range of 20% to 80% by mole, still more preferably in a range of 30% to 70% by mole, and particularly preferably 40% to 60% by mole, with respect to the total amount (100% by mole) of all constitutional units constituting the component (A1).

In regard to constitutional unit (a2):

The component (A1) may further have a constitutional unit (a2) (however, a constitutional unit corresponding to the constitutional unit (a1) is excluded) containing a lactone-containing cyclic group, an —SO₂—-containing cyclic group, or a carbonate-containing cyclic group.

The description for the constitutional unit (a2) is the same as the description of the constitutional unit (a2) in the first aspect.

The constitutional unit (a2) contained in the component (A1) may be one kind or may be two or more kinds.

In a case where the component (A1) has the constitutional unit (a2), the proportion of the constitutional unit (a2) is preferably in a range of 5% to 60% by mole, more preferably in a range of 10% to 60% by mole, still more preferably in a range of 20% to 60% by mole, and particularly preferably in a range of 30% to 60% by mole with respect to the total (100% by mole) of all constitutional units constituting the component (A1).

In a case where the proportion of the constitutional unit (a2) is equal to or larger than the lower limit value of the preferred range, the effect obtained by allowing the constitutional unit (a2) to be contained can be sufficiently achieved by the effect described above. In a case where the proportion of the constitutional unit (a2) is equal to or smaller than the upper limit value of the preferred range, balance with other constitutional units can be obtained, and various lithography characteristics are improved.

### In Regard to Constitutional Unit (a3):

The component (A1) may further have a constitutional unit (a3) (however, a constitutional unit corresponding to the constitutional unit (a1) or the constitutional unit (a2) is excluded) containing a polar group-containing aliphatic hydrocarbon group.

The description for the constitutional unit (a3) is the same as the description of the constitutional unit (a3) in the first aspect.

The constitutional unit (a3) contained in the component (A1) may be one kind or may be two or more kinds.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 20% by mole, with respect to the total (100% by mole) of all constitutional units constituting the component (A1).

In a case where the proportion of the constitutional unit (a3) is equal to or larger than the lower limit value of the preferred range, the effect obtained by allowing the constitutional unit (a3) to be contained can be sufficiently achieved by the effect described above. In a case where the proportion of the constitutional unit (a3) is equal to or smaller than the upper limit value of the preferred range, balance with other constitutional units can be obtained, and various lithography characteristics are improved.

### In Regard to Constitutional Unit (a4):

The component (A1) may further have a constitutional unit (a4) containing an acid non-dissociable aliphatic cyclic group.

The description for the constitutional unit (a4) is the same as the description of the constitutional unit (a4) in the first aspect.

The constitutional unit (a4) contained in the component (A1) may be one kind or may be two or more kinds.

In a case where the component (A1) has the constitutional unit (a4), the proportion of the constitutional unit (a4) is preferably in a range of 1% to 40% by mole and more preferably in a range of 5% to 20% by mole, with respect to the total (100% by mole) of all constitutional units constituting the component (A1).

In a case where the proportion of the constitutional unit (a4) is equal to or larger than the lower limit value of the preferred range, the effect that is obtained by allowing the constitutional unit (a4) to be contained can be sufficiently achieved. In a case where the proportion of the constitutional unit (a4) is equal to or smaller than the upper limit value of the preferred range, the balance with other constitutional units is obtained easily.

In Regard to Constitutional Unit (a10):

The constitutional unit (a10) is a constitutional unit represented by General Formula (a10-1).

The description for the constitutional unit (a10) is the same as the description of the constitutional unit (a10) in the first aspect.

The constitutional unit (a10) contained in the component (A1) may be one kind or may be two or more kinds.

In a case where the component (A1) has the constitutional unit (a10), the proportion of the constitutional unit (a10) in the component (A1) is preferably 5% to 80% by mole, more preferably 5% to 70% by mole, and still more preferably 10% to 60% by mole, with respect to the total amount (100% by mole) of all constitutional units constituting the component (A1).

In a case where the proportion of the constitutional unit (a10) is equal to or larger than the lower limit value of the above-described preferred range, the sensitivity is more easily increased. In a case where the proportion of the constitutional unit (a10) is equal to or smaller than the upper limit value of the above-described preferred range, the balance between the constitutional unit (a10) and other constitutional units is easily achieved.

### In Regard to Constitutional Unit (St):

The constitutional unit (st) is a constitutional unit derived from styrene or a styrene derivative.

The description for the constitutional unit (st) is the same as the description of the constitutional unit (st) in the first aspect.

The constitutional unit (st) contained in the component (A1) may be one kind or may be two or more kinds.

In a case where the component (A1) has the constitutional unit (st), the proportion of the constitutional unit (st) is preferably in a range of 1% to 30% by mole and more preferably in a range of 3% to 20% by mole with respect to the total (100% by mole) of all constitutional units constituting the component (A1).

The component (A1) may be used alone or in combination of two or more kinds thereof.

Examples of the component (A1) include a polymeric compound having a repeating structure of the constitutional unit (a031) and the constitutional unit (a032), where the component (A1) is preferably a polymeric compound having a repeating structure of the constitutional unit (a031), the constitutional unit (a032), and the constitutional unit (a10).

Among the above, the component (A1) is more preferably a polymeric compound consisting of a repeating structure of the constitutional unit (a031) and the constitutional unit (a032); and a polymeric compound consisting of a repeating structure of the constitutional unit (a031), the constitutional unit (a032), and the constitutional unit (a10).

In the polymeric compound having a repeating structure of the constitutional unit (a031) and the constitutional unit (a032), the proportion of the constitutional unit (a031) is preferably in a range of 10% to 90% by mole, more preferably in a range of 20% to 80% by mole, still more preferably in a range of 30% to 70% by mole, and particularly preferably in a range of 40% to 70% by mole with respect to the total (100% by mole) of all constitutional units constituting the polymeric compound.

The proportion of the constitutional unit (a032) in the polymeric compound described above is preferably in a range of 10% to 90% by mole, more preferably in a range of 20% to 80% by mole, still more preferably in a range of 30% to 70% by mole, and particularly preferably in a range of 30% to 60% by mole, with respect to the total (100% by mole) of all constitutional units constituting the polymeric compound.

In the polymeric compound having a repeating structure of the constitutional unit (a031), the constitutional unit (a032), and the constitutional unit (a10), the proportion of the constitutional unit (a031) is preferably in a range of 10% to 70% by mole, more preferably in a range of 15% to 60% by mole, still more preferably in a range of 20% to 50% by mole, and particularly preferably in a range of 25% to 45% by mole with respect to the total (100% by mole) of all constitutional units constituting the polymeric compound.

The proportion of the constitutional unit (a032) in the polymeric compound described above is preferably in a range of 5% to 60% by mole, more preferably in a range of 10% to 50% by mole, still more preferably in a range of 15% to 40% by mole, and particularly preferably in a range of 15% to 30% by mole, with respect to the total (100% by mole) of all constitutional units constituting the polymeric compound.

In addition, the proportion of the constitutional unit (a10) in the polymeric compound described above is preferably in a range of 10% to 85% by mole, more preferably in a range of 20% to 80% by mole, still more preferably in a range of 30% to 70% by mole, and particularly preferably in a range of 40% to 60% by mole, with respect to the total (100% by mole) of all constitutional units constituting the polymeric compound.

The component (A1) is a polymeric compound having a repeating structure of the constitutional unit (a031), the constitutional unit (a032), and the constitutional unit (a10), where it is preferable that:
the proportion of the constitutional unit (a031) be preferably in a range of 10% to 70% by mole, more preferably in a range of 15% to 60% by mole, still more preferably in a range of 20% to 50% by mole, and particularly preferably in a range of 25% to 40% by mole,
the proportion of the constitutional unit (a032) be preferably in a range of 5% to 60% by mole, more preferably in a range of 10% to 50% by mole, still more preferably in a range of 15% to 40% by mole, and particularly preferably in a range of 15% to 30% by mole,
the proportion of the constitutional unit (a10) be preferably in a range of 10% to 85% by mole, more preferably in a range of 20% to 80% by mole, still more preferably in a range of 30% to 70% by mole, and particularly preferably in a range of 40% to 60% by mole, and
the molar ratio of the constitutional unit (a031) to the constitutional unit (a032) (the constitutional unit (a031):the constitutional unit (a032)) be preferably in a range of 20:80 to 80:20, more preferably in a range of 30:70 to 70:30, still more preferably in a range of 40:60 to 60:40, and particularly preferably in a range of 50:50 to 60:40.

The component (A1) is a copolymer consisting of a repeating structure of the constitutional unit (a031), the constitutional unit (a032), and the constitutional unit (a10), where it is more preferable that:
the proportion of the constitutional unit (a031) be in a range of 25% to 40% by mole with respect to the total (100% by mole) of all constitutional units constituting the polymeric compound,
the proportion of the constitutional unit (a032) be in a range of 15% to 30% by mole with respect to the total (100% by mole) of all constitutional units constituting the polymeric compound,
the proportion of the constitutional unit (a10) be in a range of 40% to 60% by mole with respect to the total (100% by mole) of all constitutional units constituting the polymeric compound,
the proportion of the total of the constitutional unit (a031) and the constitutional unit (a032) be in a range of 40% to 60% by mole with respect to the total (100% by mole) of all constitutional units constituting the polymeric compound, and
the molar ratio of the constitutional unit (a031) to the constitutional unit (a032) (the constitutional unit (a031):the constitutional unit (a032)) be in a range of 50:50 to 60:40.

The component (A1) can be produced by the same method as the production method for the component (A1) described in the first aspect.

The weight-average molecular weight (Mw) (in terms of the polystyrene equivalent value determined by gel permeation chromatography (GPC)) of the component (A1), which is not particularly limited, is preferably in a range of 1,000 to 50,000, more preferably in a range of 2,000 to 30.000, and still more preferably in a range of 3,000 to 20,000. The Mw of the component (A1) is more preferably in a range of 4,000 to 15,000 and particularly preferably in a range of 5,000 to 10,000.

In a case where the Mw of the component (A1) is equal to or smaller than the upper limit value of the above-described preferred range, solubility in a resist solvent sufficient for use as a resist is likely to be obtained. In a case where the Mw of the component (A1) is equal to or larger than the lower limit value of the above-described preferred range, the dry etching resistance and the cross-sectional shape of the resist pattern are good.

The polydispersity (Mw/Mn) of the component (A1) is not particularly limited; however, it is preferably in a range of 1.0 to 4.0, more preferably in a range of 1.0 to 3.0, and particularly preferably in a range of 1.0 to 2.0. In addition, Mn indicates the number-average molecular weight.

### In Regard to Component (A2)

In the resist composition according to the present embodiment, a base material component (hereinafter, referred to as a “component (A2)”) that exhibits changed solubility in a developing solution under action of acid, which does not correspond to the component (A1), may be used in combination as the component (A).

The component (A2) is not particularly limited and may be freely selected and used from a large number of base material components for the chemical amplification-type resist composition known in the related art.

As the component (A2), a polymeric compound or a low molecular weight compound may be used alone or in a combination of two or more kinds thereof.

The proportion of the component (A1) in the component (A) is preferably 25% by mass or more, more preferably 50% by mass or more, still more preferably 75% by mass or more, and may be 100% by mass with respect to the total mass of the component (A). In a case where the proportion of the component (A1) is 25% by mass or more, a resist pattern having various excellent lithography characteristics such as high sensitivity, resolution, and roughness amelioration can be easily formed.

The content of the component (A) in the resist composition according to the present embodiment may be adjusted depending on the resist film thickness to be formed.

### <Acid Generator Component (B)>

The component (B) in the resist composition according to the present embodiment contains the compound (B0) represented by General Formula (b0) (the component (B0)).

The description for the component (B0) is the same as the description of the component (B0) in the first aspect.

In the resist composition according to the present embodiment, the component (B0) may be used alone or may be used in a combination of two or more kinds thereof.

In the resist composition according to the present embodiment, the content of the component (B0) is preferably in a range of 5 to 40 parts by mass, more preferably in a range of 10 to 40 parts by mass, still more preferably in a range of 15 to 40 parts by mass, and particularly preferably in a range of 20 to 35 parts by mass, with respect to 100 parts by mass of the component (A).

In a case where the content of the component (B0) is equal to or larger than the lower limit value of the above-described preferred range, lithography characteristics such as sensitivity, reduction of linewidth roughness (LWR), and a pattern shape are further improved in the resist pattern formation. On the other hand, in a case where the content thereof is equal to or smaller than the upper limit value of the preferred range, a homogeneous solution is easily obtained when each of the components of the resist composition is dissolved in an organic solvent, and the storage stability as a resist composition is further improved.

The proportion of the component (B0) in the total component (B) contained in the resist composition according to the present embodiment is, for example, 50% by mass or more, preferably 70% by mass or more, and more preferably 95% by mass or more. The proportion of the component (B0) in the total component (B) may be 100% by mass.

The component (B) in the resist composition according to the present embodiment may contain an acid generator component (B1) (a component (B1)) other than the above-described component (B0). The description for the component (B1) is the same as the description of the component (B1) in the first aspect.

It is preferable that the resist composition according to the present embodiment not contain the component (B1).

### <Other Components>

The resist composition according to the present embodiment may further contain other components in addition to the component (A) and the component (B) described above. Examples of the other components include the component (D), the component (E), the component (F), and the component (S), which are described above.

### <<Base Component (D)>>

It is preferable that the resist composition according to the present embodiment further contain a base component (a component (D)) that traps (that is, controls the acid diffusion) acid that is generated upon exposure, in addition to the component (A) and the component (B).

The description for the component (D) is the same as the description of the component (D) in the first aspect.

<<At Least One Compound (E) Selected from the Group Consisting of Organic Carboxylic Acid, Phosphorus Oxo Acid, and Derivatives Thereof>>

The resist composition according to the present embodiment may contain the compound (E) (the component (E)) described above.

The description for the component (E) is the same as the description of the component (E) in the first aspect.

### <<Fluorine Additive Component (F)>>

The resist composition according to the present embodiment may contain a fluorine additive component (a component (F)).

The description for the component (F) is the same as the description of the component (F) in the first aspect.

### <<Organic Solvent Component (S)>>

The resist composition according to the present embodiment may be produced by dissolving the resist materials in the organic solvent component (the component (S)).

The description for the component (S) is the same as the description of the component (S) in the first aspect.

As desired, other miscible additives can be also added to the resist composition according to the present embodiment. For example, for improving the performance of the resist film, an additive resin, a dissolution inhibitor, a plasticizer, a stabilizer, a colorant, a halation prevention agent, and a dye can be appropriately contained therein.

After dissolving the resist material in the component (S), the resist composition according to the present embodiment may be subjected to the removal of impurities and the like by using a porous polyimide membrane, a porous polyamideimide membrane, or the like. For example, the resist composition may be filtered using a filter consisting of a porous polyimide membrane, a filter consisting of a porous polyamideimide membrane, or a filter consisting of a porous polyimide membrane and a porous polyamideimide membrane. Examples of the porous polyimide membrane and the porous polyamideimide membrane include those described in Japanese Unexamined Patent Application, First Publication No. 2016-155121.

The resist composition according to the present embodiment described above contains a resin component (A1) having the constitutional unit (a031) and the constitutional unit (a032), and the compound (B0) (the component (B0)). In the resist composition in the related art, there is a trade-off relationship among sensitivity, roughness, and pattern shape, and thus there is a tendency that in a case where any characteristic is improved, the other characteristics are deteriorated. However, in the resist composition according to the present embodiment, it is possible to eliminate the trade-off relationship and favorably maintain all of the sensitivity, the roughness, and the pattern shape. The reason all of the sensitivity, the roughness, and the pattern shape are maintained favorably in the resist composition according to the present embodiment is presumed to be as follows.

The component (B0) contains an iodine atom in the anion moiety, whereby the sensitivity to EUV, EB, and the like is improved. In addition, the solubility in a developing solution is appropriately adjusted, and the deterioration of roughness is suppressed.

In a case where the constitutional unit (a032) has an acid-dissociable group containing an aromatic ring, the deprotection rate is increased. Accordingly, in a case where the resin component (A1) has the constitutional unit (a032), the sensitivity is improved.

Further, in a case where the resin component (A1) has the constitutional unit (a031), the uniformity of the resist composition is improved, and the affinity of the resin component (A1) and the component (B0) for a solvent is improved. As a result, it is conceived that it is possible to suppress the roughness and the deterioration of the pattern shape while maintaining the sensitivity improvement due to the component (B0) and the constitutional unit (a032).

### (Method for Forming Resist Pattern)

A method for forming a resist pattern according to the fourth aspect according to the present invention is a method including a step of forming a resist film on a support using the resist composition according to any one of the first to third aspects described above, a step of exposing the resist film, and a step of developing the exposed resist film to form a resist pattern.

Examples of one embodiment of such a method for forming a resist pattern include a method for forming a resist pattern carried out as described below.

First, the resist composition of the above-described embodiment is applied onto a support with a spinner or the like, and a baking (post-apply baking (PAB)) treatment is carried out, for example, at a temperature condition in a range of 80° C. to 150° C. for 40 to 120 seconds, preferably for 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure baking (PEB)) is carried out, for example, under a temperature condition in a range of 80° C. to 150° C. for 40 to 120 seconds and preferably 60 to 90 seconds.

Next, the resist film is subjected to a developing treatment. The developing treatment is carried out using an alkali developing solution in a case of an alkali developing process, and a developing solution containing an organic solvent (organic developing solution) in a case of a solvent developing process.

After the developing treatment, it is preferable to carry out a rinse treatment. As the rinse treatment, water rinsing using pure water is preferable in a case of an alkali developing process, and rinsing using a rinse liquid containing an organic solvent is preferable in a case of a solvent developing process.

In a case of a solvent developing process, after the developing treatment or the rinse treatment, the developing solution or the rinse liquid remaining on the pattern can be removed by a treatment using a supercritical fluid.

After the developing treatment or the rinse treatment, drying is carried out. As desired, baking treatment (post-baking) can be carried out following the developing treatment.

In this manner, a resist pattern can be formed.

The support is not specifically limited and a known support in the related art can be used. For example, substrates for electronic components, and such substrates having a predetermined wiring pattern formed thereon can be used. Specific examples of the material of the substrate include metals such as silicon wafer, copper, chromium, iron and aluminum; and glass. Suitable materials for the wiring pattern include copper, aluminum, nickel, and gold.

In addition, as the support, any support having the above-described substrate on which an inorganic and/or organic film is provided may be used. Examples of the inorganic film include an inorganic antireflection film (an inorganic BARC). Examples of the organic film include an organic antireflection film (organic BARC) and an organic film such as a lower-layer organic film used in a multilayer resist method.

Here, the multilayer resist method is a method in which at least one layer of an organic film (lower-layer organic film) and at least one layer of a resist film (upper-layer resist film) are provided on a substrate, and a resist pattern formed on the upper-layer resist film is used as a mask to carry out patterning of the lower-layer organic film. This method is considered as being capable of forming a pattern with a high aspect ratio. More specifically, in the multilayer resist method, a desired thickness can be ensured by the lower-layer organic film, and as a result, the thickness of the resist film can be reduced, and an extremely fine pattern with a high aspect ratio can be formed.

The multilayer resist method is basically classified into a method in which a double-layer structure consisting of an upper-layer resist film and a lower-layer organic film is formed (double-layer resist method), and a method in which a multilayer structure having at least three layers consisting of an upper-layer resist film, a lower-layer organic film and at least one intermediate layer (thin metal film or the like) provided between the upper-layer resist film and the lower-layer organic film (triple-layer resist method).

The wavelength to be used for exposure is not particularly limited, and the exposure can be carried out using radiation such as an ArF excimer laser, a KrF excimer laser, an F2 excimer laser, an extreme ultraviolet ray (EUV), a vacuum ultraviolet ray (VUV), an electron beam (EB), an X-ray, or a soft X-ray. The resist composition is highly useful for a KrF excimer laser, an ArF excimer laser, EB, or EUV, more useful for an ArF excimer laser, EB, or EUV, and particularly useful for EB or EUV. That is, the method for forming a resist pattern according to the present embodiment is a useful method particularly in a case where the step of exposing the resist film includes an operation of exposing the resist film to extreme ultraviolet (EUV) rays or electron beams (EB).

The exposure method of the resist film may be a general exposure (dry exposure) carried out in air or an inert gas such as nitrogen, or liquid immersion exposure (liquid immersion lithography); however, liquid immersion exposure is more preferable.

Liquid immersion exposure is an exposure method in which the region between the resist film and the lens at the lowermost position of the exposure apparatus is pre-filled with a solvent (liquid immersion medium) that has a larger refractive index than the refractive index of air, and the exposure (immersion exposure) is carried out in this state.

As the liquid immersion medium, a solvent that exhibits a refractive index larger than the refractive index of air but smaller than the refractive index of the resist film to be exposed is preferable. The refractive index of the solvent is not particularly limited as long as it is in the above-described range.

Examples of the solvent which exhibits a refractive index that becomes larger than the refractive index of air but smaller than the refractive index of the resist film include water, a fluorine-based inert liquid, a silicon-based solvent, and a hydrocarbon-based solvent.

Specific examples of the fluorine-based inert liquid include a liquid containing, as a main component, a fluorine-based compound such as C₃HCl₂F₅, C₄F₉OCH₃, C₄F₉OC₂H₅, or C₅H₃F₇, where the boiling point thereof is preferably in a range of 70° to 180° C. and more preferably in a range of 80° to 160° C. A fluorine-based inert liquid having a boiling point in the above-described range is advantageous in that removing the medium used in the liquid immersion after the exposure can be preferably carried out by a simple method.

The fluorine-based inert liquid is particularly preferably a perfluoroalkyl compound in which all of the hydrogen atoms of the alkyl group are substituted with a fluorine atom. Specific examples of these perfluoroalkyl compounds include perfluoroalkyl ether compounds and perfluoroalkyl amine compounds.

Further, specifically, examples of the perfluoroalkyl ether compound include perfluoro(2-butyl-tetrahydrofuran) (boiling point: 102° C.), and examples of the perfluoroalkyl amine compound include perfluorotributyl amine (boiling point: 174° C.).

As the liquid immersion medium, water is preferable in terms of cost, safety, environment, and versatility.

Examples of the alkali developing solution used for a developing treatment in an alkali developing process include a 0.1% to 10% by mass aqueous solution of tetramethylammonium hydroxide (TMAH).

As the organic solvent contained in the organic developing solution, which is used for a developing treatment in a solvent developing process, any one of the conventionally known organic solvents capable of dissolving the component (A) (component (A) prior to exposure) can be appropriately selected from the conventionally known organic solvents. Specific examples of the organic solvent include polar solvents such as a ketone-based solvent, an ester-based solvent, an alcohol-based solvent, a nitrile-based solvent, an amide-based solvent, an ether-based solvent, and a hydrocarbon-based solvent.

A ketone-based solvent is an organic solvent containing C—C(═O)—C in the structure thereof. An ester-based solvent is an organic solvent containing C—C(═O)—O—C in the structure thereof. An alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. A nitrile-based solvent is an organic solvent containing a nitrile group in the structure thereof. An amide-based solvent is an organic solvent containing an amide group in the structure thereof. An ether-based solvent is an organic solvent containing C—O—C in the structure thereof.

Some organic solvents have a plurality of the functional groups which characterize the above-described solvents in the structure thereof. In such a case, the organic solvent can be classified as any type of solvent having a characteristic functional group. For example, diethylene glycol monomethyl ether can be classified as an alcohol-based solvent or an ether-based solvent.

A hydrocarbon-based solvent consists of a hydrocarbon which may be halogenated and does not have any substituent other than a halogen atom. The halogen atom is preferably a fluorine atom.

Among those described above, the organic solvent contained in the organic developing solution is preferably a polar solvent and more preferably a ketone-based solvent, an ester-based solvent, or a nitrile-based solvent.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonyl alcohol, acetylcarbinol, acetophenone, methyl naphthyl ketone, isophorone, propylene carbonate, γ-butyrolactone, and methylamyl ketone (2-heptanone). Among these, the ketone-based solvent is preferably methylamyl ketone (2-heptanone).

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these, the ester-based solvent is preferably butyl acetate.

Examples of the nitrile-based solvent include acetonitrile, propionitrile, valeronitrile, and butyronitrile.

As desired, the organic developing solution may have a conventionally known additive blended. Examples of the additive include surfactants. The surfactant is not particularly limited, and for example, an ionic or non-ionic fluorine-based and/or a silicon-based surfactant can be used. As the surfactant, a non-ionic surfactant is preferable, and a non-ionic fluorine-based surfactant or a non-ionic silicon-based surfactant is more preferable.

In a case where a surfactant is blended, the blending amount thereof is typically in a range of 0.001% to 5% by mass, preferably in a range of 0.005% to 2% by mass, and more preferably in a range of 0.01% to 0.5% by mass with respect to the total amount of the organic developing solution.

The developing treatment can be carried out by a conventionally known developing method. Examples thereof include a method in which the support is immersed in the developing solution for a predetermined time (a dip method), a method in which the developing solution is cast upon the surface of the support by surface tension and maintained for a predetermined time (a puddle method), a method in which the developing solution is sprayed onto the surface of the support (spray method), and a method in which a developing solution is continuously ejected from a developing solution ejecting nozzle and applied onto a support which is scanned at a constant rate while being rotated at a constant rate (dynamic dispense method).

As the organic solvent contained in the rinse liquid used in the rinse treatment after the developing treatment in a case of a solvent developing process, for example, an organic solvent hardly dissolving the resist pattern can be appropriately selected and used, among the organic solvents mentioned as organic solvents that are used for the organic developing solution. In general, at least one kind of solvent selected from a hydrocarbon-based solvent, a ketone-based solvent, an ester-based solvent, an alcohol-based solvent, an amide-based solvent, and an ether-based solvent is used. Among these, at least one kind of solvent selected from a hydrocarbon-based solvent, a ketone-based solvent, an ester-based solvent, an alcohol-based solvent, and an amide-based solvent is preferable, at least one kind of solvent selected from an alcohol-based solvent and an ester-based solvent is more preferable, and an alcohol-based solvent is particularly preferable.

The alcohol-based solvent used for the rinse liquid is preferably a monohydric alcohol of 6 to 8 carbon atoms, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

As the organic solvent, one kind of solvent may be used alone, or two or more kinds of solvents may be used in combination. In addition, an organic solvent other than the above-described examples or water may be mixed thereto. However, in consideration of the development characteristics, the amount of water to be blended in the rinse liquid is preferably 30% by mass or less, more preferably 10% by mass or less, still more preferably 5% by mass or less, and particularly preferably 3% by mass or less with respect to the total amount of the rinse liquid.

A conventionally known additive can be blended with the rinse liquid as necessary. Examples of the additive include surfactants. Examples of the surfactant include the same as those described above, the surfactant is preferably a non-ionic surfactant and more preferably a non-ionic fluorine-based surfactant or a non-ionic silicon-based surfactant.

In a case where a surfactant is blended, the blending amount thereof is typically in a range of 0.001% to 5% by mass, preferably in a range of 0.005% to 2% by mass, and more preferably in a range of 0.01% to 0.5% by mass with respect to the total amount of the rinse liquid.

The rinse treatment using a rinse liquid (washing treatment) can be carried out by a conventionally known rinse method. Examples of the rinse treatment method include a method in which the rinse liquid is continuously ejected and applied onto the support while rotating it at a constant rate (rotational coating method), a method in which the support is immersed in the rinse liquid for a predetermined time (dip method), and a method in which the rinse liquid is sprayed onto the surface of the support (spray method).

According to the method for forming a resist pattern according to the present embodiment described above, since the resist composition described above is used, it is possible to form a resist pattern that has good sensitivity and has good lithography characteristics such as good roughness and a good exposure margin.

Various materials that are used in the resist composition according to the above-described embodiment and the method for forming a resist pattern according to the above-described embodiment (for example, a resist solvent, a developing solution, a rinse liquid, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, where it is most preferable that the impurities be substantially free (below the detection limit of the measuring device).

### EXAMPLES

Hereinafter, the present invention will be described in more detail based on Examples; however, the present invention is not limited to these Examples.

### (Resist Composition According to First Aspect)

### <Production of Polymeric Compound>

Each of polymeric compounds (A1-11) to (A1-16) and (A2-11) to (A2-13) was obtained by carrying out radical polymerization using monomers from which constitutional units constituting each of the polymeric compounds are derived, at a predetermined molar ratio, and then carrying out a deprotection reaction.

The weight-average molecular weight (Mw) and the molecular weight polydispersity (Mw/Mn) of each of the obtained polymeric compounds were determined according to the GPC measurement (in terms of the standard polystyrene equivalent value).

The copolymerization composition ratio (the ratio (molar ratio) of each constitutional unit in the structural formula) of each of the obtained polymeric compounds was determined from the carbon 13 nuclear magnetic resonance spectrum (600 MHz ¹³C-NMR).

Polymeric compound (A1-11): Weight-average molecular weight (Mw): 7,000, molecular weight polydispersity (Mw/Mn): 1.70, l/m=60/40.

Polymeric compound (A1-12): Weight-average molecular weight (Mw): 6,900, molecular weight polydispersity (Mw/Mn): 1.68, l/m=60/40.

Polymeric compound (A1-13): Weight-average molecular weight (Mw): 7,200, molecular weight polydispersity (Mw/Mn): 1.69, l/m=60/40.

Polymeric compound (A1-14): Weight-average molecular weight (Mw): 6,700, molecular weight polydispersity (Mw/Mn): 1.72, l/m=60/40.

Polymeric compound (A1-15): Weight-average molecular weight (Mw): 6,800, molecular weight polydispersity (Mw/Mn): 1.71, l/m=60/40.

Polymeric compound (A1-16): Weight-average molecular weight (Mw): 7,100, molecular weight polydispersity (Mw/Mn): 1.61, l/m=60/40.

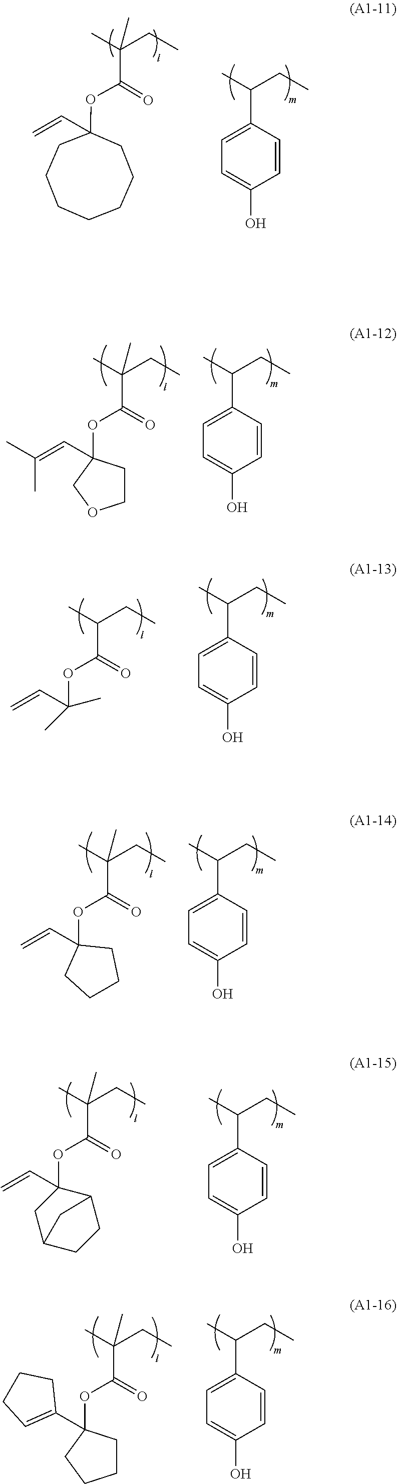

Polymeric compound (A2-11): Weight-average molecular weight (Mw): 7,500, molecular weight polydispersity (Mw/Mn): 1.68, l/m=60/40.

Polymeric compound (A2-12): Weight-average molecular weight (Mw): 6,700, molecular weight polydispersity (Mw/Mn): 1.71, l/m=60/40.

Polymeric compound (A2-13): Weight-average molecular weight (Mw): 7,000, molecular weight polydispersity (Mw/Mn): 1.61, l/m=60/40.

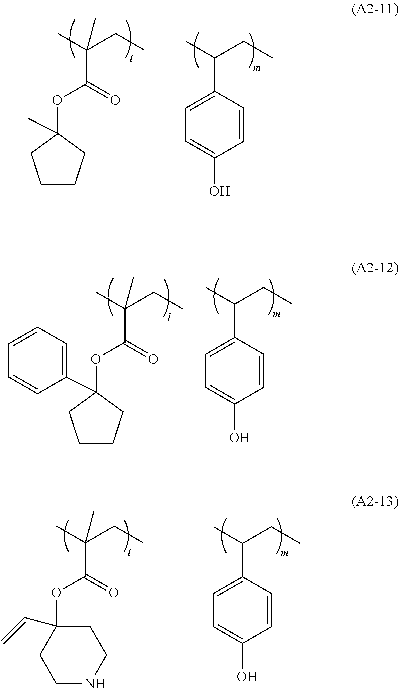

### <Preparation of Resist Composition>

### Examples 1-1 to 1-16 and Comparative Examples 1-1 to 1-5

Each of the components shown in Table 1 and Table 2 was mixed and dissolved to prepare a resist composition of each Example.

TABLE 1

| | Component | Component | Component | Component |
|---|---|---|---|---|
| | (A) | (B) | (D) | (S) |
| Example 1-1 | (A1)-14 | (B0)-11 | (D)-1 | (S)-1 |
| | [100] | [27.7] | [5] | [8000] |
| Example 1-2 | (A1)-14 | (B0)-12 | (D)-1 | (S)-1 |
| | [100] | [25.7] | [5] | [8000] |
| Example 1-3 | (A1)-14 | (B0)-13 | (D)-1 | (S)-1 |
| | [100] | [27.7] | [5] | [8000] |
| Example 1-4 | (A1)-14 | (B0)-14 | (D)-1 | (S)-1 |
| | [100] | [24.1] | [5] | [8000] |
| Example 1-5 | (A1)-14 | (B0)-15 | (D)-1 | (S)-1 |
| | [100] | [24.5] | [5] | [8000] |
| Example 1-6 | (A1)-14 | (B0)-16 | (D)-1 | (S)-1 |
| | [100] | [20.5] | [5] | [8000] |
| Example 1-7 | (A1)-14 | (B0)-17 | (D)-1 | (S)-1 |
| | [100] | [30.1] | [5] | [8000] |
| Example 1-8 | (A1)-14 | (B0)-18 | (D)-1 | (S)-1 |
| | [100] | [27.7] | [5] | [8000] |
| Example 1-9 | (A1)-14 | (B0)-19 | (D)-1 | (S)-1 |
| | [100] | [31.9] | [5] | [8000] |
| Example 1-10 | (A1)-14 | (B0)-110 | (D)-1 | (S)-1 |
| | [100] | [25.4] | [5] | [8000] |
| Example 1-11 | (A1)-14 | (B0)-111 | (D)-1 | (S)-1 |
| | [100] | [25.4] | [5] | [8000] |
| Example 1-12 | (A1)-11 | (B0)-11 | (D)-1 | (S)-1 |
| | [100] | [27.7] | [5] | [8000] |
| Example 1-13 | (A1)-12 | (B0)-11 | (D)-1 | (S)-1 |
| | [100] | [27.7] | [5] | [8000] |
| Example 1-14 | (A1)-13 | (B0)-11 | (D)-1 | (S)-1 |
| | [100] | [27.7] | [5] | [8000] |
| Example 1-15 | (A1)-15 | (B0)-11 | (D)-1 | (S)-1 |
| | [100] | [27.7] | [5] | [8000] |
| Example 1-16 | (A1)-16 | (B0)-11 | (D)-1 | (S)-1 |
| | [100] | [27.7] | [5] | [8000] |

TABLE 2

| | Component | Component | Component | Component |
|---|---|---|---|---|
| | (A) | (B) | (D) | (S) |
| Comparative | (A2)-11 | (B0)-11 | (D)-1 | (S)-1 |
| Example 1-1 | [100] | [27.7] | [5] | [8000] |
| Comparative | (A2)-12 | (B0)-11 | (D)-1 | (S)-1 |
| Example 1-2 | [100] | [27.7] | [5] | [8000] |
| Comparative | (A1)-14 | (B1)-11 | (D)-1 | (S)-1 |
| Example 1-3 | [100] | [20.5] | [5] | [8000] |
| Comparative | (A2)-13 | (B0)-11 | (D)-1 | (S)-1 |
| Example 1-4 | [100] | [27.7] | [5] | [8000] |
| Comparative | (A1)-14 | (B1)-12 | (D)-1 | (S)-1 |
| Example 1-5 | [100] | [17.3] | [5] | [8000] |

In Table 1 and Table 2, each abbreviation has the following meaning. The numerical values in the brackets are blending amounts (parts by mass).

(A1)-11 to (A1)-16: The polymeric compounds (A1-11) to (A1-16) described above.

(A2)-11 to (A2)-13: The polymeric compounds (A2-11) to (A2-13) described above.

(B0)-11 to (B0)-111: Acid generators consisting of compounds each represented by Chemical Formulae (B0-11) to (B0-111).

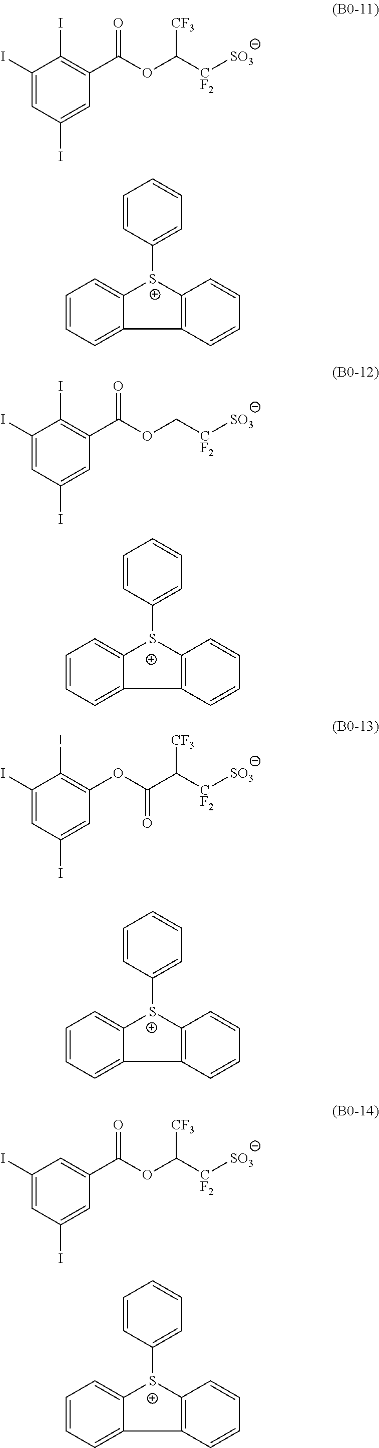

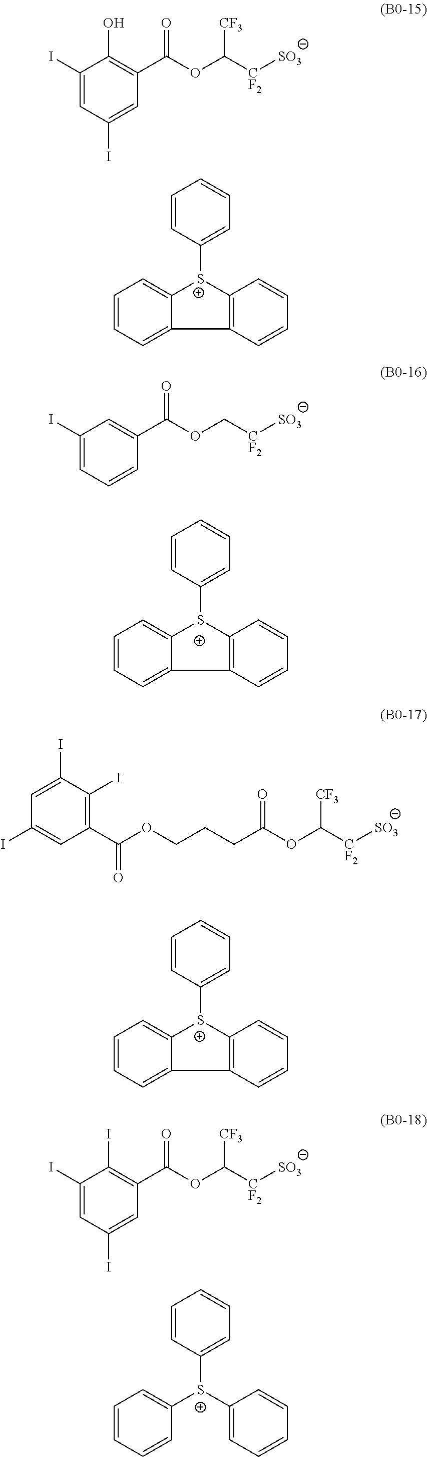

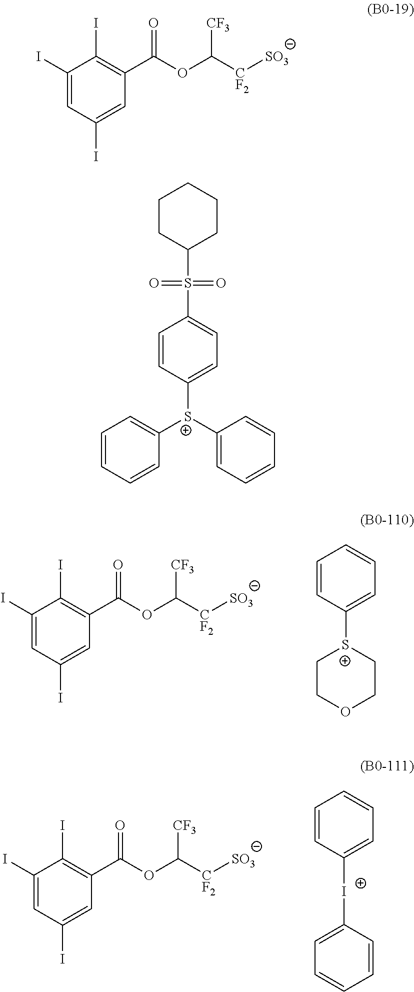

(B1)-11 to (B1)-12: Acid generators consisting of compounds each represented by Chemical Formulae (B1-11) to (B1-12).

(D)-1: An acid diffusion controlling agent composed of a compound represented by Chemical Formula (D-1).

(S)-1: A mixed solvent of propylene glycol monomethyl ether acetate/propylene glycol monomethyl ether=60/40 (in terms of mass ratio)

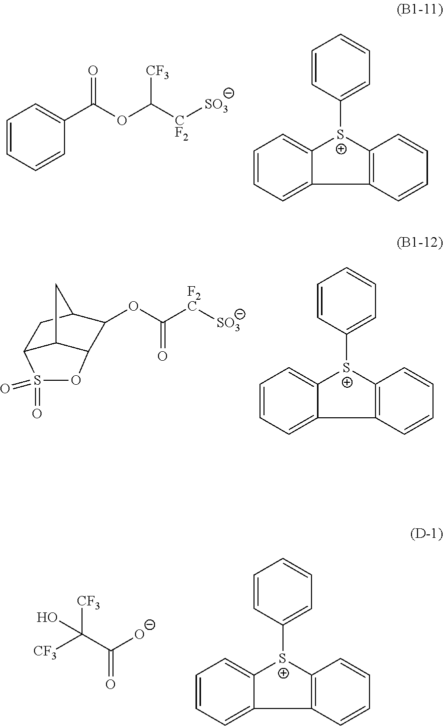

### <Resist Pattern Formation>

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 30 nm.

Next, drawing (exposure) was carried out on the resist film by using an electron beam lithography apparatus JEOL JBX-9300FS (manufactured by JEOL Ltd.), with the target size being set to a 1:1 line and space pattern (hereinafter, referred to as an “LS pattern”) of a line width of 50 nm, at an acceleration voltage of 100 kV. Thereafter, a post-exposure baking (PEB) treatment was carried out on the resist film at 110° C. for 60 seconds.

Subsequently, alkali development was carried out at 23° C. for 60 seconds using a 2.38% by mass tetramethylammonium hydroxide (TMAH) aqueous solution “NMD-3” (product name, manufactured by TOKYO OHKA KOGYO CO., LTD.).

Thereafter, rinsing was carried out with pure water for 15 seconds.

As a result of the above, a 1:1 LS pattern having a line width of 50 nm was formed.

### [Evaluation of Optimum Exposure Amount (Eop)]

According to <Resist pattern formation> described above, an optimum exposure amount Eop (μC/cm²) for forming the LS pattern having the target size was determined. The results are shown in Table 3 and Table 4 as “Eop (μC/cm²)”.

### [Evaluation of Linewidth Roughness (LWR)]

3σ of the LS pattern formed in <Resist pattern formation> described above, which is a scale indicating LWR, was determined. The results are shown in Table 3 and Table 4 as “LWR (nm)”.

“3σ” indicates a triple value (unit: nm) of the standard deviation (σ) determined from measurement results obtained by measuring 400 line positions in the longitudinal direction of the line with a scanning electron microscope (acceleration voltage: 800V, product name: S-9380, manufactured by Hitachi High-Tech Corporation).

The smaller the value of 3σ is, the smaller the roughness in the line side wall is, which means an LS pattern having a more uniform width was obtained.

### [Evaluation of Exposure Margin]

In a case where the irradiation dose was decreased from the optimum exposure amount Eop (μC/cm²) determined by the above-described method so that the space dimension was decreased, the minimum exposure amount with which a pattern could be formed without causing defects such as scum and bridge was denoted by E0. On the other hand, in a case where the irradiation dose was increased from the optimum exposure amount Eop (μC/cm²) so that the space dimension was increased, the maximum exposure amount with which a pattern could be formed without causing defects such as collapse and disconnection was denoted by E1. The ratio (E1/E0) between the minimum exposure amount E0 and the maximum exposure amount E1 is shown in Table 3 and Table 4 as the “Exposure margin”. This means that as the value of the exposure margin becomes larger, the performance of being able to form a pattern becomes higher even in a case where the irradiation dose varies.

TABLE 3

| | PAB | PEB | Eop | LWR | Exposure |
|---|---|---|---|---|---|
| | (° C.) | (° C.) | [μC/cm²] | [nm] | margin |
| Example 1-1 | 110 | 110 | 82 | 4.0 | 4.2 |
| Example 1-2 | 110 | 110 | 83 | 3.9 | 4.3 |
| Example 1-3 | 110 | 110 | 82 | 4.1 | 4.1 |
| Example 1-4 | 110 | 110 | 89 | 4.3 | 3.8 |
| Example 1-5 | 110 | 110 | 89 | 3.9 | 3.9 |
| Example 1-6 | 110 | 110 | 93 | 4.0 | 3.6 |
| Example 1-7 | 110 | 110 | 89 | 4.1 | 4.0 |
| Example 1-8 | 110 | 110 | 83 | 4.0 | 4.1 |
| Example 1-9 | 110 | 110 | 84 | 4.0 | 4.0 |
| Example 1-10 | 110 | 110 | 84 | 4.1 | 4.0 |
| Example 1-11 | 110 | 110 | 83 | 4.2 | 3.9 |
| Example 1-12 | 110 | 110 | 83 | 4.1 | 3.5 |
| Example 1-13 | 110 | 110 | 82 | 4.0 | 4.0 |
| Example 1-14 | 110 | 110 | 83 | 4.2 | 4.1 |
| Example 1-15 | 110 | 110 | 83 | 4.1 | 4.0 |
| Example 1-16 | 110 | 110 | 82 | 4.0 | 4.0 |

TABLE 4

| | PAB | PEB | Eop | LWR | Exposure |
|---|---|---|---|---|---|
| | (° C.) | (° C.) | [μC/cm²] | [nm] | margin |
| Comparative | 110 | 110 | 101 | 4.7 | 2.9 |
| Example 1-1 | | | | | |
| Comparative | 110 | 110 | 89 | 5.7 | 3.2 |
| Example 1-2 | | | | | |
| Comparative | 110 | 110 | 121 | 4.3 | 2.7 |
| Example 1-3 | | | | | |
| Comparative | 110 | 110 | 103 | 4.5 | 2.6 |
| Example 1-4 | | | | | |
| Comparative | 110 | 110 | 114 | 4.4 | 3.0 |
| Example 1-5 | | | | | |

As shown in Table 3 and Table 4, it was confirmed that the resist compositions of Examples 1-1 to 1-16 had high sensitivity and it was possible to form a resist pattern having good LWR and a good exposure margin. On the other hand, in the resist compositions of Comparative Examples 1-1 to 1-5, any one or more of the characteristics of sensitivity, LWR, and exposure margin deteriorated.

### (Resist Composition According to Second Aspect)

### <Production of Polymeric Compound>

Each of polymeric compounds (A1-21) to (A1-214), (A2-21) and (A2-22) was obtained by carrying out radical polymerization using monomers from which constitutional units constituting each of the polymeric compounds were derived, at a predetermined molar ratio, and then carrying out a deprotection reaction.

The weight-average molecular weight (Mw) and the molecular weight polydispersity (Mw/Mn) of each of the obtained polymeric compounds were determined according to the GPC measurement (in terms of the standard polystyrene equivalent value).

The copolymerization composition ratio (the ratio (molar ratio) of each constitutional unit in the structural formula) of each of the obtained polymeric compounds was determined from the carbon 13 nuclear magnetic resonance spectrum (600 MHz ¹³C-NMR).

Polymeric compound (A1-21): Weight-average molecular weight (Mw): 4,900, molecular weight polydispersity (Mw/Mn): 1.71, l/m=50/50.

Polymeric compound (A1-22): Weight-average molecular weight (Mw): 4,900, molecular weight polydispersity (Mw/Mn): 1.69, l/m=50/50.

Polymeric compound (A1-23): Weight-average molecular weight (Mw): 5,000, molecular weight polydispersity (Mw/Mn): 1.70, l/m=50/50.

Polymeric compound (A1-24): Weight-average molecular weight (Mw): 5,000, molecular weight polydispersity (Mw/Mn): 1.71, l/m=50/50.

Polymeric compound (A1-25): Weight-average molecular weight (Mw): 4,800, molecular weight polydispersity (Mw/Mn): 1.69, l/m=50/50.

Polymeric compound (A1-26): Weight-average molecular weight (Mw): 4,900, molecular weight polydispersity (Mw/Mn): 1.70, l/m=70/30.

Polymeric compound (A1-27): Weight-average molecular weight (Mw): 4,900, molecular weight polydispersity (Mw/Mn): 1.69, l/m=30/70.

Polymeric compound (A1-28): Weight-average molecular weight (Mw): 5,000, molecular weight polydispersity (Mw/Mn): 1.70, l/m=60/40.

Polymeric compound (A1-29): Weight-average molecular weight (Mw): 4,900, molecular weight polydispersity (Mw/Mn): 1.71, l/m=40/60.

Polymeric compound (A1-210): Weight-average molecular weight (Mw): 3,000, molecular weight polydispersity (Mw/Mn): 1.69, l/m=50/50.

Polymeric compound (A1-211): Weight-average molecular weight (Mw): 8,000, molecular weight polydispersity (Mw/Mn): 1.71, l/m=50/50.

Polymeric compound (A1-212): Weight-average molecular weight (Mw): 5,100, molecular weight polydispersity (Mw/Mn): 1.70, l/m=50/50.

Polymeric compound (A1-213): Weight-average molecular weight (Mw): 5,100, polydispersity (Mw/Mn): 1.73, l/m/n=40/50/10.

Polymeric compound (A1-214): Weight-average molecular weight (Mw): 5,100, polydispersity (Mw/Mn): 1.72, l/m/n=40/40/20.

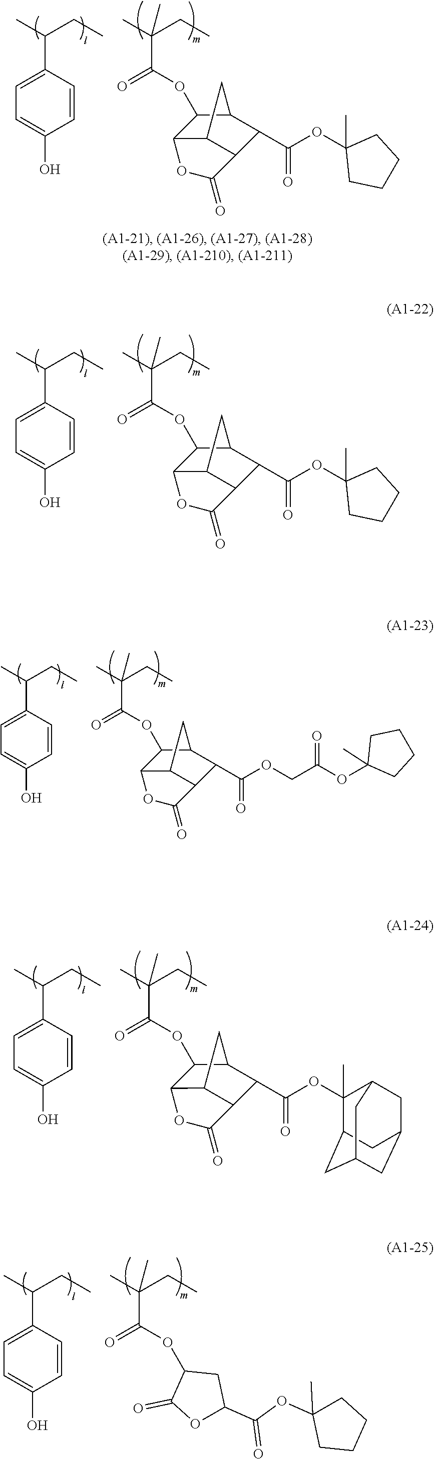

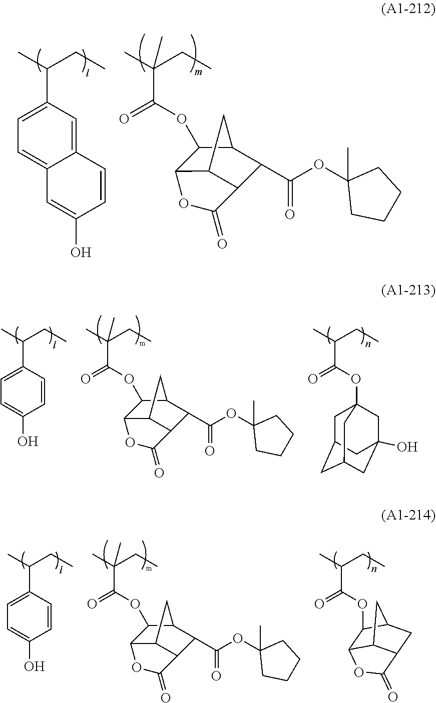

Polymeric compound (A2-21): Weight-average molecular weight (Mw): 5,000, molecular weight polydispersity (Mw/Mn): 1.68, l/m=50/50.

Polymeric compound (A2-22): Weight-average molecular weight (Mw): 5,000, polydispersity (Mw/Mn): 1.72, l/m/n=40/20/40.

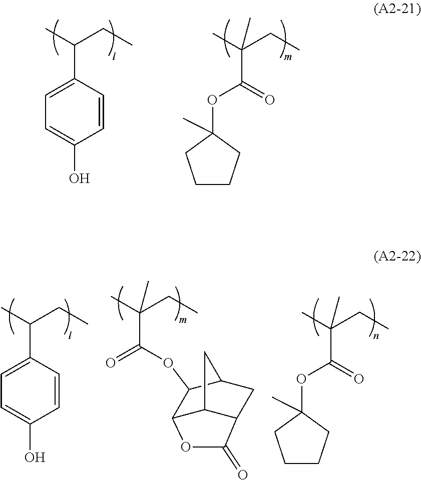

### <Preparation of Resist Composition>

### Examples 2-1 to 2-22 and Comparative Examples 2-1 to 2-3

Each of the components shown in Table 5 to Table 7 was mixed and dissolved to prepare a resist composition of each Example.

TABLE 5

| | Component | Component | Component | Component |
|---|---|---|---|---|
| | (A) | (B) | (D) | (S) |
| Example 2-1 | (A1)-21 | (B0)-21 | (D)-1 | (S)-1 |
| | [100] | [25] | [5] | [8000] |
| Example 2-2 | (A1)-22 | (B0)-21 | (D)-1 | (S)-1 |
| | [100] | [25] | [5] | [8000] |
| Example 2-3 | (A1)-23 | (B0)-21 | (D)-1 | (S)-1 |
| | [100] | [25] | [5] | [8000] |
| Example 2-4 | (A1)-24 | (B0)-21 | (D)-1 | (S)-1 |
| | [100] | [25] | [5] | [8000] |
| Example 2-5 | (A1)-25 | (B0)-21 | (D)-1 | (S)-1 |
| | [100] | [25] | [5] | [8000] |
| Example 2-6 | (A1)-26 | (B0)-21 | (D)-1 | (S)-1 |
| | [100] | [25] | [5] | [8000] |
| Example 2-7 | (A1)-27 | (B0)-21 | (D)-1 | (S)-1 |
| | [100] | [25] | [5] | [8000] |
| Example 2-8 | (A1)-28 | (B0)-21 | (D)-1 | (S)-1 |
| | [100] | [25] | [5] | [8000] |
| Example 2-9 | (A1)-29 | (B0)-21 | (D)-1 | (S)-1 |
| | [100] | [25] | [5] | [8000] |
| Example 2-10 | (A1)-210 | (B0)-21 | (D)-1 | (S)-1 |
| | [100] | [25] | [5] | [8000] |

TABLE 6

| | Component | Component | Component | Component |
|---|---|---|---|---|
| | (A) | (B) | (D) | (S) |
| Example 2-11 | (A1)-211 | (B0)-21 | (D)-1 | (S)-1 |
| | [100] | [25] | [5] | [8000] |
| Example 2-12 | (A1)-212 | (B0)-21 | (D)-1 | (S)-1 |
| | [100] | [25] | [5] | [8000] |
| Example 2-13 | (A1)-213 | (B0)-21 | (D)-1 | (S)-1 |
| | [100] | [25] | [5] | [8000] |
| Example 2-14 | (A1)-214 | (B0)-21 | (D)-1 | (S)-1 |
| | [100] | [25] | [5] | [8000] |
| Example 2-15 | (A1)-21 | (B0)-22 | (D)-1 | (S)-1 |
| | [100] | [23.3] | [5] | [8000] |
| Example 2-16 | (A1)-21 | (B0)-23 | (D)-1 | (S)-1 |
| | [100] | [25.1] | [5] | [8000] |
| Example 2-17 | (A1)-21 | (B0)-24 | (D)-1 | (S)-1 |
| | [100] | [21.8] | [5] | [8000] |
| Example 2-18 | (A1)-21 | (B0)-25 | (D)-1 | (S)-1 |
| | [100] | [22.2] | [5] | [8000] |
| Example 2-19 | (A1)-21 | (B0)-26 | (D)-1 | (S)-1 |
| | [100] | [18.6] | [5] | [8000] |
| Example 2-20 | (A1)-21 | (B0)-27 | (D)-1 | (S)-1 |
| | [100] | [27.3] | [5] | [8000] |
| Example 2-21 | (A1)-21 | (B0)-28 | (D)-1 | (S)-1 |
| | [100] | [28.8] | [5] | [8000] |
| Example 2-22 | (A1)-21 | (B0)-29 | (D)-1 | (S)-1 |
| | [100] | [22.9] | [5] | [8000] |

TABLE 7

| | Component | Component | Component | Component |
|---|---|---|---|---|
| | (A) | (B) | (D) | (S) |
| Comparative | (A2)-21 | (B0)-21 | (D)-1 | (S)-1 |
| Example 2-1 | [100] | [25] | [5] | [8000] |
| Comparative | (A2)-22 | (B0)-21 | (D)-1 | (S)-1 |
| Example 2-2 | [100] | [25] | [5] | [8000] |
| Comparative | (A1)-21 | (B1)-21 | (D)-1 | (S)-1 |
| Example 2-3 | [100] | [15.3] | [5] | [8000] |

In Table 5 to Table 7. each abbreviation has the following meaning. The numerical values in the brackets are blending amounts (parts by mass).

(A1)-21 to (A1)-214: The polymeric compounds (A1-21) to (A1-214) described above.

(A2)-21 to (A2)-22: The polymeric compounds (A2-21) to (A2-22) described above.

(B0)-21 to (B0)-29: Acid generators consisting of compounds each represented by Chemical Formulae (B0-21) to (B0-29).

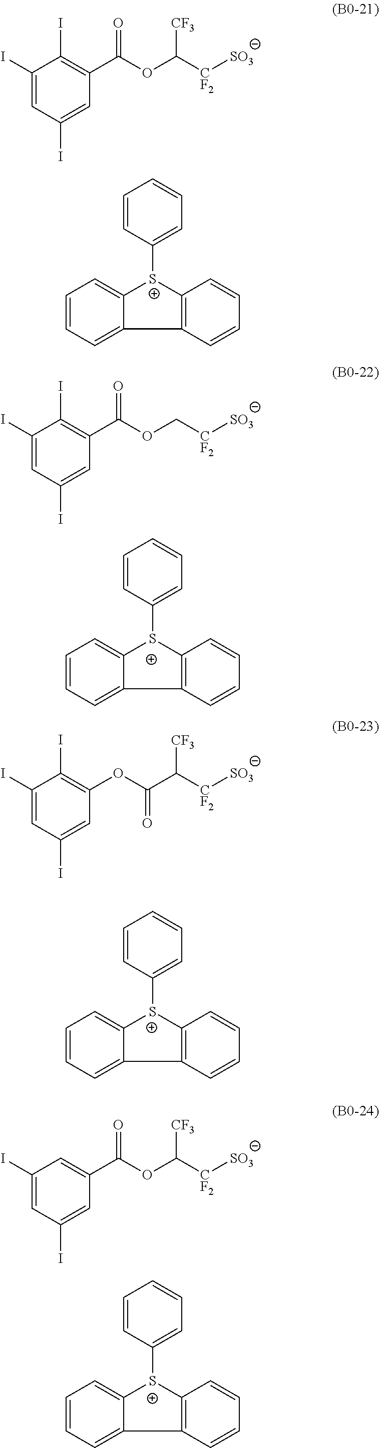

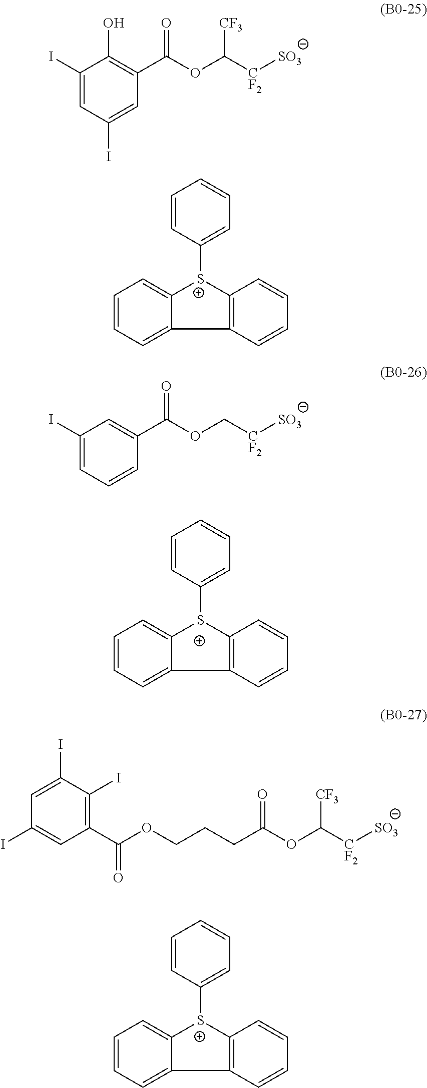

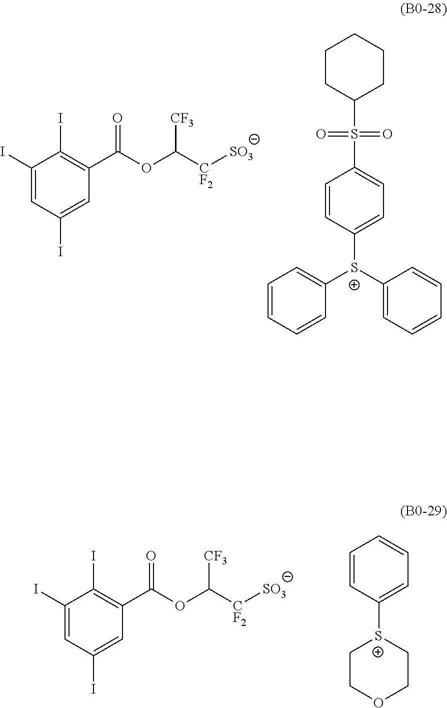

(B1)-21: An acid generator consisting of a compound represented by Chemical Formula (B1-21).

(D)-1: An acid diffusion controlling agent composed of a compound represented by Chemical Formula (D-1).

(S)-1: A mixed solvent of propylene glycol monomethyl ether acetate/propylene glycol monomethyl ether=60/40 (in terms of mass ratio)

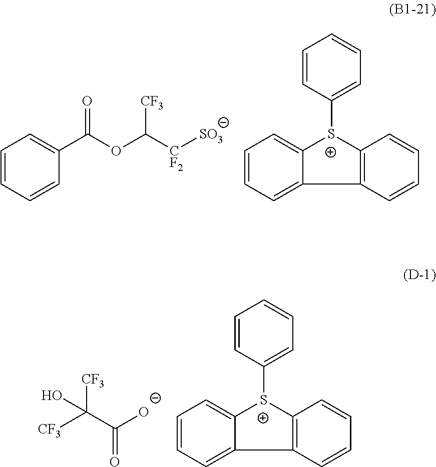

### <Resist Pattern Formation>

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 50 nm.

Next, drawing (exposure) was carried out on the resist film by using an electron beam lithography apparatus JEOL JBX-9300FS (manufactured by JEOL Ltd.), with the target size being set to a 1:1 line and space pattern (hereinafter, referred to as an “LS pattern”) of a line width of 50 nm, at an acceleration voltage of 100 kV. Thereafter, a post-exposure baking (PEB) treatment was carried out on the resist film at 100° C. for 60 seconds.

Subsequently, alkali development was carried out at 23° C. for 60 seconds using a 2.38% by mass tetramethylammonium hydroxide (TMAH) aqueous solution “NMD-3” (product name, manufactured by TOKYO OHKA KOGYO CO., LTD.).

Thereafter, rinsing was carried out with pure water for 15 seconds.

As a result of the above, a 1:1 LS pattern having a line width of 50 nm was formed.

### [Evaluation of Optimum Exposure Amount (Eop)]

According to <Resist pattern formation> described above, an optimum exposure amount Eop (μC/cm²) for forming the LS pattern having the target size was determined. The results are shown in Table 8 to Table 10 as “Eop (μC/cm²)”.

### [Evaluation of Linewidth Roughness (LWR)]

3σ of the LS pattern formed in <Resist pattern formation> described above, which is a scale indicating LWR, was determined by the same method as described above (Example of the resist composition according to the first aspect). The results are shown in Tables 8 to 10 as “LWR (nm)”.

### [Evaluation of Resolution]

The minimum size of the pattern that was resolved without being collapsed in a case where forming an LS pattern by gradually increasing the exposure amount from the optimum exposure amount Eop for forming an LS pattern having a target size according to <Resist pattern formation> described above was determined using a scanning electron microscope S-9380 (manufactured by Hitachi High-Tech Corporation). The results are shown in Tables 8 to 10 as “Resolution (nm)”.

TABLE 8

| | PAB | PEB | Eop | LWR | Resolution |
|---|---|---|---|---|---|
| | (° C.) | (° C.) | [μC/cm²] | [nm] | [nm] |
| Example 2-1 | 110 | 100 | 79.0 | 2.9 | 23.0 |
| Example 2-2 | 110 | 100 | 81.0 | 3.0 | 23.0 |
| Example 2-3 | 110 | 100 | 80.0 | 3.1 | 24.0 |
| Example 2-4 | 110 | 100 | 80.0 | 3.0 | 23.0 |
| Example 2-5 | 110 | 100 | 79.0 | 2.9 | 24.0 |
| Example 2-6 | 110 | 100 | 85.0 | 3.3 | 23.0 |
| Example 2-7 | 110 | 100 | 83.0 | 3.4 | 24.0 |
| Example 2-8 | 110 | 100 | 80.0 | 3.0 | 24.0 |
| Example 2-9 | 110 | 100 | 81.0 | 3.0 | 23.0 |
| Example 2-10 | 110 | 100 | 80.0 | 2.9 | 24.0 |

TABLE 9

| | PAB | PEB | Eop | LWR | Resolution |
|---|---|---|---|---|---|
| | (° C.) | (° C.) | [μC/cm²] | [nm] | [nm] |
| Example 2-11 | 110 | 100 | 81 | 3.1 | 23 |
| Example 2-12 | 110 | 100 | 82 | 3.0 | 24 |
| Example 2-13 | 110 | 100 | 82 | 3.1 | 24 |
| Example 2-14 | 110 | 100 | 85 | 2.9 | 23 |
| Example 2-15 | 110 | 100 | 80 | 3.0 | 24 |
| Example 2-16 | 110 | 100 | 78 | 3.0 | 23 |
| Example 2-17 | 110 | 100 | 90 | 2.9 | 26 |
| Example 2-18 | 110 | 100 | 89 | 2.9 | 26 |
| Example 2-19 | 110 | 100 | 95 | 2.7 | 27 |
| Example 2-20 | 110 | 100 | 80 | 3.0 | 24 |
| Example 2-21 | 110 | 100 | 80 | 2.9 | 23 |
| Example 2-22 | 110 | 100 | 79 | 3.0 | 23 |

TABLE 10

| | PAB | PEB | Eop | LWR | Resolution |
|---|---|---|---|---|---|
| | (° C.) | (° C.) | [μC/cm²] | [nm] | [nm] |
| Comparative | 110 | 100 | 94 | 5.5 | 29 |
| Example 2-1 | | | | | |
| Comparative | 110 | 100 | 96 | 5.4 | 31 |
| Example 2-2 | | | | | |
| Comparative | 110 | 100 | 125 | 4.4 | 30 |
| Example 2-3 | | | | | |

As shown in Table 8 to Table 10, it was confirmed that the resist compositions of Examples 2-1 to 2-22 had higher sensitivity as compared with the resist compositions of Comparative Examples 1 to 3 and made it possible to form a resist pattern having both good LWR and good resolution.

### (Example of Resist Composition According to Third Aspect)

### <Production of Polymeric Compound>

Each of polymeric compounds (A1-31) to (A1-37), (A2-31) and (A2-36) was obtained by carrying out radical polymerization using monomers from which constitutional units constituting each of the polymeric compounds are derived, at a predetermined molar ratio, and then carrying out a deprotection reaction.

The weight-average molecular weight (Mw) and the molecular weight polydispersity (Mw/Mn) of each of the obtained polymeric compounds were determined according to the GPC measurement (in terms of the standard polystyrene equivalent value).

The copolymerization composition ratio (the ratio (molar ratio) of each constitutional unit in the structural formula) of each of the obtained polymeric compounds was determined from the carbon 13 nuclear magnetic resonance spectrum (600 MHz ¹³C-NMR).

Polymeric compound (A1-31): Weight-average molecular weight (Mw): 7,000, polydispersity (Mw/Mn): 1.71, l/m/n=50/30/20.

Polymeric compound (A1-32): Weight-average molecular weight (Mw): 7,000, polydispersity (Mw/Mn): 1.66, l/m/n=50/30/20.

Polymeric compound (A1-33): Weight-average molecular weight (Mw): 6,700, polydispersity (Mw/Mn): 1.69, l/m/n=50/30/20.

Polymeric compound (A1-34): Weight-average molecular weight (Mw): 6,800, polydispersity (Mw/Mn): 1.68, l/m/n=50/30/20.

Polymeric compound (A1-35): Weight-average molecular weight (Mw): 7,100, polydispersity (Mw/Mn): 1.70, l/m/n=50/30/20.

Polymeric compound (A1-36): Weight-average molecular weight (Mw): 7,200, polydispersity (Mw/Mn): 1.70, l/m/n=50/30/20.

Polymeric compound (A1-37): Weight-average molecular weight (Mw): 7,500, polydispersity (Mw/Mn): 1.62, l/m/n=50/30/20.

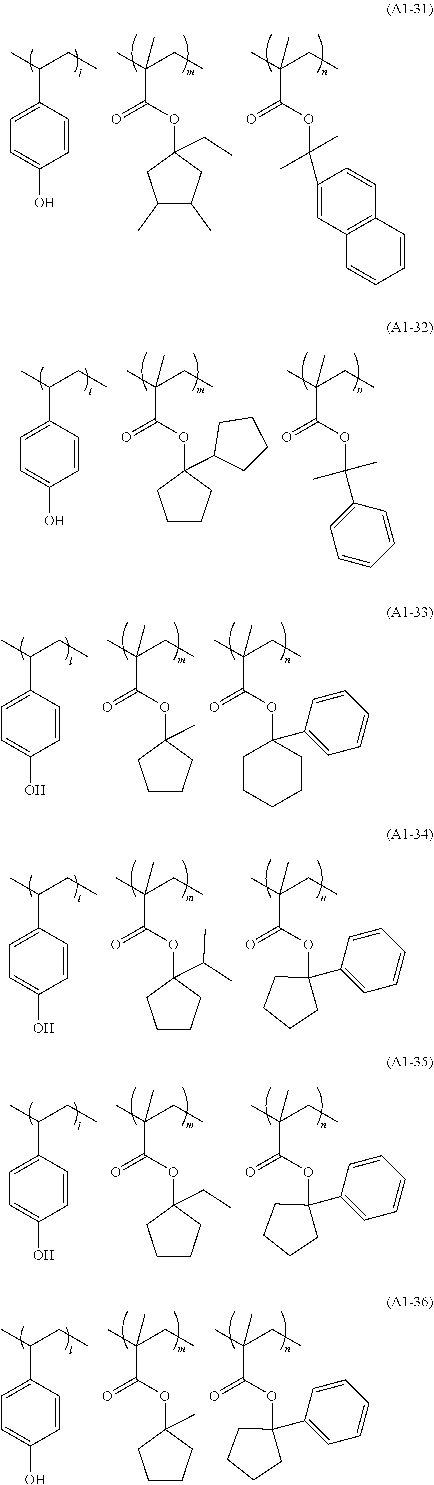

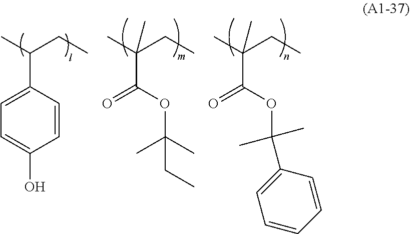

Polymeric compound (A2-31): Weight-average molecular weight (Mw): 6,700, polydispersity (Mw/Mn): 1.68, l/m/n=50/30/20.

Polymeric compound (A2-32): Weight-average molecular weight (Mw): 7,300, polydispersity (Mw/Mn): 1.72, l/m/n=50/30/20.

Polymeric compound (A2-33): Weight-average molecular weight (Mw): 7,200, polydispersity (Mw/Mn): 1.73, l/m/n=50/30/20.

Polymeric compound (A2-34): Weight-average molecular weight (Mw): 6,800, polydispersity (Mw/Mn): 1.68, l/m/n=50/30/20.

Polymeric compound (A2-35): Weight-average molecular weight (Mw): 7,000, polydispersity (Mw/Mn): 1.72, l/m/n=50/30/20.

Polymeric compound (A2-36): Weight-average molecular weight (Mw): 6,400, molecular weight polydispersity (Mw/Mn): 1.77, l/m=50/50.

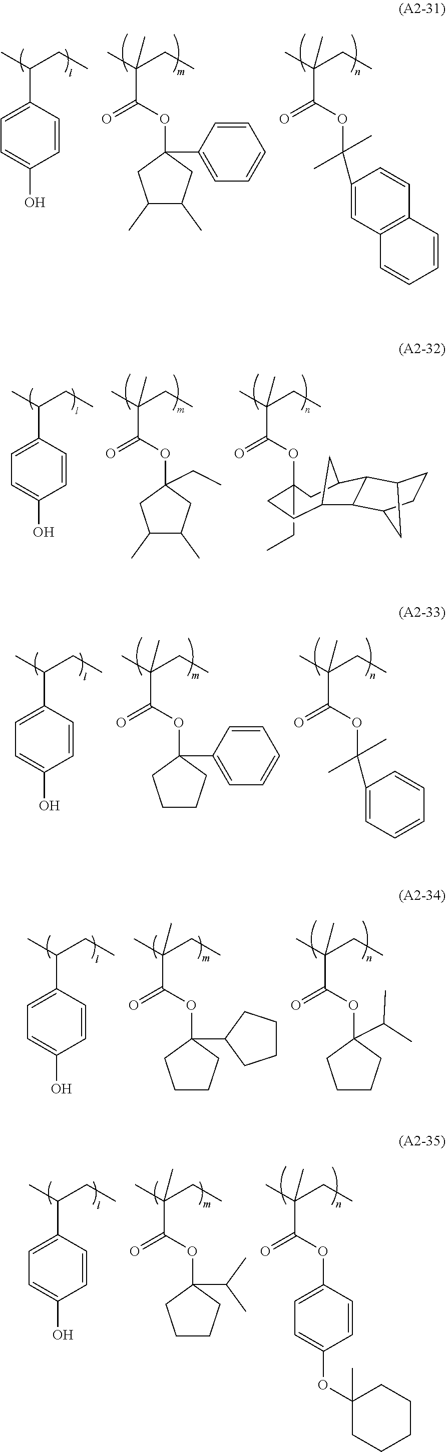

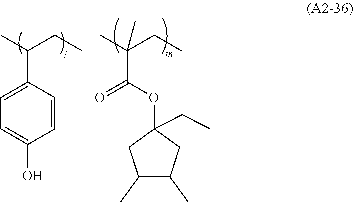

### <Preparation of Resist Composition>

### Examples 3-1 to 3-16 and Comparative Examples 3-1 to 3-8

Each of the components shown in Table 11 and Table 12 was mixed and dissolved to prepare a resist composition of each Example.

TABLE 11

| | Component | Component | Component | Component |
|---|---|---|---|---|
| | (A) | (B) | (D) | (S) |
| Example 3-1 | (A1)-31 | (B0)-31 | (D)-1 | (S)-1 |
| | [100] | [27.8] | [5.0] | [8000] |
| Example 3-2 | (A1)-32 | (B0)-31 | (D)-1 | (S)-1 |
| | [100] | [27.8] | [5.0] | [8000] |
| Example 3-3 | (A1)-33 | (B0)-31 | (D)-1 | (S)-1 |
| | [100] | [27.8] | [5.0] | [8000] |
| Example 3-4 | (A1)-34 | (B0)-31 | (D)-1 | (S)-1 |
| | [100] | [27.8] | [5.0] | [8000] |
| Example 3-5 | (A1)-35 | (B0)-31 | (D)-1 | (S)-1 |
| | [100] | [27.8] | [5.0] | [8000] |
| Example 3-6 | (A1)-36 | (B0)-31 | (D)-1 | (S)-1 |
| | [100] | [27.8] | [5.0] | [8000] |
| Example 3-7 | (A1)-34 | (B0)-32 | (D)-1 | (S)-1 |
| | [100] | [23.3] | [5.0] | [8000] |
| Example 3-8 | (A1)-34 | (B0)-33 | (D)-1 | (S)-1 |
| | [100] | [27.2] | [5.0] | [8000] |
| Example 3-9 | (A1)-34 | (B0)-34 | (D)-1 | (S)-1 |
| | [100] | [22.9] | [5.0] | [8000] |
| Example 3-10 | (A1)-34 | (B0)-35 | (D)-1 | (S)-1 |
| | [100] | [23.3] | [5.0] | [8000] |
| Example 3-11 | (A1)-34 | (B0)-36 | (D)-1 | (S)-1 |
| | [100] | [27.2] | [5.0] | [8000] |
| Example 3-12 | (A1)-34 | (B0)-37 | (D)-1 | (S)-1 |
| | [100] | [22.9] | [5.0] | [8000] |
| Example 3-13 | (A1)-36 | (B0)-32 | (D)-1 | (S)-1 |
| | [100] | [21.1] | [5.0] | [8000] |
| Example 3-14 | (A1)-36 | (B0)-33 | (D)-1 | (S)-1 |
| | [100] | [25.2] | [5.0] | [8000] |
| Example 3-15 | (A1)-36 | (B0)-34 | (D)-1 | (S)-1 |
| | [100] | [31] | [5.0] | [8000] |
| Example 3-16 | (A1)-37. | (B0)-31 | (D)-1 | (S)-1 |
| | [100] | [27.8] | [5.0] | [8000] |

TABLE 12

| | Component | Component | Component | Component |
|---|---|---|---|---|
| | (A) | (B) | (D) | (S) |
| Comparative | (A2)-31 | (B0)-31 | (D)-1 | (S)-1 |
| Example 3-1 | [100] | [27.8] | [5.0] | [8000] |
| Comparative | (A2)-32 | (B0)-31 | (D)-1 | (S)-1 |
| Example 3-2 | [100] | [27.8] | [5.0] | [8000] |
| Comparative | (A2)-33 | (B0)-31 | (D)-1 | (S)-1 |
| Example 3-3 | [100] | [27.8] | [5.0] | [8000] |
| Comparative | (A2)-34 | (B0)-31 | (D)-1 | (S)-1 |
| Example 3-4 | [100] | [27.8] | [5.0] | [8000] |
| Comparative | (A2)-35 | (B0)-31 | (D)-1 | (S)-1 |
| Example 3-5 | [100] | [27.8] | [5.0] | [8000] |
| Comparative | (A1)-34 | (B1)-31 | (D)-1 | (S)-1 |
| Example 3-6 | [100] | [15.3] | [5.0] | [8000] |
| Comparative | (A1)-36 | (B1)-31 | (D)-1 | (S)-1 |
| Example 3-7 | [100] | [15.3] | [5.0] | [8000] |
| Comparative | (A2)-36 | (B0)-31 | (D)-1 | (S)-1 |
| Example 3-8 | [100] | [27.8] | [5.0] | [8000] |

In Table 11 and Table 12, each abbreviation has the following meaning. The numerical values in the brackets are blending amounts (parts by mass).

(A1)-31 to (A1)-37: The polymeric compounds (A1-31) to (A1-37) described above.

(A2)-31 to (A2)-35: The polymeric compounds (A2-31) to (A2-35) described above.

(B0)-31 to (B0)-36: Acid generators consisting of compounds each represented by Chemical Formulae (B0-31) to (B0-36).

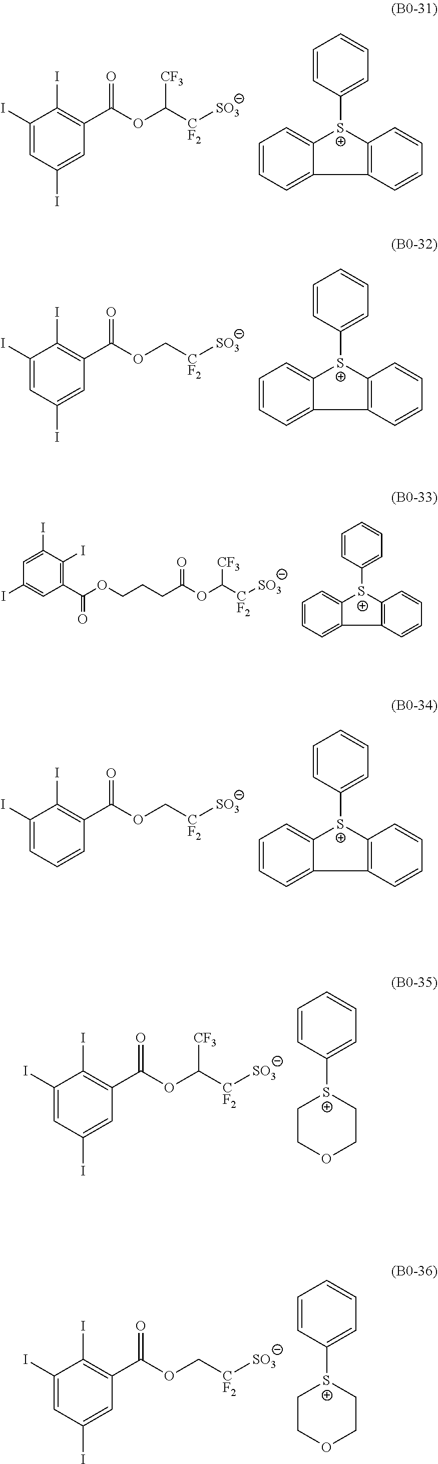

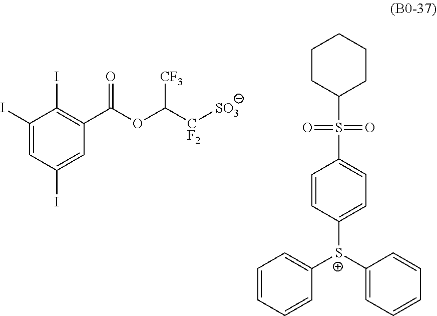

(B1)-31: An acid generator consisting of a compound represented by Chemical Formula (B1-31).

(D)-1: An acid diffusion controlling agent composed of a compound represented by Chemical Formula (D-1).

(S)-1: A mixed solvent of propylene glycol monomethyl ether acetate/propylene glycol monomethyl ether=60/40 (in terms of mass ratio)

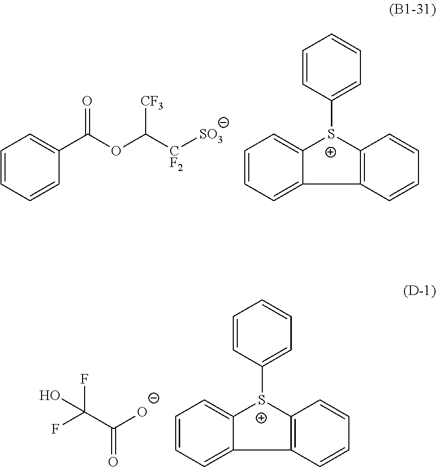

### <Resist Pattern Formation>

The resist composition of each Example was applied onto an 8-inch silicon substrate which had been subjected to a hexamethyldisilazane (HMDS) treatment using a spinner, the coated wafer was subjected to a pre-baking (PAB) treatment on a hot plate at a temperature of 110° C. for 60 seconds so that the coated wafer was dried to form a resist film having a film thickness of 50 nm.

Next, drawing (exposure) was carried out on the resist film by using an electron beam lithography apparatus JEOL JBX-9300FS (manufactured by JEOL Ltd.), with the target size being set to a 1:1 line and space pattern (hereinafter, referred to as an “LS pattern”) of a line width of 50 nm, at an acceleration voltage of 100 kV. Thereafter, a post-exposure baking (PEB) treatment was carried out on the resist film at 110° C. for 60 seconds.

Subsequently, alkali development was carried out at 23° C. for 60 seconds using a 2.38% by mass tetramethylammonium hydroxide (TMAH) aqueous solution “NMD-3” (product name, manufactured by TOKYO OHKA KOGYO CO., LTD.).

Thereafter, rinsing was carried out with pure water for 15 seconds.

As a result of the above, a 1:1 LS pattern having a line width of 50 nm was formed.

### [Evaluation of Optimum Exposure Amount (Eop)]

According to <Resist pattern formation> described above, an optimum exposure amount Eop (μC/cm²) for forming the LS pattern having the target size was determined. The results are shown in Table 13 and Table 14 as “Eop (μC/cm²)”.

### [Evaluation of Linewidth Roughness (LWR)]

3σ of the LS pattern formed in <Resist pattern formation> described above, which is a scale indicating LWR, was determined by the same method as described above (Example of the resist composition according to the first aspect). The results are shown in Table 13 and Table 14 as “LWR (nm)”.

### [Evaluation of Pattern Shape]

The shape of the LS pattern formed according to <Resist pattern formation> described above was observed with a length-measuring scanning electron microscope (SEM, acceleration voltage 800 V, trade name: SU-8000, manufactured by Hitachi High-Tech Corporation), and Table 13 and Table 14 shows the results as “Shape”.

TABLE 13

| | PAB | PEB | Eop | LWR | |
|---|---|---|---|---|---|
| | (° C.) | (° C.) | [μC/cm²] | [nm] | Shape |
| Example 3-1 | 110 | 110 | 91 | 4.6 | Rectangular |
| Example 3-2 | 110 | 110 | 87 | 5.0 | Rectangular |
| Example 3-3 | 110 | 110 | 89 | 4.9 | Rectangular |
| Example 3-4 | 110 | 110 | 85 | 4.4 | Rectangular |
| Example 3-5 | 110 | 110 | 88 | 4.7 | Rectangular |
| Example 3-6 | 110 | 110 | 86 | 4.5 | Rectangular |
| Example 3-7 | 110 | 110 | 87 | 4.5 | Rectangular |
| Example 3-8 | 110 | 110 | 88 | 4.7 | Rectangular |
| Example 3-9 | 110 | 110 | 95 | 4.5 | Rectangular |
| Example 3-10 | 110 | 110 | 84 | 4.6 | Rectangular |
| Example 3-11 | 110 | 110 | 85 | 4.7 | Rectangular |
| Example 3-12 | 110 | 110 | 88 | 4.4 | Rectangular |
| Example 3-13 | 110 | 110 | 86 | 4.5 | Rectangular |
| Example 3-14 | 110 | 110 | 86 | 4.8 | Rectangular |
| Example 3-15 | 110 | 110 | 95 | 4.5 | Rectangular |
| Example 3-16 | 110 | 110 | 84 | 5.3 | Rectangular |

TABLE 14

| | PAB | PEB | Eop | LWR | |
|---|---|---|---|---|---|
| | (° C.) | (° C.) | [μC/cm²] | [nm] | Shape |
| Comparative | 110 | 110 | 86 | 5.1 | Tapered |
| Example 3-1 | | | | | |
| Comparative | 110 | 110 | 112 | 4.3 | Rectangular |
| Example 3-2 | | | | | |
| Comparative | 110 | 110 | 84 | 5.4 | Tapered |
| Example 3-3 | | | | | |
| Comparative | 110 | 110 | 115 | 4.8 | Rectangular |
| Example 3-4 | | | | | |
| Comparative | 110 | 110 | 107 | 6.2 | Rectangular |
| Example 3-5 | | | | | |
| Comparative | 110 | 110 | 107 | 4.2 | Rectangular |
| Example 3-6 | | | | | |
| Comparative | 110 | 110 | 108 | 4.1 | Rectangular |
| Example 3-7 | | | | | |
| Comparative | 110 | 110 | 107 | 4.7 | Rectangular |
| Example 3-8 | | | | | |

As shown in Table 13 and Table 14, it was confirmed that the resist compositions of Examples 3-1 to 3-16 made it possible to form a resist pattern having good sensitivity, good LWR, and good pattern shape formation. On the other hand, in Comparative Examples 3-1 to 3-8, any one of the sensitivity, the LWR, and the pattern shape was inferior.

More specifically, in a case where the resist compositions of Examples 3-1 to 3-6 and 3-16 were compared with the resist compositions of Comparative Examples 3-1 to 3-5 and 3-8, the LWR and the pattern shape were deteriorated in the resist compositions of Comparative Examples 3-1 and 3-3, in which the polymeric compounds (A2-31) and (A2-33) having no constitutional unit (a031) were used. The sensitivity deteriorated in the resist compositions of Comparative Examples 3-2, 3-4, 3-5, and 3-8, in which each of the polymeric compounds (A2-32), and (A2-34) to (A2-36) having no constitutional unit (a032) were used.

In a case where the resist compositions of Examples 3-4, 3-6, and 3-7 to 3-15 were compared with the resist compositions of Comparative Examples 6 and 7, the sensitivity deteriorated in the resist compositions of Comparative Examples 3-6 and 3-7, which contained an acid generator consisting of the compound (B1-31) that does not correspond to the compound (B0).

The preferred Examples of the present invention have been described above; however, the present invention is not limited to these Examples. Additions, omissions, substitutions, and other modifications of the configuration can be made without departing from the spirit of the present invention. Accordingly, the present invention is not to be considered as being limited by the foregoing description and is only limited by the scope of the appended claims.

What is claimed is:

1. A resist composition that generates acid upon exposure and exhibits changed solubility in a developing solution under action of acid, the resist composition comprising:
a resin component (A1) that exhibits changed solubility in a developing solution under action of acid; and
an acid generator component (B) that generates acid upon exposure,
wherein the resin component (A1) has a constitutional unit (a01) containing an acid-dissociable group represented by General Formula (a01-r), and
the acid generator component (B) contains a compound (B0) represented by General Formula (b0), 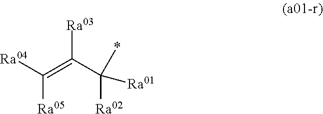
wherein Ra⁰¹ and Ra⁰² each independently represents a saturated aliphatic hydrocarbon group which may have a substituent, and Ra⁰¹ and Ra⁰² may be bonded to each other to form an alicyclic group, where the alicyclic group may contain an oxygen atom or a sulfur atom in a ring skeleton, and part or all of hydrogen atoms contained in the alicyclic group may be substituted with a substituent; Ra⁰³ to Ra⁰⁵ each independently represents a hydrogen atom or an aliphatic hydrocarbon group which may have a substituent, and two or more of Ra⁰³ to Ra⁰⁵ may be bonded to each other to form an alicyclic group, where part or all of hydrogen atoms contained in the alicyclic group may be substituted with a substituent; and * represents a bonding site, 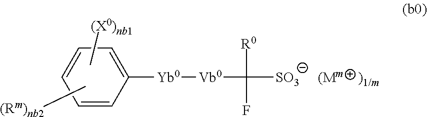
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, wherein 1≤nb1+nb2≤5 is satisfied; Yb⁰ represents a divalent linking group or a single bond; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more.

2. The resist composition according to claim 1, wherein the compound (B0) includes a compound (B01) represented by General Formula (b0-1), 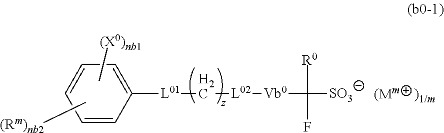
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, wherein 1≤nb1+nb2≤5 is satisfied; L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}), where R^{a}'s each independently represents a hydrogen atom or an alkyl group; z represents an integer in a range of 0 to 10; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more].

3. The resist composition according to claim 1, wherein the constitutional unit (a01) is a constitutional unit derived from a compound represented by General Formula (a01-1), 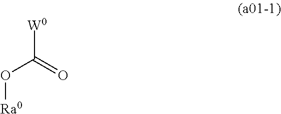
wherein W⁰ represents a polymerizable group-containing group, and Ra⁰ represents an acid-dissociable group represented by General Formula (a01-r).

4. A resist composition that generates acid upon exposure and exhibits changed solubility in a developing solution under action of acid, the resist composition comprising:
a resin component (A1) that exhibits changed solubility in a developing solution under action of acid; and
an acid generator component (B) that generates acid upon exposure,
wherein the resin component (A1) has a constitutional unit (a02) that contains a lactone-containing cyclic group containing an acid-dissociable group, an —SO₂—-containing cyclic group containing an acid-dissociable group, or a carbonate-containing cyclic group containing an acid-dissociable group, and
the acid generator component (B) contains a compound (B0) represented by General Formula (b0), 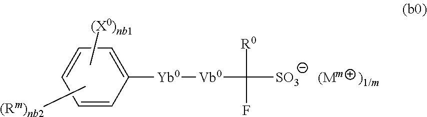
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, wherein 1≤nb1+nb2≤5 is satisfied; Yb⁰ represents a divalent linking group or a single bond; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more.

5. The resist composition according to claim 4, wherein the compound (B0) includes a compound (B01) represented by General Formula (b0-1), 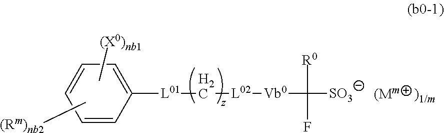
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, wherein 1≤nb1+nb2≤5 is satisfied; L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}), where R^{a}'s each independently represents a hydrogen atom or an alkyl group; z represents an integer in a range of 0 to 10; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more.

6. The resist composition according to claim 4, wherein the constitutional unit (a02) contains a lactone-containing cyclic group represented by General Formula (a02-r1-1) or (a02-r1-2): 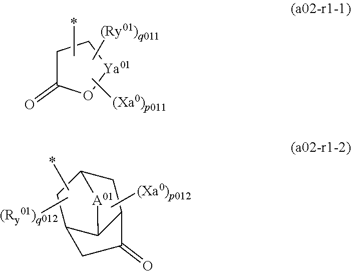
wherein Xa⁰'s each independently represents a group containing an acid-dissociable group; Ya⁰¹ represents a single bond or a linear alkylene group having n⁰¹ carbon atoms, wherein n⁰¹ represents 1 or 2; Ry⁰¹'s each independently represents an alkyl group, an alkoxy group, a halogen atom, a halogenated alkyl group, a hydroxyl group, —COOR⁰″, —OC(═O)R⁰″, a hydroxyalkyl group, or a cyano group; R⁰″ represents a hydrogen atom, an alkyl group, a lactone-containing cyclic group, a carbonate-containing cyclic group, or an —SO₂—-containing cyclic group; A⁰¹ represents an oxygen atom, a sulfur atom, or an alkylene group having 1 to 5 carbon atoms, which may contain an oxygen atom, a sulfur atom; p⁰¹¹ represents an integer in a range of 1 to (3+2n⁰¹), and q⁰¹¹ represents an integer in a range of 0 to (2+2n⁰¹), wherein p⁰¹¹+q⁰¹¹≤3+2n⁰¹ is satisfied (here, and n⁰¹=0 in a case where when Ya⁰¹ is a single bond); p⁰¹² represents an integer in a range of 1 to 7, and q⁰¹² represents an integer in a range of 0 to 6, wherein p⁰¹²+q⁰¹²≤7 is satisfied; and * represents a bonding site.

7. The resist composition according to claim 4, wherein the acid-dissociable group is an acid-dissociable group represented by General Formula (a1-r-2), 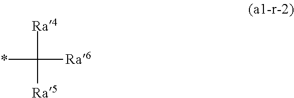
wherein Ra′⁴ to Ra′⁶ each independently represents a hydrocarbon group which may have a substituent, where Ra′⁵ and Ra′⁶ may be bonded to each other to form a ring; and * represents a bonding site.

8. A resist composition that generates acid upon exposure and exhibits changed solubility in a developing solution under the action of an acid, the resist composition comprising:
a resin component (A1) that exhibits changed solubility in a developing solution under the action of an acid; and
an acid generator component (B) that generates acid upon exposure,
wherein the resin component (A1) has a constitutional unit (a031) containing an acid-dissociable group represented by General Formula (a03-r1), and a constitutional unit (a032) containing an acid-dissociable group represented by General Formula (a03-r2), and
the acid generator component (B) contains a compound (B0) represented by General Formula (b0), 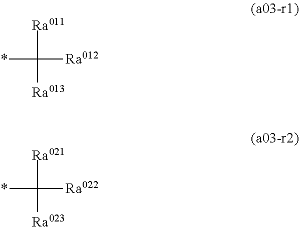
wherein, in General Formula (a03-r1), Ra⁰¹¹ to Ra⁰¹³ each independently represents a saturated aliphatic hydrocarbon group which may have a substituent, where Ra⁰¹² and Ra⁰¹³ may be bonded to each other to form a ring; and in General Formula (a03-r2), Ra⁰²¹ represents a hydrocarbon group containing an aromatic ring, which may have a substituent, and Ra⁰²² and Ra⁰²³ each independently represents a hydrocarbon group which may have a substituent, wherein Ra⁰²² and Ra⁰²³ may be bonded to each other to form a ring; and * represents a bonding site, 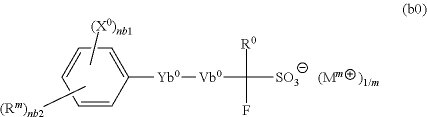
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, wherein 1≤nb1+nb2≤5 is satisfied; Yb⁰ represents a divalent linking group or a single bond; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more.

9. The resist composition according to claim 8, wherein the compound (B0) includes a compound (B01) represented by General Formula (b0-1), 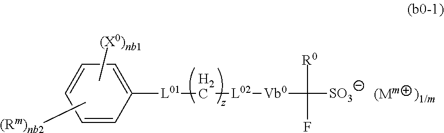
wherein X⁰ represents a bromine atom or an iodine atom; R^{m} represents a hydroxy group, an alkyl group, a fluorine atom, or a chlorine atom; nb1 represents an integer in a range of 1 to 5, and nb2 represents an integer in a range of 0 to 4, where 1≤nb1+nb2≤5 is satisfied; L⁰¹ and L⁰² each independently represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}), where each R^{a} independently represents a hydrogen atom or an alkyl group; z represents an integer in a range of 0 to 10; Vb⁰ represents a single bond, an alkylene group, or a fluorinated alkylene group; R⁰ represents a hydrogen atom, a fluorinated alkyl group having 1 to 5 carbon atoms, or a fluorine atom; and M^{m+} represents an m-valent organic cation, wherein m represents an integer of 1 or more.

10. The resist composition according to claim 8, wherein the acid-dissociable group represented by General Formula (a03-r1) is an acid-dissociable group represented by General Formula (a03-r1-1) or General Formula (a03-r1-2): 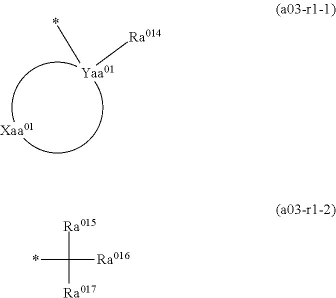
wherein, in General Formula (a03-r1-1), Yaa⁰¹ represents a carbon atom, Xaa⁰¹ represents a group that forms an aliphatic cyclic group together with Yaa⁰¹, and Ra⁰¹⁴ represents a linear, branched, or cyclic alkyl group which may have a substituent; and in General Formula (a03-r1-2), Ra⁰¹⁵ and Ra⁰¹⁶ each independently represents a linear or branched alkyl group which may have a substituent, and Ra⁰¹⁷ represents a saturated aliphatic hydrocarbon group which may have a substituent; and * represents a bonding site.

11. The resist composition according to claim 8, wherein the acid-dissociable group represented by General Formula (a03-r2) is an acid-dissociable group represented by General Formula (a03-r2-1) or General Formula (a03-r2-2): 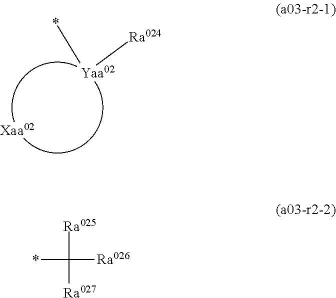
wherein, in General Formula (a03-r2-1), Yaa⁰² represents a carbon atom, Xaa⁰² represents a group that forms an aliphatic cyclic group together with Yaa⁰², and Ra⁰²⁴ represents an aromatic hydrocarbon group which may have a substituent; and in General Formula (a03-r2-2), Ra⁰²⁵ and Ra⁰²⁶ each independently represents a linear or branched alkyl group which may have a substituent, and Ra⁰²⁷ represents an aromatic hydrocarbon group which may have a substituent; and * represents a bonding site.

12. A method for forming a resist pattern, comprising:
forming a resist film on a support using the resist composition according to claim 1;
exposing the resist film; and
developing the exposed resist film to form a resist pattern.
